(19) United States
(12) Patent Application Publication
(10) Pub. No.: US 2023/0285297 A1
(43) Pub. Date: Sep. 14, 2023

(54) METHODS OF PREPARING LIPID NANOPARTICLES

(71) Applicant: ModernaTX, Inc., Cambridge, MA (US)

(72) Inventors: Michael H. SMITH, Cambridge, MA (US); Jason AUER, Cambridge, MA (US); Brie SKINNER, Cambridge, MA (US)

(21) Appl. No.: 17/792,554

(22) PCT Filed: Jan. 29, 2021
(86) PCT No.: PCT/US2021/015888

§ 371 Date: Jul. 13, 2022

Related U.S. Application Data
(60) Provisional application No. 62/968,337, filed on Jan. 31, 2020.

(51) Int. Cl. A61K 9/127; A61K 47/24; A61K 47/28; A61K 31/7105; A61K 47/26; A61K 47/12

(52) U.S. Cl. CPC A61K 9/1272; A61K 9/1271; A61K 9/1277; A61K 9/1278; A61K 31/7105; A61K 47/12; A61K 47/24; A61K 47/26; A61K 47/28

(57) ABSTRACT

The present disclosure provides methods of producing lipid nanoparticle (LNP) formulations and LNP formulations produced by using such methods. The present disclosure further provides therapeutic and diagnostic uses related to the produced LNP formulations.

### RELATED APPLICATIONS

This application claims priority to, and the benefit of, U.S. Provisional Application No. 62/968,337, filed Jan. 31, 2020, the entire content of which is incorporated herein by reference.

### FIELD OF DISCLOSURE

The present disclosure provides novel methods of producing nucleic acid lipid nanoparticles (LNP), the produced formulations thereof, and the related therapeutic and/or diagnostic uses, such as methods involving the nucleic acid lipid nanoparticles to deliver one or more therapeutics and/or prophylactics, such as a nucleic acid, to and/or produce polypeptides in mammalian cells or organs.

### BACKGROUND

The effective targeted delivery of biologically active substances such as small molecule drugs, proteins, and nucleic acids represents a continuing medical challenge. In particular, the delivery of nucleic acids to cells is made difficult by the relative instability and low cell permeability of such species. Thus, there exists a need to develop methods and compositions to facilitate the delivery of therapeutics and prophylactics such as nucleic acids to cells.

Lipid-containing nanoparticles or lipid nanoparticles, liposomes, and lipoplexes have proven effective as transport vehicles into cells and/or intracellular compartments for biologically active substances such as small molecule drugs, proteins, and nucleic acids. Though a variety of such lipid-containing nanoparticles have been demonstrated, improvements in safety, efficacy, and specificity are still lacking.

### SUMMARY

In some aspects, the present disclosure provides a method of preparing an empty lipid nanoparticle (empty LNP), comprising:
i) a mixing step, comprising mixing an ionizable lipid with a first buffering agent, thereby forming the empty LNP, wherein the empty LNP comprises from about 0.1 mol% to about 0.5 mol% of a PEG lipid.

In some aspects, the present disclosure provides a method of preparing an empty lipid nanoparticle (empty LNP), comprising:
i) a mixing step, comprising mixing an ionizable lipid with a first buffering agent, thereby forming the empty LNP.

In some embodiments, the mixing step comprises mixing a lipid solution comprising the ionizable lipid with an aqueous buffer solution comprising the first buffering agent, thereby forming an empty-lipid nanoparticle solution (empty-LNP solution) comprising the empty LNP.

In some aspects, the present disclosure provides an empty LNP comprising from about 0.1 mol% to about 0.5 mol% of a PEG lipid.

In some aspects, the present disclosure provides an empty-LNP solution comprising an empty LNP, wherein the empty LNP comprises from about 0.1 mol% to about 0.5 mol% of a PEG lipid.

In some aspects, the present disclosure provides a method of preparing a loaded lipid nanoparticle (loaded LNP) associated with a nucleic acid, comprising:
ii) a loading step, comprising mixing a nucleic acid with an empty LNP, thereby forming the loaded LNP.

In some aspects, the present disclosure provides a method of preparing a loaded lipid nanoparticle (loaded LNP) associated with a nucleic acid, comprising:
i) a mixing step, comprising mixing an ionizable lipid with a first buffering agent, thereby forming the empty LNP; and
ii) a loading step, comprising mixing a nucleic acid with an empty LNP, thereby forming the loaded LNP.

In some aspects, the present disclosure provides a method of preparing a loaded lipid nanoparticle (loaded LNP) comprising a nucleic acid, comprising:
ii) a loading step, comprising mixing a nucleic acid with an empty LNP, thereby forming the loaded LNP.

In some aspects, the present disclosure provides a method of preparing a loaded lipid nanoparticle (loaded LNP) comprising a nucleic acid, comprising:
i) a mixing step, comprising mixing an ionizable lipid with a first buffering agent, thereby forming the empty LNP; and
ii) a loading step, comprising mixing a nucleic acid with an empty LNP, thereby forming the loaded LNP.

In some aspects, the present disclosure provides a method of preparing a loaded lipid nanoparticle (loaded LNP) comprising a therapeutic agent, comprising:
ii) a loading step, comprising mixing a therapeutic agent with an empty LNP, thereby forming the loaded LNP.

In some aspects, the present disclosure provides a method of preparing a loaded lipid nanoparticle (loaded LNP) comprising a therapeutic agent, comprising:
i) a mixing step, comprising mixing an ionizable lipid with a first buffering agent, thereby forming the empty LNP; and
ii) a loading step, comprising mixing a therapeutic agent with an empty LNP, thereby forming the loaded LNP.

In some embodiments, the loading step comprises mixing the nucleic acid solution comprising the nucleic acid with the empty-LNP solution, thereby forming a loaded lipid nanoparticle solution (loaded-LNP solution) comprising the loaded LNP.

In some aspects, a method of the present disclosure further comprises:
iii) processing the empty-LNP solution or loaded-LNP solution, thereby forming a lipid nanoparticle formulation (LNP formulation).

In some aspects, the present disclosure provides an empty LNP prepared by a method of the disclosure.

In some aspects, the present disclosure provides an empty-LNP solution prepared by a method of the disclosure.

In some aspects, the present disclosure provides a loaded LNP prepared by a method of the disclosure.

In some aspects, the present disclosure provides a loaded-LNP solution prepared by a method of the disclosure.

In some aspects, the present disclosure provides a LNP formulation prepared by a method of the disclosure.

In some aspects, the present disclosure provides a preparation comprising lipid nanoparticles (LNPs) of the disclosure.

In some aspects, the present disclosure provides a preparation comprising lipid nanoparticles (LNPs) of the disclosure, wherein the LNPs are substantially free of a therapeutic or prophylactic agent, and wherein the preparation comprises an acetate buffer having a concentration of from about 2 mM to about 40 mM. In some aspects, the present disclosure provides a preparation comprising lipid nanoparticles (LNPs) of the disclosure, wherein the LNPs are substantially free of a nucleic acid, and wherein the preparation comprises an acetate buffer having a concentration of from about 2 mM to about 40 mM.

In some aspects, the present disclosure provides a preparation comprising lipid nanoparticles (LNPs) of the disclosure, wherein the LNPs do not contain a therapeutic or prophylactic agent, and wherein the preparation comprises an acetate buffer having a concentration of from about 2 mM to about 40 mM. In some embodiments the present disclosure provides a preparation comprising lipid nanoparticles (LNPs) of the disclosure, wherein the LNPs do not contain a nucleic acid, and wherein the preparation comprises an acetate buffer having a concentration of from about 2 mM to about 40 mM.

In some aspects, the present disclosure provides a preparation comprising lipid nanoparticles (LNPs), wherein
(a) the LNPs comprise:
   from about 40 mol% to about 50 mol% ionizable lipid,
   from about 30 mol% to about 50 mol% structural lipid,
   from about 5 mol% to about 20 mol% phospholipid, and
   from about 0.1 mol% to about 1.25 mol% of a PEG lipid;
(b) the LNPs are substantially free of a therapeutic or prophylactic agent; and
(c) the preparation comprises an acetate buffer having a concentration of from about 2 mM to about 40 mM.

In some aspects, the present disclosure provides a preparation comprising lipid nanoparticles (LNPs), wherein
(a) the LNPs comprise:
   from about 40 mol% to about 50 mol% ionizable lipid,
   from about 30 mol% to about 50 mol% structural lipid,
   from about 5 mol% to about 20 mol% phospholipid, and
   from about 0.1 mol% to about 1.25 mol % of a PEG lipid;
(b) the LNPs are substantially free of a nucleic acid; and
(c) the preparation comprises an acetate buffer having a concentration of from about 2 mM to about 40 mM.

In some aspects, the present disclosure provides a preparation comprising lipid nanoparticles (LNPs), wherein
(a) the LNPs comprise:
   from about 40 mol% to about 50 mol% ionizable lipid,
   from about 30 mol% to about 50 mol% structural lipid,
   from about 5 mol% to about 20 mol% phospholipid, and
   from about 0.1 mol% to about 1.25 mol% of a PEG lipid;
(b) the LNPs do not contain a therapeutic or prophylactic agent; and
(c) the preparation comprises an acetate buffer having a concentration of from about 2 mM to about 40 mM.

In some aspects, the present disclosure provides a preparation comprising lipid nanoparticles (LNPs), wherein
(a) the LNPs comprise:
   from about 40 mol% to about 50 mol% ionizable lipid,
   from about 30 mol% to about 50 mol% structural lipid,
   from about 5 mol% to about 20 mol% phospholipid, and
   from about 0.1 mol% to about 1.25 mol% of a PEG lipid;
(b) the LNPs do not contain a nucleic acid; and
(c) the preparation comprises an acetate buffer having a concentration of from about 2 mM to about 40 mM.

In some aspects, the present disclosure provides a method of treating or preventing a disease or disorder, the method comprising administering to a subject in need thereof a loaded LNP of the disclosure.

In some aspects, the present disclosure provides a method of treating or preventing a disease or disorder, the method comprising administering to a subject in need thereof a loaded-LNP solution of the disclosure.

In some aspects, the present disclosure provides a method of treating or preventing a disease or disorder, the method comprising administering to a subject in need thereof a LNP formulation of the disclosure.

In some aspects, the present disclosure provides a loaded LNP for use in treating or preventing a disease or disorder in a subject.

In some aspects, the present disclosure provides a loaded-LNP solution for use in treating or preventing a disease or disorder in a subject.

In some aspects, the present disclosure provides a LNP formulation for use in treating or preventing a disease or disorder in a subject.

In some aspects, the present disclosure provides a use of a loaded LNP in the manufacture of a medicament for treating or preventing a disease or disorder.

In some aspects, the present disclosure provides a use of a loaded-LNP solution in the manufacture of a medicament for treating or preventing a disease or disorder.

In some aspects, the present disclosure provides a pharmaceutical kit comprising an empty LNP, an empty-LNP solution, a loaded LNP, a loaded-LNP solution, or a LNP formulation. For example, in some embodiments, the present disclosure provides a pharmaceutical kit comprising an empty LNP or an empty-LNP solution. For example, in some embodiments, the present disclosure provides a pharmaceutical kit comprising a loaded LNP, a loaded-LNP solution, or a LNP formulation. In some embodiments, the present disclosure provides a pharmaceutical kit comprising a LNP formulation.

In some aspects, the present disclosure provides a pharmaceutical kit comprising an medicament comprising a loaded LNP.

In some aspects, the present disclosure provides a pharmaceutical kit comprising an medicament comprising a preparation comprising lipid nanoparticles (LNPs).

In some aspects, the present disclosure provides a pharmaceutical kit, comprising empty-LNPs and a nucleic acid solution. In some aspects, the present disclosure provides a pharmaceutical kit, comprising an empty-LNP solution and a nucleic acid solution.

In some aspects, the present disclosure provides pharmaceutical kit comprising (a) a first container comprising an empty-LNP of any one of the preceding embodiments; and (b) a second container comprising a solution comprising a therapeutic or prophylactic agent.

In some aspects, the present disclosure provides pharmaceutical kit comprising
(a) a first container comprising an empty-LNP of any one of the preceding embodiments;
(b) a second container comprising a solution comprising a therapeutic or prophylactic agent; and
(c) instructions for combining (e.g., mixing) the content of the first container and the second container.

In some embodiments the first container is a polytetrafluoroethylene (PTFE) bag. In some embodiments the second container is a polytetrafluoroethylene (PTFE) bag. In some embodiments the third container is a polytetrafluoroethylene (PTFE) bag.

In some aspects, the present disclosure provides a container comprising an empty-LNP of the disclosure. In some embodiments, the container is a polytetrafluoroethylene (PTFE) bag.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the disclosure will be apparent from the following detailed description and claims.

### BRIEF DESCRIPTION OF THE DRAWINGS

FIG. 1 is a graph demonstrating the change in diameter of loaded LNPs as a function of mol% of PEG addition.

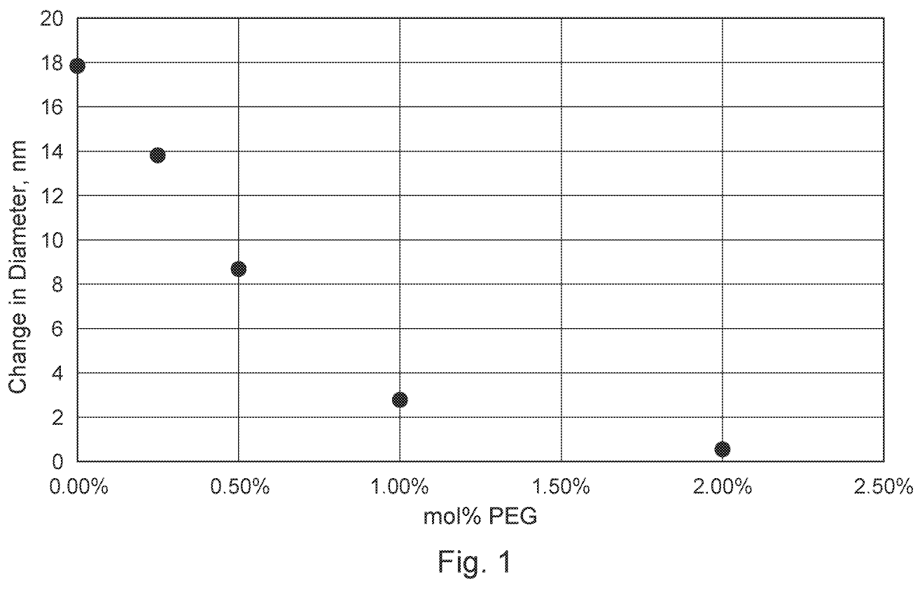

FIG. 2 is a diagram illustrating a general process of preparing an empty-LNP solution comprising an empty LNP.

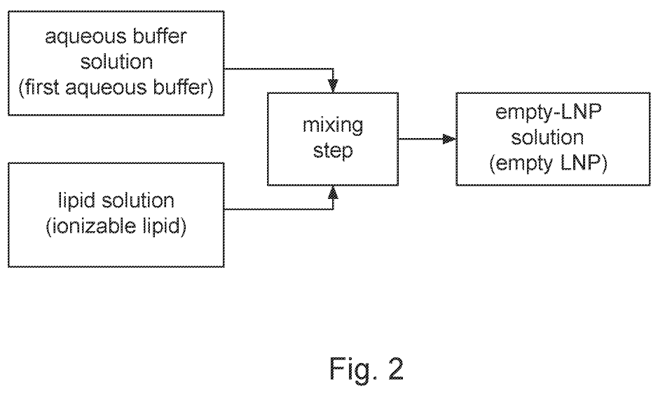

FIG. 3 is a diagram illustrating a general process of an LNP formulation from an empty-LNP solution comprising an empty LNP.

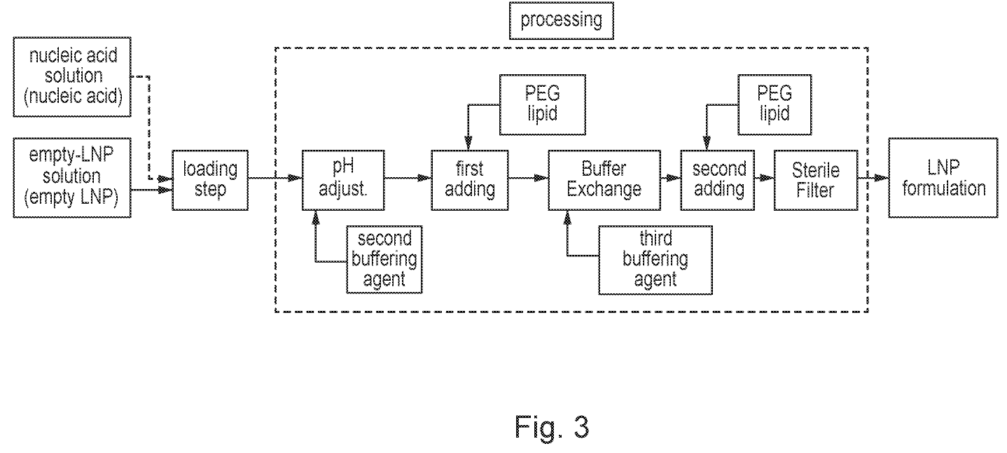

FIG. 4 is a diagram illustrating a general process of preparing an LNP formulation.

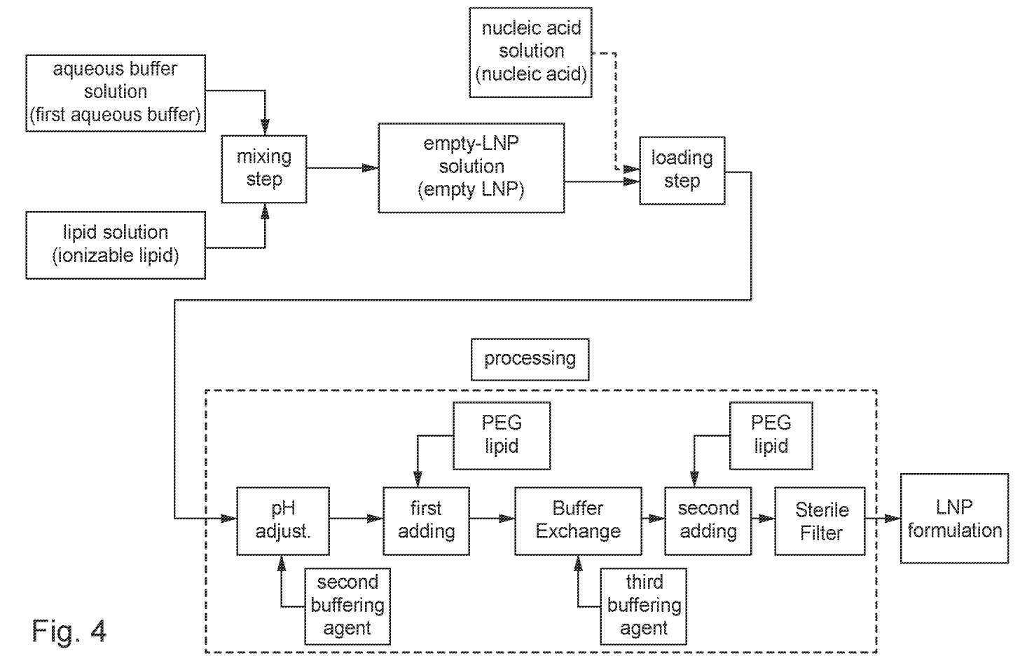

### DETAILED DESCRIPTION

The present disclosure is based, in part, on the discovery that a method of producing lipid nanoparticles (LNPs) or lipid nanoparticle formulations (LNP formulations), as disclosed herein, can influence and/or dictate distribution of certain components within the lipid nanoparticles, and that this distribution can influence and/or dictate physical (e.g., stability) and/or biological (e.g. efficacy, intracellular delivery, immunogenicity) properties of the lipid nanoparticles.

In some embodiments, the methods of the present disclosure mitigate an undesired property change from the produced lipid nanoparticles (LNPs) or lipid nanoparticle formulations (LNP formulations). In some embodiments, the methods of the present disclosure mitigate an undesired property change from the produced lipid nanoparticles (LNP) or lipid nanoparticle formulations (LNP formulations) as compared to the LNPs or LNP formulations produced by different methods (e.g., methods excluding one or more of the steps of the methods of the disclosure, or methods that differ from the methods of the disclosure in at least one step).

In some embodiments, the undesired property change is caused by a stress upon the lipid nanoparticle formulations (LNP formulations) or the lipid nanoparticles (LNPs). In some embodiments, the stress is induced during producing, purifying, packing, storing, transporting, and/or using the lipid nanoparticle formulations (LNP formulations) or lipid nanoparticles. In some embodiments, the stress is heat, shear, excessive agitation, membrane concentration polarization (change in charge state), dehydration, freezing stress, drying stress, freeze/thaw stress, and/or nebulization stress. In some embodiments, the stress is induced during storing lipid nanoparticle formulations (LNP formulations) or lipid nanoparticles (LNPs).

In some embodiments, the undesired property change is a reduction of the physical stability of an LNP formulation. In some embodiments, the undesired property change is an increase of the amount of impurities and/or sub-visible particles, or an increase in the average size of an LNP in an LNP formulation.

In some embodiments, the undesired property change is a reduction of the chemical stability of the LNP formulation. In some embodiments, the undesired property change is a reduction of the integrity of the nucleic acid (e.g., RNA (e.g., mRNA)) in the LNP formulation.

In some embodiments, the undesired property change is a reduction of a biological property of the LNP formulation. In some embodiments, the undesired property change is a reduction of efficacy, intracellular delivery, and/or immunogenicity of the LNP formulation.

In some embodiments, an LNP formulation produced by a method of the present disclosure is more stable (e.g., does not experience an increase in the average size of the LNP over time) than an LNP formulation produced by a different method (e.g., a method excluding one or more of the steps of the methods of the disclosure, or a method that differs from the methods of the disclosure in at least one step).

In some embodiments, an LNP produced by a method of the present disclosure has an average diameter of about 99% or less, about 98% or less, about 97% or less, about 96% or less, about 95% or less, about 90% or less, about 85% or less, about 80% or less, about 75% or less, about 70% or less, about 65% or less, about 60% or less, about 55% or less, about 50% or less, about 40% or less, about 30% or less, about 20% or less, or about 10% or less than the average LNP diameter of the LNP formulation produced by a different method (e.g., a method excluding one or more of the steps of the methods of the disclosure, or a method that differs from the methods of the disclosure in at least one step).

In some embodiments, a lipid nanoparticle (LNP) of the disclosure has an average diameter of about 15 nm to about 150 nm, about 20 nm to about 125 nm, about 25 nm to about 100 nm, about 30 nm to about 80 nm, about 35 nm to about 70 nm, about 40 nm to about 60 nm, or about 45 nm to about 50 nm.

In some embodiments, an empty LNP produced by the method of the present disclosure has an average diameter of about 99% or less, about 98% or less, about 97% or less, about 96% or less, about 95% or less, about 90% or less, about 85% or less, about 80% or less, about 75% or less, about 70% or less, about 65% or less, about 60% or less, about 55% or less, about 50% or less, about 40% or less, about 30% or less, about 20% or less, or about 10% or less than the average diameter of an empty LNP produced by a different method (e.g., a method excluding one or more of the steps of the methods of the disclosure, or a method that differs from the methods of the disclosure in at least one step).

In some embodiments, an empty LNP of the disclosure has an average diameter of about 15 nm to about 150 nm, about 20 nm to about 125 nm, about 25 nm to about 100 nm, about 30 nm to about 80 nm, about 35 nm to about 70 nm, about 40 nm to about 60 nm, or about 45 nm to about 50 nm.

In some embodiments, a LNP formulation produced by a method of the present disclosure has an efficacy, intracellular delivery, and/or immunogenicity that is higher than the efficacy, intracellular delivery, and/or immunogenicity of a LNP formulation produced by a different method (e.g., a method excluding one or more of the steps of the methods of the disclosure, or a method that differs from the methods of the disclosure in at least one step).

In some embodiments, a LNP formulation produced by a method of the present disclosure has an efficacy, intracellular delivery, and/or immunogenicity that is higher than the efficacy, intracellular delivery, and/or immunogenicity of a LNP formulation produced by a different method (e.g., a method excluding one or more of the steps of the methods of the disclosure, or a method that differs from the methods of the disclosure in at least one step) by about 5% or more, about 10% or more, about 15% or more, about 20% or more, about 30% or more, about 40% or more, about 50% or more, about 60% or more, about 70% or more, about 80% or more, or about 90% or more. In some embodiments, a LNP formulation produced by a method of the present disclosure has an efficacy, intracellular delivery, and/or immunogenicity that is higher than the efficacy, intracellular delivery, and/or immunogenicity of a LNP formulation produced by a different method by about 1 fold or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

In some embodiments, a LNP formulation produced by a method of the present disclosure exhibits a nucleic acid expression (e.g., a mRNA expression) higher than the nucleic acid expression (e.g., a mRNA expression) of a LNP formulation produced by a different method (e.g., a method excluding one or more of the steps of the methods of the disclosure, or a method that differs from the methods of the disclosure in at least one step).

In some embodiments, a LNP formulation produced by a method of the present disclosure exhibits a nucleic acid expression (e.g., a mRNA expression) that is higher than the nucleic acid expression (e.g., a mRNA expression) of a LNP formulation produced by a different method (e.g., a method excluding one or more of the steps of the methods of the disclosure, or a method that differs from the methods of the disclosure in at least one step) by about 5% or more, about 10% or more, about 15% or more, about 20% or more, about 30% or more, about 40% or more, about 50% or more, about 60% or more, about 70% or more, about 80% or more, or about 90% or more. In some embodiments, a LNP formulation produced by a method of the present disclosure exhibits a nucleic acid expression (e.g., a mRNA expression) that is higher than the nucleic acid expression (e.g., a mRNA expression) of a LNP formulation produced by a different method by about 1 fold or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

The present invention features novel “bedside” and/or “point-of-care” formulations, whereby mRNA are being encapsulated within vesicles that were prepared at an earlier point in time and that do not comprise a therapeutic agent at the time of their preparation (e.g., empty LNPs). This mode of production offers advantages in the context of clinical supply, as these vesicles (e.g., empty LNPs) may be produced and stored separately prior to combining with mRNA in a clinical setting. Specifically, bedside formulations may promote increased stability since mRNA and empty raw materials (e.g., empty LNPs) can be stored under conditions that are separately optimized for each component. For example, the mRNA can be stored under different conditions than the empty LNP). Process complexity and cost of goods may be reduced since the LNP preparation occurs independent of cargo, enabling a platform approach for multiple mRNA or active agent constructs. The principle of encapsulating a therapeutic agent (e.g. an mRNA) into a preformed nanoparticle (an “empty LNP”) to provide an LNP comprising the therapeutic agent (i.e., a “loaded LNP”) is referred herein to as “post hoc loading” (PHL), “post-hoc addition”, or “post-hoc”.

The present disclosure is based, in part, on efforts exploring the fundamental principles of post hoc loading and investigating the impact and conditions of mRNA encapsulation (i.e., formation of a loaded LNP) at timescales after empty LNP generation. The time of mRNA addition after lipid precipitation was varied by upwards of seven orders of magnitude (e.g., 1 ms to 10,000,000 ms) without detrimentally impacting the physicochemical properties of the loaded LNP (e.g., particle size, encapsulation, morphology, and/or structural integrity). Oligonucleotides are often described as participating in the early particle assembly steps. Outcomes from empirical experiments suggest that mRNA encapsulation may occur at significantly long time periods after lipid precipitation/particle formation, without detrimentally affecting loaded LNP physicochemical properties. These experiments demonstrated that the lipid particle formation and subsequent mRNA encapsulation may be separated into two reaction steps. The concept of post hoc loading as described herein may enable control and/or optimization of each step separately. Further, the post hoc loading may enable mRNA addition at timescales that allow for point-of-care formation of the loaded LNP (e.g., hours, days, months, or years following empty LNP production).

Historically, processes have not been developed to generate pre-formed empty lipid nanoparticles (empty LNPs) at scales appropriate for clinical supply. The present disclosure is based, in part, on efforts to ascertain a multitude of process parameters advantageous for scaled production including, but not limited to, lipid concentrations, PEG-lipid or polymeric lipid quantity, temperature, buffer composition (e.g., ionic strength, pH, counterion), and ethanol content.

The present disclosure is based, in part, on the discovery that methods of producing lipid nanoparticles (LNP) or lipid nanoparticle (LNP) formulations, as disclosed herein, can influence and/or dictate distribution of certain components within the lipid nanoparticles, and that this distribution can influence and/or dictate physical (e.g., stability) and/or biological (e.g. efficacy, intracellular delivery, immunogenicitiy) properties of the lipid nanoparticles.

In some embodiments, the present disclosure yields compositions comprising lipid nanoparticles having an advantageous distribution of components.

In some embodiments, a LNP formulation produced by a method of the present disclosure exhibits a nucleic acid expression (e.g., the mRNA expression) higher than the nucleic acid expression (e.g., the mRNA expression) of a LNP formulation produced by a different method (e.g., a method excluding one or more of the steps of the methods of the disclosure, or a method that differs from the methods of the disclosure in at least one step).

In some embodiments, a LNP formulation produced by the method of the present disclosure exhibits a nucleic acid expression (e.g., the mRNA expression) that is higher than the nucleic acid expression (e.g., the mRNA expression) of a LNP formulation prepared by a different method (e.g., a method excluding one or more of the steps of the methods of the disclosure, or a method that differs from the methods of the disclosure in at least one step) by about 5% or more, about 10% or more about 15% or more, about 20% or more, about 30% or more, about 40% or more, about 50% or more, about 60% or more, about 70% or more, about 80% or more, or about 90% or more. In some embodiments, a LNP formulation produced by the method of the present disclosure exhibits a nucleic acid expression (e.g., the mRNA expression) that is higher than the nucleic acid expression (e.g., the mRNA expression) of a LNP formulation prepared by a different method by about 1 fold or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

### METHODS OF THE PRESENT DISCLOSURE

The present disclosure provides a method of producing an empty lipid nanoparticle (empty LNP), the method comprising: i) a mixing step, comprising mixing an ionizable lipid with a first buffering agent, thereby forming the empty LNP, wherein the empty LNP comprises from about 0.1 mol% to about 0.5 mol% of a PEG lipid or other polymeric lipid.

In some embodiments, the mixing step comprises mixing a lipid solution comprising the ionizable lipid with an aqueous buffer solution comprising the first buffering agent, thereby forming an empty-lipid nanoparticle solution (empty-LNP solution) comprising the empty LNP.

In some aspects, the present disclosure provides a method of preparing a loaded lipid nanoparticle (loaded LNP) associated with a nucleic acid, comprising: ii) a loading step, comprising mixing a nucleic acid with an empty LNP, thereby forming the loaded LNP.

In some embodiments of the methods of the disclosure, the loading step comprises mixing the nucleic acid solution comprising the nucleic acid with the empty-LNP solution, thereby forming a loaded lipid nanoparticle solution (loaded-LNP solution) comprising the loaded LNP.

In some embodiments of the methods of the disclosure, the empty LNP or the empty-LNP solution is subjected to the loading step without holding or storage.

In some embodiments of the methods of the disclosure, the empty LNP or the empty-LNP solution is subjected to the loading step after holding for a period of time.

In some embodiments of the methods of the disclosure, the empty LNP or the empty-LNP solution is subjected to the loading step after holding for about 1 minute, about 2 minutes, about 3 minutes, about 4 minutes, about 5 minutes, about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, or about 24 hours.

In some embodiments of the methods of the disclosure, the empty LNP or the empty-LNP solution is subjected to the loading step after storage for about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, or about 5 years.

In some embodiments of the methods of the disclosure, upon formation, the empty LNP or the empty-LNP solution is subjected to the loading step without storage or holding for a period of time.

In some aspects, a method of the present disclosure further comprises: iii) processing the loaded-LNP solution, thereby forming a lipid nanoparticle formulation (LNP formulation).

In contrast to other techniques for production (e.g., thin film rehydration/extrusion), ethanol-drop precipitation has been the industry standard for generating nucleic acid lipid nanoparticles. Precipitation reactions are favored due to their continuous nature, scalability, and ease of adoption. Such processes usually use high energy mixers (e.g., T-junction, confined impinging jets, microfluidic mixers, vortex mixers) to introduce lipids (in ethanol) to a suitable anti-solvent (i.e. water) in a controllable fashion, driving liquid supersaturation and spontaneous precipitation into lipid particles. In some embodiments, the vortex mixers used are those described in U.S. Pat. Application Nos. 62/799,636 and 62/886,592, which are incorporated herein by reference in their entirety. In some embodiments, the microfluidic mixers used are those described in PCT Application No. WO/2014/172045, which is incorporated herein by reference in their entirety.

In some embodiments of the methods of the disclosure, the mixing step is performed with a T-junction, confined impinging jets, microfluidic mixer, or vortex mixer.

In some embodiments, the loading step is performed with a T-junction, confined impinging jets, microfluidic mixer, or vortex mixer.

In some embodiments of the methods of the disclosure, the mixing step is performed at a temperature of less than about 30° C., less than about 28° C., less than about 26° C., less than about 25° C., less than about 24° C., less than about 22° C., or less than about 20° C.

In some embodiments of the methods of the disclosure, the loading step is performed at a temperature of less than about 30° C., less than about 28° C., less than about 26° C., less than about 25° C., less than about 24° C., less than about 22° C., or less than about 20° C.

In some embodiments of the methods of the disclosure, the step of processing the empty-LNP solution or loaded-LNP solution comprises a first adding step, comprising adding a polyethylene glycol lipid (PEG lipid) to the empty LNP solution or the loaded LNP solution.

In some embodiments of the methods of the disclosure, the first adding step comprises adding a polyethylene glycol solution (PEG solution) comprising the PEG lipid to the empty-LNP solution or loaded-LNP solution.

In some embodiments of the methods of the disclosure, the step of processing the empty-LNP solution or loaded-LNP solution comprises a second adding step, comprising adding a polyethylene glycol lipid (PEG lipid) to the empty LNP solution or the loaded LNP solution.

In some embodiments of the methods of the disclosure, the second adding step comprises adding a polyethylene glycol solution (PEG solution) comprising the PEG lipid to the empty-LNP solution or loaded-LNP solution.

In some embodiments of the methods of the disclosure, first adding step comprises adding about 0.1 mol% to about 3.0 mol% PEG lipid, about 0.2 mol% to about 2.5 mol% PEG lipid, about 0.5 mol% to about 2.0 mol% PEG lipid, about 0.75 mol% to about 1.5 mol% PEG lipid, about 1.0 mol% to about 1.25 mol% PEG lipid to the empty LNP.

In some embodiments of the methods of the disclosure, the first adding step comprises adding about 0.1 mol% to about 3.0 mol% PEG lipid, about 0.2 mol% to about 2.5 mol% PEG lipid, about 0.5 mol% to about 2.0 mol% PEG lipid, about 0.75 mol% to about 1.5 mol% PEG lipid, about 1.0 mol% to about 1.25 mol% PEG lipid to the loaded-LNP.

In some embodiments, the first adding step comprises adding about 1.75 mol% PEG lipid to the empty LNP or the loaded LNP.

In some embodiments of the methods of the disclosure, the second adding step comprises adding about 0.1 mol% to about 3.0 mol% PEG lipid, about 0.2 mol% to about 2.5 mol% PEG lipid, about 0.5 mol% to about 2.0 mol% PEG lipid, about 0.75 mol% to about 1.5 mol% PEG lipid, about 1.0 mol% to about 1.25 mol% PEG lipid to the empty LNP.

In some embodiments of the methods of the disclosure, the second adding step comprises adding about 0.1 mol% to about 3.0 mol% PEG lipid, about 0.2 mol% to about 2.5 mol% PEG lipid, about 0.5 mol% to about 2.0 mol% PEG lipid, about 0.75 mol% to about 1.5 mol% PEG lipid, about 1.0 mol% to about 1.25 mol% PEG lipid to the loaded LNP.

In some embodiments of the methods of the disclosure, the second adding step comprises adding about 1.0 mol% PEG lipid to the empty LNP or the loaded LNP.

In some embodiments of the methods of the disclosure, the first adding step is performed at a temperature of less than about 30° C., less than about 28° C., less than about 26° C., less than about 25° C., less than about 24° C., less than about 22° C., or less than about 20° C.

In some embodiments of the methods of the disclosure, the second adding step is performed at a temperature of less than about 30° C., less than about 28° C., less than about 26° C., less than about 25° C., less than about 24° C., less than about 22° C., or less than about 20° C.

In some embodiments of the methods of the disclosure, the step of processing the empty-LNP solution or loaded-LNP solution further comprises at least one step selected from filtering, pH adjusting, buffer exchanging, diluting, dialyzing, concentrating, freezing, lyophilizing, storing, and packing.

In some embodiments of the methods of the disclosure, the step of processing the empty-LNP solution or loaded-LNP solution further comprises pH adjusting.

In some embodiments of the methods of the disclosure, the pH adjusting comprises adding a second buffering agent is selected from the group consisting of an acetate buffer, a citrate buffer, a phosphate buffer, and a tris buffer.

In some embodiments of the methods of the disclosure, the first adding step is performed prior to the pH adjusting.

In some embodiments of the methods of the disclosure, the first adding step is performed after the pH adjusting.

In some embodiments of the methods of the disclosure, the second adding step is performed prior to the pH adjusting.

In some embodiments of the methods of the disclosure, the second adding step is performed after the pH adjusting.

In some embodiments of the methods of the disclosure, the step of processing the empty-LNP solution or loaded-LNP solution further comprises filtering.

In some embodiments of the methods of the disclosure, the filtering is a tangential flow filtration (TFF).

In some embodiments of the methods of the disclosure, the filtering removes an organic solvent (e.g., an alcohol such as ethanol) from the LNP solution. In some embodiments, upon removal of the organic solvent (e.g. an alcohol such as ethanol), the LNP solution is converted to a solution buffered at a neutral pH, e.g., pH 6.5 to 7.8, pH 6.8 to pH 7.5, preferably, pH 7.0 to pH 7.2 (e.g., by adding a phosphate buffer or HEPES buffer). In some embodiments, the LNP solution is converted to a solution buffered at a pH of from about 7.0 to pH to about 7.2. In some embodiments, the resulting LNP solution is sterilized before storage or use, e.g., by filtration (e.g., through a 0.1-0.5 µm filter).

In some embodiments of the methods of the disclosure, the step of processing the empty-LNP solution or loaded-LNP solution further comprises buffer exchanging.

In some embodiments of the methods of the disclosure, the buffer exchanging comprises addition of an aqueous buffer solution comprising a third buffering agent.

In some embodiments, the first adding step is performed prior to the buffer exchanging.

In some embodiments of the methods of the disclosure, the first adding step is performed after the buffer exchanging.

In some embodiments of the methods of the disclosure, the second adding is performed prior to the buffer exchanging.

In some embodiments of the methods of the disclosure, the second adding step is performed after the buffer exchanging.

In some embodiments of the methods of the disclosure, the step of processing the empty-LNP solution or loaded-LNP solution further comprises diluting the empty-LNP solution or loaded-LNP solution.

In some embodiments, the step of processing the empty-LNP solution or loaded-LNP solution further comprises dialyzing the empty-LNP solution or loaded-LNP solution.

In some embodiments of the methods of the disclosure, the step of processing the empty-LNP solution or loaded-LNP solution further comprises concentrating the empty-LNP solution or loaded-LNP solution.

In some embodiments of the methods of the disclosure, the step of processing the empty-LNP solution or loaded-LNP solution further comprises freezing the empty-LNP solution or loaded-LNP solution.

In some embodiments of the methods of the disclosure, the step of processing the empty-LNP solution or loaded-LNP solution further comprises lyophilizing the empty-LNP solution or loaded-LNP solution.

In some embodiments of the methods of the disclosure, the lyophilizing comprises freezing the loaded-LNP solution at a temperature from about -100° C. to about 0° C., about -80° C. to about -10° C., about -60° C. to about -20° C., about -50° C. to about -25° C., or about -40° C. to about -30° C.

In some embodiments of the methods of the disclosure, the lyophilizing further comprises drying the frozen loaded-LNP solution to form a lyophilized empty LNP or lyophilized loaded LNP.

In some embodiments of the methods of the disclosure, the drying is performed at a vacuum ranging from about 50 mTorr to about 150 mTorr.

In some embodiments, the drying is performed at about -35° C. to about -15° C.

In some embodiments of the methods of the disclosure, the drying is performed at about 25° C.

In some embodiments of the methods of the disclosure, the step of processing the empty-LNP solution or the loaded-LNP solution further comprises storing the empty-LNP solution or the loaded-LNP solution.

In some embodiments, the step of processing the empty-LNP solution or loaded-LNP solution further comprises packing.

In some embodiments of the methods of the disclosure, the step of packing the empty-LNP solution or loaded-LNP solution comprises one or more of the following steps:
iib) adding a cryoprotectant to the empty-LNP solution or loaded-LNP solution;
iic) lyophilizing the empty-LNP solution or loaded-LNP solution, thereby forming a lyophilized LNP composition;
iid) storing the empty-LNP solution or loaded-LNP solution of the lyophilized LNP composition; and/or
iie) adding a buffering solution to the empty-LNP solution, loaded-LNP solution or the lyophilized LNP composition, thereby forming the LNP formulation.

In some embodiments of the methods of the disclosure, the cryoprotectant is added to the empty-LNP solution or loaded-LNP solution prior to the lyophilization. In some embodiments, the cryoprotectant comprises one or more cryoprotective agents, and each of the one or more cryoprotective agents is independently a polyol (e.g., a diol or a triol such as propylene glycol (i.e., 1,2-propanediol), 1,3-propanediol, glycerol, (+/-)-2-methyl-2,4-pentanediol, 1,6-hexanediol, 1,2-butanediol, 2,3-butanediol, ethylene glycol, or diethylene glycol), a nondetergent sulfobetaine (e.g., NDSB-201 (3-(1-pyridino)-1-propane sulfonate), an osmolyte (e.g., L-proline or trimethylamine N-oxide dihydrate), a polymer (e.g., polyethylene glycol 200 (PEG 200), PEG 400, PEG 600, PEG 1000, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g., polyvinylpyrrolidone K 15), pentaerythritol propoxylate, or polypropylene glycol P 400), an organic solvent (e.g., dimethyl sulfoxide (DMSO) or ethanol), a sugar (e.g., D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g., lithium acetate, lithium chloride, lithium formate, lithium nitrate, lithium sulfate, magnesium acetate, sodium chloride, sodium formate, sodium malonate, sodium nitrate, sodium sulfate, or any hydrate thereof), or any combination thereof. In some embodiments, the cryoprotectant comprises sucrose. In some embodiments, the cryoprotectant is sucrose.

In some embodiments, the empty-LNP solution, loaded-LNP solution, or the lyophilized LNP composition is stored at a temperature of from about -40° C. to about 0° C., from about -35° C. to about -5° C., from about -30° C. to about -10° C., from about -25° C. to about -15° C., from about -22° C. to about -18° C., or from about -21° C. to about -19° C. prior to adding the buffering solution.

### Lipid Solutions

In some embodiments, the methods of the present disclosure provide a lipid solution.

In some embodiments, the lipid solution comprises an ionizable lipid.

In some embodiments, the lipid solution further comprises a phospholipid, a PEG lipid, a structural lipid, or any combination thereof.

In some embodiments, the lipid solution further comprises an encapsulation agent.

In some embodiments, the lipid solution comprises an ionizable lipid. In some embodiments, the lipid solution comprises the ionizable lipid at a concentration of greater than about 0.01 mg/mL, about 0.05 mg/mL, about 0.06 mg/mL, about 0.07 mg/mL, about 0.08 mg/mL, about 0.09 mg/mL, about 0.1 mg/mL, about 0.15 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, or about 1.0 mg/mL. In some embodiments, the lipid solution comprises a ionizable lipid at a concentration of from about 0.01 mg/mL to about 1.0 mg/mL, from about 0.01 mg/mL to about 0.9 mg/mL, from about 0.01 mg/mL to about 0.8 mg/mL, from about 0.01 mg/mL to about 0.7 mg/mL, from about 0.01 mg/mL to about 0.6 mg/mL, from about 0.01 mg/mL to about 0.5 mg/mL, from about 0.01 mg/mL to about 0.4 mg/mL, from about 0.01 mg/mL to about 0.3 mg/mL, from about 0.01 mg/mL to about 0.2 mg/mL, from about 0.01 mg/mL to about 0.1 mg/mL, from about 0.05 mg/mL to about 1.0 mg/mL, from about 0.05 mg/mL to about 0.9 mg/mL, from about 0.05 mg/mL to about 0.8 mg/mL, from about 0.05 mg/mL to about 0.7 mg/mL, from about 0.05 mg/mL to about 0.6 mg/mL, from about 0.05 mg/mL to about 0.5 mg/mL, from about 0.05 mg/mL to about 0.4 mg/mL, from about 0.05 mg/mL to about 0.3 mg/mL, from about 0.05 mg/mL to about 0.2 mg/mL, from about 0.05 mg/mL to about 0.1 mg/mL, from about 0.1 mg/mL to about 1.0 mg/mL, from about 0.2 mg/mL to about 0.9 mg/mL, from about 0.3 mg/mL to about 0.8 mg/mL, from about 0.4 mg/mL to about 0.7 mg/mL, or from about 0.5 mg/mL to about 0.6 mg/mL. In some embodiments, the lipid solution comprises an ionizable lipid at a concentration of up to about 5.0 mg/mL, up to about 4.0 mg/mL, up to about 3.0 mg/mL, up to about 2.0 mg/mL, up to about 1.0 mg/mL, up to about 0.09 mg/mL, up to about 0.08 mg/mL, up to about 0.07 mg/mL, up to about 0.06 mg/mL, or up to about 0.05 mg/mL.

In some embodiments, the lipid solution comprises an ionizable lipid. In some embodiments, the lipid solution comprises the ionizable lipid at a concentration of greater than greater than about 0.1 mg/mL, greater than about 0.5 mg/mL, greater than about 0.6 mg/mL, greater than about 0.7 mg/mL, greater than about 0.8 mg/mL, greater than about 0.9 mg/mL, greater than about 1.0 mg/mL, greater than about 1.5 mg/mL, greater than about 2.0 mg/mL, greater than about 3.0 mg/mL, greater than about 4.0 mg/mL, greater than about 5.0 mg/mL, greater than about 6.0 mg/mL, greater than about 7.0 mg/mL, greater than about 8.0 mg/mL, greater than about 9.0 mg/mL, greater than about 10 mg/mL, greater than about 11 mg/mL, greater than about 12 mg/mL, greater than about 13 mg/mL, greater than about 14 mg/mL, greater than about 15 mg/mL, greater than about 20 mg/mL, greater than about 25 mg/mL or greater than about 30 mg/mL. In some embodiments, the lipid solution comprises a ionizable lipid at a concentration of from about 0.1 mg/mL to about 20.0 mg/mL, from about 0.1 mg/mL to about 19 mg/mL, from about 0.1 mg/mL to about 18 mg/mL, from about 0.1 mg/mL to about 17 mg/mL, from about 0.1 mg/mL to about 16 mg/mL, from about 0.1 mg/mL to about 15 mg/mL, from about 0.1 mg/mL to about 14 mg/mL, from about 0.1 mg/mL to about 13 mg/mL, from about 0.1 mg/mL to about 12 mg/mL, from about 0.1 mg/mL to about 11 mg/mL, from about 0.5 mg/mL to about 10.0 mg/mL, from about 0.5 mg/mL to about 9 mg/mL, from about 0.5 mg/mL to about 8 mg/mL, from about 0.5 mg/mL to about 7 mg/mL, from about 0.5 mg/mL to about 6 mg/mL, from about 0.5 mg/mL to about 5.0 mg/mL, from about 0.5 mg/mL to about 4 mg/mL, from about 0.5 mg/mL to about 3 mg/mL, from about 0.5 mg/mL to about 2 mg/mL, from about 0.5 mg/mL to about 1 mg/mL, from about 1 mg/mL to about 20 mg/mL, from about 1 mg/mL to about 15 mg/mL, from about 1 mg/mL to about 12 mg/mL, from about 1 mg/mL to about 10 mg/mL, or from about 1 mg/mL to about 8 mg/mL. In some embodiments, the lipid solution comprises an ionizable lipid at a concentration of up to about 30 mg/mL, about 25, about mg/mL, about 20 mg/mL, about 18 mg/mL, about 16 mg/mL, about 15 mg/mL, about 14 mg/mL, about 12 mg/mL, about 10 mg/mL, about 8 mg/mL, about 6 mg/mL, about 5.0 mg/mL, about 4.0 mg/mL, about 3.0 mg/mL, about 2.0 mg/mL, about 1.0 mg/mL, about 0.09 mg/mL, about 0.08 mg/mL, about 0.07 mg/mL, about 0.06 mg/mL, or about 0.05 mg/mL.

In some embodiments, the lipid solution comprises an ionizable lipid in an aqueous buffer and/or organic solution. In some embodiments, the lipid solution further comprises a buffering agent and/or a salt. Exemplary suitable buffering agents include, but are not limited to, ammonium sulfate, sodium bicarbonate, sodium citrate, sodium acetate, potassium phosphate, sodium phosphate, HEPES, and the like. In some embodiments, the lipid solution comprises a buffering agent at a concentration of from about 0.1 mM to about 100 mM, from about 0.5 mM to about 90 mM, from about 1.0 mM to about 80 mM, from about 2 mM to about 70 mM, from about 3 mM to about 60 mM, from about 4 mM to about 50 mM, from about 5 mM to about 40 mM, from about 6 mM to about 30 mM, from about 7 mM to about 20 mM, from about 8 mM to about 15 mM, from about 9 mM to about 12 mM. In some embodiments, the lipid solution comprises a buffering agent at a concentration of or greater than about 0.1 mM, about 0.5 mM, about 1 mM, about 2 mM, about 4 mM, about 6 mM, about 8 mM, about 10 mM, about 15 mM, about 20 mM, about 25 mM, about 30 mM, about 35 mM, about 40 mM, about 45 mM, or about 50 mM. Exemplary suitable salts include, but are not limited to, potassium chloride, magnesium chloride, sodium chloride, and the like.

In some embodiments, the lipid solution has a pH of from about 4.5 to about 7.0, from about 4.6 to about 7.0, from about 4.8 to about 7.0, from about 5.0 to about 7.0, from about 5.5 to about 7.0, from about 6.0 to about 7.0, from about 6.0 to about 6.9, from about 6.0 to about 6.8, from about 6.0 to about 6.7, from about 6.0 to about 6.6, from about 6.0 to about 6.5. In some embodiments, the lipid solution has a pH of from about 7.0 to about 8.0, from about 7.1 to about 7.8, from about 7.2 to about 7.6, or from about 7.3 to about 7.5.

In some embodiments, a lipid solution has a pH of or no greater than 4.5, of or no greater than 4.6, of or no greater than 4.7, of or no greater than 4.8, of or no greater than 4.9, of or no greater than 5.0, of or no greater than 5.2, of or no greater than 5.4, of or no greater than 5.6, of or no greater than 5.8, of or no greater than 6.0, of or no greater than 6.1, of or no greater than 6.2, of or no greater than 6.3, of or no greater than 6.4, of or no greater than 6.5, of or no greater than 6.6, of or no greater than 6.7, of or no greater than 6.8, of or no greater than 6.9, or of or no greater than 7.0.

In some embodiments, the lipid solution comprises from about 1% by volume to about 50% by volume of a first organic solvent relative to the total volume of the lipid solution. In some embodiments, the lipid solution comprises from about 2% by volume to about 45% by volume of the organic solvent relative to the total volume of the lipid nanoparticle formulation. In some embodiments, the lipid solution comprises from about 3% by volume to about 40% by volume of the organic solvent relative to the total volume of the lipid nanoparticle formulation. In some embodiments, the lipid solution comprises from about 4% by volume to about 35% by volume of the organic solvent relative to the total volume of the lipid nanoparticle formulation. In some embodiments, the lipid solution comprises from about 5% by volume to about 33% by volume of the organic solvent relative to the total volume of the lipid nanoparticle formulation.

In some embodiments, the first organic solvent is an alcohol.

In some embodiments, the organic solvent is ethanol.

### Buffering Agents

In some embodiments, the methods of the present disclosure provide a buffering agent. In some embodiments, the methods of the present disclosure provide a first buffering agent, a second buffering agent, a third buffering agent, or a combination thereof.

In some embodiments, the first aqueous buffer solution comprises a first buffering agent. In some embodiments, a suitable solution may further comprise one or more buffering agents and/or a salt. Exemplary buffering agents include, but are not limited to, ammonium sulfate, sodium bicarbonate, sodium citrate, sodium acetate, potassium phosphate, tris(hydroxymethyl)aminomethane (tris), sodium phosphate, HEPES, and the like. In some embodiments, the first aqueous buffer solution comprises a first buffering agent at a concentration of from about 0.1 nM to about 100 mM, from about 0.5 nM to about 90 mM, from about 1.0 nM to about 80 mM, from about 2 nM to about 70 mM, from about 3 nM to about 60 mM, from about 4 nM to about 50 mM, from about 5 nM to about 40 mM, from about 6 nM to about 30 mM, from about 7 nM to about 20 mM, from about 8 nM to about 15 mM, from about 9 mM to about 12 mM. In some embodiments, the first aqueous buffer solution comprises a first buffering agent at a concentration of or greater than about 0.1 mM, of or greater than about 0.5 mM, of or greater than about 1 mM, of or greater than about 2 mM, of or greater than about 4 mM, of or greater than about 6 mM, of or greater than about 8 mM, of or greater than about 10 mM, of or greater than about 15 mM, of or greater than about 20 mM, of or greater than about 25 mM, of or greater than about 30 mM, of or greater than about 35 mM, of or greater than about 40 mM, of or greater than about 45 mM, or of or greater than about 50 mM. Exemplary suitable salts include, but are not limited to, potassium chloride, magnesium chloride, sodium chloride, and the like.

In some embodiments, the first buffering agent comprises a first aqueous buffer. In some embodiments, a suitable solution may further comprise one or more aqueous buffer and/or a salt. Exemplary suitable aqueous buffers include, but are not limited to, ammonium sulfate, sodium bicarbonate, sodium citrate, sodium acetate, potassium phosphate, tris(hydroxymethyl)aminomethane (tris), sodium phosphate, HEPES, and the like. In some embodiments, the first aqueous buffer comprises an aqueous buffer at a concentration of from about 0.1 nM to about 100 mM, from about 0.5 nM to about 90 mM, from about 1.0 nM to about 80 mM, from about 2 nM to about 70 mM, from about 3 nM to about 60 mM, from about 4 nM to about 50 mM, from about 5 nM to about 40 mM, from about 6 nM to about 30 mM, from about 7 nM to about 20 mM, from about 8 nM to about 15 mM, from about 9-12 mM. In some embodiments, the first aqueous buffer comprises an aqueous buffer at a concentration of or greater than about 0.1 mM, of or greater than about 0.5 mM, of or greater than about 1 mM, of or greater than about 2 mM, of or greater than about 4 mM, of or greater than about 6 mM, of or greater than about 8 mM, of or greater than about 10 mM, of or greater than about 15 mM, of or greater than about 20 mM, of or greater than about 25 mM, of or greater than about 30 mM, of or greater than about 35 mM, of or greater than about 40 mM, of or greater than about 45 mM, or of or greater than about 50 mM. Exemplary suitable salts include, but are not limited to, potassium chloride, magnesium chloride, sodium chloride, and the like.

In some embodiments, the first aqueous buffer solution has a pH of from about 4.5 to about 7.0, from about 4.6 to about 7.0, from about 4.8 to about 7.0, from about 5.0 to about 7.0, from about 5.5 to about 7.0, from about 6.0 to about 7.0, from about 6.0 to about 6.9, from about 6.0 to about 6.8, from about 6.0 to about 6.7, from about 6.0 to about 6.6, from about 6.0 to about 6.5. In some embodiments, the first aqueous buffer solution has a pH of or no greater than 4.5, of or no greater than 4.6, of or no greater than 4.7, of or no greater than 4.8, of or no greater than 4.9, of or no greater than 5.0, of or no greater than 5.2, of or no greater than 5.4, of or no greater than 5.6, of or no greater than 5.8, of or no greater than 6.0, of or no greater than 6.1, of or no greater than 6.2, of or no greater than 6.3, of or no greater than 6.4, of or no greater than 6.5, of or no greater than 6.6, of or no greater than 6.7, of or no greater than 6.8, of or no greater than 6.9, or of or no greater than 7.0.

In some embodiments, the first buffering agent has a pH of from about 4.5 to about 7.0, from about 4.6 to about 7.0, from about 4.8 to about 7.0, from about 5.0 to about 7.0, from about 5.5 to about 7.0, from about 6.0 to about 7.0, from about 6.0 to about 6.9, from about 6.0 to about 6.8, from about 6.0 to about 6.7, from about 6.0 to about 6.6, from about 6.0 to about 6.5. In some embodiments, the first buffering agent has a pH of or no greater than 4.5, of or no greater than 4.6, of or no greater than 4.7, of or no greater than 4.8, of or no greater than 4.9, of or no greater than 5.0, of or no greater than 5.2, of or no greater than 5.4, of or no greater than 5.6, of or no greater than 5.8, of or no greater than 6.0, of or no greater than 6.1, of or no greater than 6.2, of or no greater than 6.3, of or no greater than 6.4, of or no greater than 6.5, of or no greater than 6.6, of or no greater than 6.7, of or no greater than 6.8, of or no greater than 6.9, or of or no greater than 7.0.

In some embodiments, the first aqueous buffer is selected from the group consisting of an acetate buffer, a citrate buffer, a phosphate buffer, and a tris buffer.

In some embodiments, the buffering agent is selected from the group consisting of an acetate buffer, a citrate buffer, a phosphate buffer, and a tris buffer.

In some embodiments, the first aqueous buffer solution comprises greater than about 1 mM citrate, acetate, phosphate or tris, greater than about 2 mM citrate, acetate, phosphate or tris, greater than about 5 mM citrate, acetate, phosphate or tris, greater than about 10 mM citrate, acetate, phosphate or tris, greater than about 15 mM citrate, acetate, phosphate or tris, greater than about 20 mM citrate, acetate, phosphate or tris, greater than about 25 mM citrate, acetate, phosphate or tris, or greater than about 30 mM citrate, acetate, phosphate or tris.

In some embodiments, the first aqueous buffer solution comprises about 1 mM to about 30 mM citrate, acetate, phosphate or tris, about 2 mM to about 20 mM citrate, acetate, phosphate or tris, about 3 mM to about 10 mM citrate, acetate, phosphate or tris, about 4 mM to about 8 mM citrate, acetate, phosphate or tris, or about 5 mM to about 6 mM citrate, acetate, phosphate or tris.

In some embodiments, the first aqueous buffer solution comprises about 5 mM citrate, acetate, phosphate or tris.

In some embodiments, the first aqueous buffer solution comprises about 5 mM acetate, wherein the aqueous buffer solution has a pH of about 5.0.

In some embodiments, the second aqueous buffer solution comprises a second buffering agent. In some embodiments, a suitable solution may further comprise one or more buffering agent and/or a salt. Exemplary suitable buffering agent include, but are not limited to, ammonium sulfate, sodium bicarbonate, sodium citrate, sodium acetate, potassium phosphate, tris(hydroxymethyl)aminomethane (tris), sodium phosphate, HEPES, and the like. In some embodiments, the second aqueous buffer solution comprises buffering agent at a concentration of from about 0.1 mM to about 100 mM, from about 0.5 mM to about 90 mM, from about 1.0 mM to about 80 mM, from about 2 mM to about 70 mM, from about 3 mM to about 60 mM, from about 4 mM to about 50 mM, from about 5 mM to about 40 mM, from about 6 mM to about 30 mM, from about 7 mM to about 20 mM, from about 8 mM to about 15 mM, from about 9 mM to about 12 mM. In some embodiments, the second aqueous buffer solution comprises a buffering agent at a concentration of or greater than about 0.1 mM, of or greater than about 0.5 mM, of or greater than about 1 mM, of or greater than about 2 mM, of or greater than about 4 mM, of or greater than about 6 mM, of or greater than about 8 mM, of or greater than about 10 mM, of or greater than about 15 mM, of or greater than about 20 mM, of or greater than about 25 mM, of or greater than about 30 mM, of or greater than about 35 mM, of or greater than about 40 mM, of or greater than about 45 mM, or of or greater than about 50 mM. Exemplary suitable salts include, but are not limited to, potassium chloride, magnesium chloride, sodium chloride, and the like.

In some embodiments, the second buffering agent comprises a second aqueous buffer. In some embodiments, a suitable solution may further comprise one or more aqueous buffer and/or a salt. Exemplary suitable aqueous buffers include, but are not limited to, ammonium sulfate, sodium bicarbonate, sodium citrate, sodium acetate, potassium phosphate, tris(hydroxymethyl)aminomethane (tris), sodium phosphate, HEPES, and the like. In some embodiments, the second aqueous buffer comprises an aqueous buffer at a concentration of from about 0.1 mM to about 100 mM, from about 0.5 mM to about 90 mM, from about 1.0 mM to about 80 mM, from about 2 mM to about 70 mM, from about 3 mM to about 60 mM, from about 4 mM to about 50 mM, from about 5 mM to about 40 mM, from about 6 mM to about 30 mM, from about 7 mM to about 20 mM, from about 8 mM to about 15 mM, from about 9 mM to about 12 mM. In some embodiments, the second aqueous buffer comprises an aqueous buffer at a concentration of or greater than about 0.1 mM, about 0.5 mM, of or greater than about 1 mM, of or greater than about 2 mM, of or greater than about 4 mM, of or greater than about 6 mM, of or greater than about 8 mM, of or greater than about 10 mM, of or greater than about 15 mM, of or greater than about 20 mM, of or greater than about 25 mM, of or greater than about 30 mM, of or greater than about 35 mM, of or greater than about 40 mM, of or greater than about 45 mM, or of or greater than about 50 mM. Exemplary suitable salts include, but are not limited to, potassium chloride, magnesium chloride, sodium chloride, and the like.

In some embodiments, the second buffering agent has a pH of from about 4.5 to about 7.0, from about 4.6 to about 7.0, from about 4.8 to about 7.0, from about 5.0 to about 7.0, from about 5.5 to about 7.0, from about 6.0 to about 7.0, from about 6.0 to about 6.9, from about 6.0 to about 6.8, from about 6.0 to about 6.7, from about 6.0 to about 6.6, from about 6.0 to about 6.5. In some embodiments, the second buffering agent has a pH of or no greater than 4.5, of or no greater than 4.6, of or no greater than 4.7, of or no greater than 4.8, of or no greater than 4.9, of or no greater than 5.0, of or no greater than 5.2, of or no greater than 5.4, of or no greater than 5.6, of or no greater than 5.8, of or no greater than 6.0, of or no greater than 6.1, of or no greater than 6.2, of or no greater than 6.3, of or no greater than 6.4, of or no greater than 6.5, of or no greater than 6.6, of or no greater than 6.7, of or no greater than 6.8, of or no greater than 6.9, or of or no greater than 7.0.

In some embodiments, the second aqueous buffer solution has a pH of from about 4.5 to about 7.0, from about 4.6 to about 7.0, from about 4.8 to about 7.0, from about 5.0 to about 7.0, from about 5.5 to about 7.0, from about 6.0 to about 7.0, from about 6.0 to about 6.9, from about 6.0 to about 6.8, from about 6.0 to about 6.7, from about 6.0 to about 6.6, from about 6.0 to about 6.5. In some embodiments, the second aqueous buffer solution has a pH of or no greater than 4.5, of or no greater than 4.6, of or no greater than 4.7, of or no greater than 4.8, of or no greater than 4.9, of or no greater than 5.0, of or no greater than 5.2, of or no greater than 5.4, of or no greater than 5.6, of or no greater than 5.8, of or no greater than 6.0, of or no greater than 6.1, of or no greater than 6.2, of or no greater than 6.3, of or no greater than 6.4, of or no greater than 6.5, of or no greater than 6.6, of or no greater than 6.7, of or no greater than 6.8, of or no greater than 6.9, or of or no greater than 7.0.

In some embodiments, the second buffering agent is selected from the group consisting of an acetate buffer, a citrate buffer, a phosphate buffer, and a tris buffer.

In some embodiments, the second aqueous buffer is selected from the group consisting of an acetate buffer, a citrate buffer, a phosphate buffer, and a tris buffer.

In some embodiments, the second aqueous buffer is a tris buffer.

In some embodiments, the second buffering agent is a tris buffer.

In some embodiments, the second buffering agent has a pH in a range of from about 6.5 to about 8.5, from about 7.0 to about 8.0, from about 7.2 to about 7.8, or from about 7.4 to about 7.6.

In some embodiments, the second aqueous buffer has a pH in a range of from about 6.5 to about 8.5, from about 7.0 to about 8.0, from about 7.2 to about 7.8, or from about 7.4 to about 7.6.

In some embodiments, the second aqueous buffer has a pH of about 7.5.

In some embodiments, the second buffering agent has a pH of about 7.5.

In some embodiments, the third aqueous buffer solution comprises a third buffering agent. In some embodiments, a suitable solution may further comprise one or more aqueous buffer and/or a salt. Exemplary suitable buffering agents include, but are not limited to, ammonium sulfate, sodium bicarbonate, sodium citrate, sodium acetate, potassium phosphate, tris(hydroxymethyl)aminomethane (tris), sodium phosphate, HEPES, and the like. In some embodiments, the third aqueous buffer solution comprises a third buffering agent at a concentration of from about 0.1 mM to about 100 mM, from about 0.5 mM to about 90 mM, from about 1.0 mM to about 80 mM, from about 2 mM to about 70 mM, from about 3 mM to about 60 mM, from about 4 mM to about 50 mM, from about 5 mM to about 40 mM, from about 6 mM to about 30 mM, from about 7 mM to about 20 mM, from about 8 mM to about 15 mM, from about 9 mM to about 12 mM. In some embodiments, the third aqueous buffer solution comprises third buffering agent at a concentration of or greater than about 0.1 mM, of or greater than about 0.5 mM, of or greater than about 1 mM, of or greater than about 2 mM, of or greater than about 4 mM, of or greater than about 6 mM, of or greater than about 8 mM, of or greater than about 10 mM, of or greater than about 15 mM, of or greater than about 20 mM, of or greater than about 25 mM, of or greater than about 30 mM, of or greater than about 35 mM, of or greater than about 40 mM, of or greater than about 45 mM, or of or greater than about 50 mM. Exemplary suitable salts include, but are not limited to, potassium chloride, magnesium chloride, sodium chloride, and the like.

In some embodiments, the third buffering agent comprises a third aqueous buffer. In some embodiments, a suitable solution may further comprise one or more aqueous buffer and/or a salt. Exemplary suitable aqueous buffers include, but are not limited to, ammonium sulfate, sodium bicarbonate, sodium citrate, sodium acetate, potassium phosphate, tris(hydroxymethyl)aminomethane (tris), sodium phosphate, HEPES, and the like. In some embodiments, the third aqueous buffer comprises an aqueous buffer at a concentration of from about 0.1 mM to about 100 mM, from about 0.5 mM to about 90 mM, from about 1.0 mM to about 80 mM, from about 2 mM to about 70 mM, from about 3 mM to about 60 mM, from about 4 mM to about 50 mM, from about 5 mM to about 40 mM, from about 6 mM to about 30 mM, from about 7 mM to about 20 mM, from about 8 mM to about 15 mM, from about 9 mM to about 12 mM. In some embodiments, the third aqueous buffer comprises an aqueous buffer at a concentration of or greater than about 0.1 mM, about 0.5 mM, about 1 mM, about 2 mM, about 4 mM, about 6 mM, about 8 mM, about 10 mM, about 15 mM, about 20 mM, about 25 mM, about 30 mM, about 35 mM, about 40 mM, about 45 mM, or about 50 mM. Exemplary suitable salts include, but are not limited to, potassium chloride, magnesium chloride, sodium chloride, and the like.

In some embodiments, the third aqueous buffer has a pH of from about 4.5 to about 7.0, from about 4.6 to about 7.0, from about 4.8 to about 7.0, from about 5.0 to about 7.0, from about 5.5 to about 7.0, from about 6.0 to about 7.0, from about 6.0 to about 6.9, from about 6.0 to about 6.8, from about 6.0 to about 6.7, from about 6.0 to about 6.6, from about 6.0 to about 6.5. In some embodiments, the third buffering agent has a pH of or no greater than 4.5, of or no greater than 4.6, of or no greater than 4.7, of or no greater than 4.8, of or no greater than 4.9, of or no greater than 5.0, of or no greater than 5.2, of or no greater than 5.4, of or no greater than 5.6, of or no greater than 5.8, of or no greater than 6.0, of or no greater than 6.1, of or no greater than 6.2, of or no greater than 6.3, of or no greater than 6.4, of or no greater than 6.5, of or no greater than 6.6, of or no greater than 6.7, of or no greater than 6.8, of or no greater than 6.9, or of or no greater than 7.0.

In some embodiments, the third aqueous buffer is selected from the group consisting of an acetate buffer, a citrate buffer, a phosphate buffer, and a tris buffer.

In some embodiments, the third aqueous buffer has a pH in a range of from about 6.5 to about 8.5, from about 7.0 to about 8.0, from about 7.2 to about 7.8, or from about 7.4 to about 7.6.

In some embodiments, the third aqueous buffer has a pH of about 7.5.

### Nucleic Acids and Active Agent Solutions

In some embodiments, the methods of the present disclosure provide an active agent solution comprising a therapeutic and/or prophylactic agent. The therapeutic and/or prophylactic agent may be provided in a solution to be mixed or added to a lipid nanoparticle or lipid nanoparticle solution such that the therapeutic and/or prophylactic agent may be encapsulated in the lipid nanoparticle.

In some embodiments, the therapeutic and/or prophylactic agent is a vaccine or a compound capable of eliciting an immune response. In some embodiments, the loaded LNP, loaded LNP solution, or LNP formulation is a vaccine.

In some embodiments, the therapeutic and/or prophylactic agent is a nucleic acid.

In some embodiments, the methods of the present disclosure provide a nucleic acid solution comprising a nucleic acid. The nucleic acid may be provided in a solution to be mixed or added to a lipid nanoparticle or lipid nanoparticle solution such that the nucleic acid may be encapsulated in the lipid nanoparticle (thereby forming the “loaded LNP”).

In some embodiments, the nucleic acid solution comprises the nucleic acid to be encapsulated at various concentrations. In some embodiments, the nucleic acid solution comprises a nucleic acid at a concentration of greater than about 0.01 mg/mL, of or greater than about 0.05 mg/mL, of or greater than about 0.06 mg/mL, of or greater than about 0.07 mg/mL, of or greater than about 0.08 mg/mL, of or greater than about 0.09 mg/mL, of or greater than about 0.1 mg/mL, of or greater than about 0.15 mg/mL, of or greater than about 0.2 mg/mL, of or greater than about 0.3 mg/mL, of or greater than about 0.4 mg/mL, of or greater than about 0.5 mg/mL, of or greater than about 0.6 mg/mL, of or greater than about 0.7 mg/mL, of or greater than about 0.8 mg/mL, of or greater than about 0.9 mg/mL, or of or greater than about 1.0 mg/mL. In some embodiments, the nucleic acid solution comprises a nucleic acid at a concentration of from about 0.01 mg/mL to about 1.0 mg/mL, from about 0.01 mg/mL to about 0.9 mg/mL, from about 0.01 mg/mL to about 0.8 mg/mL, from about 0.01 mg/mL to about 0.7 mg/mL, from about 0.01 mg/mL to about 0.6 mg/mL, from about 0.01 mg/mL to about 0.5 mg/mL, from about 0.01 mg/mL to about 0.4 mg/mL, from about 0.01 mg/mL to about 0.3 mg/mL, from about 0.01 mg/mL to about 0.2 mg/mL, from about 0.01 mg/mL to about 0.1 mg/mL, from about 0.05 mg/mL to about 1.0 mg/mL, from about 0.05 mg/mL to about 0.9 mg/mL, from about 0.05 mg/mL to about 0.8 mg/mL, from about 0.05 mg/mL to about 0.7 mg/mL, from about 0.05 mg/mL to about 0.6 mg/mL, from about 0.05 mg/mL to about 0.5 mg/mL, from about 0.05 mg/mL to about 0.4 mg/mL, from about 0.05 mg/mL to about 0.3 mg/mL, from about 0.05 mg/mL to about 0.2 mg/mL, from about 0.05 mg/mL to about 0.1 mg/mL, from about 0.1 mg/mL to about 1.0 mg/mL, from about 0.2 mg/mL to about 0.9 mg/mL, from about 0.3 mg/mL to about 0.8 mg/mL, from about 0.4 mg/mL to about 0.7 mg/mL, or from about 0.5 mg/mL to about 0.6 mg/mL. In some embodiments, the nucleic acid solution my comprise a nucleic acid at a concentration up to about 5.0 mg/mL, up to about 4.0 mg/mL, up to about 3.0 mg/mL, up to about 2.0 mg/mL, up to about 1.0 mg/mL, up to about 0.09 mg/mL, up to about 0.08 mg/mL, up to about 0.07 mg/mL, up to about 0.06 mg/mL, or up to about 0.05 mg/mL. In some embodiments, the nucleic acid solution comprises from about 0.001 to about 1.0 mg/mL of the nucleic acid, from about 0.0025 to about 0.5 mg/mL of the nucleic acid, or from about 0.005 to about 0.2 mg/mL of the nucleic acid. In some embodiments, the nucleic acid solution comprises about 0.005 to about 0.2 mg/mL of the nucleic acid.

In some embodiments, the nucleic acid solution comprises a nucleic acid in an aqueous buffer. In some embodiments, a suitable nucleic acid solution may further comprise a buffering agent and/or a salt. Exemplary suitable buffering agents include, but are not limited to, ammonium sulfate, sodium bicarbonate, sodium citrate, sodium acetate, potassium phosphate, sodium phosphate, tris(hydroxymethyl)aminomethane (tris), HEPES, and the like. In some embodiments, the nucleic acid solution comprises a buffering agent at a concentration of from about 0.1 mM to about 100 mM, from about 0.5 mM to about 90 mM, from about 1.0 mM to about 80 mM, from about 2 mM to about 70 mM, from about 3 mM to about 60 mM, from about 4 mM to about 50 mM, from about 5 mM to about 40 mM, from about 6 mM to about 30 mM, from about 7 mM to about 20 mM, from about 8 mM to about 15 mM, from about 9 mM to about 12 mM. In some embodiments, the nucleic acid solution comprises a buffering agent at a concentration of or greater than about 0.1 mM, greater than about 0.5 mM, greater than about 1 mM, greater than about 2 mM, greater than about 4 mM, greater than about 6 mM, greater than about 8 mM, greater than about 10 mM, greater than about 15 mM, greater than about 20 mM, greater than about 25 mM, greater than about 30 mM, greater than about 35 mM, greater than about 40 mM, greater than about 45 mM, or greater than about 50 mM. Exemplary suitable salts include, but are not limited to, potassium chloride, magnesium chloride, sodium chloride, and the like.

In some embodiments, the nucleic acid solution has a pH of from about 4.5 to about 7.0, from about 4.6 to about 7.0, from about 4.8 to about 7.0, from about 5.0 to about 7.0, from about 5.5 to about 7.0, from about 6.0 to about 7.0, from about 6.0 to about 6.9, from about 6.0 to about 6.8, from about 6.0 to about 6.7, from about 6.0 to about 6.6, from about 6.0 to about 6.5. In some embodiments, the nucleic acid solution has a pH of from about 4.5 to from about 6.5, from about 4.8 to about 6.25, from about 4.8 to about 6.0, from about 5.0 to about 5.8, or from about 5.2 to about 5.5. In some embodiments, the nucleic acid solution has a pH of from about 5.0 to about 6.0, from about 5.1 to about 5.75, or from about 5.2 to about 5.5. In some embodiments, the nucleic acid solution has a pH of from about 4.5 to about 6.5, from about 4.8 to about 6.25, from about 4.8 to about 6.0, from about 5.0 to about 5.8, or from about 5.2 to about 5.5. In some embodiments, a suitable nucleic acid solution has a pH of or no greater than 4.5, of or no greater than 4.6, of or no greater than 4.7, of or no greater than 4.8, of or no greater than 4.9, of or no greater than 5.0, of or no greater than 5.2, of or no greater than 5.4, of or no greater than 5.6, of or no greater than 5.8, of or no greater than 6.0, of or no greater than 6.1, of or no greater than 6.2, of or no greater than 6.3, of or no greater than 6.4, of or no greater than 6.5, of or no greater than 6.6, of or no greater than 6.7, of or no greater than 6.8, of or no greater than 6.9, or of or no greater than 7.0.

In some embodiments, the nucleic acid solution comprises an acetate buffer.

In some embodiments, the nucleic acid solution comprises from about 1 mM to about 200 mM acetate buffer, from about 2 mM to about 180 mM acetate buffer, from about 3 mM to about 160 mM acetate buffer, from about 4 mM to about 150 mM acetate buffer, from about 4 mM to about 140 mM acetate buffer, from about 5 mM to about 130 mM acetate buffer, from about 6 mM to about 120 mM acetate buffer, from about 7 mM to about 110 mM acetate buffer, from about 8 mM to about 100 mM acetate buffer, from about 9 mM to about 90 mM acetate buffer, from about 10 mM to about 80 mM acetate buffer, from about 15 mM to about 70 mM acetate buffer, from about 20 mM to about 60 mM acetate buffer, from about 25 mM to about 50 mM acetate buffer, or from about 30 mM to about 40 mM acetate buffer.

In some embodiments, the nucleic acid solution comprises about 8.8 mM acetate buffer.

In some embodiments, the nucleic acid solution comprises about 130 mM acetate buffer.

### Empty Lipid Nanoparticles (Empty LNPs)

In some aspects, the present disclosure provides an empty lipid nanoparticle (empty LNP) prepared by a method disclosed herein.

In some aspects, the present disclosure provides an empty LNP comprising a polymeric lipid.

In some aspects, the present disclosure provides an empty LNP comprising from about 0.1 mol% to about 2.5 mol%, from about 0.2 mol% to about 2.25 mol%, from about 0.25 mol% to about 2.0 mol%, from about 0.5 mol% to about 1.75 mol%, from about 0.75 mol% to about 1.5 mol%, or from about 1.0 mol% to about 1.25 mol% of a polymeric lipid.

In some aspects, the present disclosure provides an empty LNP comprising from about 0.1 mol% to about 0.5 mol% of a polymeric lipid.

In some embodiments, the polymeric lipid is a PEG lipid.

In some embodiments, the polymeric lipid is not a PEG lipid.

In some embodiments, the polymeric lipid is an amphiphilic polymer-lipid conjugate.

In some embodiments, the polymeric lipid is a PEG-lipid conjugate.

In some embodiments, the polymeric lipid is a surfactant.

In some embodiments, the polymeric lipid is Brij or OH-PEG-stearate.

In some aspects, the present disclosure provides an empty LNP comprising from about 0.1 mol% to about 0.5 mol%of a PEG lipid.

In some embodiments, the empty LNP further comprises from about 0.1 mol% to about 0.5 mol% PEG lipid, a phospholipid, a structural lipid, or any combination thereof.

In some embodiments, the empty LNP comprises about 3.0 mol% PEG lipid or less, about 2.75 mol% PEG lipid or less, about 2.5 mol% PEG lipid or less, about 2.25 mol% PEG lipid or less, about 2.0 mol% PEG lipid or less, about 1.75 mol% PEG lipid or less, about 1.5 mol% PEG lipid or less, about 1.25 mol% PEG lipid or less, about 1.0 mol% PEG lipid or less, about 0.9 mol% PEG lipid or less, about 0.8 mol% PEG lipid or less, about 0.7 mol% PEG lipid or less, about 0.6 mol% PEG lipid or less, about 0.5 mol% PEG lipid or less, about 0.4 mol% PEG lipid or less, about 0.3 mol% PEG lipid or less, about 0.2 mol% PEG lipid or less, or about 0.1 mol% PEG lipid or less.

In some embodiments, the empty LNP comprises from about 0 mol% to about 3.0 mol% PEG lipid, from about 0.1 mol% to about 2.5 mol% PEG lipid, from about 0.2 mol% to about 2.25 mol% PEG lipid, from about 0.25 mol% to about 2.0 mol% PEG lipid, from about 0.5 mol% to about 1.75 mol% PEG lipid, from about 0.75 mol% to about 1.5 mol% PEG lipid, or from about 1.0 mol% to about 1.25 mol% PEG lipid.

In some embodiments, the empty LNP comprises from about 0.050 mol% to about 0.5 mol% PEG lipid.

In some embodiments, the empty LNP, comprises from about 30 mol% to about 60 mol% ionizable lipid; from about 0 mol% to about 30 mol% phospholipid; from about 15 mol% to about 50 mol% structural lipid; and from about 0.1 mol% to about 0.5 mol% PEG lipid.

In some embodiments, the empty LNP, comprises from about 30 mol% to about 60 mol% ionizable lipid; from about 0 mol% to about 30 mol% phospholipid; from about 15 mol% to about 50 mol% structural lipid; and from about 0.1 mol% to about 10 mol% PEG lipid.

In some embodiments, the empty LNP has an average lipid nanoparticle diameter of about 200 nm or less, about 175 nm or less, about 150 nm or less, about 125 nm or less, about 100 nm or less, about 90 nm or less, about 80 nm or less, about 75 nm or less, about 70 nm or less, about 65 nm or less, about 60 nm or less, about 55 nm or less, about 50 nm or less, about 45 nm or less, about 40 nm or less, about 35 nm or less, about 30 nm or less, about 25 nm or less, or about 20 nm or less.

In some embodiments, the empty LNP has an average lipid nanoparticle diameter of from about 20 nm to about 150 nm, from about 25 nm to about 125 nm, from about 30 nm to about 110 nm, from about 35 nm to about 100 nm, from about 40 nm to about 90 nm, from about 45 nm to about 80 nm, or from about 50 nm to about 70 nm.

In some embodiments, empty LNP has an average lipid nanoparticle diameter of about 25 to about 45 nm.

### Empty Lipid Nanoparticle Solutions (Empty-LNP Solutions)

In some embodiments, the present disclosure provides an empty lipid nanoparticle solution (empty-LNP solution) prepared by a method disclosed herein.

In some embodiments, the empty-LNP solution comprises the empty LNP. In some embodiments, the empty-LNP solution comprises the empty LNP at a concentration of greater than about 0.01 mg/mL, about 0.05 mg/mL, about 0.06 mg/mL, about 0.07 mg/mL, about 0.08 mg/mL, about 0.09 mg/mL, about 0.1 mg/mL, about 0.15 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, or about 1.0 mg/mL. In some embodiments, the empty-LNP solution comprises the empty LNP at a concentration of from about 0.01 mg/mL to about 1.0 mg/mL, from about 0.01 mg/mL to about 0.9 mg/mL, from about 0.01 mg/mL to about 0.8 mg/mL, from about 0.01 mg/mL to about 0.7 mg/mL, from about 0.01 mg/mL to about 0.6 mg/mL, from about 0.01 mg/mL to about 0.5 mg/mL, from about 0.01 mg/mL to about 0.4 mg/mL, from about 0.01 mg/mL to about 0.3 mg/mL, from about 0.01 mg/mL to about 0.2 mg/mL, from about 0.01 mg/mL to about 0.1 mg/mL, from about 0.05 mg/mL to about 1.0 mg/mL, from about 0.05 mg/mL to about 0.9 mg/mL, from about 0.05 mg/mL to about 0.8 mg/mL, from about 0.05 mg/mL to about 0.7 mg/mL, from about 0.05 mg/mL to about 0.6 mg/mL, from about 0.05 mg/mL to about 0.5 mg/mL, from about 0.05 mg/mL to about 0.4 mg/mL, from about 0.05 mg/mL to about 0.3 mg/mL, from about 0.05 mg/mL to about 0.2 mg/mL, from about 0.05 mg/mL to about 0.1 mg/mL, from about 0.1 mg/mL to about 1.0 mg/mL, from about 0.2 mg/mL to about 0.9 mg/mL, from about 0.3 mg/mL to about 0.8 mg/mL, from about 0.4 mg/mL to about 0.7 mg/mL, or from about 0.5 mg/mL to about 0.6 mg/mL. In some embodiments, the empty-LNP solution comprises an empty LNP at a concentration up to about 5.0 mg/mL, up to about 4.0 mg/mL, up to about 3.0 mg/mL, up to about 2.0 mg/mL, up to about 1.0 mg/mL, up to about 0.09 mg/mL, up to about 0.08 mg/mL, up to about 0.07 mg/mL, up to about 0.06 mg/mL, or up to about 0.05 mg/mL.

In some embodiments, the empty-LNP solution comprises an empty LNP in an aqueous buffer. In some embodiments, the empty-LNP solution may further comprise a buffering agent and/or a salt. Exemplary suitable buffering agents include, but are not limited to, ammonium sulfate, sodium bicarbonate, sodium citrate, sodium acetate, potassium phosphate, sodium phosphate, HEPES, and the like. In some embodiments, the empty-LNP solution comprises a buffering agent at a concentration of from about 0.1 mM to about 100 mM, from about 0.5 mM to about 90 mM, from about 1.0 mM to about 80 mM, from about 2 mM to about 70 mM, from about 3 mM to about 60 mM, from about 4 mM to about 50 mM, from about 5 mM to about 40 mM, from about 6 mM to about 30 mM, from about 7 mM to about 20 mM, from about 8 mM to about 15 mM, from about 9 mM to about 12 mM. In some embodiments, the empty-LNP solution comprises a buffering agent at a concentration of or greater than about 0.1 mM, about 0.5 mM, about 1 mM, about 2 mM, about 4 mM, about 6 mM, about 8 mM, about 10 mM, about 15 mM, about 20 mM, about 25 mM, about 30 mM, about 35 mM, about 40 mM, about 45 mM, or about 50 mM. Exemplary suitable salts include, but are not limited to, potassium chloride, magnesium chloride, sodium chloride, and the like.

In some embodiments, the empty-LNP solution has a pH of from about 4.5 to about 7.0, from about 4.6 to about 7.0, from about 4.8 to about 7.0, from about 5.0 to about 7.0, from about 5.5 to about 7.0, from about 6.0 to about 7.0, from about 6.0 to about 6.9, from about 6.0 to about 6.8, from about 6.0 to about 6.7, from about 6.0 to about 6.6, from about 6.0 to about 6.5. In some embodiments, a suitable empty-LNP solution has a pH of or no greater than 4.5, of or no greater than 4.6, of or no greater than 4.7, of or no greater than 4.8, of or no greater than 4.9, of or no greater than 5.0, of or no greater than 5.2, of or no greater than 5.4, of or no greater than 5.6, of or no greater than 5.8, of or no greater than 6.0, of or no greater than 6.1, of or no greater than 6.2, of or no greater than 6.3, of or no greater than 6.4, of or no greater than 6.5, of or no greater than 6.6, of or no greater than 6.7, of or no greater than 6.8, of or no greater than 6.9, or of or no greater than 7.0.

In some embodiments, the empty-LNP solution has a pH in a range of from about 4.5 to about 6.25, from about 4.6 to about 6.0, from about 4.8 to about 5.8, from about 5.0 to about 5.75, from about 5.0 to about 5.5.

In some embodiments, the empty-LNP solution comprises about 5 mM acetate buffer, wherein the acetate buffer has a pH of about 5.0.

In some embodiments, the empty-LNP solution comprises an acetate buffer.

In some embodiments, empty-LNP solution further comprises a first organic solvent.

In some embodiments, the first organic solvent is an alcohol.

In some embodiments, the alcohol is ethanol.

In some embodiments, the empty-LNP solution further comprises a tonicity agent (e.g. a sugar such as sucrose).

### Loaded Lipid Nanoparticles (Loaded LNPs)

In some embodiments, the present disclosure provides a loaded lipid nanoparticle (loaded LNP) being prepared by a method disclosed herein.

In some embodiments, the loaded LNP further comprises from about 0.1 mol% to about 0.5 mol% PEG lipid, a phospholipid, a structural lipid, or any combination thereof.

In some embodiments, the loaded LNP comprises about 3.0 mol% PEG lipid or less, about 2.75 mol% PEG lipid or less, about 2.5 mol% PEG lipid or less, about 2.25 mol% PEG lipid or less, about 2.0 mol% PEG lipid or less, about 1.75 mol% PEG lipid or less, about 1.5 mol% PEG lipid or less, about 1.25 mol% PEG lipid or less, about 1.0 mol% PEG lipid or less, about 0.9 mol% PEG lipid or less, about 0.8 mol% PEG lipid or less, about 0.7 mol% PEG lipid or less, about 0.6 mol% PEG lipid or less, about 0.5 mol% PEG lipid or less, about 0.4 mol% PEG lipid or less, about 0.3 mol% PEG lipid or less, about 0.2 mol% PEG lipid or less, or about 0.1 mol% PEG lipid or less.

In some embodiments, the loaded LNP comprises about 0 mol% to about 3.0 mol% PEG lipid, 0.1 mol% to about 2.5 mol% PEG lipid, about 0.2 mol% to about 2.25 mol% PEG lipid, about 0.25 mol% to about 2.0 mol% PEG lipid, about 0.5 mol% to about 1.75 mol% PEG lipid, about 0.75 mol% to about 1.5 mol% PEG lipid, or about 1.0 mol% to about 1.25 mol% PEG lipid.

In some embodiments, the loaded LNP comprises about 0.050 mol% to about 0.5 mol% PEG lipid.

In some embodiments, the loaded LNP, comprises from about 30 mol% to about 60 mol% ionizable lipid; from about 0 mol% to about 30 mol% phospholipid; from about 15 mol% to about 50 mol% structural lipid; and from about 0.1 mol% to about 0.5 mol% PEG lipid.

In some embodiments, the loaded LNP, comprises from about 30 mol% to about 60 mol% ionizable lipid; from about 0 mol% to about 30 mol% phospholipid; from about 15 mol% to about 50 mol% structural lipid; and from about 0.1 mol% to about 10 mol% PEG lipid.

In some embodiments, the loaded LNP has an average lipid nanoparticle diameter of about 200 nm or less, about 175 nm or less, about 150 nm or less, about 125 nm or less, about 100 nm or less, about 90 nm or less, about 80 nm or less, about 75 nm or less, about 70 nm or less, about 65 nm or less, about 60 nm or less, about 55 nm or less, about 50 nm or less, about 45 nm or less, about 40 nm or less, about 35 nm or less, about 30 nm or less, about 25 nm or less, or about 20 nm or less.

In some embodiments, the loaded LNP has an average lipid nanoparticle diameter of from about 20 nm to about 150 nm, from about 25 nm to about 125 nm, from about 30 nm to about 110 nm, from about 35 nm to about 100 nm, from about 40 nm to about 90 nm, from about 45 nm to about 80 nm, or from about 50 nm to about 70 nm.

In some embodiments, loaded LNP has an average lipid nanoparticle diameter of from about 25 to about 45 nm.

### Loaded Lipid Nanoparticle Solution (Loaded-LNP Solutions)

In some embodiments, the present disclosure provides a loaded-LNP solution being prepared by a method disclosed herein.

In some embodiments, the loaded-LNP solution comprises the loaded LNP. In some embodiments, the loaded-LNP solution comprises the loaded LNP at a concentration of greater than about 0.01 mg/mL, about 0.05 mg/mL, about 0.06 mg/mL, about 0.07 mg/mL, about 0.08 mg/mL, about 0.09 mg/mL, about 0.1 mg/mL, about 0.15 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, or about 1.0 mg/mL. In some embodiments, the loaded-LNP solution comprises the loaded LNP at a concentration of from about 0.01 mg/mL to about 1.0 mg/mL, 0.01 mg/mL to about 0.9 mg/mL, 0.01 mg/mL to about 0.8 mg/mL, 0.01 mg/mL to about 0.7 mg/mL, 0.01 mg/mL to about 0.6 mg/mL, 0.01 mg/mL to about 0.5 mg/mL, 0.01 mg/mL to about 0.4 mg/mL, 0.01 mg/mL to about 0.3 mg/mL, 0.01 mg/mL to about 0.2 mg/mL, 0.01 mg/mL to about 0.1 mg/mL, 0.05 mg/mL to about 1.0 mg/mL, 0.05 mg/mL to about 0.9 mg/mL, 0.05 mg/mL to about 0.8 mg/mL, 0.05 mg/mL to about 0.7 mg/mL, 0.05 mg/mL to about 0.6 mg/mL, 0.05 mg/mL to about 0.5 mg/mL, 0.05 mg/mL to about 0.4 mg/mL, 0.05 mg/mL to about 0.3 mg/mL, 0.05 mg/mL to about 0.2 mg/mL, 0.05 mg/mL to about 0.1 mg/mL, 0.1 mg/mL to about 1.0 mg/mL, 0.2 mg/mL to about 0.9 mg/mL, 0.3 mg/mL to about 0.8 mg/mL, 0.4 mg/mL to about 0.7 mg/mL, or 0.5 mg/mL to about 0.6 mg/mL. In some embodiments, the loaded-LNP solution comprises a loaded LNP at a concentration up to about 5.0 mg/mL, up to about 4.0 mg/mL, up to about 3.0 mg/mL, up to about 2.0 mg/mL, up to about 1.0 mg/mL, up to about 0.09 mg/mL, up to about 0.08 mg/mL, up to about 0.07 mg/mL, up to about 0.06 mg/mL, or up to about 0.05 mg/mL.

In some embodiments, the loaded-LNP solution comprises a loaded LNP in an aqueous buffer. In some embodiments, the loaded-LNP solution may further comprise a buffering agent and/or a salt. Exemplary suitable buffering agents include, but are not limited to, ammonium sulfate, sodium bicarbonate, sodium citrate, sodium acetate, potassium phosphate, sodium phosphate, HEPES, and the like. In some embodiments, the loaded-LNP solution comprises a buffering agent at a concentration of from about 0.1 mM to about 100 mM, from about 0.5 mM to about 90 mM, from about 1.0 mM to about 80 mM, from about 2 mM to about 70 mM, from about 3 mM to about 60 mM, from about 4 mM to about 50 mM, from about 5 mM to about 40 mM, from about 6 mM to about 30 mM, from about 7 mM to about 20 mM, from about 8 mM to about 15 mM, from about 9 mM to about 12 mM. In some embodiments, the loadedmM to about LNP solution comprises a buffering agent at a concentration of or greater than about 0.1 mM, 0.5 mM, 1 mM, 2 mM, 4 mM, 6 mM, 8 mM, 10 mM, 15 mM, 20 mM, 25 mM, 30 mM, 35 mM, 40 mM, 45 mM, or 50 mM. Exemplary suitable salts include, but are not limited to, potassium chloride, magnesium chloride, sodium chloride, and the like.

In some embodiments, the loaded-LNP solution has a pH of from about 4.5 to about 7.0, from about 4.6 to about 7.0, from about 4.8 to about 7.0, from about 5.0 to about 7.0, from about 5.5 to about 7.0, from about 6.0 to about 7.0, from about 6.0 to about 6.9, from about 6.0 to about 6.8, from about 6.0 to about 6.7, from about 6.0 to about 6.6, or from about 6.0 to about 6.5. In some embodiments, a suitable loaded-LNP solution has a pH of or no greater than 4.5, 4.6, of or no greater than 4.7, of or no greater than 4.8, of or no greater than 4.9, of or no greater than 5.0, of or no greater than 5.2, of or no greater than 5.4, of or no greater than 5.6, of or no greater than 5.8, of or no greater than 6.0, of or no greater than 6.1, of or no greater than 6.2, of or no greater than 6.3, of or no greater than 6.4, of or no greater than 6.5, of or no greater than 6.6, of or no greater than 6.7, of or no greater than 6.8, of or no greater than 6.9, or of or no greater than 7.0.

In some embodiments, the loaded-LNP solution has a pH in a range of from about 4.5 to about 6.25, from about 4.6 to about 6.0, from about 4.8 to about 5.8, from about 5.0 to about 5.75, or from about 5.0 to about 5.5.

In some embodiments, the loaded-LNP solution comprises about 5 mM acetate buffer, wherein the acetate buffer has a pH of about 5.0.

In some embodiments, the loaded-LNP solution comprises an acetate buffer.

In some embodiments, loaded-LNP solution further comprises a first organic solvent.

In some embodiments, the first organic solvent is an alcohol.

In some embodiments, the alcohol is ethanol.

In some embodiments, the loaded-LNP solution further comprises a tonicity agent.

### Lipid Nanoparticle Formulations (LNP Formulations)

In some embodiments, the present disclosure provides lipid nanoparticle formulations (LNP formulations) prepared by a method disclosed herein.

In some embodiments, the LNP formulation, comprises about 30-60 mol% ionizable lipid; from about 0 mol% to about 30 mol% phospholipid; from about 15 mol% to about 50 mol% structural lipid; and from about 0.1 mol% to about 0.5 mol% PEG lipid.

In some embodiments, the LNP formulation, comprises from about 30 mol% to about 60 mol% ionizable lipid; from about 0 mol% to about 30 mol% phospholipid; about 15 mol% to about 50 mol% structural lipid; and from about 0.1 mol% to about 10 mol% PEG lipid.

In some embodiments, the LNP formulation has an average lipid nanoparticle diameter of about 200 nm or less, about 175 nm or less, about 150 nm or less, about 125 nm or less, about 100 nm or less, about 90 nm or less, about 80 nm or less, about 75 nm or less, about 70 nm or less, about 65 nm or less, about 60 nm or less, about 55 nm or less, about 50 nm or less, about 45 nm or less, about 40 nm or less, about 35 nm or less, about 30 nm or less, about 25 nm or less, or about 20 nm or less.

In some embodiments, the LNP formulation has an average lipid nanoparticle diameter of from about 20 nm to about 150 nm, from about 25 nm to about 125 nm, from about 30 nm to about 110 nm, from about 35 nm to about 100 nm, from about 40 nm to about 90 nm, from about 45 nm to about 80 nm, or from about 50 nm to about 70 nm.

In some embodiments, LNP formulation has an average lipid nanoparticle diameter of from about 25 to about 45 nm.

In some embodiments, the pH of the LNP formulation is in a range of from about 5.0 to about 6.0, from about 5.1 to about 5.75, or from about 5.2 to about 5.5.

### Administering LNP Formulations

In some embodiments, the administering comprises: (i) providing an active agent solution having a pH in a range of from about 4.5 to about 7.0 comprising a therapeutic and/or prophylactic agent and an empty-LNP solution having a pH in a range of from about 4.5 to about 6.5 comprising an empty LNP, the empty LNP comprising an ionizable lipid; (ii) forming a LNP formulation comprising the loaded LNP encapsulating the therapeutic and/or prophylactic agent by mixing the empty-LNP solution and the active agent solution such that the LNP formulation has a pH in a range of about 4.5 to about less than 7.0; and (iii) administering the LNP formulation to a patient less than about 72 hours after the mixing.

In some embodiments, the first pH and the second pH are in a range of from about 7.0 to about 8.1, or from about 7.1 to about 7.8, or from about 7.2 to about 7.7, or from about 7.3 to about 7.6, or from about 7.4 to about 7.5.

In some embodiments, the first pH and the second pH are in a range of from about 4.5 to about 6.5, or from about 4.6 to about 6.0, or from about 4.8 to about 5.5.

In some embodiments, the administering is performed less than about 72 hours after the mixing. In some embodiments, the administering is performed less than about 60 hours after the mixing. In some embodiments, the administering is performed less than about 48 hours after the mixing. In some embodiments, the administering is performed less than about 36 hours after the mixing. In some embodiments, the administering is performed less than about 24 hours after the mixing. In some embodiments, the administering is performed less than about 20 hours after the mixing. In some embodiments, the administering is performed less than about 16 hours after the mixing. In some embodiments, the administering is performed less than about 12 hours after the mixing. In some embodiments, the administering is performed less than about 8 hours after the mixing.

In some embodiments, the administering is performed less than about 120 minutes after the mixing. In some embodiments, the administering is performed less than about 100 minutes after the mixing. In some embodiments, the administering is performed less than about 90 minutes after the mixing. In some embodiments, the administering is performed less than about 80 minutes after the mixing. In some embodiments, the administering is performed less than about 70 minutes after the mixing. In some embodiments, the administering is performed less than about 60 minutes after the mixing. In some embodiments, the administering is performed less than about 50 minutes after the mixing. In some embodiments, the administering is performed less than about 40 minutes after the mixing. In some embodiments, the administering is performed less than about 30 minutes after the mixing. In some embodiments, the administering is performed less than about 20 minutes after the mixing. In some embodiments, the administering is performed less than about 15 minutes after the mixing. In some embodiments, the administering is performed less than about 10 minutes after the mixing.

In some embodiments, the pH of the aqueous buffer solution and the pH of the lipid nanoparticle formulation are about the same.

In some embodiments, the LNP formulation comprises from about 1% by volume to about 50% by volume of the organic solvent relative to the total volume of the lipid nanoparticle formulation. In some embodiments, the LNP formulation comprises from about 2% by volume to about 45% by volume of the organic solvent relative to the total volume of the LNP formulation. In some embodiments, the LNP formulation comprises from about 3% by volume to about 40% by volume of the organic solvent relative to the total volume of the LNP formulation. In some embodiments, the LNP formulation comprises from about 4% by volume to about 35% by volume of the organic solvent relative to the total volume of the LNP formulation. In some embodiments, the LNP formulation comprises from about 5% by volume to about 33% by volume of the organic solvent relative to the total volume of the LNP formulation.

In some embodiments, the organic solvent is an alcohol.

In some embodiments, the organic solvent is ethanol.

In some embodiments, the organic solvent comprises a first organic solvent and a second organic solvent.

In some embodiments, the first organic solvent is an alcohol and the second organic solvent is an alcohol.

In some embodiments, the first organic solvent is ethanol and the second organic solvent is benzyl alcohol.

In some embodiments, a wt/wt ratio of the first organic solvent to the second organic solvent is in a range of from about 100:1 to about 1:1, or from about 50:1 to about 1:1, or from about 20:1 to about 1:1, or from about 10:1 to about 1:1.

In some embodiments, the organic solution further comprises a wetting agent. As used herein, a wetting agent may refer to an agent that increases, decreases or improves the ability of a liquid to maintain contact with a surface, such as a solid surface and/or liquid surface.

In some embodiments, the wetting agent is an organic solvent.

In some embodiments, the wetting agent is dimethyl sulfoxide (DMSO).

In some embodiments, a wt/wt ratio of the wetting agent to the organic solvent is in a range of from about 1000:1 to about 1:1, or from about 500:1 to about 5:1, or from about 100:1 to about 10:1.

In some embodiments, the aqueous buffer solution is at least one selected from the group consisting of an acetate buffer, citrate buffer, phosphate buffer, and a tris buffer. In some embodiments, the aqueous buffer solution may be any buffer suitable for maintaining a physiological pH. In some embodiments, the aqueous buffer solution may be any buffer suitable for maintaining a pH suitable for administering to a patient. In some embodiments, the patient is a mammalian patient. In some embodiments, the patient is a human patient.

In some embodiments, the aqueous buffer solution further comprises a tonicity agent. As used herein, a tonicity agent may refer to an agent that increases, decreases, or improves the effective osmotic pressure gradient, as defined by the water potential of two solutions, or a relative concentration of solutes dissolve in solution impacting the direction and extent of diffusion.

In some embodiments, the empty-LNP solution or loaded-LNP solution further comprises a tonicity agent.

In some embodiments, the tonicity agent is a sugar.

In some embodiments, the sugar is sucrose.

In some embodiments, the empty-LNP solution or loaded-LNP solution further comprises from about 0.01 g/mL to about 1.0 g/mL, from about 0.05 g/mL to about 0.5 g/mL, from about 0.1 g/mL to about 0.4 g/mL, from about 0.15 g/mL to about 0.3 g/mL, or from about 0.2 g/mL to about 0.25 g/mL tonicity agent.

In some embodiments, the empty-LNP solution or loaded-LNP solution further comprises from about 0.2 g/mL to about 0.25 g/mL tonicity agent.

### Exemplary Embodiments of Empty LNPs, Empty-LNP Solutions, Loaded LNPs, Loaded-LNP Solutions, and LNP Formulations

In some embodiments, the empty LNPs, empty-LNP solutions, loaded LNPs, loaded-LNP solutions, or LNP formulations of the present disclosure comprise a plurality of LNPs, wherein the loaded LNPs or LNP formulations comprise a nucleic acid and an ionizable lipid.

Suitable nucleic acids for the methods of the present disclosure are further disclosed herein. In some embodiments, the nucleic acid is RNA (e.g., mRNA).

Suitable ionizable lipids for the methods of the present disclosure are further disclosed herein.

In some embodiments, the empty LNP, empty-LNP solution, loaded LNP, loaded-LNP solution, or LNP formulation further comprises a phospholipid, a PEG lipid, a structural lipid, or any combination thereof. Suitable phospholipids, PEG lipids, and structural lipids for the methods of the present disclosure are further disclosed herein.

In some embodiments, the empty LNP, empty-LNP solution, loaded LNP, loaded-LNP solution, or LNP formulation of the disclosure includes at least one lipid nanoparticle component. Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

The lipid component of the empty LNP, empty-LNP solution, loaded LNP, loaded-LNP solution, or LNP formulation may include, for example, a lipid according to Formula (IL-I), (IL-IA), (IL-IB), (IL-II), (IL-IIa), (IL-IIb), (IL-IIc), (IL-IId), (IL-IIe), (IL-IIf), (IL-IIg), (IL-VI), (IL-VIIa), (IL-VIIIa), (IL-vmb), (IL-VIIb-1), (IL-VIIb-2), (IL-VIIb-3), (IL-VIIc), (IL-VIId), (IL-VIIIc), (IL-VIIId), (IL-VIVa), (IL-VIVb), (IL-III), (IL-IIIal), (IL-IIIa2), (IL-IIIa3), (IL-IIIa4), (IL-IIIa5), (IL-IIIa6), (IL-IIIa7), or (IL-IIIa8), a phospholipid (such as an unsaturated lipid, e.g., DOPE or DSPC), a PEG lipid, and a structural lipid. The lipid component of the empty LNP, empty-LNP solution, loaded LNP, loaded-LNP solution, or LNP formulation may include, for example, a lipid according to Formula (IL-I), (IL-IA), (IL-IB), (IL-II), (IL-IIa), (IL-IIb), (IL-IIc), (IL-IId), (IL-IIe), (IL-IIf), (IL-IIg), (IL-VI), (IL-VIIa), (IL-VIIIa), (IL-vmb), (IL-VIIb-1), (IL-VIIb-2), (IL-VIIb-3), (IL-VIIc), (IL-VIId), (IL-VIIIc), (IL-VIIId), (IL-VIVa), (IL-VIVb), (IL-III), (IL-IIIal), (IL-IIIa2), (IL-IIIa3), (IL-IIIa4), (IL-IIIa5), (IL-IIIa6), (IL-IIIa7), or (IL-IIIa8), a phospholipid (such as an unsaturated lipid, e.g., DOPE or DSPC), and a structural lipid. The elements of the lipid component may be provided in specific fractions.

In some embodiments, the lipid component of the empty LNP, empty-LNP solution, loaded LNP, loaded-LNP solution, or LNP formulation includes a lipid according to Formula (IL-I), (IL-IA), (IL-IB), (IL-II), (IL-IIa), (IL-IIb), (IL-IIc), (IL-IId), (IL-IIe), (IL-IIf), (IL-IIg), (IL-VI), (IL-VIIa), (IL-VIIIa), (IL-vmb), (IL-VIIb-1), (IL-VIIb-2), (IL-VIIb-3), (IL-VIIc), (IL-VIId), (IL-VIIIc), (IL-VIIId), (IL-VIVa), (IL-VIVb), (IL-III), (IL-IIIal), (IL-IIIa2), (IL-IIIa3), (IL-IIIa4), (IL-IIIa5), (IL-IIIa6), (IL-IIIa7), or (IL-IIIa8), a phospholipid, a PEG lipid, and a structural lipid. In some embodiments, the lipid component of the empty LNP, empty-LNP solution, loaded LNP, loaded-LNP solution, or LNP formulation includes about 30 mol% to about 60 mol% compound of Formula (IL-I), (IL-IA), (IL-IB), (IL-II), (IL-IIa), (IL-IIb), (IL-IIc), (IL-IId), (IL-IIe), (IL-IIf), (IL-IIg), (IL-VI), (IL-VIIa), (IL-VIIIa), (IL-vmb), (IL-VIIb-1), (IL-VIIb-2), (IL-VIIb-3), (IL-VIIc), (IL-VIId), (IL-VIIIc), (IL-VIIId), (IL-VIVa), (IL-VIVb), (IL-III), (IL-IIIal), (IL-IIIa2), (IL-IIIa3), (IL-IIIa4), (IL-IIIa5), (IL-IIIa6), (IL-IIIa7), or (IL-IIIa8), about 0 mol% to about 30 mol% phospholipid, about 18.5 mol% to about 48.5 mol% structural lipid, and about 0 mol% to about 10 mol% of PEG lipid, provided that the total mol% does not exceed 100%. In some embodiments, the lipid component of the empty LNP, empty-LNP solution, loaded LNP, loaded-LNP solution, or LNP formulation includes about 35 mol% to about 55 mol% compound of Formula (IL-I), (IL-IA), (IL-IB), (IL-II), (IL-IIa), (IL-IIb), (IL-IIc), (IL-IId), (IL-IIe), (IL-IIf), (IL-IIg),(IL-VI), (IL-VIIa), (IL-VIIIa), (IL-vmb), (IL-VIIb-1), (IL-VIIb-2), (IL-VIIb-3), (IL-VIIc), (IL-VIId), (IL- VIIIc), (IL-VIIId), (IL-VIVa), (IL-VIVb), (IL-111), (IL-IIIal), (IL-IIIa2), (IL-IIIa3), (IL-IIIa4), (IL-IIIa5), (IL-IIIa6), (IL-IIIa7), or (IL-IIIa8), about 5 mol% to about 25 mol% phospholipid, about 30 mol% to about 40 mol% structural lipid, and about 0 mol% to about 10 mol% of PEG lipid. In a particular embodiment, the lipid component includes about 50 mol% said compound, about 10 mol% phospholipid, about 38.5 mol% structural lipid, and about 1.5 mol% of PEG lipid. In another particular embodiment, the lipid component includes about 40 mol% said compound, about 20 mol% phospholipid, about 38.5 mol% structural lipid, and about 1.5 mol% of PEG lipid. In some embodiments, the phospholipid may be DOPE or DSPC. In some embodiments, the PEG lipid may be PEG-DMG and/or the structural lipid may be cholesterol.

Lipid nanoparticles may be designed for one or more specific applications or targets. In some embodiments, a LNP may be designed to deliver a therapeutic and/or prophylactic such as an RNA to a particular cell, tissue, organ, or system or group thereof in a mammal’s body. Physiochemical properties of lipid nanoparticles may be altered in order to increase selectivity for particular bodily targets. For instance, particle sizes may be adjusted based on the fenestration sizes of different organs. The therapeutic and/or prophylactic included in a LNP may also be selected based on the desired delivery target or targets. In some embodiments, a therapeutic and/or prophylactic may be selected for a particular indication, condition, disease, or disorder and/or for delivery to a particular cell, tissue, organ, or system or group thereof (e.g., localized or specific delivery). In some embodiments, a LNP may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver.

The amount of a therapeutic and/or prophylactic in a LNP may depend on the size, composition, desired target and/or application, or other properties of the lipid nanoparticle as well as on the properties of the therapeutic and/or prophylactic. In some embodiments, the amount of an RNA useful in a LNP may depend on the size, sequence, and other characteristics of the RNA. The relative amounts of a therapeutic and/or prophylactic and other elements (e.g., lipids) in a LNP may also vary. In some embodiments, the wt/wt ratio of the lipid component to a therapeutic and/or prophylactic, such as a nucleic acid, in a LNP may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. In some embodiments, the wt/wt ratio of the lipid component to a therapeutic and/or prophylactic may be from about 10:1 to about 40:1. In some embodiments, the wt/wt ratio is about 20:1. The amount of a therapeutic and/or prophylactic in a LNP may, for example, be measured using absorption spectroscopy (e.g., ultraviolet-visible spectroscopy).

In some embodiments, a LNP includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In some embodiments, the N:P ratio may be from about 2:1 to about 8:1. In some embodiments, the N:P ratio is from about 5:1 to about 8:1. In some embodiments, the N:P ratio may be about 5.0:1, about 5.5:1, about 5.67:1, about 6.0:1, about 6.5:1, or about 7.0:1. In some embodiments, the N:P ratio may be about 5.67:1.

In some embodiments, the formulation including a LNP may further include a salt, such as a chloride salt.

In some embodiments, the formulation including a LNP may further include a sugar such as a disaccharide. In some embodiments, the formulation further includes a sugar but not a salt, such as a chloride salt.

### Physical Properties

The physical properties of the LNP of the present disclosure may be characterized by a variety of methods. In some embodiments, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and zeta potential.

The average LNP diameter of the empty LNP, empty-LNP solution, loaded LNP, loaded-LNP solution, or LNP formulation may be between 10 s of nm and 100 s of nm, e.g., measured by dynamic light scattering (DLS). In some embodiments, the average LNP diameter of the empty LNP, empty-LNP solution, loaded LNP, loaded-LNP solution, or LNP formulation has an average lipid nanoparticle diameter of about 200 nm or less, about 175 nm or less, about 150 nm or less, about 125 nm or less, about 100 nm or less, about 90 nm or less, about 80 nm or less, about 75 nm or less, about 70 nm or less, about 65 nm or less, about 60 nm or less, about 55 nm or less, about 50 nm or less, about 45 nm or less, about 40 nm or less, about 35 nm or less, about 30 nm or less, about 25 nm or less, or about 20 nm or less. In some embodiments, the average LNP diameter of the empty LNP, empty-LNP solution, loaded LNP, loaded-LNP solution, or LNP formulation has an average lipid nanoparticle diameter of about 20 nm to about 150 nm, about 25 nm to about 125 nm, about 30 nm to about 110 nm, about 35 nm to about 100 nm, about 40 nm to about 90 nm, about 45 nm to about 80 nm, or about 50 nm to about 70 nm. In some embodiments, the average LNP diameter of the empty LNP, empty-LNP solution, loaded LNP, loaded-LNP solution, or LNP formulation has an average lipid nanoparticle diameter of about 15 nm to about 55 nm, about 20 nm to about 50 nm, about 25 nm to about 45 nm, or about 30 nm to about 40 nm.

In some embodiments, the average LNP diameter of the empty LNP, empty-LNP solution, loaded LNP, loaded-LNP solution, or LNP formulation has an average lipid nanoparticle diameter of about 25 to about 45 nm.

In some embodiments, the average LNP diameter of the empty LNP, empty-LNP solution, loaded LNP, loaded-LNP solution, or LNP formulation may be from about 70 nm to about 100 nm. In a particular embodiment, the average LNP diameter of the empty LNP, empty-LNP solution, loaded LNP, loaded-LNP solution, or LNP formulation may be about 80 nm. In some embodiments, the average LNP diameter of the empty LNP, empty-LNP solution, loaded LNP, loaded-LNP solution, or LNP formulation may be about 100 nm.

In some embodiments, the average LNP diameter of the empty LNP, empty-LNP solution, loaded LNP, loaded-LNP solution, or LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

In some embodiments, the average LNP diameter of the empty LNP, empty-LNP solution, loaded LNP, loaded-LNP solution, or LNP formulation is about 99% or less, about 98% or less, about 97% or less, about 96% or less, about 95% or less, about 90% or less, about 85% or less, about 80% or less, about 75% or less, about 70% or less, about 65% or less, about 60% or less, about 55% or less, about 50% or less, about 40% or less, about 30% or less, about 20% or less, or about 10% or less as compared to the empty LNP, empty-LNP solution, loaded LNP, loaded-LNP solution, or LNP formulation produced by a different method (e.g., a method excluding one or more of the steps of the methods of the disclosure, or a method that differs from the methods of the disclosure in at least one step).

A LNP may be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The efficiency of encapsulation of a therapeutic and/or prophylactic, such as a nucleic acid describes the amount of therapeutic and/or prophylactic that is encapsulated or otherwise associated with a LNP after preparation, relative to the initial amount provided. The encapsulation efficiency is desirably high (e.g., close to 100%). The encapsulation efficiency may be measured, for example, by comparing the amount of therapeutic and/or prophylactic in a solution containing the lipid nanoparticle before and after breaking up the lipid nanoparticle with one or more organic solvents or detergents. An anion exchange resin may be used to measure the amount of free therapeutic and/or prophylactic (e.g., RNA) in a solution. Fluorescence may be used to measure the amount of free therapeutic and/or prophylactic (e.g., RNA) in a solution. For the lipid nanoparticles described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

### Definitions

As used herein, the term “alkyl” or “alkyl group” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1-14 carbon atoms. Unless otherwise specified, an alkyl group described herein refers to both unsubstituted and substituted alkyl groups.

As used herein, the term “alkenyl” or “alkenyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C₂₋₁₄ alkenyl” means an optionally substituted linear or branched hydrocarbon including 2-14 carbon atoms and at least one carbon-carbon double bond. An alkenyl group may include one, two, three, four, or more carbon-carbon double bonds. In some embodiments, C₁₈ alkenyl may include one or more double bonds. A C₁₈ alkenyl group including two double bonds may be a linoleyl group. Unless otherwise specified, an alkenyl group described herein refers to both unsubstituted and substituted alkenyl groups.

As used herein, the term “alkynyl” or “alkynyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C₂₋₁₄ alkynyl” means an optionally substituted linear or branched hydrocarbon including 2-14 carbon atoms and at least one carbon-carbon triple bond. An alkynyl group may include one, two, three, four, or more carbon-carbon triple bonds. For example, C₁₈ alkynyl may include one or more carbon-carbon triple bonds. Unless otherwise specified, an alkynyl group described herein refers to both unsubstituted and substituted alkynyl groups.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The notation “C₃₋₆ carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2-dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles. In some embodiments, the carbocycle is a C₃₋₈ cycloalkyl. In some embodiments, the carbocycle is a C₃-₆ cycloalkyl. In some embodiments, the carbocycle is a C₆₋₁₀ aryl.

“Aryl” includes groups with aromaticity, including “conjugated,” or multicyclic systems with at least one aromatic ring and do not contain any heteroatom in the ring structure. Examples include phenyl, benzyl, 1,2,3,4-tetrahydronaphthalenyl, etc. In some embodiments, an “aryl” is a C₆₋₁₀ carbocycle with aromaticity (e.g., an “aryl” is a C₆₋₁₀ aryl).

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles. In some embodiments, the heterocycle is a 4 to 12-membered heterocycloalkyl. In some embodiments, the heterocycle is a 5- or 6-membered heteroaryl.

“Heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g.,1, 2, 3, 4, 5, or 6 heteroatoms, independently selected from the group consisting of nitrogen, oxygen sulfur, and boron. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p = 1 or 2). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

As used herein, a “biodegradable group” is a group that may facilitate faster metabolism of a lipid in a mammalian entity. A biodegradable group may be selected from the group consisting of, but is not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)_{2–}, an aryl group, and a heteroaryl group. As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. In some embodiments, M and M′ can be selected from the non-limiting group consisting of optionally substituted phenyl, oxazole, and thiazole. In the formulas herein, M and M′ can be independently selected from the list of biodegradable groups above. Unless otherwise specified, aryl or heteroaryl groups described herein refers to both unsubstituted and substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., —C(O)OH), an alcohol (e.g., a hydroxyl, —OH), an ester (e.g., —C(O)OR or —OC(O)R), an aldehyde (e.g.,—C(O)H), a carbonyl (e.g., —C(O)R, alternatively represented by C═O), an acyl halide (e.g.,—C(O)X, in which X is a halide selected from bromide, fluoride, chloride, and iodide), a carbonate (e.g., —OC(O)OR), an alkoxy (e.g., —OR), an acetal (e.g.,—C(OR)₂R⁗, in which each OR are alkoxy groups that can be the same or different and R⁗ is an alkyl or alkenyl group), a phosphate (e.g., P(O)₄³⁻), a thiol (e.g., -SH), a sulfoxide (e.g., —S(O)R), a sulfinic acid (e.g., —S(O)OH), a sulfonic acid (e.g., —S(O)₂), a thial (e.g., —C(S)H), a sulfate (e.g., S(O)₄²⁻), a sulfonyl (e.g., —S(O)₂—), an amide (e.g., —C(O)NR₂, or —N(R)C(O)R), an azido (e.g., —N₃), a nitro (e.g., —NO₂), a cyano (e.g., —CN), an isocyano (e.g., —NC), an acyloxy (e.g.,—OC(O)R), an amino (e.g., —NR, -NRH, or —NH₂), a carbamoyl (e.g., —OC(O)NR₂, —OC(O)NRH, or —OC(O)NH₂), a sulfonamide (e.g., —S(O)₂NR₂, —S(O)₂NRH, —S(O)₂NH, —N(R)S(O)₂R, —N(H)S(O)₂R, —N(R)S(O)₂H, or —N(H)S(O)₂H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. In some embodiments, a C₁₋₆ alkyl group may be further substituted with one, two, three, four, five, or six substituents as described herein.

Compounds of the disclosure that contain nitrogens can be converted to N-oxides by treatment with an oxidizing agent (e.g., 3-chloroperoxybenzoic acid (mCPBA) and/or hydrogen peroxides) to afford other compounds of the disclosure. Thus, all shown and claimed nitrogen-containing compounds are considered, when allowed by valency and structure, to include both the compound as shown and its N-oxide derivative (which can be designated as N□O or N⁺—O⁻). Furthermore, in other instances, the nitrogens in the compounds of the disclosure can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

As used herein, the terms “approximately” and “about,” as applied to one or more values of interest, refer to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In some embodiments, when used in the context of an amount of a given compound in a lipid component of a LNP, “about” may mean +/- 10% of the recited value. For instance, a LNP including a lipid component having about 40% of a given compound may include 30-50% of the compound.

As used herein, the term “compound,” is meant to include all isomers and isotopes of the structure depicted. “Isotopes” refers to atoms having the same atomic number but different mass numbers resulting from a different number of neutrons in the nuclei. In some embodiments, isotopes of hydrogen include tritium and deuterium. Further, a compound, salt, or complex of the present disclosure can be prepared in combination with solvent or water molecules to form solvates and hydrates by routine methods.

As used herein, the term “contacting” means establishing a physical connection between two or more entities. In some embodiments, contacting a mammalian cell with a LNP means that the mammalian cell and a nanoparticle are made to share a physical connection. Methods of contacting cells with external entities both in vivo and ex vivo are well known in the biological arts. In some embodiments, contacting a LNP and a mammalian cell disposed within a mammal may be performed by varied routes of administration (e.g., intravenous, intramuscular, intradermal, and subcutaneous) and may involve varied amounts of lipid nanoparticles. Moreover, more than one mammalian cell may be contacted by a LNP.

As used herein, the term “comparable method” refers to a method with comparable parameters or steps, as of the method being compared (e.g., the producing the LNP formulation of the present disclosure). In some embodiments, the “comparable method” is a method with one or more of steps i), ia), iaa), ib), ii), iia), iib), iic), iid), and iie) of the method being compared. In some embodiments, the “comparable method” is a method without one or more of steps i), ia), iaa), ib), ii), iia), iib), iic), iid), and iie) of the method being compared. In some embodiments, the “comparable method” is a method without one or more of steps ia) and ib) of the method being compared. In some embodiments, the “comparable method” is a method employing a water-soluble salt of a nucleic acid. In some embodiments, the “comparable method” is a method employing an organic solution that does not comprise an organic solvent-soluble nucleic acid. In some embodiments, the “comparable method” is a method comprising processing the lipid nanoparticle prior to administering the lipid nanoparticle formulation.

As used herein, the term “delivering” means providing an entity to a destination. In some embodiments, delivering a therapeutic and/or prophylactic to a subject may involve administering a LNP including the therapeutic and/or prophylactic to the subject (e.g., by an intravenous, intramuscular, intradermal, or subcutaneous route). Administration of a LNP to a mammal or mammalian cell may involve contacting one or more cells with the lipid nanoparticle.

As used herein, the term “enhanced delivery” means delivery of more(e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target tissue of interest (e.g., mammalian liver) compared to the level of delivery of a therapeutic and/or prophylactic by a control nanoparticle to a target tissue of interest (e.g., MC3, KC2, or DLinDMA). The level of delivery of a nanoparticle to a particular tissue may be measured by comparing the amount of protein produced in a tissue to the weight of said tissue, comparing the amount of therapeutic and/or prophylactic in a tissue to the weight of said tissue, comparing the amount of protein produced in a tissue to the amount of total protein in said tissue, or comparing the amount of therapeutic and/or prophylactic in a tissue to the amount of total therapeutic and/or prophylactic in said tissue. It will be understood that the enhanced delivery of a nanoparticle to a target tissue need not be determined in a subject being treated, it may be determined in a surrogate such as an animal model (e.g., a rat model).

As used herein, the term “specific delivery,” “specifically deliver,” or “specifically delivering” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target tissue of interest (e.g., mammalian liver) compared to an off-target tissue (e.g., mammalian spleen). The level of delivery of a nanoparticle to a particular tissue may be measured by comparing the amount of protein produced in a tissue to the weight of said tissue, comparing the amount of therapeutic and/or prophylactic in a tissue to the weight of said tissue, comparing the amount of protein produced in a tissue to the amount of total protein in said tissue, or comparing the amount of therapeutic and/or prophylactic in a tissue to the amount of total therapeutic and/or prophylactic in said tissue. In some embodiments, for renovascular targeting, a therapeutic and/or prophylactic is specifically provided to a mammalian kidney as compared to the liver and spleen if 1.5, 2-fold, 3-fold, 5-fold, 10-fold, 15 fold, or 20 fold more therapeutic and/or prophylactic per 1 g of tissue is delivered to a kidney compared to that delivered to the liver or spleen following systemic administration of the therapeutic and/or prophylactic. It will be understood that the ability of a nanoparticle to specifically deliver to a target tissue need not be determined in a subject being treated, it may be determined in a surrogate such as an animal model (e.g., a rat model).

As used herein, “encapsulation efficiency” refers to the amount of a therapeutic and/or prophylactic that becomes part of a LNP, relative to the initial total amount of therapeutic and/or prophylactic used in the preparation of a LNP. In some embodiments, if 97 mg of therapeutic and/or prophylactic are encapsulated in a LNP out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement. As used herein, “encapsulation”, “encapsulated”, “loaded”, and “associated” may refer to complete, substantial, or partial enclosure, confinement, surrounding, or encasement. As used herein, “encapsulation” or “association” may refer to the process of confining an individual nucleic acid molecule within a nanoparticle and/or establishing a physiochemical relationship between an individual nucleic acid molecule and a nanoparticle. As used herein, an “empty nanoparticle” may refer to a nanoparticle that is substantially free of a therapeutic or prophylactic agent. As used herein, the term “substantially free of a therapeutic or prophylactic agent” means that the nanoparticle contains no significant amount of therapeutic or prophylactic agent. As used herein, “empty nanoparticle” may refer to a lipid nanoparticle that comprises less than less than 5 wt.%, less than 4 wt.%, less than 3 wt.%, less than 2 wt.%, less than 1 wt.%, less than 0.9 wt.%, less than 0.8 wt.%, less than 0.7 wt.%, less than 0.6 wt.%, less than 0.5 wt.%, less than 0.4 wt.%, less than 0.3 wt.%, less than 0.2 wt.%, or less than 0.1% of a therapeutic or prophylactic agent. As used herein, an “empty nanoparticle” or an “empty lipid nanoparticle” may refer to a nanoparticle that is substantially free of a nucleic acid. As used herein, the term “substantially free of a nucleic acid” means that the nanoparticle contains no significant amount of nucleic acid (e.g., an mRNA). As used herein, an “empty nanoparticle” may refer to a nanoparticle that consists substantially of only lipid components. As used herein, “empty nanoparticle” may refer to a lipid nanoparticle that comprises less than less than 5 wt.%, less than 4 wt.%, less than 3 wt.%, less than 2 wt.%, less than 1 wt.%, less than 0.9 wt.%, less than 0.8 wt.%, less than 0.7 wt.%, less than 0.6 wt.%, less than 0.5 wt.%, less than 0.4 wt.%, less than 0.3 wt.%, less than 0.2 wt.%, or less than 0.1% of a nucleic acid (e.g., an mRNA). As used herein, an “empty nanoparticle” or an “empty lipid nanoparticle” may refer to a nanoparticle that is substantially free of a nucleotide or a polypeptide. As used herein, an “empty nanoparticle” or an “empty lipid nanoparticle” may refer to a nanoparticle that consists substantially of only lipid components. As used herein, a “loaded LNP”, “loaded nanoparticle” or a “loaded lipid nanoparticle” (also referred to as a “full nanoparticle” or a “full lipid nanoparticle”) may refer to a nanoparticle comprising the components of the empty nanoparticle, and a substantial amount of a therapeutic or prophylactic agent. In some embodiments, the loaded LNP comprises a therapeutic or prophylactic agent that is at least partially in the interior of the LNP. In some embodiments, the loaded LNP comprises a substantial amount of a therapeutic or prophylactic agent that is associated with the suface of the LNP or conjugated to the exterior of the LNP. As used herein, a “loaded LNP As used herein, a “loaded LNP”, “loaded nanoparticle” or a “loaded lipid nanoparticle” (also referred to as a “full nanoparticle” or a “full lipid nanoparticle”) may refer to a nanoparticle comprising the components of the empty nanoparticle, and a substantial amount of a nucleotide or polypeptide. In some embodiments, the loaded LNP comprises a nucleotide or polypeptide that is at least partially in the interior of the LNP. In some embodiments, the loaded LNP comprises a nucleotide or polypeptide that is associated with the suface of the LNP or conjugated to the exterior of the LNP. As used herein, a “loaded LNP”, “loaded nanoparticle” or a “loaded lipid nanoparticle” (also referred to as a “full nanoparticle” or a “full lipid nanoparticle”) may refer to a nanoparticle comprising the components of the empty nanoparticle, and a substantial amount of a nucleic acid. In some embodiments, the loaded LNP comprises a nucleic acid (e.g., an mRNA) that is at least partially in the interior of the LNP. In some embodiments, the loaded LNP comprises nucleic acid (e.g., an mRNA) that is associated with the suface of the LNP or conjugated to the exterior of the LNP.

As used herein, “expression” of a nucleic acid sequence refers to translation of an mRNA into a polypeptide or protein and/or post-translational modification of a polypeptide or protein.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

As used herein, the term “isomer” means any geometric isomer, tautomer, zwitterion, stereoisomer, enantiomer, or diastereomer of a compound. Compounds may include one or more chiral centers and/or double bonds and may thus exist as stereoisomers, such as double-bond isomers (i.e., geometric E/Z isomers) or diastereomers (e.g., enantiomers (i.e., (+) or (-)) or cisltrans isomers). The present disclosure encompasses any and all isomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerization is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. An example of tautomerism in disubstituted guanidine is shown below.

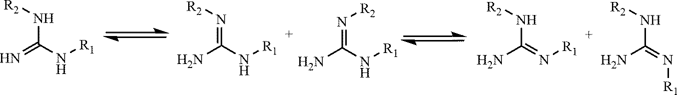

It is to be understood that the compounds of the disclosure may be depicted as different tautomers. It should also be understood that when compounds have tautomeric forms, all tautomeric forms are intended to be included in the scope of the disclosure, and the naming of the compounds does not exclude any tautomer form.

As used herein, a “lipid component” is that component of a lipid nanoparticle that includes one or more lipids. In some embodiments, the lipid component may include one or more cationic/ionizable, PEGylated, structural, or other lipids, such as phospholipids.

As used herein, a “linker” is a moiety connecting two moieties, for example, the connection between two nucleosides of a cap species. A linker may include one or more groups including but not limited to phosphate groups (e.g., phosphates, boranophosphates, thiophosphates, selenophosphates, and phosphonates), alkyl groups, amidates, or glycerols. In some embodiments, two nucleosides of a cap analog may be linked at their 5’ positions by a triphosphate group or by a chain including two phosphate moieties and a boranophosphate moiety.

As used herein, “methods of administration” may include intravenous, intramuscular, intradermal, subcutaneous, or other methods of delivering a composition to a subject. A method of administration may be selected to target delivery (e.g., to specifically deliver) to a specific region or system of a body.

As used herein, “modified” means non-natural. In some embodiments, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. In some embodiments, a modified nucleobase species may include one or more substitutions that are not naturally occurring.

As used herein, the “N:P ratio” is the molar ratio of ionizable (in the physiological pH range) nitrogen atoms in a lipid to phosphate groups in an RNA, e.g., in a LNP including a lipid component and an RNA.

As used herein, a “lipid nanoparticle” is a composition comprising one or more lipids. Lipid nanoparticles are typically sized on the order of micrometers or smaller and may include a lipid bilayer. Lipid nanoparticles, as used herein, unless otherwise specified, encompass lipid nanoparticles (LNPs), liposomes (e.g., lipid vesicles), and lipoplexes. In some embodiments, a LNP may be a liposome having a lipid bilayer with a diameter of 500 nm or less.

As used herein, “naturally occurring” means existing in nature without artificial aid.

As used herein, “patient” refers to a subject who may seek or be in need of treatment, requires treatment, is receiving treatment, will receive treatment, or a subject who is under care by a trained professional for a particular disease or condition.

As used herein, a “PEG lipid” or “PEGylated lipid” refers to a lipid comprising a polyethylene glycol component.

As used herein, a “polymeric lipid” refers to a lipid comprising repeating subunits in its chemical structure. In some embodiments, the polymeric lipid is a lipid comprising a polymer component. In some embodiments, the polymeric lipid is a PEG lipid. In some embodiments, the polymeric lipid is not a PEG lipid. In some embodiments, the polymeric lipid is Brij or OH-PEG-stearate.

The phrase “pharmaceutically acceptable” is used herein to refer to those compounds, materials, composition, and/or dosage forms which are, within the scope of sound medical judgment, suitable for use in contact with the tissues of human beings and animals without excessive toxicity, irritation, allergic response, or other problems or complication, commensurate with a reasonable benefit/risk ratio.

The phrase “pharmaceutically acceptable excipient,” as used herein, refers to any ingredient other than the compounds described herein (for example, a vehicle capable of suspending, complexing, or dissolving the active compound) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. In addition, a crystal polymorphism may be present for the compounds represented by the formula. It is noted that any crystal form, crystal form mixture, or anhydride or hydrate thereof is included in the scope of the present disclosure.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Compositions may also include salts of one or more compounds. Salts may be pharmaceutically acceptable salts. As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. In some embodiments, the nonaqueous media are ether, ethyl acetate, ethanol, isopropanol, or acetonitrile. Lists of suitable salts are found in Remington’s Pharmaceutical Sciences, 17^{th} ed., Mack Publishing Company, Easton, Pa., 1985, p. 1418, Pharmaceutical Salts: Properties, Selection, and Use, P.H. Stahl and C.G. Wermuth (eds.), Wiley-VCH, 2008, and Berge et al., Journal of Pharmaceutical Science, 66, 1-19 (1977), each of which is incorporated herein by reference in its entirety.

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). A phospholipid or an analog or derivative thereof may include choline. A phospholipid or an analog or derivative thereof may not include choline. Particular phospholipids may facilitate fusion to a membrane. In some embodiments, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

As used herein, the “polydispersity index” is a ratio that describes the homogeneity of the particle size distribution of a system. A small value, e.g., less than 0.3, indicates a narrow particle size distribution.

As used herein, an amphiphilic “polymer” is an amphiphilic compound that comprises an oligomer or a polymer. In some embodiments, an amphiphilic polymer can comprise an oligomer fragment, such as two or more PEG monomer units. In some embodiments, an amphiphilic polymer described herein can be PS 20.

As used herein, the term “polypeptide” or “polypeptide of interest” refers to a polymer of amino acid residues typically joined by peptide bonds that can be produced naturally (e.g., isolated or purified) or synthetically.

As used herein, an “RNA” refers to a ribonucleic acid that may be naturally or non-naturally occurring. In some embodiments, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. In some embodiments, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (IncRNA) and mixtures thereof.

As used herein, a “single unit dose” is a dose of any therapeutic administered in one dose/at one time/single route/single point of contact, i.e., single administration event.

As used herein, a “split dose” is the division of a single unit dose or total daily dose into two or more doses.

As used herein, a “total daily dose” is an amount given or prescribed in a 24 hour period. It may be administered as a single unit dose.

As used herein, the term “subject” refers to any organism to which a composition or formulation in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

As used herein, “Tₓ” refers to the amount of time lasted for the nucleic acid integrity (e.g., mRNA integrity) of a LNP, LNP solution, lyophilized LNP composition, or LNP formulation to degrade to about X of the initial integrity of the nucleic acid (e.g., mRNA) used for the preparation of the LNP, LNP solution, lyophilized LNP composition, or LNP formulation. For example, “T_{80%}” refers to the amount of time lasted for the nucleic acid integrity (e.g., mRNA integrity) of a LNP, LNP solution, lyophilized LNP composition, or LNP formulation to degrade to about 80% of the initial integrity of the nucleic acid (e.g., mRNA) used for the preparation of the LNP, LNP solution, lyophilized LNP composition, or LNP formulation. For another example, “T_{½}” refers to the amount of time lasted for the nucleic acid integrity (e.g., mRNA integrity) of a LNP, LNP solution, lyophilized LNP composition, or LNP formulation to degrade to about ½ of the initial integrity of the nucleic acid (e.g., mRNA) used for the preparation of the LNP, LNP solution, lyophilized LNP composition, or LNP formulation.

As used herein, “targeted cells” refers to any one or more cells of interest. The cells may be found in vitro, in vivo, in situ, or in the tissue or organ of an organism. The organism may be an animal. In some embodiments, the organism is a mammal. In some embodiments, the organism is a human. In some embodiments, the organism is a patient.

As used herein, “target tissue” refers to any one or more tissue types of interest in which the delivery of a therapeutic and/or prophylactic would result in a desired biological and/or pharmacological effect. Examples of target tissues of interest include specific tissues, organs, and systems or groups thereof. In particular applications, a target tissue may be a kidney, a lung, a spleen, vascular endothelium in vessels (e.g., intra-coronary or intra-femoral), or tumor tissue (e.g., via intratumoral injection). An “off-target tissue” refers to any one or more tissue types in which the expression of the encoded protein does not result in a desired biological and/or pharmacological effect. In particular applications, off-target tissues may include the liver and the spleen.

The term “therapeutic agent” or “prophylactic agent” refers to any agent that, when administered to a subject, has a therapeutic, diagnostic, and/or prophylactic effect and/or elicits a desired biological and/or pharmacological effect. Therapeutic agents are also referred to as “actives” or “active agents.” Such agents include, but are not limited to, cytotoxins, radioactive ions, chemotherapeutic agents, small molecule drugs, proteins, and nucleic acids.

As used herein, the term “therapeutically effective amount” means an amount of an agent to be delivered (e.g., nucleic acid, drug, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, “transfection” refers to the introduction of a species (e.g., an RNA) into a cell. Transfection may occur, for example, in vitro, ex vivo, or in vivo.

As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. In some embodiments, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the “zeta potential” is the electrokinetic potential of a lipid, e.g., in a particle composition.

### Ionizable Lipids

The present disclosure provides ionizable lipids. In some embodiments, the ionizable lipids include a central amine moiety and at least one biodegradable group. In some embodiments, the ionizable lipid is an amino lipid. The lipids described herein may be advantageously used in lipid nanoparticles and lipid nanoparticle formulations for the delivery of therapeutic and/or prophylactics, such as a nucleic acid, to mammalian cells or organs.

In some aspects, the ionizable lipids of the present disclosure may be one or more of compounds of Formula (IL-1):

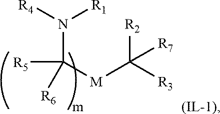

or their N-oxides, or salts or isomers thereof, wherein:
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of hydrogen, a C₃₋₆ carbocycle, —(CH₂)_{nQ}, —(CH₂)ₙCHQR, —(CH₂)ₒC(R¹⁰)₂(CH₂)_{n-oQ}, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)2, —N(R)C(S)N(R)2, —N(R)R⁸, —N(R)S(O)2R⁸, —O(CH2)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;
each R⁵ is independently selected from the group consisting of OH, C₁₋₃ alkyl, C₂-₃ alkenyl, and H;
each R⁶ is independently selected from the group consisting of OH, C₁₋₃ alkyl, C₂-₃ alkenyl, and H;
M and M′ are independently selected from —C(O)O—, —OC(O)—, —OC(O)—M″—C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R⁷ is selected from the group consisting of C₁₋₃ alkyl, C₂₋₃ alkenyl, and H;
R⁸ is selected from the group consisting of C₃₋₆ carbocycle and heterocycle;
R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
R¹⁰ is selected from the group consisting of H, OH, C₁₋₃ alkyl, and C₂₋₃ alkenyl;
each R is independently selected from the group consisting of C₁₋₃ alkyl, C₂₋₃ alkenyl, (CH₂)_{q}OR*, and H,
and each q is independently selected from 1, 2, and 3;
each R′ is independently selected from the group consisting of C₁₋₁₈ alkyl, C₂₋₁₈ alkenyl, —R*YR″″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13; and wherein when R⁴ is —(CH₂)_{nQ}, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some aspects, the ionizable lipids of the present disclosure may be one or more of compounds of Formula (IL-X):

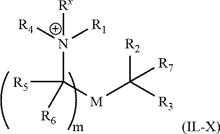

or its N-oxide, or a salt or isomer thereof, wherein or a salt or isomer thereof, wherein
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of hydrogen, a C₃₋₆ carbocycle, —(CH₂)ₙQ, —(CH2)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, N(R)R⁸ —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;
R^{x} is selected from the group consisting of C₁₋₆ alkyl, C₂₋₆ alkenyl, —(CH₂)ᵥOH, and —(CH₂)ᵥN(R)₂,
wherein v is selected from 1, 2, 3, 4, 5, and 6;
each R⁵ is independently selected from the group consisting of OH, C₁₋₃ alkyl, C₂₋₃ alkenyl, and H;
each R⁶ is independently selected from the group consisting of OH, C₁₋₃ alkyl, C₂₋₃ alkenyl, and H;
M and M′ are independently selected from —C(O)O—, —OC(O)—, —OC(O)—M″—C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R⁷ is selected from the group consisting of C₁₋₃ alkyl, C₂₋₃ alkenyl, and H;
R⁸ is selected from the group consisting of C₃₋₆ carbocycle and heterocycle;
R⁹ is selected from the group consisting of H, CN, NO₂, C₁₋₆ alkyl, -OR, —S(O)₂R, —S(O)₂N(R)₂, C₂₋₆ alkenyl, C₃₋₆ carbocycle and heterocycle;
R¹⁰ is selected from the group consisting of H, OH, C₁₋₃ alkyl, and C₂₋₃ alkenyl;
each R is independently selected from the group consisting of C₁₋₃ alkyl, C₂₋₃ alkenyl, (CH₂)_{q}OR*, and H,
and each q is independently selected from 1, 2, and 3;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, a subset of compounds of Formula (IL-I) includes those of Formula (IL-IA):

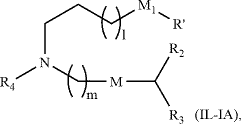

or its N-oxide, or a salt or isomer thereof, wherein 1 is selected from 1, 2, 3, 4, and 5; m is selected from 5, 6, 7, 8, and 9; M₁ is a bond or M′; R⁴ is hydrogen, unsubstituted C₁₋₃ alkyl, —(CH₂)ₒC(R¹⁰)₂(CH₂)ₙ₋ₒQ, or —(CH₂)ₙQ, in which Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected from —C(O)O—, —OC(O)—, —OC(O)—M″—C(O)O—, —C(O)N(R′)—, —P(O)(OR′)O—, —S—S—, an aryl group, and a heteroaryl group,; and R² and R³ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In some embodiments, a subset of compounds of Formula (I) includes those of Formula (IL-IB):

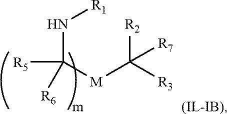

or its N-oxide, or a salt or isomer thereof, in which all variables are as defined herein. In some embodiments, m is selected from 5, 6, 7, 8, and 9; R₄ is hydrogen, unsubstituted C₁₋₃ alkyl, or —(CH₂)ₙQ, in which Q is —OH, —NHC(S)N(R)₂, —NHC(O)N(R)₂, —N(R)C(O)R, —N(R)S(O)₂R, —N(R)R⁸, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)—M″— C(O)O—, —C(O)N(R′)—, —P(O)(OR′)O—, —S—S—, an aryl group, and a heteroaryl group,; and R₂ and R₃ are independently selected from the group consisting of H, C₁₋₁₄ alkyl, and C₂₋₁₄ alkenyl. In some embodiments, m is 5, 7, or 9. In some embodiments, Q is OH, —NHC(S)N(R)₂, or —NHC(O)N(R)₂. In some embodiments, Q is —N(R)C(O)R, or —N(R)S(O)₂R.

In some embodiments, a subset of compounds of Formula (IL-I) includes those of Formula (IL-II):

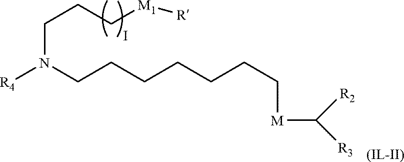

or its N-oxide, or a slat or isomer thereof, wherein 1 is selected from 1, 2, 3, 4 and 5; M1 is a bond or M′; R₄ is hydrogen, unsubstituted C₁₋₃ alkyl, or —(CH₂)ₙQ, in which n is 2, 3, or 4, and Q is —OH, — NHC(S)N(R)₂, —NHC(O)N(R)₂, —N(R)C(O)R, —N(R)S(O)₂R, —N(R)R⁸, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)—M″—C(O)O—, —C(O)N(R′)—, —P(O)(OR′)O—, —S—S—, an aryl group, and a heteroaryl group,; and R₂ and R₃ are independently selected from the group consisting of H, C₁₋₁₄ alkyl, and C₂₋₁₄ alkenyl.

In some aspects, the ionizable lipids of the present disclosure may be one or more of compounds of Formula (IL-VI):

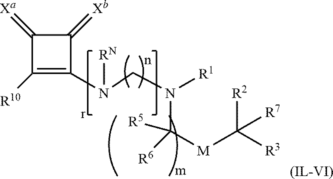

or its N-oxide, or a salt or isomer thereof, wherein
R¹ is selected from the group consisting of C₅₋₃₀ alkyl, C₅₋₂₀ alkenyl, -R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
each R⁵ is independently selected from the group consisting of OH, C₁₋₃ alkyl, C₂₋₃ alkenyl, and H;
each R⁶ is independently selected from the group consisting of OH, C₁₋₃ alkyl, C₂₋₃ alkenyl, and H;
M and M′ are independently selected from —C(O)O—, —OC(O)—, —OC(O)—M″—C(O)O—, —C(O)N(R′)—, —N(R′)C(O)—, —C(O)—, —C(S)—, —C(S)S—, —SC(S)—, —CH(OH)—, —P(O)(OR′)O—, —S(O )₂—, —S—S—, an aryl group, and a heteroaryl group, in which M″ is a bond, C₁₋₁₃ alkyl or C₂₋₁₃ alkenyl;
R⁷ is selected from the group consisting of C₁₋₃ alkyl, C₂₋₃ alkenyl, and H; each R is independently selected from the group consisting of H, C₁₋₃ alkyl, and C₂₋₃ alkenyl;
R^{N} is H, or C₁₋₃ alkyl;
each R′ is independently selected from the group consisting of C₁₋₁₈ alkyl, C₂₋₁₈ alkenyl, —R*YR″, —YR, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I;
X^{a} and X^{b} are each independently O or S;
R¹⁰ is selected from the group consisting of H, halo, —OH, R, —N(R)₂, —CN, —N₃, —C(O)OH, —C(O)OR, —OC(O)R, —OR, —SR, —S(O)R, —S(O)OR, —S(O)₂OR, —NO₂, —S(O)₂N(R)₂, —N(R)S(O)₂R, —NH(CH₂)ₜₗN(R)₂, —NH(CH₂)ₚₗO(CH₂)_{ql}N(R)₂, —NH(CH₂)ₛₗOR, —N((CH₂)ₛₗOR)₂, a carbocycle, a heterocycle, aryl and heteroaryl;
m is selected from 5, 6, 7, 8, 9, 10, 11, 12, and 13;
n is selected from 1, 2, 3, 4, 5, 6, 7, 8, 9, and 10;
r is 0 or 1;
t¹ is selected from 1, 2, 3, 4, and 5;
p¹ is selected from 1, 2, 3, 4, and 5;
q¹ is selected from 1, 2, 3, 4, and 5; and
s¹ is selected from 1, 2, 3, 4, and 5.

In some embodiments, a subset of compounds of Formula (IL-VI) includes those of Formula (IL-VI-a):

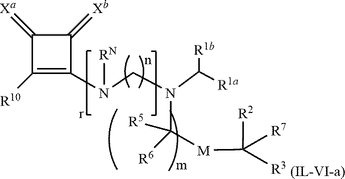

or its N-oxide, or a salt or isomer thereof, wherein
R^{1a} and R^{1b} are independently selected from the group consisting of C₁₋₁₄ alkyl and C₂₋₁₄ alkenyl; and
R² and R³ are independently selected from the group consisting of C₁₋₁₄ alkyl, C₂₋₁₄ alkenyl, —R*YR″, —YR″, and —R*OR″, or R² and R³, together with the atom to which they are attached, form a heterocycle or carbocycle.

In another embodiment, a subset of compounds of Formula (IL-VI) includes those of Formula (IL-VII):

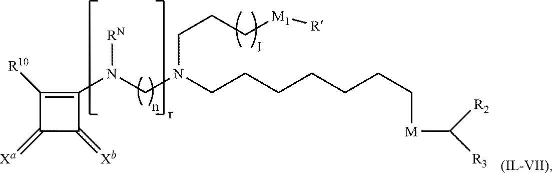

or its N-oxide, or a salt or isomer thereof, wherein
1 is selected from 1, 2, 3, 4, and 5;
M₁ is a bond or M′; and
R² and R³ are independently selected from the group consisting of H, C₁₋₁₄ alkyl, and C₂₋₁₄ alkenyl.

In another embodiment, a subset of compounds of Formula (IL-VI) includes those of Formula (IL-VIII):

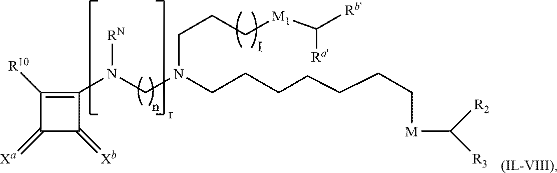

or its N-oxide, or a salt or isomer thereof, wherein
1 is selected from 1, 2, 3, 4, and 5;
M₁ is a bond or M′; and
R^{a′} and R^{b′} are independently selected from the group consisting of C ₁₋₁₄ alkyl and C₂₋₁₄ alkenyl; and
R² and R³ are independently selected from the group consisting of C₁₋₁₄ alkyl, and C₂₋₁₄ alkenyl.

The compounds of any one of formula (IL-I), (IL-IA), (IL-VI), (IL-VI-a), (IL-VII) or (IL-VIII) include one or more of the following features when applicable.

In some embodiments, M₁ is M′.

In some embodiments, M and M′ are independently —C(O)O— or —OC(O)—.

In some embodiments, at least one of M and M′ is —C(O)O— or —OC(O)—.

In certain embodiments, at least one of M and M′ is —OC(O)—.

In certain embodiments, M is —OC(O)— and M′ is —C(O)O—. In some embodiments, M is —C(O)O— and M′ is —OC(O)—. In certain embodiments, M and M′ are each —OC(O)—. In some embodiments, M and M′ are each —C(O)O—.

In certain embodiments, at least one of M and M′ is —OC(O)—M″—C(O)O—.

In some embodiments, M and M′ are independently —S—S—.

In some embodiments, at least one of M and M′ is —S—S—.

In some embodiments, one of M and M′ is —C(O)O— or —OC(O)— and the other is —S—S—. For example, M is —C(O)O— or —OC(O)— and M′ is —S—S— or M′ is —C(O)O—, or —OC(O)— and M is —S—S—.

In some embodiments, one of M and M′ is —OC(O)—M″—C(O)O—, in which M″ is a bond, C₁₋₁₃ alkyl or C₂₋₁₃ alkenyl. In other embodiments, M″ is C₁₋₆ alkyl or C₂₋₆ alkenyl. In certain embodiments, M″ is C₁₋₄ alkyl or C₂₋₄ alkenyl. For example, in some embodiments, M″ is C₁ alkyl. For example, in some embodiments, M″ is C₂ alkyl. For example, in some embodiments, M″ is C₃ alkyl. For example, in some embodiments, M″ is C₄ alkyl. For example, in some embodiments, M″ is C₂ alkenyl. For example, in some embodiments, M″ is C₃ alkenyl. For example, in some embodiments, M″ is C₄ alkenyl.

In some embodiments, 1 is 1, 3, or 5.

In some embodiments, R⁴ is hydrogen.

In some embodiments, R⁴ is not hydrogen.

In some embodiments, R⁴ is unsubstituted methyl or —(CH₂)ₙQ, in which Q is OH, —NHC(S)N(R)₂, —NHC(O)N(R)₂, —N(R)C(O)R, or —N(R)S(O)₂R.

In some embodiments, Q is OH.

In some embodiments, Q is —NHC(S)N(R)₂.

In some embodiments, Q is —NHC(O)N(R)₂.

In some embodiments, Q is —N(R)C(O)R.

In some embodiments, Q is —N(R)S(O)₂R.

In some embodiments, Q is —O(CH₂)ₙN(R)₂.

In some embodiments, Q is —O(CH₂)ₙOR.

In some embodiments, Q is —N(R)R⁸.

In some embodiments, Q is —NHC(═NR⁹)N(R)₂.

In some embodiments, Q is —NHC(═CHR⁹)N(R)₂.

In some embodiments, Q is —OC(O)N(R)₂.

In some embodiments, Q is —N(R)C(O)OR.

In some embodiments, n is 2.

In some embodiments, n is 3.

In some embodiments, n is 4.

In some embodiments, M₁ is absent.

In some embodiments, at least one R⁵ is hydroxyl. For example, one R⁵ is hydroxyl.

In some embodiments, at least one R⁶ is hydroxyl. For example, one R⁶ is hydroxyl.

In some embodiments one of R⁵ and R⁶ is hydroxyl. For example, one R⁵ is hydroxyl and each R⁶ is hydrogen. For example, one R⁶ is hydroxyl and each R⁵ is hydrogen.

In some embodiments, R^{x} is C₁₋₆ alkyl. In some embodiments, R^{x} is C₁₋₃ alkyl. For example, R^{x} is methyl. For example, R^{x} is ethyl. For example, R^{x} is propyl.

In some embodiments, R^{x} is —(CH₂)ᵥOH and, v is 1, 2 or 3. For example, R^{x} is methanoyl. For example, R^{x} is ethanoyl. For example, R^{x} is propanoyl.

In some embodiments, R^{x} is —(CH₂)ᵥN(R)₂, v is 1, 2 or 3 and each R is H or methyl. For example, R^{x} is methanamino, methylmethanamino, or dimethylmethanamino. For example, R^{x} is aminomethanyl, methylaminomethanyl, or dimethylaminomethanyl. For example, R^{x} is aminoethanyl, methylaminoethanyl, or dimethylaminoethanyl. For example, R^{x} is aminopropanyl, methylaminopropanyl, or dimethylaminopropanyl.

In some embodiments, R′ is C₁₋₁₈ alkyl, C₂₋₁₈ alkenyl, —R*YR″, or —YR″.

In some embodiments, R² and R³ are independently C₃₋₁₄ alkyl or C₃₋₁₄ alkenyl.

In some embodiments, R^{1b} is C₁₋₁₄ alkyl. In some embodiments, R^{1b} is C₂₋₁₄ alkyl. In some embodiments, R^{1b} is C₃₋₁₄ alkyl. In some embodiments, R^{1b} is C₁₋₈ alkyl. In some embodiments, R^{1b} is C₁₋₅ alkyl. In some embodiments, R^{1b} is C₁₋₃ alkyl. In some embodiments, R^{1b} is selected from C₁ alkyl, C₂ alkyl, C₃ alkyl, C₄ alkyl, and C₅ alkyl. For example, in some embodiments, R^{1b} is C₁ alkyl. For example, in some embodiments, R^{1b} is C₂ alkyl. For example, in some embodiments, R^{1b} is C₃ alkyl. For example, in some embodiments, R^{1b} is C₄ alkyl. For example, in some embodiments, R^{1b} is C₅ alkyl.

In some embodiments, R¹ is different from —(CHR⁵R⁶)ₘ₋M—CR²R³R⁷.

In some embodiments, —CHR^{1a}R^{1b}— is different from —(CHR⁵R⁶)ₘ₋M—CR²R³R⁷.

In some embodiments, R⁷ is H. In some embodiments, R⁷ is selected from C₁₋₃ alkyl. For example, in some embodiments, R⁷ is C₁ alkyl. For example, in some embodiments, R⁷ is C₂ alkyl. For example, in some embodiments, R⁷ is C₃ alkyl. In some embodiments, R⁷ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and C₁₈ alkenyl.

In some embodiments, Rb′ is C₁₋₁₄ alkyl. In some embodiments, Rb′ is C2-14 alkyl. In some embodiments, R^{b′} is C₃-₁₄ alkyl. In some embodiments, R^{b′} is C₁₋₈ alkyl. In some embodiments, R^{b′} is C₁₋₅ alkyl. In some embodiments, R^{b′} is C₁₋₃ alkyl. In some embodiments, R^{b′} is selected from C₁ alkyl, C₂ alkyl, C₃ alkyl, C₄ alkyl and C₅ alkyl. For example, in some embodiments, R^{b′} is C₁ alkyl. For example, in some embodiments, R^{b′} is C₂ alkyl. For example, some embodiments, R^{b′} is C₃ alkyl. For example, some embodiments, R^{b′} is C₄ alkyl.

In one embodiment, the compounds of Formula (IL-I) are of Formula (IL-IIa):

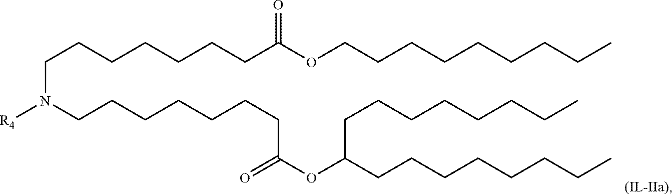

or their N-oxides, or salts or isomers thereof, wherein R₄ is as described herein.

In another embodiment, the compounds of Formula (IL-I) are of Formula (IL-IIb):

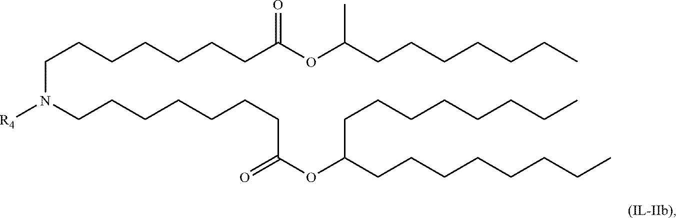

or their N-oxides, or salts or isomers thereof, wherein R₄ is as described herein.

In another embodiment, the compounds of Formula (IL-I) are of Formula (IL-IIc) or (IL-IIe):

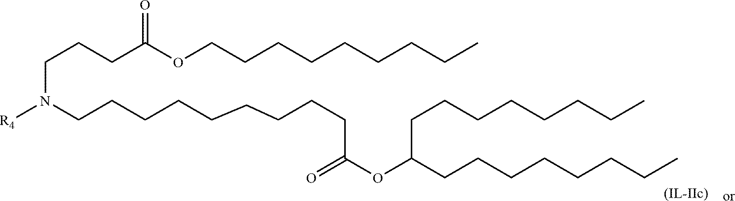

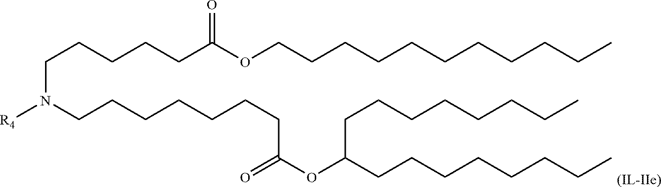

or their N-oxides, or salts or isomers thereof, wherein R₄ is as described herein.

In another embodiment, the compounds of Formula (IL-I) are of Formula (IL-IIf):

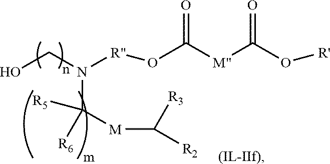

or their N-oxides, or salts or isomers thereof, wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (IL-I) are of Formula (IL-IId):

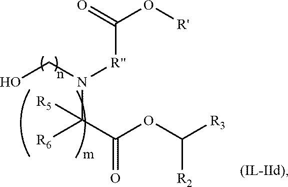

or their N-oxides, or salts or isomers thereof, wherein n is 2, 3, or 4; and m, R′, R″, and R₂ through R₆ are as described herein. In some embodiments, each of R₂ and R₃ may be independently selected from the group consisting of C₅₋₁₄ alky and C₅₋₁₄ alkenyl.

In a further embodiment, the compounds of Formula (IL-I) are of Formula (IL-IIg):

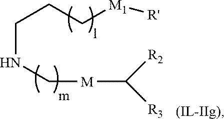

or their N-oxides, or salts or isomers thereof, wherein 1 is selected from 1, 2, 3, 4, and 5; m is selected from 5, 6, 7, 8, and 9; M₁ is a bond or M′; M and M′ are independently selected from from —C(O)O—, —OC(O)—, —OC(O)—M″—C(O)O—, —C(O)N(R′, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. In some embodiments, M″ is C₁₋₆ alkyl (e.g., C₁₋₄ alkyl) or C₂₋₆ alkenyl (e.g. C₂-₄ alkenyl). In some embodiments, R₂ and R₃ are independently selected from the group consisting of C₅₋₁₄ alkyl and C₅₋₁₄ alkenyl.

In another embodiment, a subset of compounds of Formula (IL-VI) includes those of Formula (IL-VIIa):

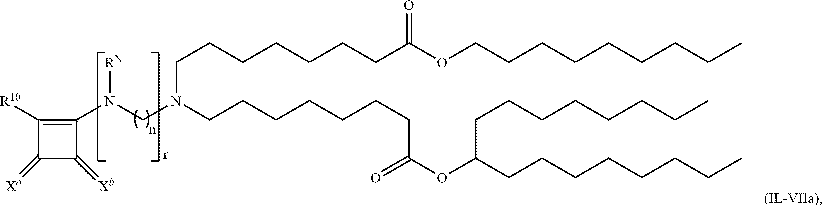

or its N-oxide, or a salt or isomer thereof.

In another embodiment, a subset of compounds of Formula (VI) includes those of Formula (IL-VIIIa):

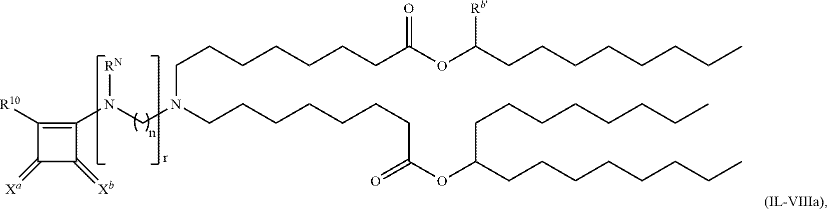

or its N-oxide, or a salt or isomer thereof.

In another embodiment, a subset of compounds of Formula (IL-VI) includes those of Formula (IL-VIIIb):

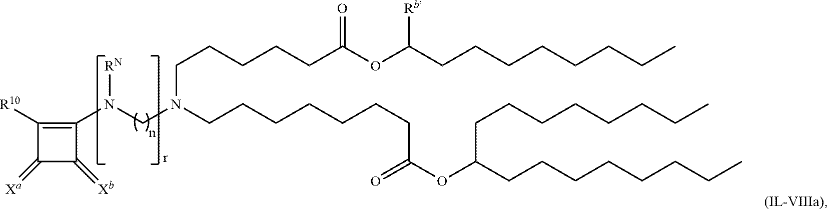

or its N-oxide, or a salt or isomer thereof.

In another embodiment, a subset of compounds of Formula (IL-VI) includes those of Formula (IL-VIIb-1):

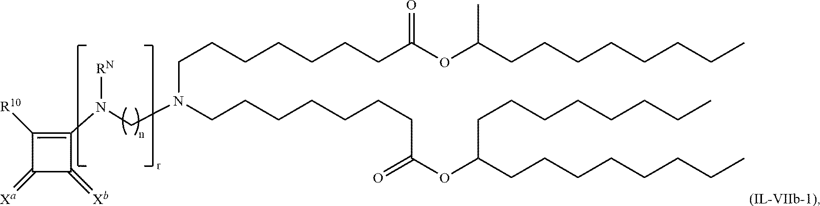

or its N-oxide, or a salt or isomer thereof.

In another embodiment, a subset of compounds of Formula (IL-VI) includes those of Formula (IL-VIIb-2):

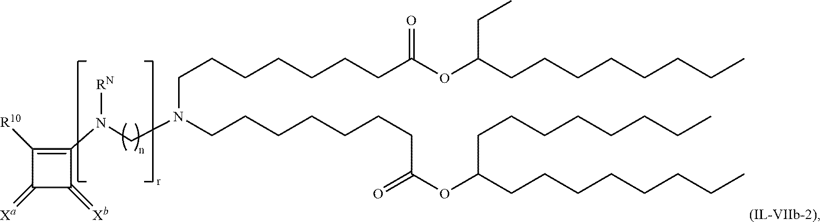

or its N-oxide, or a salt or isomer thereof.

In another embodiment, a subset of compounds of Formula (IL-VI) includes those of Formula (IL-VIIb-3):

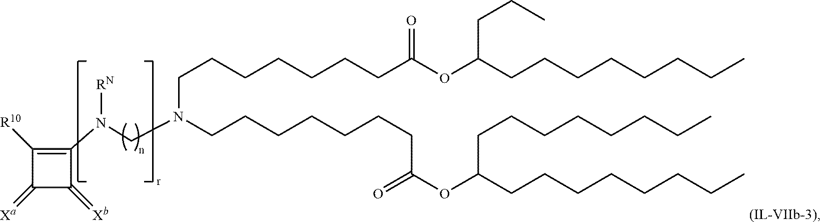

or its N-oxide, or a salt or isomer thereof.

In another embodiment, a subset of compounds of Formula (IL-VI) includes those of Formula (IL-VIIc):

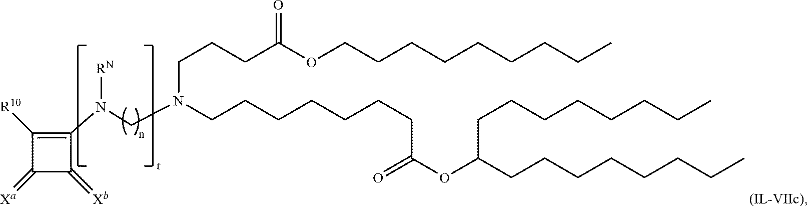

In another embodiment, a subset of compounds of Formula (IL-VI) includes those of Formula (IL-VIId):

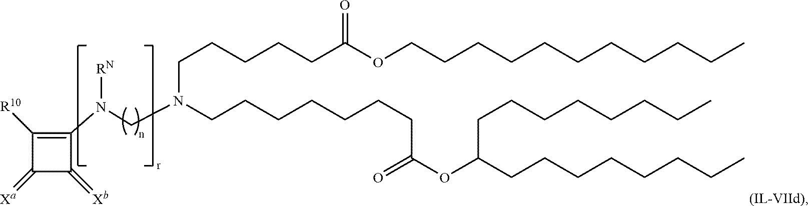

or its N-oxide, or a salt or isomer thereof.

In another embodiment, a subset of compounds of Formula (IL-VI) includes those of Formula (IL-VIIIc):

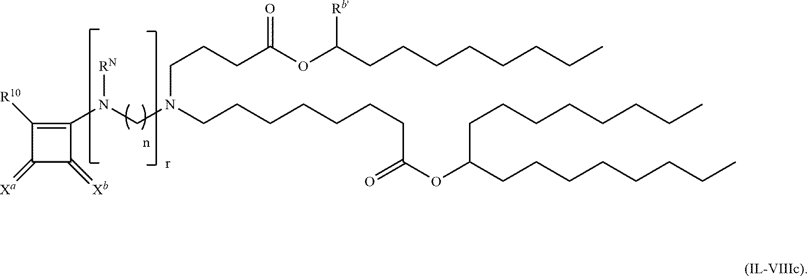

In another embodiment, a subset of compounds of Formula (IL-VI) includes those of Formula (IL-VIIId):

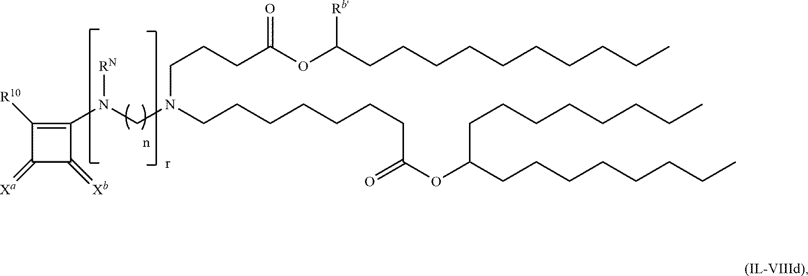

or its N-oxide, or a salt or isomer thereof.

In some embodiments, the ionizable lipids are one or more of the compounds described in PCT Application Nos. PCT/US2020/051613, PCT/US2020/051613, and PCT/US2020/051629, and in PCT Publication Nos. WO 2017/049245, WO 2018/170306, WO 2018/170336, and WO 2020/061367.

In some embodiments, the ionizable lipids are selected from Compounds 1-280 described in U.S. Application No. 62/475,166.

In some embodiments, the ionizable lipid is

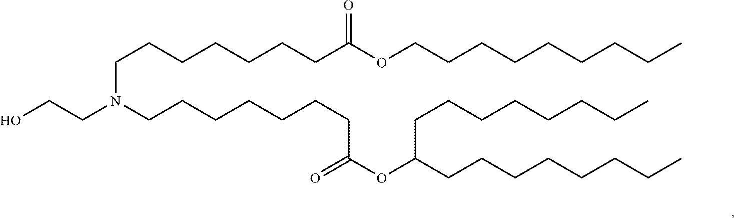

or a salt thereof.

In some embodiments, the ionizable lipid is

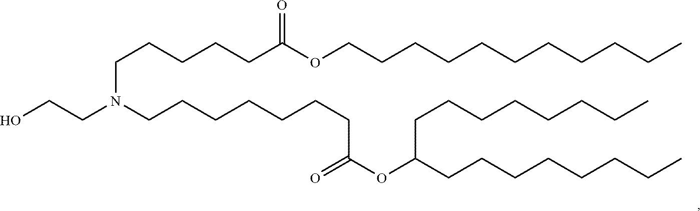

or a salt thereof.

In some embodiments, the ionizable lipid is

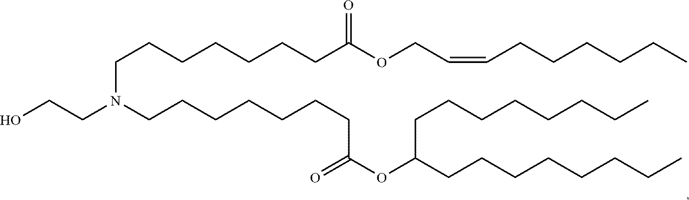

or a salt thereof.

In some embodiments, the ionizable lipid is

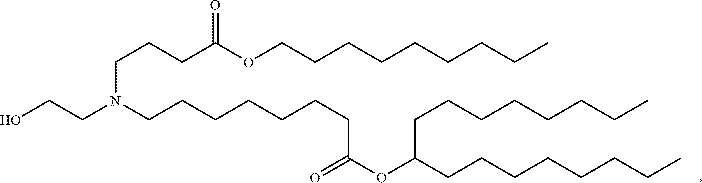

or a salt thereof.

In some aspects, the ionizable lipids of the present disclosure may be one or more of compounds of formula (IL-VIVa):

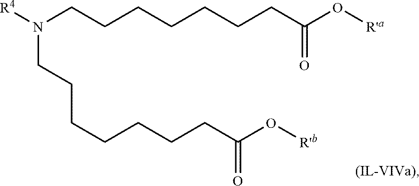

or its N-oxide, or a salt or isomer thereof,
wherein R′^{a} is R’^{branched} or R’^{cyclic}; wherein R^{aγ}
R’^{branched} is

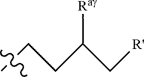

R′ and R’^{cyclic} is:

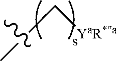

and
R′^{b} is:

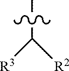

or;

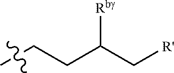

;
wherein

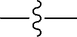

denotes a point of attachment;
wherein R^{aγ} and R^{bγ} are each independently a C₂₋₁₂ alkyl or C₂₋₁₂ alkenyl;
R² and R³ are each independently selected from the group consisting of C₁₋₁₄ alkyl and C₂₋₁₄ alkenyl;
R⁴ is -(CH₂)₂OH;
each R′ independently is a C₁₋₁₂ alkyl or C₂₋₁₂ alkenyl;
Y^{a} is a C₃₋₆ carbocycle;
R*′′^{a} is selected from the group consisting of C₁₋₁₅ alkyl and C₂₋₁₅ alkenyl; and
s is 2 or 3.

In some aspects, the ionizable lipids of the present disclosure may be one or more of compounds of formula (IL-VIVb):

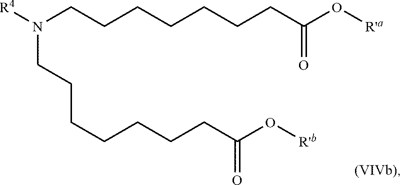

or its N-oxide, or a salt or isomer thereof,
wherein R′^{a} is R’^{branched} or R’^{cyclic}; wherein
R’^{branched} is

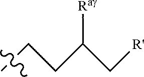

and R’^{cyclic} is:

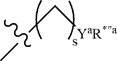

; and R′^{b} is:

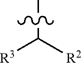

or;

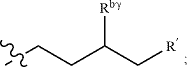

;
wherein

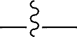

denotes a point of attachment;
wherein R^{aγ} and R^{bγ} are each independently a C₂₋₁₂ alkyl or C₂₋₁₂ alkenyl;
R² and R³ are each independently selected from the group consisting of C₁₋₁₄ alkyl and C₂₋₁₄ alkenyl;
R⁴ is

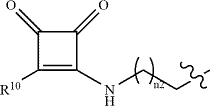

, wherein

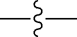

denotes a point of attachment;
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3.

In some embodiments, the ionizable lipid is selected from:

| | |
|---|---|
| 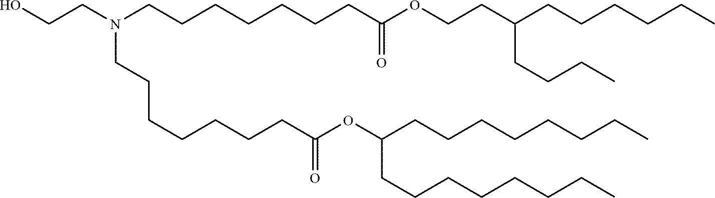 [SMILES: CCCCCCCCC(CCCCCCCC)OC(=O)CCCCCCCN(CCO)CCCCCCCC(=O)OCCC(CCCC)CCCCCC] | 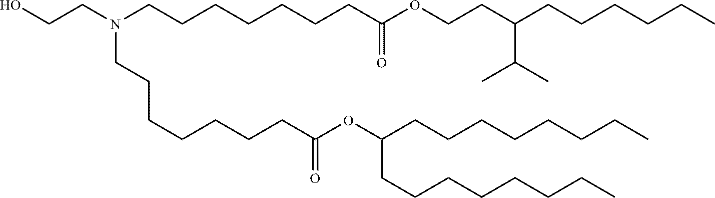 [SMILES: CCCCCCCCC(CCCCCCCC)OC(=O)CCCCCCCN(CCO)CCCCCCCC(=O)OCCC(CCCCCC)C(C)C] |
| 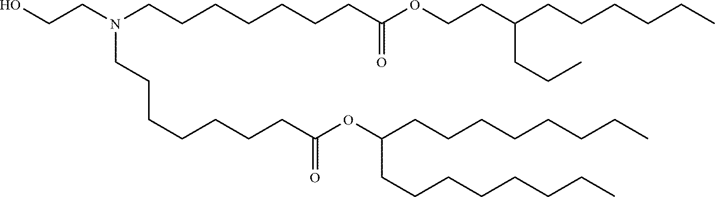 [SMILES: CCCCCCCCC(CCCCCCCC)OC(=O)CCCCCCCN(CCO)CCCCCCCC(=O)OCCC(CCC)CCCCCC] | 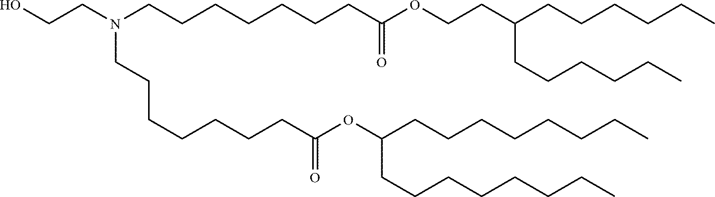 [SMILES: CCCCCCCCC(CCCCCCCC)OC(=O)CCCCCCCN(CCO)CCCCCCCC(=O)OCCC(CCCCCC)CCCCCC] |
| 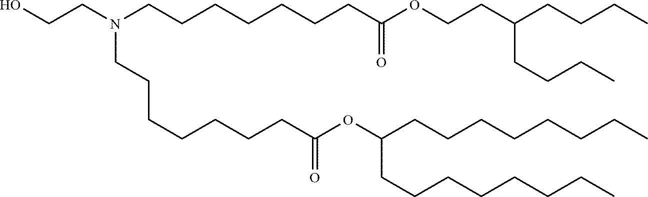 [SMILES: CCCCCCCCC(CCCCCCCC)OC(=O)CCCCCCCN(CCO)CCCCCCCC(=O)OCCC(CCCC)CCCC] | 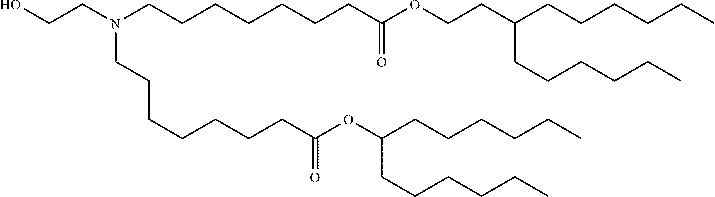 [SMILES: CCCCCCC(CCCCCC)CCOC(=O)CCCCCCCN(CCO)CCCCCCCC(=O)OC(CCCCCC)CCCCCC] |
| 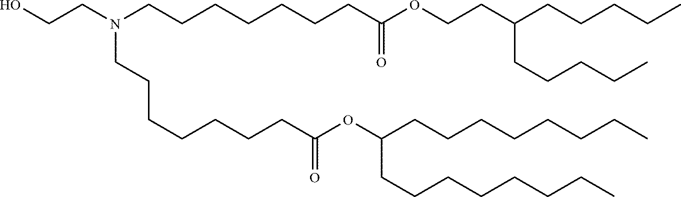 [SMILES: CCCCCCCCC(CCCCCCCC)OC(=O)CCCCCCCN(CCO)CCCCCCCC(=O)OCCC(CCCCC)CCCCC] | 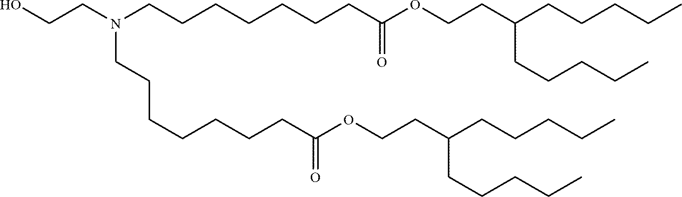 [SMILES: CCCCCC(CCCCC)CCOC(=O)CCCCCCCN(CCO)CCCCCCCC(=O)OCCC(CCCCC)CCCCC] |
| 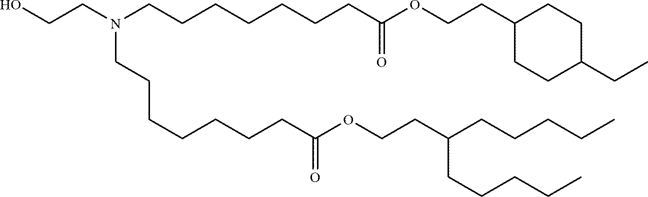 [SMILES: CCCCCC(CCCCC)CCOC(=O)CCCCCCCN(CCO)CCCCCCCC(=O)OCCC1CCC(CC)CC1] | 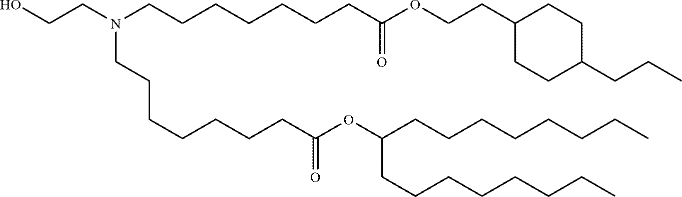 [SMILES: CCCCCCCCC(CCCCCCCC)OC(=O)CCCCCCCN(CCO)CCCCCCCC(=O)OCCC1CCC(CCC)CC1] |
| 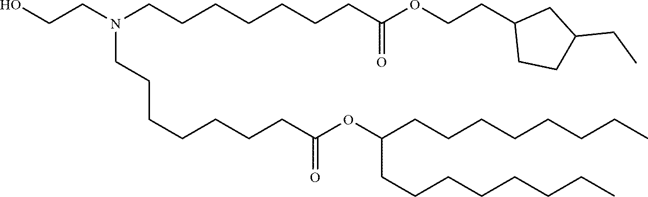 [SMILES: CCCCCCCCC(CCCCCCCC)OC(=O)CCCCCCCN(CCO)CCCCCCCC(=O)OCCC1CCC(CC)C1] | 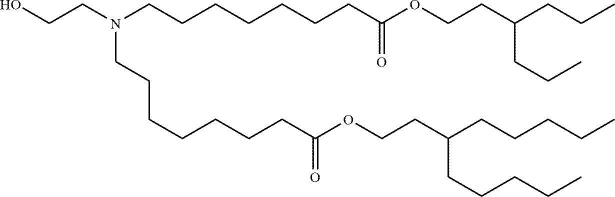 [SMILES: CCCCCC(CCCCC)CCOC(=O)CCCCCCCN(CCO)CCCCCCCC(=O)OCCC(CCC)CCC] |
| 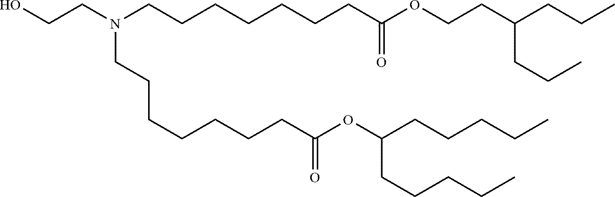 [SMILES: CCCCCC(CCCCC)OC(=O)CCCCCCCN(CCO)CCCCCCCC(=O)OCCC(CCC)CCC] | 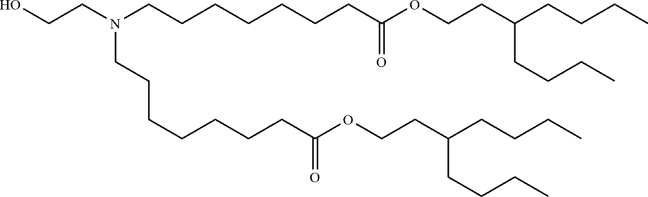 [SMILES: CCCCC(CCCC)CCOC(=O)CCCCCCCN(CCO)CCCCCCCC(=O)OCCC(CCCC)CCCC] |
| 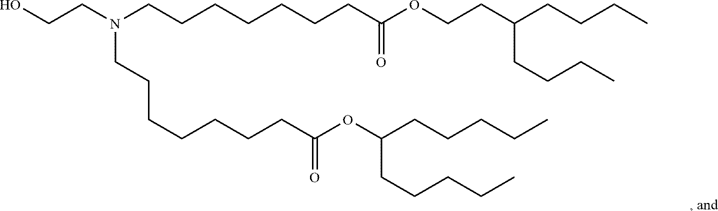 | 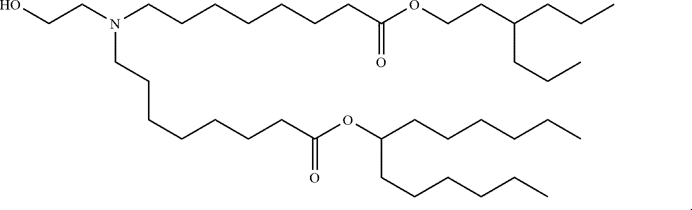 |

In some aspects, the ionizable lipids of the present disclosure may be one or more of compounds of formula (IL-III):

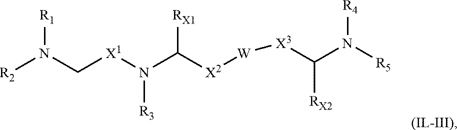

or salts or isomers therof, wherein,

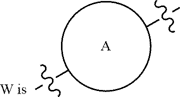

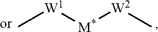

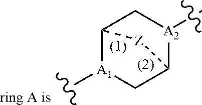

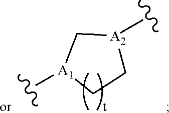

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
Rₓ₁ and Rₓ₂ are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃-₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

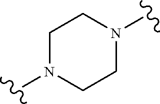

,then
i) at least one of X¹, X², and X³ is not —CH₂—; and/or
ii) at least one of R₁, R₂, R₃, R₄, and R₅ is —R″MR′.

In some embodiments, the compound is of any of formulae (IL-IIIa1)-(IL-IIIa8):

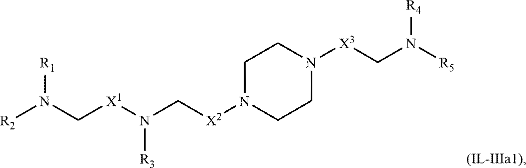

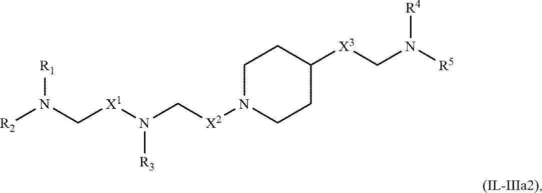

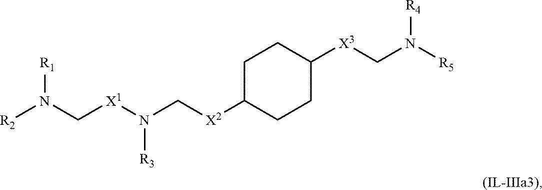

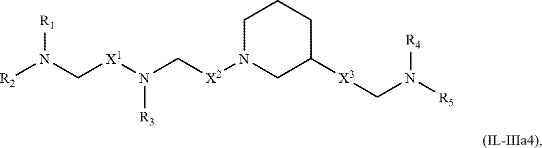

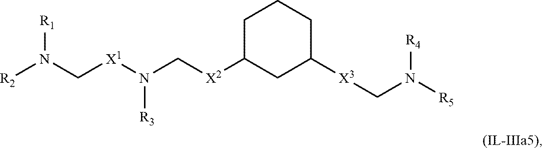

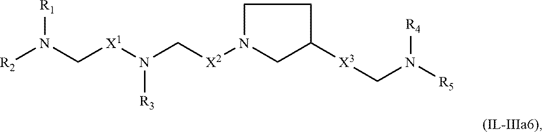

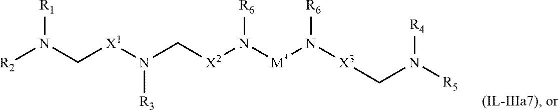

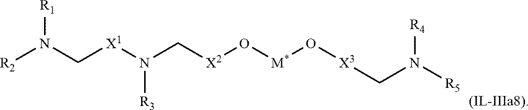

In some embodiments, the ionizable lipids are one or more of the compounds described in PCT Publication Nos. WO 2017/112865, WO 2018/232120.

In some embodiments, the ionizable lipids are selected from Compound 1-156 described in PCT Publication No. WO 2018/232120.

In some embodiments, the ionizable lipids are selected from Compounds 1-16, 42-66, 68-76, and 78-156 described in PCT Publication Nos. WO 2017/112865.

In some embodiments, the ionizable lipid is

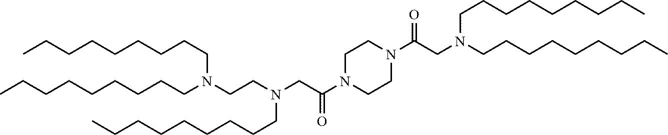 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (IL-1), (IL-IA), (IL-IB), (IL-II), (IL-IIa), (IL-IIb), (IL-IIc), (IL-IId), (IL-IIe), (IL-IIf), (IL-IIg), (IL-VI), (IL-VIIa), (IL-VIIIa), (IL-VIIIb), (IL-VIIb-1), (IL-VIIb-2), (IL-VIIb-3), (IL-VIIc), (IL-VIId), (IL-VIIIc), (IL-VIIId), (IL-VIVa), (IL-VIVb), (IL-III), (IL-IIIa1), (IL-IIIa2), (IL-IIIa3), (IL-IIIa4), (IL-IIIa5), (IL-IIIa6), (IL-IIIa7), or (IL-IIIa8) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some embodiments, the ionizable lipid is selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3P)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).

### Polyethylene Glycol (PEG) Lipids

As used herein, the term “PEG lipid” refers to polyethylene glycol (PEG)-modified lipids. Non-limiting examples of PEG lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. In some embodiments, a PEG lipid can be PEG-c-DOMG, PEG-DMG, PEG-DLPE, PEG-DMPE, PEG-DPPC, or a PEG-DSPE lipid.

In some embodiments, the PEG lipid includes, but are not limited to, 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG lipids includes those having lengths of from about C₁₄ to about C₂₂, In some embodiments, the lipid moiety of the PEG lipids includes those having lengths of from about C₁₄ to about C₁₆. In some embodiments, a PEG moiety, for example an mPEG-NH2, has a size of about 1000, 2000, 5000, 10,000, 15,000 or 20,000 daltons. In one embodiment, the PEG lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG lipids are known in the art, such as those described in U.S. Pat. No. 8158601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle or lipid nanoparticle formulation may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some embodiments, a PEG lipid may be PEG-c-DOMG, PEG-DMG, PEG-DLPE, PEG-DMPE, PEG-DPPC, or a PEG-DSPE lipid.

In some embodiments, the PEG-modified lipids are a modified form of PEG DMG. PEG-DMG has the following structure:

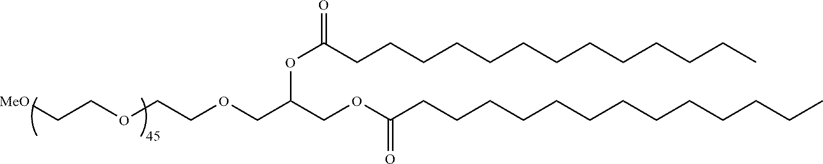

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In some embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In some embodiments, the PEG-OH lipid includes one or more hydroxyl groups on the PEG chain. In some embodiments, a PEG-OH or hydroxy-PEGylated lipid comprises an —OH group at the terminus of the PEG chain. Each possibility represents a separate embodiment of the present invention.

In some embodiments, a PEG lipid useful in the present invention is a compound of Formula (PL-I). Provided herein are compounds of Formula (PL-I):

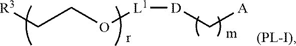

or salts thereof, wherein:
R³ is -OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, -OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10; L²-R²
A is of the formula:

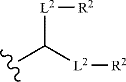

or

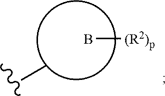

;
each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), -NR^{N}C(O), or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), -NR^{N}C(O)N(R^{N}), C(O)O, OC(O), OC(O)O, OC(O)N(R^{N}), NR^{N}C(O)O, C(O)S, SC(O), -C(=NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), -S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In some embodiments, the compound of Formula (PL-I) is a PEG-OH lipid (i.e., R³ is —OR^{O}, and R^{O} is hydrogen). In some embodiments, the compound of Formula (PL-I) is of Formula (PL-I-OH):

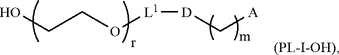

or a salt thereof.

In some embodiments, a PEG lipid useful in the present invention is a PEGylated fatty acid. In some embodiments, a PEG lipid useful in the present invention is a compound of Formula (PL-II). Provided herein are compounds of Formula (PL-II):

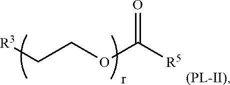

or a salt thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), -C(O)N(R^{N}), NR^{N}C(O), NR^{N}C(O)N(R^{N}), C(O)O, OC(O), OC(O)O, OC(O)N(R^{N}), -NR^{N}C(O)O, C(O)S, SC(O), C(═NR^{N}), C(═NR^{N})N(R^{N}, NR^{N}C(═NR^{N}, NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, -S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In some embodiments, the compound of Formula (PL-II) is of Formula (PL-II-OH):

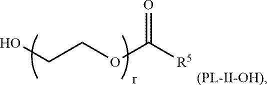

or a salt thereof, wherein:
r is an integer between 1 and 100;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), -C(O)N(R^{N}), NR^{N}C(O), NR^{N}C(O)N(R^{N}), C(O)O, OC(O), OC(O)O, OC(O)N(R^{N}), -NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, -S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In some embodiments, r is an integer between 10 to 80, between 20 to 70, between 30 to 60, or between 40 to 50.

In some embodiments, r is 45.

In some embodiments, R⁵ is C₁₇ alkyl.

In yet other embodiments the compound of Formula (PL-II) is:

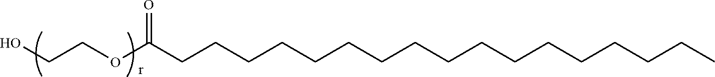

or a salt thereof.

In one embodiment, the compound of Formula (PL-II) is

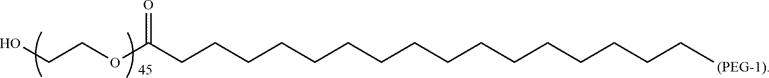

In some aspects, the lipid composition of the pharmaceutical compositions described herein does not comprise a PEG lipid.

In some embodiments, the PEG lipids may be one or more of the PEG lipids described in U.S. Application No. 62/520,530.

In some embodiments, the PEG lipid is a compound of Formula (PL-III):

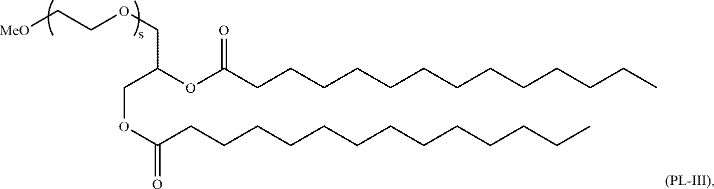

or a salt or isomer thereof, wherein s is an integer between 1 and 100.

In some embodiments, the PEG lipid is a compound of the following formula:

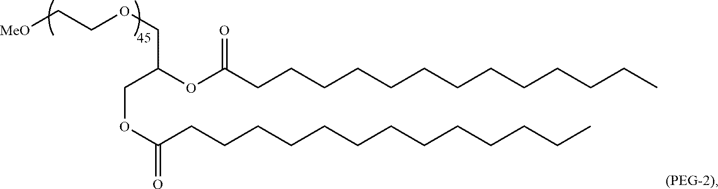

or a salt or isomer thereof.

### Structural Lipids

As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a mixture of two or more components each independently selected from cholesterol, fecosterol, sitosterol, ergosterol, campesterol, stigmasterol, brassicasterol, tomatidine, tomatine, ursolic acid, alpha-tocopherol, hopanoids, phytosterols, and steroids. In some embodiments, the structural lipid is a sterol. In some embodiments, the structural lipid is a mixture of two or more sterols. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol.

In some embodiments, the structural lipids may be one or more structural lipids described in U.S. Application No. 62/520,530.

### Encapsulation Agent

In some embodiments of the present disclosure, the encapsulation agent is a compound of Formula (EA-I):

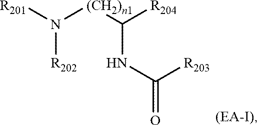

or salts or isomers thereof, wherein
R₂₀₁ and R₂₀₂ are each independently selected from the group consisting of H, C₁-C₆ alkyl, C₂-C₆ alkenyl, and (C═NH)N(R₁₀₁)₂ wherein each R₁₀₁ is independently selected from the group consisting of H, C₁-C₆ alkyl, and C₂-C₆ alkenyl;
R₂₀₃ is selected from the group consisting of C₁-C₂₀ alkyl and C₂-C₂₀ alkenyl;
R₂₀₄ is selected from the group consisting of H, C₁-C₂₀ alkyl, C₂-C₂₀ alkenyl, C(O)(OC₁-C₂₀ alkyl), C(O)(OC₂-C₂₀ alkenyl), C(O)(NHC₁-C₂₀ alkyl), and C(O)(NHC₂-C₂₀ alkenyl);
n1 is selected from 1, 2, 3, 4, 5, 6, 7, 8, 9, and 10.

In some embodiments, R₂₀₁ and R₂₀₂ are each independently selected from the group consisting of H and CH₃.

In some embodiments, R₂₀₁ and R₂₀₂ are each independently selected from the group consisting of (C═NH)NH₂ and (C═NH)N(CH₃)₂

In some embodiments, R₂₀₃ is selected from the group consisting of C₁-C₂₀ alkyl, Cs-C₁₈ alkyl, and C₁₂-C₁₆ alkyl.

In some embodiments, R₂₀₄ is selected from the group consisting of H, C₁-C₂₀ alkyl, C₂-C₂₀ alkenyl, C(O)(OC₁-C₂₀ alkyl), C(O)(OC₂-C₂₀ alkenyl), C(O)(NHC₁-C₂₀ alkyl), and C(O)(NHC₂-C₂₀ alkenyl); C₈-C₁₈ alkyl, C₈-C₁₈ alkenyl, C(O)(OC₈-C₁₈ alkyl), C(O)(OC₈-C₁₈ alkenyl), C(O)(NHC₈-C₁₈ alkyl), and C(O)(NHC₈-C₁₈ alkenyl); and C₁₂-C₁₆ alkyl, C₁₂-C₁₆ alkenyl, C(O)(OC₁₂-C₁₆ alkyl), C(O)(OC₁₂-C₁₆ alkenyl), C(O)(NHC₁₂-C₁₆ alkyl), and C(O)(NHC₁₂-C₁₆ alkenyl);

In some embodiments, n1 is selected from 1, 2, 3, 4, 5, 6, 7, 8, 9, and 10; n1 is selected from 1, 2, 3, 4, 5, and 6; n1 is selected from 2, 3, and 4.

In some embodiments, n1 is 3.

In some embodiments of the present disclosure, the encapsulation agent is a compound of Formula (EA-II):

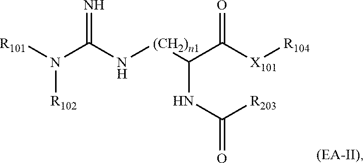

or salts or isomers thereof, wherein
X₁₀₁ is a bond, NH, or O;
R₁₀₁ and R₁₀₂ are each independently selected from the group consisting of H, C₁-C₆ alkyl, and C₂-C₆ alkenyl;
R₁₀₃ and R₁₀₄ are each independently selected from the group consisting of C₁-C₂₀ alkyl and C₂-C₂₀ alkenyl; and
n1 is selected from 1, 2, 3, 4, 5, 6, 7, 8, 9, and 10.

In some embodiments, X₁₀₁ is a bond.

In some embodiments, X₁₀₁ is NH.

In some embodiments, X₁₀₁ is O.

In some embodiments, R₁₀₁ and R₁₀₂ are each independently selected from the group consisting of H and CH₃.

In some embodiments, R₁₀₃ is selected from the group consisting of C₁-C₂₀ alkyl, Cs-C₁₈ alkyl, and C₁₂-C₁₆ alkyl.

In some embodiments, R₁₀₄ is selected from the group consisting of C₁-C₂₀ alkyl, Cs-C₁₈ alkyl, and C₁₂-C₁₆ alkyl.

In some embodiments, n1 is selected from 1, 2, 3, 4, 5, 6, 7, 8, 9, and 10; n1 is selected from 1, 2, 3, 4, 5, and 6; n1 is selected from 2, 3, and 4.

In some embodiments, n1 is 3.

Exemplary encapsulation agents include, but are not limited to, ethyl lauroyl arginate, ethyl myristoyl arginate, ethyl palmitoyl arginate, ethyl cholesterol-arginate, ethyl oleic arginate, ethyl capric arginate, and ethyl carprylic arginate.

In certain embodiments, the encapsulation agent is ethyl lauroyl arginate,

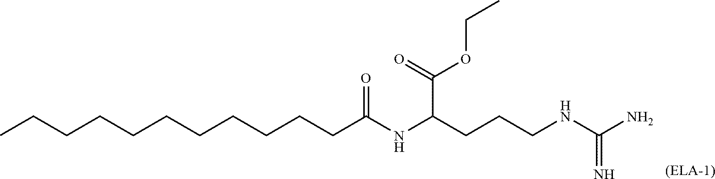

or a salt or isomer thereof.

In certain embodiments, the encapsulation agent is at least one compound selected from the group consisting of:

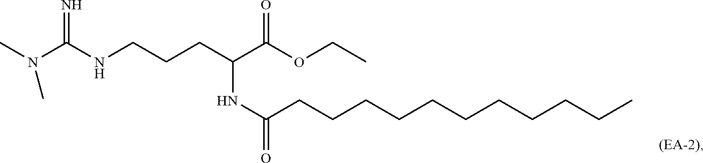

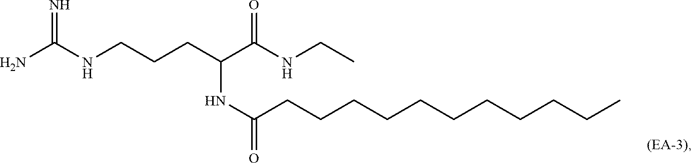

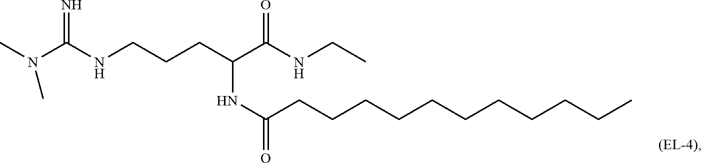

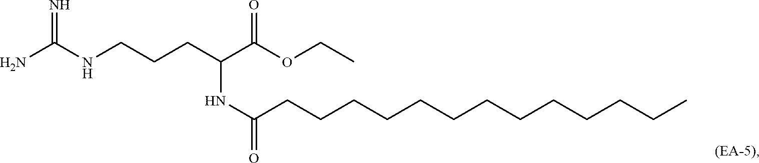

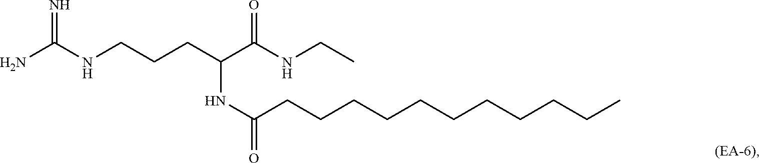

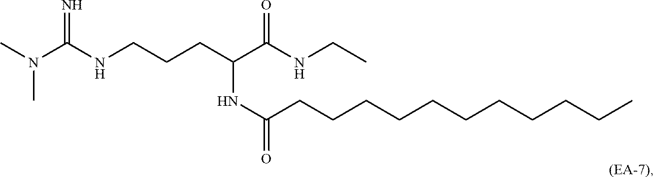

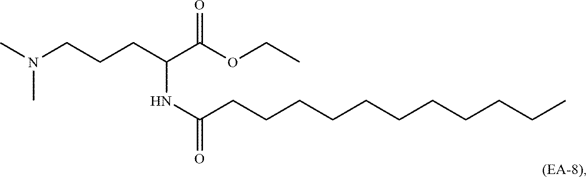

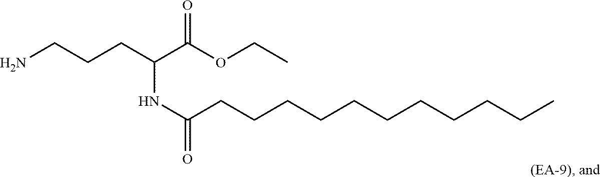

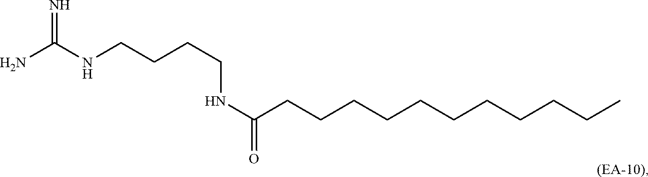

or salts and isomers thereof, such as, for example free bases, TFA salts, and/or HCl salts.

### Phospholipids

Phospholipids may assemble into one or more lipid bilayers. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. In some embodiments, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. In some embodiments, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid useful or potentially useful in the present invention is an analog or variant of DSPC. In some embodiments, a phospholipid useful or potentially useful in the present invention is a compound of Formula (PL-I):

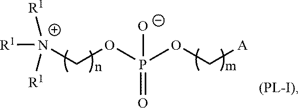

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

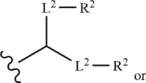

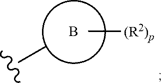

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

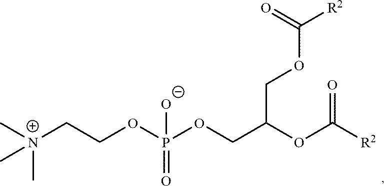

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530.

In some embodiments, the phospholipids may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In some embodiments, a LNP includes DSPC. In some embodiments, a LNP includes DOPE. In some embodiments, a LNP includes both DSPC and DOPE.

### I) Phospholipid Head Modifications

In some embodiments, a phospholipid useful or potentially useful in the present invention comprises a modified phospholipid head (e.g., a modified choline group). In some embodiments, a phospholipid with a modified head is DSPC, or analog thereof, with a modified quaternary amine. In some embodiments, in embodiments of Formula (PL-I), at least one of R¹ is not methyl. In some embodiments, at least one of R¹ is not hydrogen or methyl. In some embodiments, the compound of Formula (PL-I) is one of the following formulae:

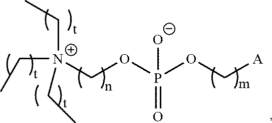

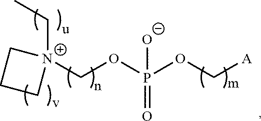

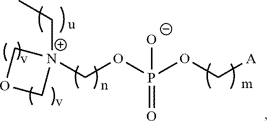

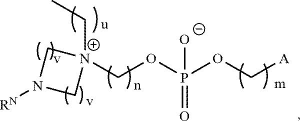

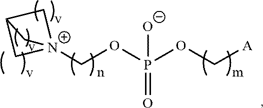

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In some embodiments, a compound of Formula (PL-I) is of Formula (PL-I-a):

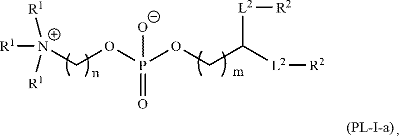

or a salt thereof.

In some embodiments, a phospholipid useful or potentially useful in the present invention comprises a cyclic moiety in place of the glyceride moiety. In some embodiments, a phospholipid useful in the present invention is DSPC, or analog thereof, with a cyclic moiety in place of the glyceride moiety. In some embodiments, the compound of Formula (PL-I) is of Formula (PL-I-b):

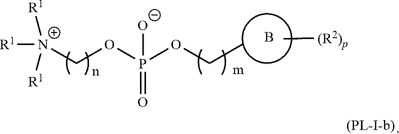

or a salt thereof.

### II) Phospholipid Tail Modifications

In some embodiments, a phospholipid useful or potentially useful in the present invention comprises a modified tail. In some embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. In some embodiments, In some embodiments, the compound of (PL-I) is of Formula (PL-I-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In some embodiments, the compound of Formula (PL-I) is of Formula (PL-I-c):

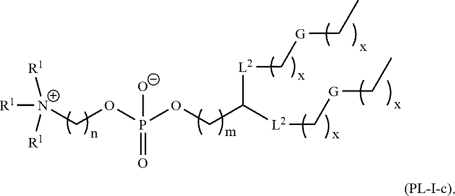

or a salt thereof, wherein:
each x is independently an integer between 0-30, inclusive; and
each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In some embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in some embodiments, a phospholipid useful or potentially useful in the present invention is a compound of Formula (PL-I), wherein n is 1, 3, 4, 5, 6, 7, 8, 9, or 10. In some embodiments, a compound of Formula (PL-I) is of one of the following formulae:

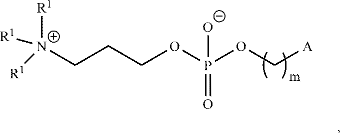

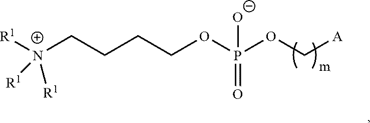

or a salt thereof.

### Alternative Lipids

In some embodiments, an alternative lipid is used in place of a phospholipid of the present disclosure. Non-limiting examples of such alternative lipids include the following:

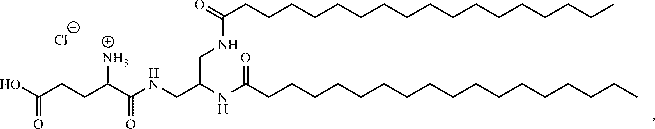

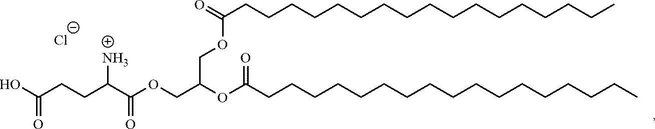

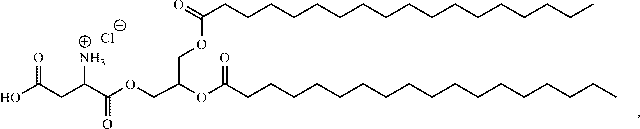

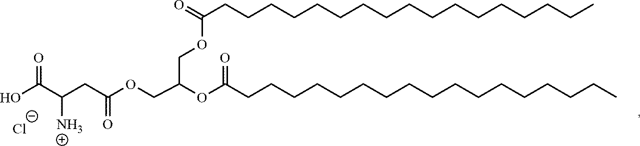

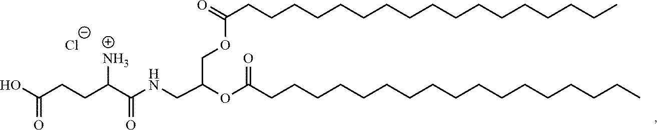

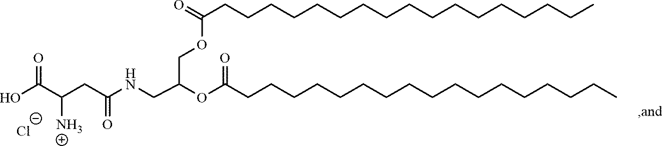

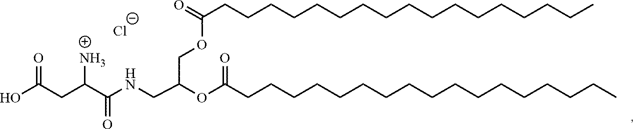

### Adjuvants

In some embodiments, a LNP that includes one or more lipids described herein may further include one or more adjuvants, e.g., Glucopyranosyl Lipid Adjuvant (GLA), CpG oligodeoxynucleotides (e.g., Class A or B), poly(I:C), aluminum hydroxide, and Pam3CSK4.

### Therapeutic Agents

Lipid nanoparticles (e.g., empty LNPs or loaded LNPs) may include one or more therapeutic and/or prophylactics. The disclosure features methods of delivering a therapeutic and/or prophylactic to a mammalian cell or organ, producing a polypeptide of interest in a mammalian cell, and treating a disease or disorder in a mammal in need thereof comprising administering to a mammal and/or contacting a mammalian cell with a lipid nanoparticle (e.g., an empty LNP or a loaded LNP) including a therapeutic and/or prophylactic.

Therapeutic and/or prophylactics include biologically active substances and are alternately referred to as “active agents.” A therapeutic and/or prophylactic may be a substance that, once delivered to a cell or organ, brings about a desirable change in the cell, organ, or other bodily tissue or system. Such species may be useful in the treatment of one or more diseases, disorders, or conditions. In some embodiments, a therapeutic and/or prophylactic is a small molecule drug useful in the treatment of a particular disease, disorder, or condition.

In some embodiments, a therapeutic and/or prophylactic is a vaccine, a compound (e.g., a polynucleotide or nucleic acid molecule that encodes a protein or polypeptide or peptide or a protein or polypeptide or protein) that elicits an immune response, and/or another therapeutic and/or prophylactic. Vaccines include compounds and preparations that are capable of providing immunity against one or more conditions related to infectious diseases and can include mRNAs encoding infectious disease derived antigens and/or epitopes. Vaccines also include compounds and preparations that direct an immune response against cancer cells and can include mRNAs encoding tumor cell derived antigens, epitopes, and/or neoepitopes. In some embodiments, a vaccine and/or a compound capable of eliciting an immune response is administered intramuscularly via a composition of the disclosure.

In other embodiments, a therapeutic and/or prophylactic is a protein, for example a protein needed to augment or replace a naturally-occurring protein of interest. Such proteins or polypeptides may be naturally occurring, or may be modified using methods known in the art, e.g., to increase half life. Exemplary proteins are intracellular, transmembrane, or secreted.

### Polynucleotides and Nucleic Acids

In some embodiments, the therapeutic agent is an agent that enhances (i.e., increases, stimulates, upregulates) protein expression. Non-limiting examples of types of therapeutic agents that can be used for enhancing protein expression include RNAs, mRNAs, dsRNAs, CRISPR/Cas9 technology, ssDNAs and DNAs (e.g., expression vectors). The agent that upregulates protein expression may upregulate expression of a naturally occurring or non-naturally occurring protein (e.g., a chimeric protein that has been modified to improve half life, or one that comprises desirable amino acid changes). Exemplary proteins include intracellular, transmembrane, or secreted proteins, peptides, or polypeptides.

In some embodiments, the therapeutic agent is a DNA therapeutic agent. The DNA molecule can be a double-stranded DNA, a single-stranded DNA (ssDNA), or a molecule that is a partially double-stranded DNA, i.e., has a portion that is double-stranded and a portion that is single-stranded. In some cases the DNA molecule is triple-stranded or is partially triple-stranded, i.e., has a portion that is triple stranded and a portion that is double stranded. The DNA molecule can be a circular DNA molecule or a linear DNA molecule.

A DNA therapeutic agent can be a DNA molecule that is capable of transferring a gene into a cell, e.g., that encodes and can express a transcript. In other embodiments, the DNA molecule is a synthetic molecule, e.g., a synthetic DNA molecule produced in vitro. In some embodiments, the DNA molecule is a recombinant molecule. Non-limiting exemplary DNA therapeutic agents include plasmid expression vectors and viral expression vectors.

The DNA therapeutic agents described herein, e.g., DNA vectors, can include a variety of different features. The DNA therapeutic agents described herein, e.g., DNA vectors, can include a non-coding DNA sequence. For example, a DNA sequence can include at least one regulatory element for a gene, e.g., a promoter, enhancer, termination element, polyadenylation signal element, splicing signal element, and the like. In some embodiments, the non-coding DNA sequence is an intron. In some embodiments, the non-coding DNA sequence is a transposon. In some embodiments, a DNA sequence described herein can have a non-coding DNA sequence that is operatively linked to a gene that is transcriptionally active. In other embodiments, a DNA sequence described herein can have a non-coding DNA sequence that is not linked to a gene, i.e., the non-coding DNA does not regulate a gene on the DNA sequence.

In some embodiments, in the loaded LNP of the disclosure, the one or more therapeutic and/or prophylactic agents is a nucleic acid. In some embodiments, the one or more therapeutic and/or prophylactic agents is selected from the group consisting of a ribonucleic acid (RNA) and a deoxyribonucleic acid (DNA).

For example, in some embodiments, when the therapeutic and/or prophylactic agents is a DNA, the DNA is selected from the group consisting of a double-stranded DNA, a single-stranded DNA (ssDNA), a partially double-stranded DNA, a triple stranded DNA, and a partially triple-stranded DNA. In some embodiments, the DNA is selected from the group consisting of a circular DNA, a linear DNA, and mixtures thereof.

In some embodiments, in the loaded LNP of the disclosure, the one or more therapeutic and/or prophylactic agents is selected from the group consisting of a plasmid expression vector, a viral expression vector, and mixtures thereof.

For example, in some embodiments, when the therapeutic and/or prophylactic agents is a RNA, the RNA is selected from the group consisting of a single-stranded RNA, a double-stranded RNA (dsRNA), a partially double-stranded RNA, and mixtures thereof. In some embodiments, the RNA is selected from the group consisting of a circular RNA, a linear RNA, and mixtures thereof.

For example, in some embodiments, when the therapeutic and/or prophylactic agents is a RNA, the RNA is selected from the group consisting of a short interfering RNA (siRNA), an asymmetrical interfering RNA (aiRNA), a RNA interference (RNAi) molecule, a microRNA (miRNA), an antagomir, an antisense RNA, a ribozyme, a Dicer-substrate RNA (dsRNA), a small hairpin RNA (shRNA), a messenger RNA (mRNA), locked nucleic acids (LNAs) and CRISPR/Cas9 technology, and mixtures thereof.

For example, in some embodiments, when the therapeutic and/or prophylactic agents is a RNA, the RNA is selected from the group consisting of a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and mixtures thereof.

In some embodiments, the one or more therapeutic and/or prophylactic agents is an mRNA. In some embodiments, the one or more therapeutic and/or prophylactic agents is a modified mRNA (mmRNA).

In some embodiments, the one or more therapeutic and/or prophylactic agents is an mRNA that incorporates a micro-RNA binding site (miR binding site). Further, in some embodiments, an mRNA includes one or more of a stem loop, a chain terminating nucleoside, a polyA sequence, a polyadenylation signal, and/or a 5′ cap structure.

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. In some embodiments, all uracils or uridines are modified. When all nucleobases, nucleosides, or nucleotides are modified, e.g., all uracils or uridines, the mRNA can be referred to as “fully modified”, e.g., for uracil or uridine.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,O3′GpppG, m27,O3′GppppG, m27,O2′GppppG, m7Gpppm7G, m73′dGpppG, m27,O3′GpppG, m27,O3′GppppG, and m27,O2′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′ deoxyadenosine (cordycepin), 3′ deoxyuridine, 3′ deoxycytosine, 3′ deoxyguanosine, 3′ deoxythymine, and 2′,3′ dideoxynucleosides, such as 2′,3′ dideoxyadenosine, 2′,3′ dideoxyuridine, 2′,3′ dideoxycytosine, 2′,3′ dideoxyguanosine, and 2′,3′ dideoxythymine. In some embodiments, incorporation of a chain terminating nucleotide into an mRNA, for example at the 3′-terminus, may result in stabilization of the mRNA.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A poly A sequence may also comprise stabilizing nucleotides or analogs. For example, a poly A sequence can include deoxythymidine, e.g., inverted (or reverse linkage) deoxythymidine (dT), as a stabilizing nucleotide or analog. Detials on using inverted dT and other stabilizing poly A sequence modifications can be found, for example, in WO2017/049275 A2, the content of which is incoported herein by reference. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site. MicroRNA binding sites (or miR binding sites) can be used to regulate mRNA expression in various tissues or cell types. In exemplary embodiments, miR binding sites are engineered into 3′ UTR sequences of an mRNA to regulate, e.g., enhance degradation of mRNA in cells or tissues expressing the cognate miR. Such regulation is useful to regulate or control “off-target” expression ir mRNAs, i.e., expression in undesired cells or tissues in vivo. Detials on using mir binding sites can be found, for example, in WO 2017/062513 A2, the content of which is incoported herein by reference.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (Ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (im5U), 1-taurinomethyl-pseudouridine, 5-taurinomethyl-2-thio-uridine(τm5s2U), 1-taurinomethyl-4-thio-pseudouridine, 5-methyl-uridine (m5U, i.e., having the nucleobase deoxythymine), 1-methyl-pseudouridine (m1Ψ), 5-methyl-2-thio-uridine (m5s2U), 1-methyl-4-thio-pseudouridine (m1s4Ψ), 4-thio-1-methyl-pseudouridine, 3-methyl-pseudouridine (m3Ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 Ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (Ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1 -E-propenylamino)] uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include a-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include a-thio-guanosine, inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 2′-O-ribosylguanosine (phosphate) (Gr(p)), 1-thio-guanosine, O6-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is pseudouridine (Ψ), N1-methylpseudouridine (m1Ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases.) In some embodiments, the modified nucleobase is N1-methylpseudouridine (m1Ψ) and the mRNA of the disclosure is fully modified with N1-methylpseudouridine (m1Ψ). In some embodiments, N1-methylpseudouridine (m1Ψ) represents from 75-100% of the uracils in the mRNA. In some embodiments, N1-methylpseudouridine (m1Ψ) represents 100% of the uracils in the mRNA.

In some embodiments, the modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified adenine. Exemplary nucleobases and nucleosides having a modified adenine include 7-deaza-adenine, 1-methyl-adenosine (m1A), 2-methyl-adenine (m2A), N6-methyl-adenosine (m6A). In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is 1-methyl-pseudouridine (m1Ψ), 5-methoxy-uridine (mo5U), 5-methyl-cytidine (m5C), pseudouridine (Ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases.)

In some embodiments, the mRNA comprises pseudouridine (Ψ). In some embodiments, the mRNA comprises pseudouridine (Ψ) and 5-methyl-cytidine (m5C). In some embodiments, the mRNA comprises 1-methyl-pseudouridine (m1Ψ). In some embodiments, the mRNA comprises 1-methyl-pseudouridine (m1Ψ) and 5-methyl-cytidine (m5C). In some embodiments, the mRNA comprises 2-thiouridine (s2U). In some embodiments, the mRNA comprises 2-thiouridine and 5-methyl-cytidine (m5C). In some embodiments, the mRNA comprises 5-methoxy-uridine (mo5U). In some embodiments, the mRNA comprises 5-methoxy-uridine (mo5U) and 5-methyl-cytidine (m5C). In some embodiments, the mRNA comprises 2′-O-methyl uridine. In some embodiments, the mRNA comprises 2′-O-methyl uridine and 5-methyl-cytidine (m5C). In some embodiments, the mRNA comprises comprises N6-methyl-adenosine (m6A). In some embodiments, the mRNA comprises N6-methyl-adenosine (m6A) and 5-methyl-cytidine (m5C).

In certain embodiments, an mRNA of the disclosure is uniformly modified (i.e., fully modified, modified through-out the entire sequence) for a particular modification. For example, an mRNA can be uniformly modified with N1-methylpseudouridine (m1Ψ) or 5-methyl-cytidine (m5C), meaning that all uridines or all cytosine nucleosides in the mRNA sequence are replaced with N1-methylpseudouridine (m1Ψ) or 5-methyl-cytidine (m5C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

The mmRNAs of the disclosure can include a combination of modifications to the sugar, the nucleobase, and/or the internucleoside linkage. These combinations can include any one or more modifications described herein.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP + 75% CTP/ 25% 5-Methoxy-UTP + 75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, CA) and/or proprietary methods. In some embodiments, the mRNA sequence is optimized using optimization algorithms, e.g., to optimize expression in mammalian cells or enhance mRNA stability.

In certain embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In some embodiments, mRNAs are made using IVT enzymatic synthesis methods. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme.

Either enzymatic or chemical ligation methods may be used to conjugate polynucleotides or their regions with different functional moieties, such as targeting or delivery agents, fluorescent labels, liquids, nanoparticles, etc. Therapeutic Agents for Reducing Protein Expression

In some embodiments, the therapeutic agent is a therapeutic agent that reduces (i.e., decreases, inhibits, downregulates) protein expression. Non-limiting examples of types of therapeutic agents that can be used for reducing protein expression include mRNAs that incorporate a micro-RNA binding site(s) (miR binding site), microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNAs), locked nucleic acids (LNAs) and CRISPR/Cas9 technology.

### Sensor Sequences and MicroRNA (MiRNA) Binding Sites

Sensor sequences include, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. Non-limiting examples of sensor sequences are described in U.S. Publication 2014/0200261, the contents of which are incorporated herein by reference in their entirety.

In some embodiments, a polyribonucleotide (e.g., a ribonucleic acid (RNA), e.g., a messenger RNA (mRNA)) of the disclosure comprising an open reading frame (ORF) encoding a polypeptide further comprises a sensor sequence. In some embodiments, the sensor sequence is a miRNA binding site.

A miRNA is a 19-25 nucleotide long noncoding RNA that binds to a polyribonucleotide and down-regulates gene expression either by reducing stability or by inhibiting translation of the polyribonucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh KK, Johnston WK, Garrett-Engele P, Lim LP, Bartel DP; Mol Cell. 2007 Jul 6;27(1):91-105. miRNA profiling of the target cells or tissues can be conducted to determine the presence or absence of miRNA in the cells or tissues. In some embodiments, a polyribonucleotide (e.g., a ribonucleic acid (RNA), e.g., a messenger RNA (mRNA)) of the disclosure comprises one or more microRNA target sequences, microRNA sequences, or microRNA seeds. Such sequences can correspond to any known microRNA such as those taught in U.S. Publication US2005/0261218 and U.S. Publication US2005/0059005, the contents of each of which are incorporated herein by reference in their entirety.

As used herein, the term “microRNA (miRNA or miR) binding site” refers to a sequence within a polyribonucleotide, e.g., within a DNA or within an RNA transcript, including in the 5′UTR and/or 3′UTR, that has sufficient complementarity to all or a region of a miRNA to interact with, associate with or bind to the miRNA. In some embodiments, a polyribonucleotide of the disclosure comprising an ORF encoding a polypeptide further comprises a miRNA binding site. In exemplary embodiments, a 5′UTR and/or 3′UTR of the polyribonucleotide (e.g., a ribonucleic acid (RNA), e.g., a messenger RNA (mRNA)) comprises a miRNA binding site.

A miRNA binding site having sufficient complementarity to a miRNA refers to a degree of complementarity sufficient to facilitate miRNA-mediated regulation of a polyribonucleotide, e.g., miRNA-mediated translational repression or degradation of the polyribonucleotide. In exemplary aspects of the disclosure, a miRNA binding site having sufficient complementarity to the miRNA refers to a degree of complementarity sufficient to facilitate miRNA-mediated degradation of the polyribonucleotide, e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. In some embodiments, the desired regulation is mRNA degradation. In some embodiments, the miRNA binding site has full or complete complementarity (e.g., full complementarity or complete complementarity over all or a significant portion of the length of a naturally-occurring miRNA). In some embodiments, the mRNA degradation has full or complete complementarity.

In some embodiments, a miRNA binding site includes a sequence that has complementarity (e.g., partial or complete complementarity) with an miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In some embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds to the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the polyribonucleotide comprising the miRNA binding site. In some embodiments, the miRNA binding site has imperfect complementarity so that a RISC complex comprising the miRNA induces instability in the polyribonucleotide comprising the miRNA binding site. In another embodiment, the miRNA binding site has imperfect complementarity so that a RISC complex comprising the miRNA represses transcription of the polyribonucleotide comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a polyribonucleotide of the disclosure, the polyribonucleotide can be targeted for degradation or reduced translation, provided the miRNA in question is available. This can reduce off-target effects upon delivery of the polyribonucleotide. In some embodiments, if a polyribonucleotide of the disclosure is not intended to be delivered to a tissue or cell but ends up there, then a miRNA abundant in the tissue or cell can inhibit the expression of the gene of interest if one or multiple binding sites of the miRNA are engineered into the 5′UTR and/or 3′UTR of the polyribonucleotide.

Conversely, miRNA binding sites can be removed from polyribonucleotide sequences in which they naturally occur in order to increase protein expression in specific tissues. In some embodiments, a binding site for a specific miRNA can be removed from a polyribonucleotide to improve protein expression in tissues or cells containing the miRNA.

In one embodiment, a polyribonucleotide of the disclosure can include at least one miRNA-binding site in the 5′UTR and/or 3′UTR in order to direct cytotoxic or cytoprotective mRNA therapeutics to specific cells such as, but not limited to, normal and/or cancerous cells. In another embodiment, a polyribonucleotide of the disclosure can include two, three, four, five, six, seven, eight, nine, ten, or more miRNA-binding sites in the 5′-UTR and/or 3′-UTR in order to direct cytotoxic or cytoprotective mRNA therapeutics to specific cells such as, but not limited to, normal and/or cancerous cells.

Regulation of expression in multiple tissues can be accomplished through introduction or removal of one or more miRNA binding sites. The decision whether to remove or insert a miRNA binding site can be made based on miRNA expression patterns and/or their profilings in diseases. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and macrophages), macrophages, monocytes, B lymphocytes, T lymphocytes, granulocytes, natural killer cells, etc. Immune cell specific miRNAs are involved in immunogenicity, autoimmunity, the immune-response to infection, inflammation, as well as unwanted immune response after gene therapy and tissue/organ transplantation. Immune cells specific miRNAs also regulate many aspects of development, proliferation, differentiation and apoptosis of hematopoietic cells (immune cells). In some embodiments, miR-142 and miR-146 are exclusively expressed in immune cells, particularly abundant in myeloid dendritic cells. It has been demonstrated that the immune response to a polyribonucleotide can be shut-off by adding miR-142 binding sites to the 3′-UTR of the polyribonucleotide, enabling more stable gene transfer in tissues and cells. miR-142 efficiently degrades exogenous polyribonucleotides in antigen presenting cells and suppresses cytotoxic elimination of transduced cells (e.g., Annoni A et al., blood, 2009, 114, 5152-5161; Brown BD, et al., Nat med. 2006, 12(5), 585-591; Brown BD, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′UTR and/or 3′UTR of a polyribonucleotide of the disclosure can selectively repress gene expression in antigen presenting cells through miR-142 mediated degradation, limiting antigen presentation in antigen presenting cells (e.g., dendritic cells) and thereby preventing antigen-mediated immune response after the delivery of the polyribonucleotide. The polyribonucleotide is then stably expressed in target tissues or cells without triggering cytotoxic elimination.

In one embodiment, binding sites for miRNAs that are known to be expressed in immune cells, in particular, antigen presenting cells, can be engineered into a polyribonucleotide of the disclosure to suppress the expression of the polyribonucleotide in antigen presenting cells through miRNA mediated RNA degradation, subduing the antigen-mediated immune response. Expression of the polyribonucleotide is maintained in non-immune cells where the immune cell specific miRNAs are not expressed. In some embodiments, in some embodiments, to prevent an immunogenic reaction against a liver specific protein, any miR-122 binding site can be removed and a miR-142 (and/or mirR-146) binding site can be engineered into the 5′UTR and/or 3′UTR of a polyribonucleotide of the disclosure.

To further drive the selective degradation and suppression in APCs and macrophage, a polyribonucleotide of the disclosure can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1--3p, hsa-let-7f-2--5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-Sp,miR-24-2-Sp, miR-24-3p, miR-26a-1-3p, miR-26a-2-3p, miR-26a-5p, miR-26b-3p, miR-26b-5p, miR-27a-3p, miR-27a-5p, miR-27b-3p,miR-27b-5p, miR-28-3p, miR-28-5p, miR-2909, miR-29a-3p, miR-29a-5p, miR-29b-1-5p, miR-29b-2-5p, miR-29c-3p, miR-29c-5p,, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima DD et al, Blood, 2010, 116:e118-e127; Vaz C et al., BMC Genomics, 2010, 11,288, the content of each of which is incorporated herein by reference in its entirety.)

miRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, and miR-939-5p. miRNA binding sites from any liver specific miRNA can be introduced to or removed from a polyribonucleotide of the disclosure to regulate expression of the polyribonucleotide in the liver. Liver specific miRNA binding sites can be engineered alone or further in combination with immune cell (e.g., APC) miRNA binding sites in a polyribonucleotide of the disclosure.

miRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, and miR-381-5p. MiRNA binding sites from any lung specific miRNA can be introduced to or removed from a polyribonucleotide of the disclosure to regulate expression of the polyribonucleotide in the lung. Lung specific miRNA binding sites can be engineered alone or further in combination with immune cell (e.g., APC) miRNA binding sites in a polyribonucleotide of the disclosure.

miRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p, and miR-92b-5p. MiRNA binding sites from any heart specific microRNA can be introduced to or removed from a polyribonucleotide of the disclosure to regulate expression of the polyribonucleotide in the heart. Heart specific miRNA binding sites can be engineered alone or further in combination with immune cell (e.g., APC) miRNA binding sites in a polyribonucleotide of the disclosure.

miRNAs that are known to be expressed in the nervous system include, but are not limited to, miR-124-5p, miR-125a-3p, miR-125a-5p, miR-125b-1-3p, miR-125b-2-3p, miR-125b-5p,miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p,miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p, and miR-9-5p. MiRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, and miR-657. MiRNA binding sites from any CNS specific miRNA can be introduced to or removed from a polyribonucleotide of the disclosure to regulate expression of the polyribonucleotide in the nervous system. Nervous system specific miRNA binding sites can be engineered alone or further in combination with immune cell (e.g., APC) miRNA binding sites in a polyribonucleotide of the disclosure.

miRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p, and miR-944. MiRNA binding sites from any pancreas specific miRNA can be introduced to or removed from a polyribonucleotide of the disclosure to regulate expression of the polyribonucleotide in the pancreas. Pancreas specific miRNA binding sites can be engineered alone or further in combination with immune cell (e.g., APC) miRNA binding sites in a polyribonucleotide of the disclosure.

miRNAs that are known to be expressed in the kidney include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p, and miR-562. MiRNA binding sites from any kidney specific miRNA can be introduced to or removed from a polyribonucleotide of the disclosure to regulate expression of the polyribonucleotide in the kidney. Kidney specific miRNA binding sites can be engineered alone or further in combination with immune cell (e.g., APC) miRNA binding sites in a polyribonucleotide of the disclosure.

miRNAs that are known to be expressed in the muscle include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p, and miR-25-5p. MiRNA binding sites from any muscle specific miRNA can be introduced to or removed from a polyribonucleotide of the disclosure to regulate expression of the polyribonucleotide in the muscle. Muscle specific miRNA binding sites can be engineered alone or further in combination with immune cell (e.g., APC) miRNA binding sites in a polyribonucleotide of the disclosure.

miRNAs are also differentially expressed in different types of cells, such as, but not limited to, endothelial cells, epithelial cells, and adipocytes.

miRNAs that are known to be expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p, and miR-92b-5p. Many novel miRNAs are discovered in endothelial cells from deep-sequencing analysis (e.g., Voellenkle C et al., RNA, 2012, 18, 472-484, herein incorporated by reference in its entirety). MiRNA binding sites from any endothelial cell specific miRNA can be introduced to or removed from a polyribonucleotide of the disclosure to regulate expression of the polyribonucleotide in the endothelial cells.

miRNAs that are known to be expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells, let-7 family, miR-133a, miR-133b, miR-126 specific in lung epithelial cells, miR-382-3p, miR-382-5p specific in renal epithelial cells, and miR-762 specific in corneal epithelial cells. MiRNA binding sites from any epithelial cell specific miRNA can be introduced to or removed from a polyribonucleotide of the disclosure to regulate expression of the polyribonucleotide in the epithelial cells.

In addition, a large group of miRNAs are enriched in embryonic stem cells, controlling stem cell self-renewal as well as the development and/or differentiation of various cell lineages, such as neural cells, cardiac, hematopoietic cells, skin cells, osteogenic cells and muscle cells (e.g., Kuppusamy KT et al., Curr. Mol Med, 2013, 13(5), 757-764; Vidigal JA and Ventura A, Semin Cancer Biol. 2012, 22(5-6), 428-436; Goff LA et al., PLoS One, 2009, 4:e7192; Morin RD et al., Genome Res,2008,18, 610-621; Yoo JK et al., Stem Cells Dev. 2012, 21(11), 2049-2057, each of which is herein incorporated by reference in its entirety). MiRNAs abundant in embryonic stem cells include, but are not limited to, let-7a-2-3p, let-a-3p, let-7a-5p, let7d-3p, let-7d-5p, miR-103a-2-3p, miR-103a-5p, miR-106b-3p, miR-106b-5p, miR-1246, miR-1275, miR-138-1-3p, miR-138-2-3p, miR-138-5p, miR-154-3p, miR-154-5p, miR-200c-3p, miR-200c-5p, miR-290, miR-301a-3p, miR-301a-5p, miR-302a-3p, miR-302a-5p, miR-302b-3p, miR-302b-5p, miR-302c-3p, miR-302c-5p, miR-302d-3p, miR-302d-5p, miR-302e, miR-367-3p, miR-367-5p, miR-369-3p, miR-369-5p, miR-370, miR-371, miR-373, miR-380-5p, miR-423-3p, miR-423-5p, miR-486-5p, miR-520c-3p, miR-548e, miR-548f, miR-548g-3p, miR-548g-5p, miR-548i, miR-548k, miR-5481, miR-548m, miR-548n, miR-548o-3p, miR-548o-5p, miR-548p, miR-664a-3p, miR-664a-5p, miR-664b-3p, miR-664b-5p, miR-766-3p, miR-766-5p, miR-885-3p, miR-885-5p,miR-93-3p, miR-93-5p, miR-941,miR-96-3p, miR-96-5p, miR-99b-3p and miR-99b-5p. Many predicted novel miRNAs are discovered by deep sequencing in human embryonic stem cells (e.g., Morin RD et al., Genome Res,2008,18, 610-621; Goff LA et al., PLoS One, 2009, 4:e7192; Bar M et al., Stem cells, 2008, 26, 2496-2505, the content of each of which is incorporated herein by reference in its entirety).

In one embodiment, the binding sites of embryonic stem cell specific miRNAs can be included in or removed from the 3′UTR of a polyribonucleotide of the disclosure to modulate the development and/or differentiation of embryonic stem cells, to inhibit the senescence of stem cells in a degenerative condition (e.g., degenerative diseases), or to stimulate the senescence and apoptosis of stem cells in a disease condition (e.g., cancer stem cells).

Many miRNA expression studies are conducted to profile the differential expression of miRNAs in various cancer cells/tissues and other diseases. Some miRNAs are abnormally over-expressed in certain cancer cells and others are under-expressed. In some embodiments, miRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, US8389210); asthma and inflammation (US8415096); prostate cancer (US2013/0053264); hepatocellular carcinoma (WO2012/151212, US2012/0329672, WO2008/054828, US8252538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lymph nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells ( US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563, the content of each of which is incorporated herein by reference in its entirety.)

As a non-limiting example, miRNA binding sites for miRNAs that are over-expressed in certain cancer and/or tumor cells can be removed from the 3′UTR of a polyribonucleotide of the disclosure, restoring the expression suppressed by the over-expressed miRNAs in cancer cells, thus ameliorating the corresponsive biological function, for instance, transcription stimulation and/or repression, cell cycle arrest, apoptosis and cell death. Normal cells and tissues, wherein miRNAs expression is not up-regulated, will remain unaffected.

MiRNA can also regulate complex biological processes such as angiogenesis (e.g., miR-132) (Anand and Cheresh Curr Opin Hematol 2011 18:171-176). In the polyribonucleotides of the disclosure, miRNA binding sites that are involved in such processes can be removed or introduced, in order to tailor the expression of the polyribonucleotides to biologically relevant cell types or relevant biological processes. In this context, the polyribonucleotides of the disclosure are defined as auxotrophic polyribonucleotides.

### Peptide/Polypeptide Therapeutic Agents

In some embodiments, the therapeutic agent is a peptide therapeutic agent. In some embodiments the therapeutic agent is a polypeptide therapeutic agent.

In some embodiments, the peptide or polypeptide is naturally-derived, e.g., isolated from a natural source. In other embodiments, the peptide or polypeptide is a synthetic molecule, e.g., a synthetic peptide or polypeptide produced in vitro. In some embodiments, the peptide or polypeptide is a recombinant molecule. In some embodiments, the peptide or polypeptide is a chimeric molecule. In some embodiments, the peptide or polypeptide is a fusion molecule. In some embodiments, the peptide or polypeptide therapeutic agent of the composition is a naturally occurring peptide or polypeptide. In some embodiments, the peptide or polypeptide therapeutic agent of the composition is a modified version of a naturally occurring peptide or polypeptide (e.g., contains less than 3, less than 5, less than 10, less than 15, less than 20, or less than 25 amino substitutions, deletions, or additions compared to its wild type, naturally occurring peptide or polypeptide counterpart).

In some embodiments, in the loaded LNP of the disclosure, the one or more therapeutic and/or prophylactic agents is a polynucleotide or a polypeptide.

### Genome Editing Techniques

In some embodiments, the nucleic acid is suitable for a genome editing technique.

In some embodiments, the genome editing technique is clustered regularly interspaced short palindromic repeats (CRISPR) or transcription activator-like effector nuclease (TALEN).

In some embodiments, the nucleic acid is at least one nucleic acid suitable for a genome editing technique selected from the group consisting of a CRISPR RNA (crRNA), a trans-activating crRNA (tracrRNA), a single guide RNA (sgRNA), and a DNA repair template.

### Vaccines

In some embodiments, the therapeutic and/or prophylactic is a ribonucleic acid (RNA) cancer vaccine of an RNA (e.g., messenger RNA (mRNA)) that can safely direct the body’s cellular machinery to produce nearly any cancer protein or fragment thereof of interest. In some embodiments, the RNA is a modified RNA. The RNA vaccines of the present disclosure may be used to induce a balanced immune response against cancers, comprising both cellular and humoral immunity, without risking the possibility of insertional mutagenesis, for example.

The RNA vaccines may be utilized in various settings depending on the prevalence of the cancer or the degree or level of unmet medical need. The RNA vaccines may be utilized to treat and/or prevent a cancer of various stages or degrees of metastasis. The RNA vaccines have superior properties in that they produce much larger antibody titers and produce responses earlier than alternative anti-cancer therapies including cancer vaccines. While not wishing to be bound by theory, it is believed that the RNA vaccines, as mRNA polynucleotides, are better designed to produce the appropriate protein conformation upon translation as the RNA vaccines co-opt natural cellular machinery. Unlike traditional vaccines which are manufactured ex vivo and may trigger unwanted cellular responses, the RNA vaccines are presented to the cellular system in a more native fashion.

Some embodiments of the present disclosure provide cancer vaccines that include at least one ribonucleic acid (RNA) polynucleotide having an open reading frame encoding at least one cancer antigenic polypeptide or an immunogenic fragment thereof {e.g., an immunogenic fragment capable of inducing an immune response to cancer). Other embodiments include at least one ribonucleic acid (RNA) polynucleotide having an open reading frame encoding two or more antigens or epitopes capable of inducing an immune response to cancer.

The invention in some aspects is a vaccine of a mRNA having an open reading frame encoding a cancer antigen and a mRNA having an open reading frame encoding an immune checkpoint modulator. In some embodiments the immune checkpoint modulator is an inhibitory checkpoint polypeptide. In some embodiments, the inhibitory checkpoint polypeptide is an antibody or fragment thereof that specifically binds to a molecule selected from the group consisting of PD-1, TIM-3, VISTA, A2AR, B7-H3, B7-H4, BTLA, CTLA-4, IDO, KIR and LAG3. The inhibitory checkpoint polypeptide is an anti-CTLA4 or anti-PDl antibody in some embodiments. Optionally the vaccine includes a lipid nanoparticle. In some embodiments a vaccine of a mRNA having an open reading frame encoding a cancer antigen is administered to a subject. In other embodiments a checkpoint inhibitor 3-10 weeks later. In some embodiments the checkpoint inhibitor is administered 4 weeks later.

In other aspects the invention is a personalized cancer vaccine of a mRNA having an open reading frame encoding at least 2 cancer antigens, wherein the at least 2 cancer antigens are patient specific cancer antigens, and a lipid nanoparticle carrier. In some embodiments the lipid nanoparticle has a mean diameter of 50-200 nm.

In yet other aspects, the invention is a personalized cancer vaccine of a mRNA having an open reading frame encoding at least 2 cancer antigens wherein the at least 2 cancer antigens are representative of antigens of a patient. In some embodiments, the antigens of a patient are exosome identified antigens of the patient. In some embodiments a single mRNA encodes the cancer antigens. In other embodiments a plurality of mRNA encode the cancer antigens.

Each mRNA may encode 5-10 cancer antigens or a single cancer antigen in other embodiments. In some embodiments the mRNA encodes 2-100 cancer antigens. In other embodiments mRNA encodes 10-100, 20-100, 50-100, 100-200, 300-400, 500-600, 600-700, 700-800, 900-1,000, or 1,000-10,000 cancer antigens.

In some embodiments,
a) the mRNA encoding each cancer antigen is interspersed by cleavage sensitive sites;
b) the mRNA encoding each cancer antigen is linked directly to one another without a linker ;
c) the mRNA encoding each cancer antigen is linked to one another with a single nucleotide linker;
d) each cancer antigen comprises a 25-35 amino acids and includes a centrally located SNP mutation;
e) at least 30% of the cancer antigens have a highest affinity for class I MHC molecules from the subject;
f) at least 30% of the cancer antigens have a highest affinity for class II MHC molecules from the subject;
g) at least 50% of the cancer antigens have a predicted binding affinity of IC >500nM for HLA-A, HLA-B and/or DRB 1;
h) the mRNA encodes 20 cancer antigens;
i) 50% of the cancer antigens have a binding affinity for class I MHC and 50% of the cancer antigens have a binding affinity for class II MHC; and/or
j) the mRNA encoding the cancer antigens is arranged such that the cancer antigens are ordered to minimize pseudo-epitopes.

In some embodiments, each cancer antigen comprises 31 amino acids and includes a centrally located SNP mutation with 15 flanking amino acids on each side of the SNP mutation.

In some embodiments the vaccine is a personalized cancer vaccine and wherein the cancer antigen is a subject specific cancer antigen. In some embodiments, the subject specific cancer antigen may be representative of an exome of a tumor sample of the subject, or of a transcriptome of a tumor sample of the subject. In some embodiments, the subject specific cancer antigen may be representative of an exosome of the subject.

In some embodiments, the open reading frame further encodes one or more traditional cancer antigens. In some embodiments, the traditional cancer antigen is a non-mutated antigen. In some embodiments, the traditional cancer antigen is a mutated antigen.

In some embodiments, the mRNA vaccine further comprises an mRNA having an open reading frame encoding one or more traditional cancer antigens.

In some embodiments a single mRNA encodes the cancer antigens. In other embodiments a plurality of mRNA encode the cancer antigens. Each cancer antigen is 10-50 amino acids in length in some embodiments. In other embodiments each cancer antigen is 15-20 amino acids in length. In other embodiments the cancer antigen is 20-50, 25-100, 100-200, 200-300, 300-400, 400-500, 500-1,000, or 1,000-10,000 amino acids in length.

In some embodiments, the vaccines further comprise an adjuvant.

Some embodiments of the present disclosure provide a cancer vaccine that includes at least one ribonucleic acid (RNA) polynucleotide having an open reading frame encoding at least one cancer polypeptide, at least one 5′ terminal cap and at least one chemical modification, formulated within a lipid nanoparticle. In some embodiments, a 5′ terminal cap is 7mG(5′)ppp(5′)NlmpNp.

In some embodiments, at least one chemical modification is selected from pseudouridine, Nl-methylpseudouridine, Nl-ethylpseudouridine, 2-thiouridine, 4′- thiouridine, 5-methylcytosine, 2-thio-1-methyl- 1-deaza-pseudouridine, 2-thio-1-methyl- pseudouridine, 2-thio-5-aza-uridine, 2-thio-dihydropseudouridine, 2-thio-dihydrouridine, 2- thio-pseudouridine, 4-methoxy-2-thio-pseudouridine, 4-methoxy-pseudouridine, 4-thio-1- methyl-pseudouridine, 4-thio-pseudouridine, 5-aza-uridine, dihydropseudouridine, 5- methyluridine, 5-methoxyuridine and 2′ -O-methyl uridine. In some embodiments the extent of incorporation of chemically modified nucleotides has been optimized for improved immune responses to the vaccine formulation.

In some embodiments, a lipid nanoparticle (e.g., an empty LNP or a loaded LNP of the disclosure) comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from 2,2-dilinoleyl-4-dimethylaminoethyl- [1,3]-dioxolane (DLin-KC2-DMA), dilinoleyl-methyl-4-dimethylaminobutyrate (DLin-MC3- DMA), and di((Z)-non-2-en-1-yl) 9-((4-(dimethylamino)butanoyl)oxy)heptadecanedioate (L319).

In some embodiments the lipid nanoparticle formulation includes an immune potentiator (e.g., TLR agonist) to enhance immunogenicity of the vaccine (formulation).

In some embodiments, 100% of the uracil in the open reading frame have a chemical modification. In some embodiments, a chemical modification is in the 5-position of the uracil. In some embodiments, a chemical modification is a N1-methyl pseudouridine.

In other embodiments a mRNA encoding an APC reprograming molecule is included in the vaccine or coadministered with the vaccine. The APC reprograming molecule may be a CIITA, a chaperone protein such as CLIP, HLA-DO, HLA-DM, a costimulatory molecule such as CD40, CD80, CD86, a CIITA fragment such as amino acids 26-137 of CIITA or a protein having 80% sequence identity to CIITA.

In other aspects a method of eliciting an immune response in a subject by identifying at least 2 cancer antigens from a sample of a subject, wherein the at least 2 cancer antigens include mutations selected from the group consisting of frame-shift mutations and recombinations, and administering a mRNA vaccine having an open reading frame encoding the at least 2 cancer antigens to the subject is provided.

In some embodiments, the cancer antigens are identified from an exosome of the subject. In some embodiments 2-100 antigens are identified from the exosome. In other embodiments the mRNA vaccine has an open reading frame encoding the 2-100 antigens. A single mRNA or a plurality of mRNA may encode the antigens.

In some embodiments the antigens are cancer antigens. The cancer antigens may have mutations selected from point mutations, frame-shift mutations and recombinations. The method may further involve confirming that the cancer antigens are subject specific by exome analysis.

In some embodiments the method may further involve confirming that the cancer antigens are subject specific by transcriptome analysis.

In some embodiments the method also involves at least one month after the administration of the mRNA vaccine, identifying at least 2 cancer antigens from a sample of the subject to produce a second set of cancer antigens, and administering to the subject a mRNA vaccine having an open reading frame encoding the second set of cancer antigens to the subject.

In other embodiments the sample of the subject is a tumor sample.

In other aspects the invention comprises a method of eliciting an immune response in a subject by identifying at least 2 cancer antigens from a sample of a subject to produce a first set of cancer antigens, administering to the subject a mRNA vaccine having an open reading frame encoding the first set of cancer antigens to the subject, at least one month after the administration of the mRNA vaccine, identifying at least 2 cancer antigens from a sample of a subject to produce a second set of cancer antigens, and administering to the subject a mRNA vaccine having an open reading frame encoding the second set of cancer antigens to the subject.

The mRNA vaccine having an open reading frame encoding second set of antigens, in some embodiments, is administered to the subject 6 months to 1 year after the mRNA vaccine having an open reading frame encoding first set of cancer antigens. In other embodiments the mRNA vaccine having an open reading frame encoding second set of antigens is administered to the subject 1-2 years after the mRNA vaccine having an open reading frame encoding first set of cancer antigens.

In some embodiments a single mRNA has an open reading frame encoding the cancer antigens. In other embodiments a plurality of mRNA encode the antigens. In some embodiments the second set of cancer antigens includes 2-100 antigens. In other embodiments the cancer antigens have mutations selected from point mutations, frame-shift mutations and recombinations.

In other aspects the invention comprises a method of eliciting an immune response in a subject, by identifying at least 2 cancer antigens from a sample of a subject, administering a mRNA having an open reading frame encoding the at least 2 cancer antigens to the subject, and administering a cancer therapeutic agent to the subject. In some embodiments the cancer therapeutic agent is a targeted therapy. The targeted therapy may be a BRAF inhibitor such as vemurafenib (PLX4032) or dabrafenib.

In other embodiments the cancer therapeutic agent is a T-cell therapeutic agent. The T-cell therapeutic agent may be a checkpoint inhibitor such as an anti-PD- 1 antibody or an anti-CTLA-4 antibody. In some embodiments the anti-PD- 1 antibody is BMS-936558 (nivolumab). In other embodiments the anti-CTLA-4 antibody is ipilimumab. The T-cell therapeutic agent in other embodiments is OX40L. In yet other embodiments the cancer therapeutic agent is a vaccine comprising a population based tumor specific antigen.

In other embodiments the cancer therapeutic agent is a vaccine comprising an mRNA having an open reading frame encoding one or more traditional cancer antigens.

In some embodiments, the mRNA having an open reading frame encoding the at least 2 cancer antigens is administered to the subject simultaneously with the cancer therapeutic agent. In some embodiments, the mRNA having an open reading frame encoding the at least 2 cancer antigens is administered to the subject before administration of the cancer therapeutic agent. In some embodiments, the mRNA having an open reading frame encoding the at least 2 cancer antigens is administered to the subject after administration of the cancer therapeutic agent.

A method comprising mixing a mRNA having an open reading frame encoding a cancer antigen with a lipid nanoparticle formulation to produce a mRNA cancer vaccine, and administering the mRNA cancer vaccine to a subject within 24 hours of mixing is provided in other aspects of the invention. In some embodiments the mRNA cancer vaccine is administered to the subject within 12 hours of mixing. In other embodiments the mRNA cancer vaccine is administered to the subject within 1 hour of mixing. The mRNA cancer vaccine encodes 2-100 cancer antigens or 10-100 cancer antigens in some embodiments.

In some embodiments the vaccine is a personalized cancer vaccine and wherein the cancer antigen is a subject specific cancer antigen.

In some embodiments a single mRNA encodes the cancer antigens. In other embodiments a plurality of mRNA encode the cancer antigens. Each mRNA encodes 5-10 cancer antigens or a single cancer antigen in other embodiments. In yet other embodiments each cancer antigen is 10-50 amino acids in length or 15-20 amino acids in length.

Further provided herein are uses of cancer vaccines in the manufacture of a medicament for use in a method of inducing an antigen specific immune response in a subject, the method comprising administering the cancer vaccine to the subject in an amount effective to produce an antigen specific immune response.

A method of treating cancer in a subject in need thereof by identifying at least 2 cancer antigens from an exosome isolated from the subject; producing, based on the identified antigens, a mRNA vaccine having an open reading frame encoding the antigens; and administering the mRNA vaccine to the subject, wherein the mRNA vaccine induces a tumor- specific immune response in the subject, thereby treating cancer in the subject is provided in other aspects. The invention in other aspects is a RNA vaccine preparable according to a method involving identifying at least 2 cancer antigens from an exosome isolated from a subject; producing, based on the identified antigens, a mRNA vaccine having an open reading frame encoding the antigens.

A method of eliciting an immune response in a subject against a cancer antigen is provided in aspects of the invention. The method involves administering to the subject a RNA vaccine comprising at least one RNA polynucleotide having an open reading frame encoding at least one antigenic polypeptide or an immunogenic fragment thereof, thereby inducing in the subject an immune response specific to the antigenic polypeptide or an immunogenic fragment thereof, wherein the anti-antigenic polypeptide antibody titer in the subject is increased following vaccination relative to anti-antigenic polypeptide antibody titer in a subject vaccinated with a prophylactically effective dose of a traditional vaccine against the cancer. An “anti-antigenic polypeptide antibody” is a serum antibody the binds specifically to the antigenic polypeptide.

A prophylactically effective dose is a therapeutically effective dose that prevents advancement of cancer at a clinically acceptable level. In some embodiments the therapeutically effective dose is a dose listed in a package insert for the vaccine. A traditional vaccine, as used herein, refers to a vaccine other than the mRNA vaccines of the invention. For instance, a traditional vaccine includes but is not limited to live microorganism vaccines, killed microorganism vaccines, subunit vaccines, protein antigen vaccines, DNA vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA.)

In some embodiments the anti-antigenic polypeptide antibody titer in the subject is increased 1 log to 10 log following vaccination relative to anti-antigenic polypeptide antibody titer in a subject vaccinated with a prophylactically effective dose of a traditional vaccine against the cancer.

In some embodiments the anti-antigenic polypeptide antibody titer in the subject is increased 1 log following vaccination relative to anti-antigenic polypeptide antibody titer in a subject vaccinated with a prophylactically effective dose of a traditional vaccine against the cancer.

In some embodiments the anti-antigenic polypeptide antibody titer in the subject is increased 2 log following vaccination relative to anti-antigenic polypeptide antibody titer in a subject vaccinated with a prophylactically effective dose of a traditional vaccine against the cancer.

In some embodiments the anti-antigenic polypeptide antibody titer in the subject is increased 3 log following vaccination relative to anti-antigenic polypeptide antibody titer in a subject vaccinated with a prophylactically effective dose of a traditional vaccine against the cancer.

In some embodiments the anti-antigenic polypeptide antibody titer in the subject is increased 5 log following vaccination relative to anti-antigenic polypeptide antibody titer in a subject vaccinated with a prophylactically effective dose of a traditional vaccine against the or cancer.

In some embodiments the anti-antigenic polypeptide antibody titer in the subject is increased 10 log following vaccination relative to anti-antigenic polypeptide antibody titer in a subject vaccinated with a prophylactically effective dose of a traditional vaccine against the or cancer.

A method of eliciting an immune response in a subject against a cancer antigen is provided in other aspects of the invention. The method involves administering to the subject a RNA vaccine comprising at least one RNA polynucleotide having an open reading frame encoding at least one antigenic polypeptide or an immunogenic fragment thereof, thereby inducing in the subject an immune response specific to antigenic polypeptide or an immunogenic fragment thereof, wherein the immune response in the subject is equivalent to an immune response in a subject vaccinated with a traditional vaccine against the cancer antigen at 2 times to 100 times the dosage level relative to the RNA vaccine.

In some embodiments the immune response in the subject is equivalent to an immune response in a subject vaccinated with a traditional vaccine at twice the dosage level relative to the RNA vaccine.

In some embodiments the immune response in the subject is equivalent to an immune response in a subject vaccinated with a traditional vaccine at three times the dosage level relative to the RNA vaccine.

In some embodiments the immune response in the subject is equivalent to an immune response in a subject vaccinated with a traditional vaccine at 4 times the dosage level relative to the RNA vaccine.

In some embodiments the immune response in the subject is equivalent to an immune response in a subject vaccinated with a traditional vaccine at 5 times the dosage level relative to the RNA vaccine. In some embodiments the immune response in the subject is equivalent to an immune response in a subject vaccinated with a traditional vaccine at 10 times the dosage level relative to the RNA vaccine.

In some embodiments the immune response in the subject is equivalent to an immune response in a subject vaccinated with a traditional vaccine at 50 times the dosage level relative to the RNA vaccine.

In some embodiments the immune response in the subject is equivalent to an immune response in a subject vaccinated with a traditional vaccine at 100 times the dosage level relative to the RNA vaccine.

In some embodiments the immune response in the subject is equivalent to an immune response in a subject vaccinated with a traditional vaccine at 10 times to 1000 times the dosage level relative to the RNA vaccine.

In some embodiments the immune response in the subject is equivalent to an immune response in a subject vaccinated with a traditional vaccine at 100 times to 1000 times the dosage level relative to the RNA vaccine.

In other embodiments the immune response is assessed by determining antibody titer in the subject.

In other aspects the invention comprises a method of eliciting an immune response in a subject against a by administering to the subject a RNA vaccine comprising at least one RNA polynucleotide having an open reading frame encoding at least one cancer antigenic polypeptide or an immunogenic fragment thereof, thereby inducing in the subject an immune response specific to the antigenic polypeptide or an immunogenic fragment thereof, wherein the immune response in the subject is induced 2 days to 10 weeks earlier relative to an immune response induced in a subject vaccinated with a prophylactically effective dose of a traditional vaccine against the cancer antigen. In some embodiments the immune response in the subject is induced in a subject vaccinated with a prophylactically effective dose of a traditional vaccine at 2 times to 100 times the dosage level relative to the RNA vaccine.

In some embodiments the immune response in the subject is induced 2 days earlier relative to an immune response induced in a subject vaccinated with a prophylactically effective dose of a traditional vaccine.

In some embodiments the immune response in the subject is induced 3 days earlier relative to an immune response induced in a subject vaccinated a prophylactically effective dose of a traditional vaccine. In some embodiments the immune response in the subject is induced 1 week earlier relative to an immune response induced in a subject vaccinated with a prophylactically effective dose of a traditional vaccine.

In some embodiments the immune response in the subject is induced 2 weeks earlier relative to an immune response induced in a subject vaccinated with a prophylactically effective dose of a traditional vaccine.

In some embodiments the immune response in the subject is induced 3 weeks earlier relative to an immune response induced in a subject vaccinated with a prophylactically effective dose of a traditional vaccine.

In some embodiments the immune response in the subject is induced 5 weeks earlier relative to an immune response induced in a subject vaccinated with a prophylactically effective dose of a traditional vaccine.

In some embodiments the immune response in the subject is induced 10 weeks earlier relative to an immune response induced in a subject vaccinated with a prophylactically effective dose of a traditional vaccine.

A method of eliciting an immune response in a subject against an cancer by administering to the subject a cancer RNA vaccine having an open reading frame encoding a first antigenic polypeptide, wherein the RNA polynucleotide does not include a stabilization element, and wherein an adjuvant is not coformulated or co-administered with the vaccine.

In yet other aspects the invention comprises a method of producing an mRNA encoding a concatemeric cancer antigen comprising between 1000 and 3000 nucleotides, the method by
(a) binding a first polynucleotide comprising an open reading frame encoding the concatemeric cancer antigen and a second polynucleotide comprising a 5′-UTR to a polynucleotide conjugated to a solid support;
(b) ligating the 3 ′-terminus of the second polynucleotide to the 5 ′-terminus of the first polynucleotide under suitable conditions, wherein the suitable conditions comprise a DNA Ligase, thereby producing a first ligation product;
(c) ligating the 5′ terminus of a third polynucleotide comprising a 3′-UTR to the 3′- terminus of the first ligation product under suitable conditions, wherein the suitable conditions comprise an RNA Ligase, thereby producing a second ligation product; and
(d) releasing the second ligation product from the solid support, thereby producing an mRNA encoding the concatemeric cancer antigen comprising between 1000 and 3000 nucleotides. In some embodiments of any one of the provided compositions or methods,the mRNA encodes one or more recurrent polymorphisms. In some embodiments, the one or more recurrent polymorphisms comprises a recurrent somatic cancer mutation in p53. In some such embodiments, the one or more recurrent somatic cancer mutation in p53 are selected from the group consisting of:
   (1) mutations at the canonical 5′ splice site neighboring codon p.T125;
   (2) mutations at the canonical 5′ splice site neighboring codon p.331;
   (3) mutations at the canonical 3′ splice site neighboring codon p. 126;
   (4) mutations at the canonical 5′ splice site neighboring codon p.224, inducing a cryptic alternative intronic 5′ splice site.

In one embodiment, the invention provides a cancer therapeutic vaccine comprising mRNA encoding an open reading frame (ORF) coding for one or more of neoantigen peptides (1) through (4). In one embodiment, the invention provides the selective administration of a vaccine containing or coding for one or more of peptides (1)-(4), based on the patient’s tumor containing any of the above mutations. In one embodiment, the invention provides the selective administration of the vaccine based on the dual criteria of the subject’s tumor containing any of the above mutations and the subject’s normal HLA type containing the corresponding HLA allele predicted to bind to the resulting neoantigen.

A method for treating a subject with a personalized mRNA cancer vaccine, by isolating a sample from a subject, identifying a set of neoepitopes by analyzing a patient transcriptome and/or a patient exome from the sample to produce a patient specific mutanome, selecting a set of neoepitopes for the vaccine from the mutanome based on MHC binding strength, MHC binding diversity, predicted degree of immunogenicity, low self reactivity, and/or T cell reactivity, preparing the mRNA vaccine to encode the set of neoepitopes and administering the mRNA vaccine to the subject within two months of isolating the sample from the subject is provided in other aspects of the invention. In some embodiments the mRNA vaccine is administered to the subject within one month of isolating the sample from the subject.

In other aspects the invention comprises a method of identifying a set of neoepitopes for use in a personalized mRNA cancer vaccine having one or more polynucleotides that encode the set of neoepitopes by a. identifying a patient specific mutanome by analyzing a patient transcriptome and a patient exome, b. selecting a subset of 15-500 neoepitopes from the mutanome using a weighted value for the neoepitopes based on at least three of: an assessment of gene or transcript-level expression in patient RNA-seq; variant call confidence score; RNA-seq allele- specific expression; conservative vs. non-conservative amino acid substitution; position of point mutation (Centering Score for increased TCR engagement); position of point mutation (Anchoring Score for differential HLA binding); Selfness: <100% core epitope homology with patient WES data; HLA-A and -B IC50 for 8 mers-11 mers; HLA-DRB 1 IC50 for 15 mers-20 mers; promiscuity Score (i.e. number of patient HLAs predicted to bind); HLA-C IC50 for 8 mers-11 mers;HLA-DRB3-5 IC50 for 15 mers-20 mers; HLA-DQB 1/A1 IC50 for 15 mers-20 mers; HLA-DPB 1/A1 IC50 for 15 mers-20 mers; Class I vs Class II proportion; Diversity of patient HLA-A, -B and DRB 1 allotypes covered; proportion of point mutation vs complex epitopes (e.g. frameshifts); and /or pseudo-epitope HLA binding scores, and c. selecting the set of neoepitopes for use in a personalized mRNA cancer vaccine from the subset based on the highest weighted value, wherein the set of neoepitopes comprise 15-40 neoepitopes.

In some embodiments the nucleic acid vaccines described herein are chemically modified. In other embodiments the nucleic acid vaccines are unmodified.

Yet other aspects provide compositions for and methods of vaccinating a subject comprising administering to the subject a nucleic acid vaccine comprising one or more RNA polynucleotides having an open reading frame encoding a first antigenic polypeptide or a concatemeric polypeptide, wherein the RNA polynucleotide does not include a stabilization element, and wherein an adjuvant is not co-formulated or co-administered with the vaccine.

In other aspects the invention is a composition for or method of vaccinating a subject comprising administering to the subject a nucleic acid vaccine comprising one or more RNA polynucleotides having an open reading frame encoding a first antigenic polypeptide wherein a dosage of between 10 ug/kg and 400 ug/kg of the nucleic acid vaccine is administered to the subject. In some embodiments the dosage of the RNA polynucleotide is 1-5 ug, 5-10 ug, 10-15 ug, 15-20 ug, 10-25 ug, 20-25 ug, 20-50 ug, 30-50 ug, 40-50 ug, 40-60 ug, 60-80 ug, 60-100 ug, 50-100 ug, 80-120 ug, 40-120 ug, 40-150 ug, 50-150 ug, 50-200 ug, 80-200 ug, 100-200 ug, 120-250 ug, 150-250 ug, 180-280 ug, 200-300 ug, 50-300 ug, 80-300 ug, 100- 300 ug, 40-300 ug, 50-350 ug, 100-350 ug, 200-350 ug, 300-350 ug, 320-400 ug, 40-380 ug, 40-100 ug, 100-400 ug, 200-400 ug, or 300-400 ug per dose. In some embodiments, the nucleic acid vaccine is administered to the subject by intradermal or intramuscular injection. In some embodiments, the nucleic acid vaccine is administered to the subject on day zero. In some embodiments, a second dose of the nucleic acid vaccine is administered to the subject on day twenty one.

In some embodiments, a dosage of 25 micrograms of the RNA polynucleotide is included in the nucleic acid vaccine administered to the subject. In some embodiments, a dosage of 100 micrograms of the RNA polynucleotide is included in the nucleic acid vaccine administered to the subject. In some embodiments, a dosage of 50 micrograms of the RNA polynucleotide is included in the nucleic acid vaccine administered to the subject. In some embodiments, a dosage of 75 micrograms of the RNA polynucleotide is included in the nucleic acid vaccine administered to the subject. In some embodiments, a dosage of 150 micrograms of the RNA polynucleotide is included in the nucleic acid vaccine administered to the subject. In some embodiments, a dosage of 400 micrograms of the RNA polynucleotide is included in the nucleic acid vaccine administered to the subject. In some embodiments, a dosage of 200 micrograms of the RNA polynucleotide is included in the nucleic acid vaccine administered to the subject. In some embodiments, the RNA polynucleotide accumulates at a 100 fold higher level in the local lymph node in comparison with the distal lymph node. In other embodiments the nucleic acid vaccine is chemically modified and in other embodiments the nucleic acid vaccine is not chemically modified.

Aspects of the invention provide a nucleic acid vaccine comprising one or more RNA polynucleotides having an open reading frame encoding a first antigenic polypeptide or a concatemeric polypeptide, wherein the RNA polynucleotide does not include a stabilization element, and a pharmaceutically acceptable carrier or excipient, wherein an adjuvant is not included in the vaccine. In some embodiments, the stabilization element is a histone stem-loop. In some embodiments, the stabilization element is a nucleic acid sequence having increased GC content relative to wild type sequence.

Aspects of the invention provide nucleic acid vaccines comprising one or more RNA polynucleotides having an open reading frame encoding a first antigenic polypeptide, wherein the RNA polynucleotide is present in the formulation for in vivo administration to a host, which confers an antibody titer superior to the criterion for seroprotection for the first antigen for an acceptable percentage of human subjects. In some embodiments, the antibody titer produced by the mRNA vaccines of the invention is a neutralizing antibody titer. In some embodiments the neutralizing antibody titer is greater than a protein vaccine. In other embodiments the neutralizing antibody titer produced by the mRNA vaccines of the invention is greater than an adjuvanted protein vaccine. In yet other embodiments the neutralizing antibody titer produced by the mRNA vaccines of the invention is 1,000- 10,000, 1,200-10,000, 1,400- 10,000, 1,500- 10,000, 1,000- 5,000, 1,000- 4,000, 1,800- 10,000, 2000-10,000, 2,000- 5,000, 2,000- 3,000, 2,000- 4,000, 3,000- 5,000, 3,000- 4,000, or 2,000- 2,500. A neutralization titer is typially expressed as the highest serum dilution required to achieve a 50% reduction in the number of plaques.

In certain aspects, vaccines of the invention (e.g., LNP-encapsulated mRNA vaccines) produce prophylactically- and/or therapeutically- efficacious levels, concentrations and/or titers of antigen- specific antibodies in the blood or serum of a vaccinated subject. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, etc.

In exemplary embodiments of the invention, an efficacious vaccine produces an antibody titer of greater than 1:40, greater that 1: 100, greater than 1:400, greater than 1: 1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.)

In exemplary aspects of the invention, antigen- specific antibodies are measured in units of µg/ml or are measured in units of IU/L (International Units per liter) or mlU/ml (milli International Units per ml). In exemplary embodiments of the invention, an efficacious vaccine produces >0.5 µg/ml, >0.1 µg/ml, >0.2 µg/ml, >0.35 µg/ml, >0.5 µg/ml, >1 µg/ml, >2 µg/ml, >5 µg/ml or >10 µg/ml. In exemplary embodiments of the invention, an efficacious vaccine produces >10 mlU/ml, >20 mlU/ml, >50 mlU/ml, >100 mlU/ml, >200 mlU/ml, >500 mlU/ml or >1000 mlU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay. Also provided are nucleic acid vaccines comprising one or more RNA polynucleotides having an open reading frame encoding a first antigenic polypeptide or a concatemeric polypeptide, wherein the RNA polynucleotide is present in a formulation for in vivo administration to a host for eliciting a longer lasting high antibody titer than an antibody titer elicited by an mRNA vaccine having a stabilizing element or formulated with an adjuvant and encoding the first antigenic polypeptide. In some embodiments, the RNA polynucleotide is formulated to produce a neutralizing antibodies within one week of a single administration. In some embodiments, the adjuvant is selected from a cationic peptide and an immunostimulatory nucleic acid. In some embodiments, the cationic peptide is protamine.

Aspects provide nucleic acid vaccines comprising one or more RNA polynucleotides having an open reading frame comprising at least one chemical modification or optionally no nucleotide modification, the open reading frame encoding a first antigenic polypeptide or a concatemeric polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the first antigenic polypeptide.

Other aspects provide nucleic acid vaccines comprising one or more RNA polynucleotides having an open reading frame comprising at least one chemical modification or optionally no nucleotide modification, the open reading frame encoding a first antigenic polypeptide or a concatemeric polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

Aspects of the invention also provide a unit of use vaccine, comprising between 10 ug and 400 ug of one or more RNA polynucleotides having an open reading frame comprising at least one chemical modification or optionally no nucleotide modification, the open reading frame encoding a first antigenic polypeptide or a concatemeric polypeptide, and a pharmaceutically acceptable carrier or excipient, formulated for delivery to a human subject. In some embodiments, the vaccine further comprises a cationic lipid nanoparticle.

Aspects of the invention provide methods of creating, maintaining or restoring antigenic memory to a tumor in an individual or population of individuals comprising administering to said individual or population an antigenic memory booster nucleic acid vaccine comprising (a) at least one RNA polynucleotide, said polynucleotide comprising at least one chemical modification or optionally no nucleotide modification and two or more codon-optimized open reading frames, said open reading frames encoding a set of reference antigenic polypeptides, and (b) optionally a pharmaceutically acceptable carrier or excipient. In some embodiments, the vaccine is administered to the individual via a route selected from the group consisting of intramuscular administration, intradermal administration and subcutaneous administration. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

Aspects of the invention provide methods of vaccinating a subject comprising administering to the subject a single dosage of between 25 ug/kg and 400 ug/kg of a nucleic acid vaccine comprising one or more RNA polynucleotides having an open reading frame encoding a first antigenic polypeptide or a concatemeric polypeptide in an effective amount to vaccinate the subject.

Other aspects provide nucleic acid vaccines comprising one or more RNA polynucleotides having an open reading frame comprising at least one chemical modification, the open reading frame encoding a first antigenic polypeptide or a concatemeric polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

Other aspects provide nucleic acid vaccines comprising an LNP formulated RNA polynucleotide having an open reading frame comprising no nucleotide modifications (unmodified), the open reading frame encoding a first antigenic polypeptide or a

concatemeric polypeptide, wherein the vaccine has at least 10 fold less RNA polynucleotide than is required for an unmodified mRNA vaccine not formulated in a LNP to produce an equivalent antibody titer. In some embodiments, the RNA polynucleotide is present in a dosage of 25-100 micrograms.

In other aspects the invention encompasses a method of treating an elderly subject age 60 years or older comprising administering to the subject a nucleic acid vaccine comprising one or more RNA polynucleotides having an open reading frame encoding an antigenic polypeptide or a concatemeric polypeptide in an effective amount to vaccinate the subject.

In other aspects the invention encompasses a method of treating a young subject age 17 years or younger comprising administering to the subject a nucleic acid vaccine comprising one or more RNA polynucleotides having an open reading frame encoding an antigenic polypeptide or a concatemeric polypeptide in an effective amount to vaccinate the subject.

In other aspects the invention encompasses a method of treating an adult subject comprising administering to the subject a nucleic acid vaccine comprising one or more RNA polynucleotides having an open reading frame encoding an antigenic polypeptide or a concatemeric polypeptide in an effective amount to vaccinate the subject.

In some aspects the invention comprises a method of vaccinating a subject with a combination vaccine including at least two nucleic acid sequences encoding antigens wherein the dosage for the vaccine is a combined therapeutic dosage wherein the dosage of each individual nucleic acid encoding an antigen is a sub therapeutic dosage. In some embodiments, the combined dosage is 25 micrograms of the RNA polynucleotide in the nucleic acid vaccine administered to the subject. In some embodiments, the combined dosage is 100 micrograms of the RNA polynucleotide in the nucleic acid vaccine administered to the subject. In some embodiments the combined dosage is 50 micrograms of the RNA polynucleotide in the nucleic acid vaccine administered to the subject. In some embodiments, the combined dosage is 75 micrograms of the RNA polynucleotide in the nucleic acid vaccine administered to the subject. In some embodiments, the combined dosage is 150 micrograms of the RNA polynucleotide in the nucleic acid vaccine administered to the subject. In some embodiments, the combined dosage is 400 micrograms of the RNA polynucleotide in the nucleic acid vaccine administered to the subject. In some embodiments, the sub therapeutic dosage of each individual nucleic acid encoding an antigen is 1, 2, 3, 4, 5, 6, 7, 8, 9, 10, 11, 12, 13, 14, 15, 16, 17, 18, 19, or 20 micrograms. In other embodiments the nucleic acid vaccine is chemically modified and in other embodiments the nucleic acid vaccine is not chemically modified.

### Other Components

A LNP (e.g., an empty LNP or a loaded LNP of the disclosure) may include one or more components in addition to those described in the preceding sections. In some embodiments, a LNP (e.g., an empty LNP or a loaded LNP of the disclosure)may include one or more small hydrophobic molecules such as a vitamin (e.g., vitamin A or vitamin E) or a sterol.

Lipid nanoparticles (e.g., empty LNPs or loaded LNPs of the disclosure) may also include one or more permeability enhancer molecules, carbohydrates, polymers, surface altering agents, or other components. A permeability enhancer molecule may be a molecule described by U.S. Pat. Application Publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a LNP. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. In some embodiments, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(A-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, domase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A LNP (e.g., an empty LNP or a loaded LNP of the disclosure) may also comprise one or more functionalized lipids. In some embodiments, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP (e.g., an empty LNP or a loaded LNP of the disclosure) may also be conjugated with one or more useful antibodies. Functional groups and conjugates useful in targeted cell delivery, imaging, and membrane permeation are well known in the art.

In addition to these components, lipid nanoparticles (e.g., empty LNPs or loaded LNPs of the disclosure) may include any substance useful in pharmaceutical compositions. In some embodiments, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington’s The Science and Practice of Pharmacy, 21^{st} Edition, A. R. Gennaro; Lippincott, Williams & Wilkins, Baltimore, MD, 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEENⓇ20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMERⓇ 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALLⓇ 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer’s solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademianut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### Pharmaceutical Compositions

Formulations comprising lipid nanoparticles may be formulated in whole or in part as pharmaceutical compositions. Pharmaceutical compositions may include one or more lipid nanoparticles. In some embodiments, a pharmaceutical composition may include one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington’s The Science and Practice of Pharmacy, 21^{st} Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP in the formulation of the disclosure. An excipient or accessory ingredient may be incompatible with a component of a LNP of the formulation if its combination with the component or LNP may result in any undesirable biological effect or otherwise deleterious effect.

In some embodiments, one or more excipients or accessory ingredients may make up greater than 50% of the total mass or volume of a pharmaceutical composition including a LNP. In some embodiments, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition comprises between 0.1% and 100% (wt/wt) of one or more lipid nanoparticles. As another example, a pharmaceutical composition comprises between 0.1% and 15% (wt/vol) of one or more amphiphilic polymers (e.g., 0.5%, 1%, 2.5%, 5%, 10%, or 12.5% w/v).

In some embodiments, the lipid nanoparticles and/or pharmaceutical compositions of the disclosure are refrigerated or frozen for storage and/or shipment (e.g., being stored at a temperature of 4° C. or lower, such as a temperature between about -150° C. and about 0° C. or between about -80° C. and about -20° C. (e.g., about -5° C., -10° C., -15° C., -20° C., -25° C., -30° C., -40° C., -50° C., -60° C., -70° C., -80° C., -90° C., -130° C. or -150° C.). For example, the pharmaceutical composition comprising one or more lipid nanoparticles is a solution or solid (e.g., via lyophilization) that is refrigerated for storage and/or shipment at, for example, about -20° C., -30° C., -40° C., -50° C., -60° C., -70° C., or -80° C. In certain embodiments, the disclosure also relates to a method of increasing stability of the lipid nanoparticles and by storing the lipid nanoparticles and/or pharmaceutical compositions thereof at a temperature of 4° C. or lower, such as a temperature between about -150° C. and about 0° C. or between about -80° C. and about -20° C., e.g., about -5° C., -10° C., -15° C., -20° C., -25° C., -30° C., -40° C., -50° C., -60° C., -70° C., -80° C., -90° C., -130° C. or -150° C.).

Lipid nanoparticles and/or pharmaceutical compositions including one or more lipid nanoparticles may be administered to any patient or subject, including those patients or subjects that may benefit from a therapeutic effect provided by the delivery of a therapeutic and/or prophylactic to one or more particular cells, tissues, organs, or systems or groups thereof, such as the renal system. Although the descriptions provided herein of lipid nanoparticles and pharmaceutical compositions including lipid nanoparticles are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more lipid nanoparticles may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multidose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., lipid nanoparticle). The amount of the active ingredient is generally equal to the dosage of the active ingredient which would be administered to a subject and/or a convenient fraction of such a dosage such as, for example, one-half or one-third of such a dosage.

Pharmaceutical compositions may be prepared in a variety of forms suitable for a variety of routes and methods of administration. In some embodiments, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutics and/or prophylactics, additional agents such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer’s solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, films, powders, and granules. In such solid dosage forms, an active ingredient is mixed with at least one inert, pharmaceutically acceptable excipient such as sodium citrate or dicalcium phosphate and/or fillers or extenders (e.g., starches, lactose, sucrose, glucose, mannitol, and silicic acid), binders (e.g., carboxymethylcellulose, alginates, gelatin, polyvinylpyrrolidone, sucrose, and acacia), humectants (e.g., glycerol), disintegrating agents (e.g., agar, calcium carbonate, potato or tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay, silicates), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only. In some embodiments, the solid compositions may optionally comprise opacifying agents and can be of a composition that they release the active ingredient(s) in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pats. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pats. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (wt/wt) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 µm to 500 µm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

### Methods of Producing Polypeptides in Cells

The present disclosure provides methods of producing a polypeptide of interest in a mammalian cell. Methods of producing polypeptides involve contacting a cell with a formulation of the disclosure comprising a LNP including an mRNA encoding the polypeptide of interest. Upon contacting the cell with the lipid nanoparticle, the mRNA may be taken up and translated in the cell to produce the polypeptide of interest.

In general, the step of contacting a mammalian cell with a LNP including an mRNA encoding a polypeptide of interest may be performed in vivo, ex vivo, in culture, or in vitro. The amount of lipid nanoparticle contacted with a cell, and/or the amount of mRNA therein, may depend on the type of cell or tissue being contacted, the means of administration, the physiochemical characteristics of the lipid nanoparticle and the mRNA (e.g., size, charge, and chemical composition) therein, and other factors. In general, an effective amount of the lipid nanoparticle will allow for efficient polypeptide production in the cell. Metrics for efficiency may include polypeptide translation (indicated by polypeptide expression), level of mRNA degradation, and immune response indicators.

The step of contacting a LNP including an mRNA with a cell may involve or cause transfection. A phospholipid including in the lipid component of a LNP may facilitate transfection and/or increase transfection efficiency, for example, by interacting and/or fusing with a cellular or intracellular membrane. Transfection may allow for the translation of the mRNA within the cell.

In some embodiments, the lipid nanoparticles described herein may be used therapeutically. For example, an mRNA included in a LNP may encode a therapeutic polypeptide (e.g., in a translatable region) and produce the therapeutic polypeptide upon contacting and/or entry (e.g., transfection) into a cell. In other embodiments, an mRNA included in a LNP may encode a polypeptide that may improve or increase the immunity of a subject. In some embodiments, an mRNA may encode a granulocyte-colony stimulating factor or trastuzumab.

In some embodiments, an mRNA included in a LNP may encode a recombinant polypeptide that may replace one or more polypeptides that may be substantially absent in a cell contacted with the lipid nanoparticle. The one or more substantially absent polypeptides may be lacking due to a genetic mutation of the encoding gene or a regulatory pathway thereof. Alternatively, a recombinant polypeptide produced by translation of the mRNA may antagonize the activity of an endogenous protein present in, on the surface of, or secreted from the cell. An antagonistic recombinant polypeptide may be desirable to combat deleterious effects caused by activities of the endogenous protein, such as altered activities or localization caused by mutation. In another alternative, a recombinant polypeptide produced by translation of the mRNA may indirectly or directly antagonize the activity of a biological moiety present in, on the surface of, or secreted from the cell. Antagonized biological moieties may include, but are not limited to, lipids (e.g., cholesterol), lipoproteins (e.g., low density lipoprotein), nucleic acids, carbohydrates, and small molecule toxins. Recombinant polypeptides produced by translation of the mRNA may be engineered for localization within the cell, such as within a specific compartment such as the nucleus, or may be engineered for secretion from the cell or for translocation to the plasma membrane of the cell.

In some embodiments, contacting a cell with a LNP including an mRNA may reduce the innate immune response of a cell to an exogenous nucleic acid. A cell may be contacted with a first lipid nanoparticle including a first amount of a first exogenous mRNA including a translatable region and the level of the innate immune response of the cell to the first exogenous mRNA may be determined. Subsequently, the cell may be contacted with a second composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second compositions may be repeated one or more times. Additionally, efficiency of polypeptide production (e.g., translation) in the cell may be optionally determined, and the cell may be re-contacted with the first and/or second composition repeatedly until a target protein production efficiency is achieved.

### Methods of Delivering Therapeutic Agents to Cells and Organs

The present disclosure provides methods of delivering a therapeutic and/or prophylactic, such as a nucleic acid, to a mammalian cell or organ. Delivery of a therapeutic and/or prophylactic to a cell involves administering a formulation of the disclosure that comprises a LNP including the therapeutic and/or prophylactic, such as a nucleic acid, to a subject, where administration of the composition involves contacting the cell with the composition. In some embodiments, a protein, cytotoxic agent, radioactive ion, chemotherapeutic agent, or nucleic acid (such as an RNA, e.g., mRNA) may be delivered to a cell or organ. In the instance that a therapeutic and/or prophylactic is an mRNA, upon contacting a cell with the lipid nanoparticle, a translatable mRNA may be translated in the cell to produce a polypeptide of interest. However, mRNAs that are substantially not translatable may also be delivered to cells. Substantially non-translatable mRNAs may be useful as vaccines and/or may sequester translational components of a cell to reduce expression of other species in the cell.

In some embodiments, a LNP may target a particular type or class of cells (e.g., cells of a particular organ or system thereof). In some embodiments, a LNP including a therapeutic and/or prophylactic of interest may be specifically delivered to a mammalian liver, kidney, spleen, femur, or lung. Specific delivery to a particular class of cells, an organ, or a system or group thereof implies that a higher proportion of lipid nanoparticles including a therapeutic and/or prophylactic are delivered to the destination (e.g., tissue) of interest relative to other destinations, e.g., upon administration of a LNP to a mammal. In some embodiments, specific delivery may result in a greater than 2 fold, 5 fold, 10 fold, 15 fold, or 20 fold increase in the amount of therapeutic and/or prophylactic per 1 g of tissue of the targeted destination (e.g., tissue of interest, such as a liver) as compared to another destination (e.g., the spleen). In some embodiments, the tissue of interest is selected from the group consisting of a liver, kidney, a lung, a spleen, a femur, vascular endothelium in vessels (e.g., intra-coronary or intra-femoral) or kidney, and tumor tissue (e.g., via intratumoral injection).

As another example of targeted or specific delivery, an mRNA that encodes a protein-binding partner (e.g., an antibody or functional fragment thereof, a scaffold protein, or a peptide) or a receptor on a cell surface may be included in a LNP. An mRNA may additionally or instead be used to direct the synthesis and extracellular localization of lipids, carbohydrates, or other biological moieties. Alternatively, other therapeutics and/or prophylactics or elements (e.g., lipids or ligands) of a LNP may be selected based on their affinity for particular receptors (e.g., low density lipoprotein receptors) such that a LNP may more readily interact with a target cell population including the receptors. In some embodiments, ligands may include, but are not limited to, members of a specific binding pair, antibodies, monoclonal antibodies, Fv fragments, single chain Fv (scFv) fragments, Fab′ fragments, F(ab′)2 fragments, single domain antibodies, camelized antibodies and fragments thereof, humanized antibodies and fragments thereof, and multivalent versions thereof; multivalent binding reagents including mono- or bi-specific antibodies such as disulfide stabilized Fv fragments, scFv tandems, diabodies, tribodies, or tetrabodies; and aptamers, receptors, and fusion proteins.

In some embodiments, a ligand may be a surface-bound antibody, which can permit tuning of cell targeting specificity. This is especially useful since highly specific antibodies can be raised against an epitope of interest for the desired targeting site. In one embodiment, multiple antibodies are expressed on the surface of a cell, and each antibody can have a different specificity for a desired target. Such approaches can increase the avidity and specificity of targeting interactions.

A ligand can be selected, e.g., by a person skilled in the biological arts, based on the desired localization or function of the cell.

In some embodiments, a LNP may target hepatocytes. Apolipoproteins such as apolipoprotein E (apoE) have been shown to associate with neutral or near neutral lipid-containing lipid nanoparticles in the body, and are known to associate with receptors such as low-density lipoprotein receptors (LDLRs) found on the surface of hepatocytes. Thus, a LNP including a lipid component with a neutral or near neutral charge that is administered to a subject may acquire apoE in a subject’s body and may subsequently deliver a therapeutic and/or prophylactic (e.g., an RNA) to hepatocytes including LDLRs in a targeted manner.

### Methods of Treating Diseases and Disorders

In some aspects, the present disclosure provides a method of treating or preventing a disease or disorder, the method comprising administering to a subject in need thereof an empty LNP described herein.

In some aspects, the present disclosure provides a method of treating or preventing a disease or disorder, the method comprising administering to a subject in need thereof an empty-LNP solution described herein.

In some aspects, the present disclosure provides a method of treating or preventing a disease or disorder, the method comprising administering to a subject in need thereof a loaded LNP described herein.

In some aspects, the present disclosure provides a method of treating or preventing a disease or disorder, the method comprising administering to a subject in need thereof a loaded-LNP solution described herein.

In some aspects, the present disclosure provides a method of treating or preventing a disease or disorder, the method comprising administering to a subject in need thereof a LNP formulation described herein.

In some aspects, the present disclosure provides an empty LNP disclosed herein for use in treating or preventing a disease or disorder in a subject.

In some aspects, the present disclosure provides an empty-LNP solution disclosed herein for use in treating or preventing a disease or disorder in a subject.

In some aspects, the present disclosure provides a loaded LNP disclosed herein for use in treating or preventing a disease or disorder in a subject.

In some aspects, the present disclosure provides a loaded-LNP solution disclosed herein for use in treating or preventing a disease or disorder in a subject.

In some aspects, the present disclosure provides a LNP formulation disclosed herein for use in treating or preventing a disease or disorder in a subject.

In some aspects, the present disclosure provides a use of an empty LNP disclosed herein in the manufacture of a medicament for treating or preventing a disease or disorder.

In some aspects, the present disclosure provides a use of an empty-LNP solution disclosed herein in the manufacture of a medicament for treating or preventing a disease or disorder.

In some aspects, the present disclosure provides a use of a loaded LNP disclosed herein in the manufacture of a medicament for treating or preventing a disease or disorder.

In some aspects, the present disclosure provides a use of a loaded-LNP solution disclosed herein in the manufacture of a medicament for treating or preventing a disease or disorder.

In some aspects, the present disclosure provides a method of administering an empty LNP disclosed herein to a subject.

In some aspects, the present disclosure provides a method of administering an empty-LNP solution disclosed herein to a subject.

In some aspects, the present disclosure provides a method of administering a loaded LNP disclosed herein to a subject.

In some aspects, the present disclosure provides a method of administering a loaded-LNP solution disclosed herein to a subject.

In some aspects, the present disclosure provides a method of administering a LNP formulation disclosed herein to a subject.

Lipid nanoparticles may be useful for treating a disease, disorder, or condition. In particular, such compositions may be useful in treating a disease, disorder, or condition characterized by missing or aberrant protein or polypeptide activity. In some embodiments, a formulation of the disclosure that comprises a LNP including an mRNA encoding a missing or aberrant polypeptide may be administered or delivered to a cell. Subsequent translation of the mRNA may produce the polypeptide, thereby reducing or eliminating an issue caused by the absence of or aberrant activity caused by the polypeptide. Because translation may occur rapidly, the methods and compositions may be useful in the treatment of acute diseases, disorders, or conditions such as sepsis, stroke, and myocardial infarction. A therapeutic and/or prophylactic included in a LNP may also be capable of altering the rate of transcription of a given species, thereby affecting gene expression.

The disclosure provides methods involving administering lipid nanoparticles including one or more therapeutic and/or prophylactic agents, such as a nucleic acid, and pharmaceutical compositions including the same. The terms therapeutic and prophylactic can be used interchangeably herein with respect to features and embodiments of the present disclosure. Therapeutic compositions, or imaging, diagnostic, or prophylactic compositions thereof, may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject; the purpose of the administration; the particular composition; the mode of administration; and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment. The specific therapeutically effective, prophylactically effective, or otherwise appropriate dose level (e.g., for imaging) for any particular patient will depend upon a variety of factors including the severity and identify of a disorder being treated, if any; the one or more therapeutics and/or prophylactics employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

A LNP including one or more therapeutics and/or prophylactics, such as a nucleic acid, may be administered by any route. In some embodiments, compositions, including prophylactic, diagnostic, or imaging compositions including one or more lipid nanoparticles described herein, are administered by one or more of a variety of routes, including oral, intravenous, intramuscular, intra-arterial, intramedullary, intrathecal, subcutaneous, intraventricular, trans- or intra-dermal, interdermal, rectal, intravaginal, intraperitoneal, topical (e.g., by powders, ointments, creams, gels, lotions, and/or drops), mucosal, nasal, buccal, enteral, intravitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. However, the present disclosure encompasses the delivery or administration of compositions described herein by any appropriate route taking into consideration likely advances in the sciences of drug delivery. In general, the most appropriate route of administration will depend upon a variety of factors including the nature of the lipid nanoparticle including one or more therapeutics and/or prophylactics (e.g., its stability in various bodily environments such as the bloodstream and gastrointestinal tract), the condition of the patient (e.g., whether the patient is able to tolerate particular routes of administration), etc.

In certain embodiments, compositions in accordance with the present disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of a therapeutic and/or prophylactic (e.g., an mRNA) in a given dose, where a dose of 1 mg/kg (mpk) provides 1 mg of a therapeutic and/or prophylactic per 1 kg of subject body weight. In some embodiments, a dose of about 0.001 mg/kg to about 10 mg/kg of a therapeutic and/or prophylactic (e.g., mRNA) of a LNP may be administered. In other embodiments, a dose of about 0.005 mg/kg to about 2.5 mg/kg of a therapeutic and/or prophylactic may be administered. In certain embodiments, a dose of about 0.1 mg/kg to about 1 mg/kg may be administered. In other embodiments, a dose of about 0.05 mg/kg to about 0.25 mg/kg may be administered. A dose may be administered one or more times per day, in the same or a different amount, to obtain a desired level of mRNA expression and/or therapeutic, diagnostic, prophylactic, or imaging effect. The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition.

Lipid nanoparticles including one or more therapeutics and/or prophylactics, such as a nucleic acid, may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. In some embodiments, one or more lipid nanoparticles including one or more different therapeutics and/or prophylactics may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects, such as infusion related reactions).

A LNP may be used in combination with an agent to increase the effectiveness and/or therapeutic window of the composition. Such an agent may be, for example, an antiinflammatory compound, a steroid (e.g., a corticosteroid), a statin, an estradiol, a BTK inhibitor, an S1P1 agonist, a glucocorticoid receptor modulator (GRM), or an anti-histamine. In some embodiments, a LNP may be used in combination with dexamethasone, methotrexate, acetaminophen, an H1 receptor blocker, or an H2 receptor blocker. In some embodiments, a method of treating a subject in need thereof or of delivering a therapeutic and/or prophylactic to a subject (e.g., a mammal) may involve pre-treating the subject with one or more agents prior to administering a LNP. In some embodiments, a subject may be pre-treated with a useful amount (e.g., 10 mg, 20 mg, 30 mg, 40 mg, 50 mg, 60 mg, 70 mg, 80 mg, 90 mg, 100 mg, or any other useful amount) of dexamethasone, methotrexate, acetaminophen, an H1 receptor blocker, or an H2 receptor blocker. Pre-treatment may occur 24 or fewer hours (e.g., 24 hours, 20 hours, 16 hours, 12 hours, 8 hours, 4 hours, 2 hours, 1 hour, 50 minutes, 40 minutes, 30 minutes, 20 minutes, or 10 minutes) before administration of the lipid nanoparticle and may occur one, two, or more times in, for example, increasing dosage amounts.

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all, of the group members are present in, employed in, or otherwise relevant to a given product or process.

It is also noted that the term “comprising” is intended to be open and permits but does not require the inclusion of additional elements or steps. When the term “comprising” is used herein, the terms “consisting essentially of” and “consisting of” are thus also encompassed and disclosed. Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein.

All cited sources, for example, references, publications, patent applications, databases, database entries, and art cited herein, are incorporated into this application by reference, even if not expressly stated in the citation. In case of conflicting statements of a cited source and the instant application, the statement in the instant application shall control.

The disclosure having been described, the following examples are offered by way of illustration and not limitation.

### Equivalents

The details of one or more embodiments of the invention are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the invention to the precise form disclosed, but by the claims appended hereto.

### Enumerated Embodiments

Embodiment 1. A method of preparing an empty-lipid nanoparticle solution (empty-LNP solution) comprising an empty lipid nanoparticle (empty LNP), comprising:

(i) a mixing step, comprising mixing a lipid solution comprising an ionizable lipid, a phospholipid, a PEG lipid and a structural lipid with an aqueous buffer solution comprising a first buffering agent, thereby forming the empty-LNP solution comprising the empty LNP, wherein the empty-LNP solution comprises an acetate buffer and has a pH in the range of about 4.6 to about 6.0

Embodiment 2. A method of preparing an empty-lipid nanoparticle solution (empty-LNP solution) comprising an empty lipid nanoparticle (empty LNP), comprising:
(i) a mixing step, comprising mixing a lipid solution comprising an ionizable lipid, a phospholipid, a PEG lipid and a structural lipid with an aqueous buffer solution comprising a first buffering agent, thereby forming the empty-LNP solution comprising the empty LNP, wherein the empty LNP comprises from about 0.1 mol% to about 0.5 mol% of the PEG lipid,
wherein the empty-LNP solution comprises an acetate buffer and has a pH in the range of about 4.6 to about 6.0.

Embodiment 3. The method of embodiment 1, further comprising processing the empty-LNP solution.

Embodiment 4. A method of preparing a loaded lipid nanoparticle solution (loaded-LNP solution) comprising a loaded lipid nanoparticle (loaded LNP), comprising:
(i) a mixing step, comprising mixing a lipid solution comprising an ionizable lipid, a phospholipid, a PEG lipid and a structural lipid with an aqueous buffer solution comprising a first buffering agent, thereby forming the empty-LNP solution comprising the empty LNP, wherein the empty-LNP solution comprises an acetate buffer and has a pH in the range of about 4.6 to about 6.0; and
(ii) a loading step, comprising mixing a nucleic acid solution comprising a nucleic acid with the empty-LNP solution, thereby forming a loaded-LNP solution comprising a loaded LNP.

Embodiment 5. A method of preparing a loaded lipid nanoparticle solution (loaded-LNP solution) comprising a loaded lipid nanoparticle (loaded LNP), comprising:
(i) a mixing step, comprising mixing a lipid solution comprising an ionizable lipid, a phospholipid, a PEG lipid and a structural lipid with an aqueous buffer solution comprising a first buffering agent, thereby forming the empty-LNP solution comprising the empty LNP, wherein the empty LNP comprises from about 0.1 mol% to about 0.5 mol% of the PEG lipid, wherein the empty-LNP solution comprises an acetate buffer and has a pH in the range of about 4.6 to about 6.0; and
(ii) a loading step, comprising mixing a nucleic acid solution comprising a nucleic acid with the empty-LNP solution, thereby forming a loaded-LNP solution comprising a loaded LNP.

Embodiment 6. The method of any one of the preceding embodiments, further comprising processing the loaded-LNP solution, thereby forming a lipid nanoparticle formulation (LNP formulation).

Embodiment 7. The method of any one of the preceding embodiments, wherein the step of processing the loaded-LNP solution comprises a first adding step, comprising adding a polyethylene glycol lipid (PEG lipid) to the loaded LNP.

Embodiment 8. The method of any one of the preceding embodiments, wherein the first adding step comprises adding a polyethylene glycol solution (PEG solution) comprising the PEG lipid to the loaded-LNP solution.

Embodiment 9. The method of any one of the preceding embodiments, wherein the first adding step comprises adding from about 0.1 mol% to about 3.0 mol% PEG lipid, from about 0.2 mol% to about 2.5 mol% PEG lipid, from about 0.5 mol% to about 2.0 mol% PEG lipid, from about 0.75 mol% to about 1.5 mol%PEG lipid, or from about 1.0 mol% to about 1.25 mol% PEG lipid to the empty LNP or the loaded LNP.

Embodiment 10. The method of any one of the preceding embodiments, wherein the step of processing the empty-LNP solution further comprises pH adjusting.

Embodiment 11. The method of any one of the preceding embodiments, wherein the pH adjusting comprises adding a second buffering agent.

Embodiment 12. The method of any one of the preceding embodiments, wherein the second buffering agent comprises a second aqueous buffer solution.

Embodiment 13. The method of any one of the preceding embodiments, wherein the second aqueous buffer is selected from the group consisting of an acetate buffer, a citrate buffer, a phosphate buffer, and a tris buffer.

Embodiment 14. The method of any one of the preceding embodiments, wherein the buffering agent is selected from the group consisting of an acetate buffer, a citrate buffer, a phosphate buffer, and a tris buffer.

Embodiment 15. The method of any one of the preceding embodiments, wherein the buffering agent is acetate

Embodiment 16. The method of any one of the preceding embodiments, wherein the step of processing the empty-LNP solution further comprises filtering.

Embodiment 17. The method of any one of the preceding embodiments, wherein the filtering is performed by a tangential flow filtration.

Embodiment 18. The method of any one of the preceding embodiments, wherein the step of processing the loaded-LNP solution further comprises buffer exchanging.

Embodiment 19. The method of any one of the preceding embodiments, wherein the buffer exchanging comprises addition of an aqueous buffer solution comprising a third buffering agent.

Embodiment 20. The method of any one of the preceding embodiments, wherein the third buffering agent comprises a third aqueous buffer solution.

Embodiment 21. The method of any one of the preceding embodiments, wherein the third aqueous buffer solution is selected from the group consisting of an acetate buffer, a citrate buffer, a phosphate buffer, and a tris buffer.

Embodiment 22. The method of any one of the preceding embodiments, wherein the third aqueous buffer solution has a pH in a range of about 6.5 to about 8.5, about 7.0 to about 8.0, about 7.2 to about 7.8, or about 7.4 to about 7.6.

Embodiment 23. The method of any one of the preceding embodiments, wherein the third aqueous buffer solution has a pH of about 7.5.

Embodiment 24. The method of any one of the preceding embodiments, wherein the first adding step is performed prior to the buffer exchanging.

Embodiment 25. The method of any one of the preceding embodiments, wherein the first adding step is performed after the buffer exchanging.

Embodiment 26. The method of any one of the preceding embodiments, wherein the step of processing the loaded-LNP solution comprises a second adding step, comprising adding a polyethylene glycol lipid (PEG lipid) to the loaded LNP.

Embodiment 27. The method of any one of the preceding embodiments, wherein the second adding step is performed prior to the buffer exchanging.

Embodiment 28. The method of any one of the preceding embodiments, wherein the second adding step is performed after the buffer exchanging.

Embodiment 29. The method of any one of the preceding embodiments, wherein the step of processing the empty-LNP solution further comprises diluting the empty-LNP solution.

Embodiment 30. The method of any one of the preceding embodiments, wherein the step of processing the empty-LNP solution or loaded-LNP solution further comprises freezing the empty-LNP solution or loaded-LNP solution.

Embodiment 31. The method of any one of the preceding embodiments, wherein the step of processing the empty-LNP solution or loaded-LNP solution further comprises lyophilizing the empty-LNP solution or loaded-LNP solution.

Embodiment 32. The method of any one of the preceding embodiments, wherein the step of processing the empty-LNP solution or loaded-LNP solution further comprises storing the empty-LNP solution or loaded-LNP solution.

Embodiment 33. The method of any one of the preceding embodiments, wherein the mixing step is performed with a T-junction, confined impinging jets, microfluidic mixer, or vortex mixer.

Embodiment 34. The method of any one of the preceding embodiments, wherein the loading step is performed with a T-junction, confined impinging jets, microfluidic mixer, or vortex mixer.

Embodiment 35. The method of any one of the preceding embodiments, wherein the aqueous buffer solution has a pH in a range of from about 4.5 to about 6.5, from about 4.6 to about 6.0, from about 4.7 to about 5.75, from about 4.8 to about 5.5, or from about 4.9 to about 5.25.

Embodiment 36. The method of any one of the preceding embodiments, wherein the aqueous buffer solution has a pH of about 5.0.

Embodiment 37. The method of any one of the preceding embodiments, wherein the empty-LNP solution has a pH in a range of from about 4.8 to about 5.8, from about 5.0 to about 5.75, or from about 5.0 to about 5.5.

Embodiment 38. The method of any one of the preceding embodiments, wherein the nucleic acid solution has a pH in a range of from about 4.5 to about 6.5, from about 4.8 to about 6.25, from about 4.8 to about 6.0, from about 5.0 to about 5.8, or from about 5.2 to about 5.5.

Embodiment 39. The method of any one of the preceding embodiments, wherein the pH of the nucleic acid solution, the empty-LNP solution, and the LNP formulation are in a range of from about 5.0 to about 6.0, from about 5.1 to about 5.75, or from about 5.2 to about 5.5.

Embodiment 40. The method of any one of the preceding embodiments, wherein the loaded-LNP solution has a pH in a range of from about 4.5 to about 6.0, from about 4.6 to about 5.8, from about 4.8 to about 5.6, from about 5.0 to about 5.5, or from about 5.1 to about 5.4.

Embodiment 41. The method of any one of the preceding embodiments, wherein the lipid solution further comprises a first organic solvent.

Embodiment 42. The method of any one of the preceding embodiments, wherein the empty-LNP solution or loaded-LNP solution further comprises a first organic solvent.

Embodiment 43. The method of any one of the preceding embodiments, wherein the first organic solvent is an alcohol.

Embodiment 44. The method of any one of the preceding embodiments, wherein the first organic solvent is ethanol.

Embodiment 45. The method of any one of the preceding embodiments, wherein the first buffering agent comprises a first aqueous buffer solution.

Embodiment 46. The method of any one of the preceding embodiments, wherein the first aqueous buffer is selected from the group consisting of an acetate buffer, a citrate buffer, a phosphate buffer, and a tris buffer.

Embodiment 47. The method of any one of the preceding embodiments, wherein the first aqueous buffer solution comprises greater than about 1 mM citrate, acetate, phosphate or tris, greater than about 2 mM citrate, acetate, phosphate or tris, greater than about 5 mM citrate, acetate, phosphate or tris, greater than about 10 mM citrate, acetate, phosphate or tris, greater than about 15 mM citrate, acetate, phosphate or tris, greater than about 20 mM citrate, acetate, phosphate or tris, greater than about 25 mM citrate, acetate, phosphate or tris, or greater than about 30 mM citrate, acetate, phosphate or tris.

Embodiment 48. The method of any one of the preceding embodiments, wherein the first aqueous buffer solution comprises about 1 mM to about 30 mM citrate, acetate, phosphate or tris, about 2 mM to about 20 mM citrate, acetate, phosphate or tris, about 3 mM to about 10 mM citrate, acetate, phosphate or tris, about 4 mM to about 8 mM citrate, acetate, phosphate or tris, or about 5 mM to about 6 mM citrate, acetate, phosphate or tris.

Embodiment 49. The method of any one of the preceding embodiments, wherein the first aqueous buffer solution comprises about 5 mM citrate, acetate, phosphate or tris.

Embodiment 50. The method of any one of the preceding embodiments, wherein the first aqueous buffer solution comprises about 5 mM acetate, wherein the aqueous buffer solution has a pH of about 5.0.

Embodiment 51. The method of any one of the preceding embodiments, wherein the empty-LNP solution or loaded-LNP solution further comprises a tonicity agent.

Embodiment 52. The method of any one of the preceding embodiments, wherein the tonicity agent is a sugar.

Embodiment 53. The method of any one of the preceding embodiments, wherein the sugar is sucrose.

Embodiment 54. The method of any one of the preceding embodiments, wherein the empty-LNP solution or loaded-LNP solution comprises from about 0.01 g/mL to about 1.0 g/mL, from about 0.05 g/mL to about 0.5 g/mL, from about 0.1 g/mL to about 0.4 g/mL, from about 0.15 g/mL to about 0.3 g/mL, or from about 0.2 g/mL to about 0.25 g/mL tonicity agent.

Embodiment 55. The method of any one of the preceding embodiments, wherein the empty-LNP solution or loaded-LNP solution further comprises from about 0.2 g/mL to about 0.25 g/mL tonicity agent.

Embodiment 56. The method of any one of the preceding embodiments, wherein the empty-LNP solution or loaded-LNP solution further comprises about 0.2 g/mL sucrose.

Embodiment 57. The method of any one of the preceding embodiments, wherein the nucleic acid solution comprises about 0.01 to about 1.0 mg/mL of the nucleic acid, about 0.05 to about 0.5 mg/mL of the nucleic acid, or about 0.1 to about 0.25 mg/mL of the nucleic acid.

Embodiment 58. The method of any one of the preceding embodiments, wherein the nucleic acid solution comprises a buffer selected from the group consisting of an acetate buffer, a citrate buffer, a phosphate buffer, and a tris buffer.

Embodiment 59. The method of any one of the preceding embodiments, wherein the nucleic acid solution comprises an acetate buffer.

Embodiment 60. The method of any one of the preceding embodiments, wherein the nucleic acid solution comprises from about 1 mM to about 200 mM acetate buffer, from about 2 mM to about 180 mM acetate buffer, from about 3 mM to about 160 mM acetate buffer, from about 4 mM to about 150 mM acetate buffer, from about 4 mM to about 140 mM acetate buffer, from about 5 mM to about 130 mM acetate buffer, from about 6 mM to about 120 mM acetate buffer, from about 7 mM to about 110 mM acetate buffer, from about 8 mM to about 100 mM acetate buffer, from about 9 mM to about 90 mM acetate buffer, from about 10 mM to about 80 mM acetate buffer, from about 15 mM to about 70 mM acetate buffer, from about 20 mM to about 60 mM acetate buffer, from about 25 mM to about 50 mM acetate buffer, or from about 30 mM to about 40 mM acetate buffer.

Embodiment 61. The method of any one of the preceding embodiments, wherein the nucleic acid solution and the empty-LNP solution are mixed at a volumetric flow ratio of from about 5:1 to about 7:1, from about 4:1 to about 6:1, from about 3:1 to about 5:1, or from about 2:1 to about 4:1 during the loading step.

Embodiment 62. The method of any one of the preceding embodiments, wherein the loaded-LNP solution comprises an acetate buffer.

Embodiment 63. The method of any one of the preceding embodiments, wherein the lipid solution, the empty LNP, the empty-LNP solution, the loaded LNP, the loaded-LNP solution, and/or the LNP formulation further comprises an encapsulation agent.

Embodiment 64. The method of any one of the preceding embodiments, wherein the lipid solution, the empty-LNP solution, the loaded LNP, the loaded-LNP solution, and/or the LNP formulation further comprises a phospholipid, a PEG lipid, a structural lipid, or any combination thereof.

Embodiment 65. The method of any one of the preceding embodiments, wherein the empty LNP comprises
about 30-60 mol% ionizable lipid;
about 0-30 mol% phospholipid;
about 15-50 mol% structural lipid; and
about 0.1-0.5 mol% PEG lipid.

Embodiment 66. The method of any one of the preceding embodiments, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol.

Embodiment 67. The method of any one of the preceding embodiments, wherein the structural lipid is selected from the group consisting of cholesterol, fecosterol, sitosterol, ergosterol, campesterol, stigmasterol, brassicasterol, tomatidine, ursolic acid, alpha-tocopherol, and derivatives thereof.

Embodiment 68. The method of any one of the preceding embodiments, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine,1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and derivatives thereof.

Embodiment 69. The method of any one of the preceding embodiments, wherein the ionizable lipid comprises an ionizable amino lipid.

Embodiment 70. The method of any one of the preceding embodiments, wherein the nucleic acid is a ribonucleic acid.

Embodiment 71. The method of any one of the preceding embodiments, wherein the ribonucleic acid is at least one ribonucleic acid selected from the group consisting of a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and a long non-coding RNA (1ncRNA).

Embodiment 72. The method of any one of the preceding embodiments, wherein the nucleic acid is a messenger RNA (mRNA).

Embodiment 73. The method of any one of the preceding embodiments, wherein the mRNA includes at least one motif selected from the group consisting of a stem loop, a chain terminating nucleoside, a poly A sequence, a polyadenylation signal, and a 5′ cap structure.

Embodiment 74. The method of any one of the preceding embodiments, wherein the mRNA is at least 30 nucleotides in length.

Embodiment 75. The method of any one of the preceding embodiments, wherein the mRNA is at least 300 nucleotides in length.

Embodiment 76. The method of any one of the preceding embodiments, wherein the LNP formulation has a N:P ratio from about 1.1:1 to about 30.1.

Embodiment 77. The method of any one of the preceding embodiments, wherein the LNP formulation has a N:P ratio from about 2:1 to about 20:1.

Embodiment 78. The method of any one of the preceding embodiments, wherein the LNP formulation has a N:P ratio from about 2:1 to about 10:1 or about 2:1 to about 5:1.

Embodiment 79. The method of any one of the preceding embodiments, wherein the LNP formulation comprises from about 0.01 to about 500 mg/mL of the nucleic acid, from about 0.1 to about 100 mg/mL, from about to about 50 mg/mL, from about 0.5 to about 10 mg/mL, or from about 1.0 to about 10 mg/mL of the nucleic acid.

Embodiment 80. An empty LNP comprising from about 0.1 mol% to about 0.5 mol% of a PEG lipid.

Embodiment 81. An empty LNP prepared by the method of any one of the preceding embodiments.

Embodiment 82. An empty-LNP solution prepared by the method of any one of the preceding embodiments.

Embodiment 83. An empty-LNP solution comprising an empty LNP, wherein the empty LNP comprises from about 0.1 mol% to about 0.5 mol% of a PEG lipid.

Embodiment 84. A loaded LNP prepared by the method of any one of the preceding embodiments.

Embodiment 85. A loaded-LNP solution prepared by the method of any one of the preceding embodiments.

Embodiment 86. A LNP formulation prepared by the method of any one of the preceding embodiments.

Embodiment 87. A method of treating or preventing a disease or disorder, the method comprising administering to a subject in need thereof the loaded LNP of any one of the preceding embodiments.

Embodiment 88. A method of treating or preventing a disease or disorder, the method comprising administering to a subject in need thereof the loaded-LNP solution of any one of the preceding embodiments.

Embodiment 89. A method of treating or preventing a disease or disorder, the method comprising administering to a subject in need thereof the LNP formulation of any one of the preceding embodiments.

Embodiment 90. The method of any one of the preceding embodiments, wherein the administering is performed parenterally.

Embodiment 91. The method of any one of the preceding embodiments, wherein the administering is performed intramuscularly, intradermally, subcutaneously, and/or intravenously.

Embodiment 92. The loaded LNP of any one of the preceding embodiments for use in treating or preventing a disease or disorder in a subject.

Embodiment 93. The loaded-LNP solution any one of the preceding embodiments for use in treating or preventing a disease or disorder in a subject.

Embodiment 94. The LNP formulation of any one of the preceding embodiments for use in treating or preventing a disease or disorder in a subject.

Embodiment 95. Use of the loaded LNP of any one of the preceding embodiments in the manufacture of a medicament for treating or preventing a disease or disorder.

Embodiment 96. Use of the loaded-LNP solution of any one of the preceding embodiments in the manufacture of a medicament for treating or preventing a disease or disorder.

Embodiment 97. Use of the LNP formulation of any one of the preceding embodiments in the manufacture of a medicament for treating or preventing a disease or disorder.

Embodiment 98. A pharmaceutical kit, comprising the empty LNP, empty-LNP solution, loaded LNP, loaded-LNP solution, or LNP formulation of any one of embodiments 77-83.

Embodiment 99. An empty LNP comprising from about 0.1 mol% to about 1.25 mol% of a PEG lipid.

Embodiment 100. An empty LNP comprising from about 0.1 mol% to about 0.5 mol% of a PEG lipid.

Embodiment 101. The empty LNP of any one of the preceding embodiments, further comprising an ionizable lipid.

Embodiment 102. The empty LNP of any one of the preceding embodiments, further comprising a phospholipid and a structural lipid.

Embodiment 103. An empty LNP comprising about 30-60 mol% ionizable lipid; about 0-30 mol% phospholipid; about 15-50 mol% structural lipid; and about 0.1-10 mol% PEG lipid.

Embodiment 104. An empty-LNP solution comprising the empty LNP of any one of the preceding embodiments.

Embodiment 105. The empty-LNP solution of any one of the preceding embodiments, further comprising an acetate buffer.

Embodiment 106. The empty-LNP solution of any one of the preceding embodiments, further comprising a tonicity agent.

Embodiment 107. The empty-LNP solution of any one of the preceding embodiments, wherein the tonicity agent is sucrose.

Embodiment 108. An empty-LNP solution comprising:
(i) an empty LNP comprising from about 0.1 mol% to about 1.25 mol% of a PEG lipid; and
(ii) an acetate buffer.

Embodiment 109. An empty-LNP solution comprising:
(i) an empty LNP comprising from about 0.1 mol% to about 1.25 mol% of a PEG lipid;
(ii) an acetate buffer; and
(iii) sucrose.

Embodiment 110. An empty-LNP solution comprising:
(i) an empty LNP comprising from about 0.1 mol% to about 0.5 mol% of a PEG lipid; and
(ii) an acetate buffer.

Embodiment 111. An empty-LNP solution comprising:
(i) an empty LNP comprising from about 0.1 mol% to about 0.5 mol% of a PEG lipid;
(ii) an acetate buffer; and
(iii) sucrose

Embodiment 112. The empty-LNP solution of any one of the preceding embodiments, having a pH value of from about 4.5 to about 6.25, from about 4.6 to about 6.0, from about 4.8 to about 5.8, from about 5.0 to about 5.75, or from about 5.0 to about 5.5.

Embodiment 113. The empty-LNP solution of any one of the preceding embodiments, comprising about 5 mM acetate buffer, wherein the acetate buffer has a pH of about 5.0.

Embodiment 114. The empty-LNP solution of any one of the preceding embodiments, comprising about 0.2 g/mL sucrose.

Embodiment 115. The empty-LNP solution of any one of the preceding embodiments, wherein the empty LNP comprises from about 30 mol% to about 60 mol% of the ionizable lipid, from about 0 mol% to about 30 mol% of the phospholipid, from about 15 mol% to about 50 mol%% of the structural lipid, and from about 0.1 mol% to about 0.5 mol% of the PEG lipid.

Embodiment 116. The empty-LNP solution of any one of the preceding embodiments, wherein the empty LNP comprises from about 40 mol% to about 60 mol% of the ionizable lipid, from about 5 mol% to about 20 mol% of the phospholipid, from about 30 mol% to about 50 mol% of the structural lipid, and from about 0.1 mol% to about 1.25 mol% of the PEG lipid.

Embodiment 117. The empty-LNP solution of any one of the preceding embodiments, wherein the PEG lipid is present at a concentration of about 0.2 mol% to about 0.7 mol%.

Embodiment 118. The empty-LNP solution of any one of the preceding embodiments, wherein the PEG lipid is present at a concentration of about 0.5 mol%.

Embodiment 119. The empty-LNP solution of any one of the preceding embodiments, wherein the empty-LNP solution comprises an acetate buffer having a concentration of from about 2 mM to about 40 mM.

Embodiment 120. The empty-LNP solution of any one of the preceding embodiments, wherein the empty-LNP solution comprises an acetate buffer having a concentration of from about 2 mM to about 30 mM.

Embodiment 121. The empty-LNP solution of any one of the preceding embodiments, wherein the empty-LNP solution comprises an acetate buffer having a concentration of from about 2 mM to about 20 mM.

Embodiment 122. The empty-LNP solution of any one of the preceding embodiments, wherein the empty-LNP solution comprises an acetate buffer having a concentration of from about 2 mM to about 10 mM.

Embodiment 123. The empty-LNP solution of any one of the preceding embodiments, wherein the empty-LNP solution comprises an acetate buffer having a concentration of about 5 mM.

Embodiment 124. The empty-LNP solution of any one of the preceding embodiments, wherein the buffer has a pH of at least 1 unit less than the pKa of the ionizable lipid.

mbodiment 125. The empty-LNP solution of any one of the preceding embodiments, wherein the buffer has a pH of less than 5.5.

Embodiment 126. The empty-LNP solution of any one of the preceding embodiments, wherein the buffer has a pH of about 5.0.

Embodiment 127. The empty-LNP solution of any one of the preceding embodiments, wherein the empty-LNP solution has a pH of at least 1 unit less than the pKa of the ionizable lipid.

Embodiment 128. The empty-LNP solution of any one of the preceding embodiments, wherein the empty-LNP solution has a pH of less than 5.5.

Embodiment 129. The empty-LNP solution of any one of the preceding embodiments, wherein the empty-LNP solution has a pH of about 5.0.

Embodiment 130. The empty-LNP solution of any one of the preceding embodiments, wherein the LNPs comprise about 45 mol% to about 50 mol% ionizable lipid.

Embodiment 131. The empty-LNP solution of any one of the preceding embodiments, wherein the ionizable lipid is

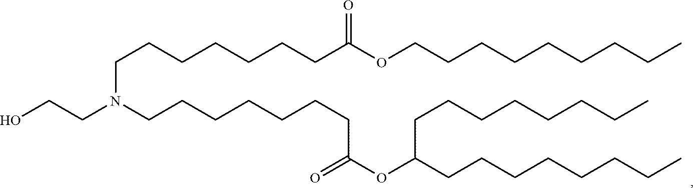

or a salt thereof.

Embodiment 132. The empty-LNP solution of any one of the preceding embodiments, wherein the ionizable lipid is

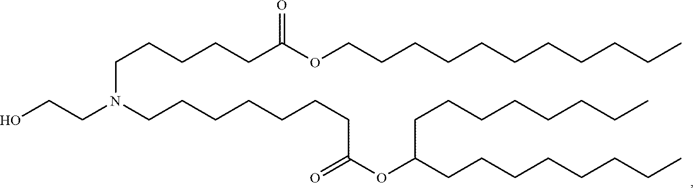

or a salt thereof.

Embodiment 133. The empty-LNP solution of any one of the preceding embodiments, wherein the PEG lipid is PEG₂ₖ-DMG.

Embodiment 134. The empty-LNP solution of any one of the preceding embodiments, wherein the structural lipid is cholesterol.

Embodiment 135. The empty-LNP solution of any one of the preceding embodiments, wherein the phospholipid is 1,2-distearoyl-sn-glycero-3-phosphocholine (DSPC). Embodiment 136. An empty-LNP or empty-LNP solution of any one of the preceding embodiments, wherein the empty LNP is substantially free of a therapeutic or prophylactic agent.

Embodiment 137. An empty-LNP or empty-LNP solution of any one of the preceding embodiments, wherein the empty LNP does not comprise a therapeutic or prophylactic agent.

Embodiment 138. An empty-LNP or empty-LNP solution of any one of the preceding embodiments, wherein the empty LNP comprises less than 5 wt.%, less than 4 wt.%, less than 3 wt.%, less than 2 wt.%, or less than 1 wt.% of therapeutic or prophylactic agent.

Embodiment 139. An empty-LNP or empty-LNP solution of any one of the preceding embodiments, wherein the empty LNP comprises less than 0.9 wt.%, less than 0.8 wt.%, less than 0.7 wt.%, less than 0.6 wt.%, less than 0.5 wt.%, less than 0.4 wt.%, less than 0.3 wt.%, less than 0.2 wt.%, or less than 0.1% of therapeutic or prophylactic agent.

Embodiment 140. An empty-LNP or empty-LNP solution of any one of the preceding embodiments, wherein the empty LNP is substantially free of a nucleic acid.

Embodiment 141. An empty-LNP or empty-LNP solution of any one of the preceding embodiments, wherein the empty LNP does not comprise a nucleic acid.

Embodiment 142. An empty-LNP or empty-LNP solution of any one of the preceding embodiments, wherein the empty LNP comprises less than 5 wt.%, less than 4 wt.%, less than 3 wt.%, less than 2 wt.%, or less than 1 wt.% of nucleic acid.

Embodiment 143. An empty-LNP or empty-LNP solution of any one of the preceding embodiments, wherein the empty LNP comprises less than 0.9 wt.%, less than 0.8 wt.%, less than 0.7 wt.%, less than 0.6 wt.%, less than 0.5 wt.%, less than 0.4 wt.%, less than 0.3 wt.%, less than 0.2 wt.%, or less than 0.1% of nucleic acid.

Embodiment 144. A preparation comprising an empty-LNP of any one of the preceding embodiments.

Embodiment 145. A preparation comprising an empty-LNP of any one of the preceding embodiments and an acetate buffer having a concentration of from about 2 mM to about 40 mM.

Embodiment 146. A preparation comprising lipid nanoparticles (LNPs).

Embodiment 147. A preparation comprising lipid nanoparticles (LNPs), wherein the LNPs are substantially free of a therapeutic or prophylactic agent, and wherein the preparation comprises an acetate buffer having a concentration of from about 2 mM to about 40 mM.

Embodiment 148. A preparation comprising lipid nanoparticles (LNPs), wherein the LNPs no not comprise a therapeutic or prophylactic agent, and wherein the preparation comprises an acetate buffer having a concentration of from about 2 mM to about 40 mM.

Embodiment 149. A preparation comprising lipid nanoparticles (LNPs), wherein the LNPs comprise less than 5 wt.%, less than 4 wt.%, less than 3 wt.%, less than 2 wt.%, or less than 1 wt.% of a therapeutic or prophylactic agent, and wherein the preparation comprises an acetate buffer having a concentration of from about 2 mM to about 40 mM.

Embodiment 150. A preparation comprising lipid nanoparticles (LNPs), wherein the LNPs comprise less than 0.9 wt.%, less than 0.8 wt.%, less than 0.7 wt.%, less than 0.6 wt.%, less than 0.5 wt.%, less than 0.4 wt.%, less than 0.3 wt.%, less than 0.2 wt.%, or less than 0.1% of a therapeutic or prophylactic agent, and wherein the preparation comprises an acetate buffer having a concentration of from about 2 mM to about 40 mM.

Embodiment 151. A preparation comprising lipid nanoparticles (LNPs), wherein the LNPs are substantially free of a nucleic acid, and wherein the preparation comprises an acetate buffer having a concentration of from about 2 mM to about 40 mM.

Embodiment 152. A preparation comprising lipid nanoparticles (LNPs), wherein the LNPs no not comprise a nucleic acid, and wherein the preparation comprises an acetate buffer having a concentration of from about 2 mM to about 40 mM.

Embodiment 153. A preparation comprising lipid nanoparticles (LNPs), wherein the LNPs comprise less than wt.%, less than 4 wt.%, less than 3 wt.%, less than 2 wt.%, or less than 1 wt.% of a nucleic acid, and wherein the preparation comprises an acetate buffer having a concentration of from about 2 mM to about 40 mM.

Embodiment 154. A preparation comprising lipid nanoparticles (LNPs), wherein the LNPs comprise less than 0.9 wt.%, less than 0.8 wt.%, less than 0.7 wt.%, less than 0.6 wt.%, less than 0.5 wt.%, less than 0.4 wt.%, less than 0.3 wt.%, less than 0.2 wt.%, or less than 0.1% of a nucleic acid, and wherein the preparation comprises an acetate buffer having a concentration of from about 2 mM to about 40 mM.

Embodiment 155. A preparation comprising lipid nanoparticles (LNPs), wherein:
(a) the LNPs comprise
   from about 40 mol% to about 50 mol% ionizable lipid,
   from about 30 mol% to about 50 mol% structural lipid,
   from about 5 mol% to about 20 mol% phospholipid, and
   from about 0.1 mol% to about 1.25 mol% of a PEG lipid;
(b) the LNPs are substantially free of a therapeutic or prophylactic agent; and
(c) the preparation comprises an acetate buffer having a concentration of from about 2 mM to about 40 mM.

Embodiment 156. A preparation comprising lipid nanoparticles (LNPs), wherein:
(a) the LNPs comprise
   from about 40 mol% to about 50 mol% ionizable lipid,
   from about 30 mol% to about 50 mol% structural lipid,
   from about 5 mol% to about 20 mol% phospholipid, and
   from about 0.1 mol% to about 1.25 mol% of a PEG lipid;
(b) the LNPs do not comprise a therapeutic or prophylactic agent; and
(c) the preparation comprises an acetate buffer having a concentration of from about 2 mM to about 40 mM.

Embodiment 157. A preparation comprising lipid nanoparticles (LNPs), wherein:
(a) the LNPs comprise
   from about 40 mol% to about 50 mol% ionizable lipid,
   from about 30 mol% to about 50 mol% structural lipid,
   from about 5 mol% to about 20 mol% phospholipid, and
   from about 0.1 mol% to about 1.25 mol% of a PEG lipid;
(b) the LNPs comprise less than 5 wt.%, less than 4 wt.%, less than 3 wt.%, less than 2 wt.%, or less than 1 wt.% of a therapeutic or prophylactic agent; and
(c) the preparation comprises an acetate buffer having a concentration of from about 2 mM to about 40 mM.

Embodiment 158. A preparation comprising lipid nanoparticles (LNPs), wherein:
(a) the LNPs comprise
   from about 40 mol% to about 50 mol% ionizable lipid,
   from about 30 mol% to about 50 mol% structural lipid,
   from about 5 mol% to about 20 mol% phospholipid, and
   from about 0.1 mol% to about 1.25 mol% of a PEG lipid;
(b) the LNPs comprise less than 0.9 wt.%, less than 0.8 wt.%, less than 0.7 wt.%, less than 0.6 wt.%, less than 0.5 wt.%, less than 0.4 wt.%, less than 0.3 wt.%, less than 0.2 wt.%, or less than 0.1% of a therapeutic or prophylactic agent; and
(c) the preparation comprises an acetate buffer having a concentration of from about 2 mM to about 40 mM.

Embodiment 159. A preparation comprising lipid nanoparticles (LNPs), wherein:
(a) the LNPs comprise
   from about 40 mol% to about 50 mol% ionizable lipid,
   from about 30 mol% to about 50 mol% structural lipid,
   from about 5 mol% to about 20 mol% phospholipid, and
   from about 0.1 mol% to about 1.25 mol% of a PEG lipid;
(b) the LNPs are substantially free of a nucleic acid; and
(c) the preparation comprises an acetate buffer having a concentration of from about 2 mM to about 40 mM.

Embodiment 160. A preparation comprising lipid nanoparticles (LNPs), wherein:
(a) the LNPs comprise
   from about 40 mol% to about 50 mol% ionizable lipid,
   from about 30 mol% to about 50 mol% structural lipid,
   from about 5 mol% to about 20 mol% phospholipid, and
   from about 0.1 mol% to about 1.25 mol% of a PEG lipid;
(b) the LNPs do not comprise a nucleic acid; and
(c) the preparation comprises an acetate buffer having a concentration of from about 2 mM to about 40 mM.

Embodiment 161. A preparation comprising lipid nanoparticles (LNPs), wherein:
(a) the LNPs comprise
   from about 40 mol% to about 50 mol% ionizable lipid,
   from about 30 mol% to about 50 mol% structural lipid,
   from about 5 mol% to about 20 mol% phospholipid, and
   from about 0.1 mol% to about 1.25 mol% of a PEG lipid;
(b) the LNPs comprise less than 5 wt.%, less than 4 wt.%, less than 3 wt.%, less than 2 wt.%, or less than 1 wt.% of a nucleic acid; and
(c) the preparation comprises an acetate buffer having a concentration of from about 2 mM to about 40 mM.

Embodiment 162. A preparation comprising lipid nanoparticles (LNPs), wherein:
(a) the LNPs comprise
   from about 40 mol% to about 50 mol% ionizable lipid,
   from about 30 mol% to about 50 mol% structural lipid,
   from about 5 mol% to about 20 mol% phospholipid, and
   from about 0.1 mol% to about 1.25 mol% of a PEG lipid;
(b) the LNPs comprise less than 0.9 wt.%, less than 0.8 wt.%, less than 0.7 wt.%, less than 0.6 wt.%, less than 0.5 wt.%, less than 0.4 wt.%, less than 0.3 wt.%, less than 0.2 wt.%, or less than 0.1% of a nucleic acid; and
(c) the preparation comprises an acetate buffer having a concentration of from about 2 mM to about 40 mM.

Embodiment 163. The preparation of any one of the preceding embodiments, wherein the PEG lipid is present at a concentration of about 0.2 mol% to about 0.7 mol%.

Embodiment 164. The preparation of any one of the preceding embodiments, wherein the PEG lipid is present at a concentration of about 0.5 mol%.

Embodiment 165. The preparation of any one of the preceding embodiments, wherein the preparation comprises an acetate buffer having a concentration of from about 2 mM to about 30 mM.

Embodiment 166. The preparation of any one of the preceding embodiments, wherein the preparation comprises an acetate buffer having a concentration of from about 2 mM to about 20 mM.

Embodiment 167. The preparation of any one of the preceding embodiments, wherein the preparation comprises an acetate buffer having a concentration of from about 2 mM to about 10 mM.

Embodiment 168. The preparation of any one of the preceding embodiments, wherein the preparation comprises an acetate buffer having a concentration of about 5 mM.

Embodiment 169. The preparation of any one of the preceding embodiments, wherein the buffer has a pH of at least 1 unit less than the pKa of the ionizable lipid.

Embodiment 170. The preparation of any one of the preceding embodiments, wherein the buffer has a pH of less than 5.5.

Embodiment 171. The preparation of any one of the preceding embodiments, wherein the buffer has a pH of about 5.0.

Embodiment 172. The preparation of any one of the preceding embodiments, wherein the LNPs comprise about 45 mol% to about 50 mol% ionizable lipid.

Embodiment 173. The preparation of any one of the preceding embodiments, wherein the ionizable lipid is

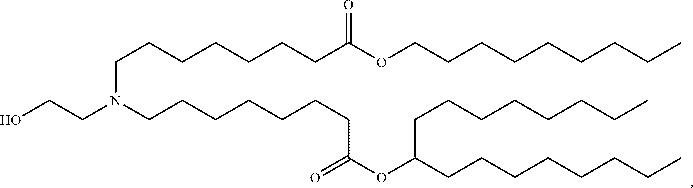

or a salt thereof.

Embodiment 174. The preparation of any one of the preceding embodiments, wherein the ionizable lipid is

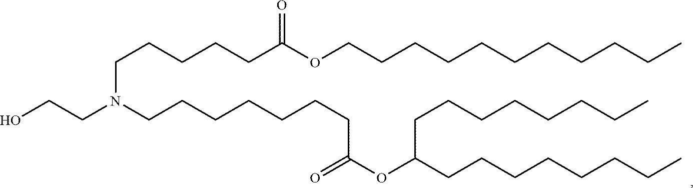

or a salt thereof.

Embodiment 175. The preparation of any one of the preceding embodiments, wherein the PEG lipid is PEG₂ₖ-DMG.

Embodiment 176. The preparation of any one of the preceding embodiments, wherein the structural lipid is cholesterol.

Embodiment 177. The preparation of any one of the preceding embodiments, wherein the phospholipid is 1,2-distearoyl-sn-glycero-3-phosphocholine (DSPC).

Embodiment 178. A population of empty LNPs of any one of the preceding embodiments.

Embodiment 179. A method of preparing an empty lipid nanoparticle (empty LNP), comprising:

i) a mixing step, comprising mixing an ionizable lipid with a first buffering agent, thereby forming the empty LNP, wherein the empty LNP comprises from about 0.1 mol% to about 0.5 mol% of a PEG lipid.

Embodiment 180. The method of any one of the preceding embodiments, wherein the mixing step comprises mixing a lipid solution comprising the ionizable lipid with an aqueous buffer solution comprising the first buffering agent, thereby forming an empty-lipid nanoparticle solution (empty-LNP solution) comprising the empty LNP.

Embodiment 181. An empty LNP comprising from about 0.1 mol% to about 0.5 mol% of a PEG lipid.

Embodiment 182. An empty-LNP solution comprising an empty LNP, wherein the empty LNP comprises from about 0.1 mol% to about 0.5 mol% of a PEG lipid.

Embodiment 183. A method of preparing a loaded lipid nanoparticle (loaded LNP) associated with a nucleic acid, comprising:

ii) a loading step, comprising mixing a nucleic acid with an empty LNP, thereby forming the loaded LNP.

Embodiment 184. The method of any one of the preceding embodiments, wherein the loading step comprises mixing the nucleic acid solution comprising the nucleic acid with the empty-LNP solution, thereby forming a loaded lipid nanoparticle solution (loaded-LNP solution) comprising the loaded LNP.

Embodiment 185. The method of any one of the preceding embodiments, wherein, upon formation, the empty LNP or the empty-LNP solution is subjected to the loading step without holding or storage.

Embodiment 186. The method of any one of the preceding embodiments, wherein, the empty LNP or the empty-LNP solution is subjected to the loading step after holding for a period of time.

Embodiment 187. The method of any one of the preceding embodiments, wherein, the empty LNP or the empty-LNP solution is subjected to the loading step after storage for a period of time.

Embodiment 188. The method of any one of the preceding embodiments, wherein, upon formation, the empty LNP or the empty-LNP solution is subjected to the loading step without storage or holding for a period of time.

Embodiment 189. The method of any one of the preceding embodiments, further comprising:

iii) processing the empty-LNP solution or loaded-LNP solution, thereby forming a lipid nanoparticle formulation (LNP formulation).

Embodiment 190. An empty LNP being prepared by the method of any one of the preceding embodiments.

Embodiment 191. An empty-LNP solution being prepared by the method of any one of the preceding embodiments.

Embodiment 192. A loaded LNP being prepared by the method of any one of the preceding embodiments.

Embodiment 193. A loaded-LNP solution being prepared by the method of any one of the preceding embodiments.

Embodiment 194. A LNP formulation being prepared by the method of any one of the preceding embodiments.

Embodiment 195. The method, empty LNP, empty-LNP solution, loaded LNP, loaded-LNP solution, or LNP formulation of any one of the preceding embodiments, wherein the empty LNP or the loaded-LNP further comprises about 0.1-0.5 mol% PEG lipid, a phospholipid, a structural lipid, or any combination thereof.

Embodiment 196. The method, empty LNP, empty-LNP solution, loaded LNP, loaded-LNP solution, or LNP formulation of any one of the preceding embodiments, wherein the step of processing the empty-LNP solution or loaded-LNP solution comprises a first adding step, comprising adding a polyethylene glycol lipid (PEG lipid) to the empty LNP or the loaded LNP.

Embodiment 197. The method, empty LNP, empty-LNP solution, loaded LNP, loaded-LNP solution, or LNP formulation of any one of the preceding embodiments, wherein the first adding step comprises adding a polyethylene glycol solution (PEG solution) comprising the PEG lipid to the empty-LNP solution or loaded-LNP solution.

Embodiment 198. The method, empty LNP, empty-LNP solution, loaded LNP, loaded-LNP solution, or LNP formulation of any one of the preceding embodiments, wherein the step of processing the empty-LNP solution or loaded-LNP solution comprises a second adding step, comprising adding a polyethylene glycol lipid (PEG lipid) to the empty LNP or the loaded LNP.

Embodiment 199. The method, empty LNP, empty-LNP solution, loaded LNP, loaded-LNP solution, or LNP formulation of any one of the preceding embodiments, wherein the second adding step comprises adding a polyethylene glycol solution (PEG solution) comprising the PEG lipid to the empty-LNP solution or loaded-LNP solution.

Embodiment 200. The method, empty LNP, empty-LNP solution, loaded LNP, loaded-LNP solution, or LNP formulation of any one of the preceding embodiments, wherein the first adding step comprises adding about 0.1 mol% to about 3.0 mol% PEG, about 0.2 mol% to about 2.5 mol% PEG, about 0.5 mol% to about 2.0 mol% PEG, about 0.75 mol% to about 1.5 mol% PEG, about 1.0 mol% to about 1.25 mol% PEG to the empty LNP or the loaded LNP.

Embodiment 201. The method, empty LNP, empty-LNP solution, loaded LNP, loaded-LNP solution, or LNP formulation of any one of the preceding embodiments, wherein the first adding step comprises adding about 0.1 mol% to about 3.0 mol% PEG, about 0.2 mol% to about 2.5 mol% PEG, about 0.5 mol% to about 2.0 mol% PEG, about 0.75 mol% to about 1.5 mol% PEG, about 1.0 mol% to about 1.25 mol% PEG to the empty-LNP or the loaded-LNP.

Embodiment 202. The method, empty LNP, empty-LNP solution, loaded LNP, loaded-LNP solution, or LNP formulation of any one of the preceding embodiments, wherein the first adding step comprises adding about 1.75 mol% PEG lipid to the empty LNP or the loaded LNP.

Embodiment 203. The method, empty LNP, empty-LNP solution, loaded LNP, loaded-LNP solution, or LNP formulation of any one of the preceding embodiments, wherein the second adding step comprises adding about 0.1 mol% to about 3.0 mol% PEG, about 0.2 mol% to about 2.5 mol% PEG, about 0.5 mol% to about 2.0 mol% PEG, about 0.75 mol% to about 1.5 mol% PEG, about 1.0 mo%l to about 1.25 mol% PEG to the empty LNP or the loaded LNP.

Embodiment 204. The method, empty LNP, empty-LNP solution, loaded LNP, loaded-LNP solution, or LNP formulation of any one of the preceding embodiments, wherein the second adding step comprises adding about 0.1 mol% to about 3.0 mol% PEG, about 0.2 mol% to about 2.5 mol% PEG, about 0.5 mol% to about 2.0 mol% PEG, about 0.75 mol% to about 1.5 mol% PEG, about 1.0 mol% to about 1.25 mol% PEG to the empty LNP or the loaded LNP.

Embodiment 205. The method, empty LNP, empty-LNP solution, loaded LNP, loaded-LNP solution, or LNP formulation of any one of the preceding embodiments, wherein the second adding step comprises adding about 1.0 mol% PEG lipid to the empty LNP or the loaded LNP.

Embodiment 206. The method, empty LNP, empty-LNP solution, loaded LNP, loaded-LNP solution, or LNP formulation of any one of the preceding embodiments, wherein the empty LNP or the loaded LNP comprises about 3.0 mol% PEG lipid or less, about 2.75 mol% PEG lipid or less, about 2.5 mol% PEG lipid or less, about 2.25 mol% PEG lipid or less, about 2.0 mol% PEG lipid or less, about 1.75 mol% PEG lipid or less, about 1.5 mol% PEG lipid or less, about 1.25 mol% PEG lipid or less, about 1.0 mol% PEG lipid or less, about 0.9 mol% PEG lipid or less, about 0.8 mol% PEG lipid or less, about 0.7 mol% PEG lipid or less, about 0.6 mol% PEG lipid or less,about 0.5 mol% PEG lipid or less, about 0.4 mol% PEG lipid or less, about 0.3 mol% PEG lipid or less, about 0.2 mol% PEG lipid or less, or about 0.1 mol% PEG lipid or less.

Embodiment 207. The method, empty LNP, empty-LNP solution, loaded LNP, loaded-LNP solution, or LNP formulation of any one of the preceding embodiments, wherein the empty LNP or the loaded LNP comprises about 0 mol% to about 3.0 mol% PEG lipid, 0.1 mol% to about 2.5 mol% PEG lipid, about 0.2 mol% to about 2.25 mol% PEG lipid, about 0.25 mol% to about 2.0 mol% PEG lipid, about 0.5 mol% to about 1.75 mol% PEG lipid, about 0.75 mol% to about 1.5 mol% PEG lipid, or about 1.0 mol% to about 1.25 mol% PEG lipid.

Embodiment 208. The method, empty LNP, empty-LNP solution, loaded LNP, loaded-LNP solution, or LNP formulation of any one of the preceding embodiments, wherein the empty LNP or the loaded LNP comprises about 0 mol% to about 0.5 mol% PEG lipid.

Embodiment 209. The method, empty LNP, empty-LNP solution, loaded LNP, loaded-LNP solution, or LNP formulation of any one of the preceding embodiments, wherein the step of processing the empty-LNP solution or loaded-LNP solution further comprises at least one step selected from filtering, pH adjusting, buffer exchanging, diluting, dialyzing, concentrating, freezing, lyophilizing, storing, and packing.

Embodiment 210. The method, empty LNP, empty-LNP solution, loaded LNP, loaded-LNP solution, or LNP formulation of any one of the preceding embodiments, wherein the step of processing the empty-LNP solution or loaded-LNP solution further comprises pH adjusting.

Embodiment 211. The method, empty LNP, empty-LNP solution, loaded LNP, loaded-LNP solution, or LNP formulation of any one of the preceding embodiments, wherein the pH adjusting comprises adding a second buffering agent.

Embodiment 212. The method, empty LNP, empty-LNP solution, loaded LNP, loaded-LNP solution, or LNP formulation of any one of the preceding embodiments, the second buffering agent comprises a second aqueous buffer.

Embodiment 213. The method, empty LNP, empty-LNP solution, loaded LNP, loaded-LNP solution, or LNP formulation of any one of the preceding embodiments, the second aqueous buffer is selected from the group consisting of an acetate buffer, a citrate buffer, a phosphate buffer, and a tris buffer.

Embodiment 214. The method, empty LNP, empty-LNP solution, loaded LNP, loaded-LNP solution, or LNP formulation of any one of the preceding embodiments, wherein the second aqueous buffer is a tris buffer.

Embodiment 215. The method, empty LNP, empty-LNP solution, loaded LNP, loaded-LNP solution, or LNP formulation of any one of the preceding embodiments, wherein the second aqueous buffer has a pH in a range of about 6.5 to about 8.5, about 7.0 to about 8.0, about 7.2 to about 7.8, or about 7.4 to about 7.6.

Embodiment 216. The method, empty LNP, empty-LNP solution, loaded LNP, loaded-LNP solution, or LNP formulation of any one of the preceding embodiments, wherein the second aqueous buffer has a pH of about 7.5.

Embodiment 217. The method, empty LNP, empty-LNP solution, loaded LNP, loaded-LNP solution, or LNP formulation of any one of the preceding embodiments, wherein the first adding step is performed prior to the pH adjusting.

Embodiment 218. The method, empty LNP, empty-LNP solution, loaded LNP, loaded-LNP solution, or LNP formulation of any one of the preceding embodiments, wherein the first adding step is performed after the pH adjusting.

Embodiment 219. The method, empty LNP, empty-LNP solution, loaded LNP, loaded-LNP solution, or LNP formulation of any one of the preceding embodiments, wherein the second adding step is performed prior to the pH adjusting.

Embodiment 220. The method, empty LNP, empty-LNP solution, loaded LNP, loaded-LNP solution, or LNP formulation of any one of the preceding embodiments, wherein the second adding step is performed after the pH adjusting.

Embodiment 221. The method, empty LNP, empty-LNP solution, loaded LNP, loaded-LNP solution, or LNP formulation of any one of the preceding embodiments, wherein the step of processing the empty-LNP solution or loaded-LNP solution further comprises filtering.

Embodiment 222. The method, empty LNP, empty-LNP solution, loaded LNP, loaded-LNP solution, or LNP formulation of any one of the preceding embodiments, wherein the filtering is a tangential flow filtration.

Embodiment 223. The method, empty LNP, empty-LNP solution, loaded LNP, loaded-LNP solution, or LNP formulation of any one of the preceding embodiments, wherein the step of processing the empty-LNP solution or loaded-LNP solution further comprises buffer exchanging.

Embodiment 224. The method, empty LNP, empty-LNP solution, loaded LNP, loaded-LNP solution, or LNP formulation of any one of the preceding embodiments, wherein the buffer exchanging comprises addition of an aqueous buffer solution comprising a third buffering agent.

Embodiment 225. The method, empty LNP, empty-LNP solution, loaded LNP, loaded-LNP solution, or LNP formulation of any one of the preceding embodiments, wherein the third buffering agent comprises a third aqueous buffer.

Embodiment 226. The method, empty LNP, empty-LNP solution, loaded LNP, loaded-LNP solution, or LNP formulation of any one of the preceding embodiments, wherein the third aqueous buffer is selected from the group consisting of an acetate buffer, a citrate buffer, a phosphate buffer, and a tris buffer.

Embodiment 227. The method, empty LNP, empty-LNP solution, loaded LNP, loaded-LNP solution, or LNP formulation of any one of the preceding embodiments, wherein the third aqueous buffer has a pH in a range of about 6.5 to about 8.5, about 7.0 to about 8.0, about 7.2 to about 7.8, or about 7.4 to about 7.6.

Embodiment 228. The method, empty LNP, empty-LNP solution, loaded LNP, loaded-LNP solution, or LNP formulation of any one of the preceding embodiments, wherein the third aqueous buffer has a pH of about 7.5.

Embodiment 229. The method, empty LNP, empty-LNP solution, loaded LNP, loaded-LNP solution, or LNP formulation of any one of the preceding embodiments, wherein the first adding step is performed prior to the buffer exchanging.

Embodiment 230. The method, empty LNP, empty-LNP solution, loaded LNP, loaded-LNP solution, or LNP formulation of any one of the preceding embodiments, wherein the first adding step is performed after the buffer exchanging.

Embodiment 231. The method, empty LNP, empty-LNP solution, loaded LNP, loaded-LNP solution, or LNP formulation of any one of the preceding embodiments, wherein the second adding is performed prior to the buffer exchanging.

Embodiment 232. The method, empty LNP, empty-LNP solution, loaded LNP, loaded-LNP solution, or LNP formulation of any one of the preceding embodiments, wherein the second adding step is performed after the buffer exchanging.

Embodiment 233. The method, empty LNP, empty-LNP solution, loaded LNP, loaded-LNP solution, or LNP formulation of any one of the preceding embodiments, wherein the step of processing the empty-LNP solution or loaded-LNP solution further comprises diluting.

Embodiment 234. The method, empty LNP, empty-LNP solution, loaded LNP, loaded-LNP solution, or LNP formulation of any one of the preceding embodiments, wherein the step of processing the empty-LNP solution or loaded-LNP solution further comprises dialyzing.

Embodiment 235. The method, empty LNP, empty-LNP solution, loaded LNP, loaded-LNP solution, or LNP formulation of any one of the preceding embodiments, wherein the step of processing the empty-LNP solution or loaded-LNP solution further comprises concentrating.

Embodiment 236. The method, empty LNP, empty-LNP solution, loaded LNP, loaded-LNP solution, or LNP formulation of any one of the preceding embodiments, wherein the step of processing the empty-LNP solution or loaded-LNP solution further comprises freezing.

Embodiment 237. The method, empty LNP, empty-LNP solution, loaded LNP, loaded-LNP solution, or LNP formulation of any one of the preceding embodiments, wherein the step of processing the empty-LNP solution or loaded-LNP solution further comprises lyophilizing.

Embodiment 238. The method, empty LNP, empty-LNP solution, loaded LNP, loaded-LNP solution, or LNP formulation of any one of the preceding embodiments, wherein the lyophilizing comprises freezing the loaded-LNP solution at a temperature from about -100° C. to about 0° C., about -80° C. to about -10° C., about -60° C. to about -20° C., about -50° C. to about -25° C., or about -40° C. to about -30° C.

Embodiment 239. The method, empty LNP, empty-LNP solution, loaded LNP, loaded-LNP solution, or LNP formulation of any one of the preceding embodiments, wherein the lyophilizing further comprises drying the frozen loaded-LNP solution to form a lyophilized empty LNP or lyophilized loaded LNP.

Embodiment 240. The method, empty LNP, empty-LNP solution, loaded LNP, loaded-LNP solution, or LNP formulation of any one of the preceding embodiments, wherein the drying is performed at a vacuum ranging from about 50 mTorr to about 150 mTorr.

Embodiment 241. The method, empty LNP, empty-LNP solution, loaded LNP, loaded-LNP solution, or LNP formulation of any one of the preceding embodiments, wherein the drying is performed at about -35° C. to about -15° C.

Embodiment 242. The method, empty LNP, empty-LNP solution, loaded LNP, loaded-LNP solution, or LNP formulation of any one of the preceding embodiments, wherein the drying is performed at about room temperature to about 25° C.

Embodiment 243. The method, empty LNP, empty-LNP solution, loaded LNP, loaded-LNP solution, or LNP formulation of any one of the preceding embodiments, wherein the step of processing the empty-LNP solution or loaded-LNP solution further comprises storing.

Embodiment 244. The method, empty LNP, empty-LNP solution, loaded LNP, loaded-LNP solution, or LNP formulation of any one of the preceding embodiments, wherein the storing comprises storing the empty LNP or the loaded LNP at a temperature of about -80° C., about -78° C., about -76° C., about -74° C., about -72° C., about -70° C., about -65° C., about -60° C., about -55° C., about -50° C., about -45° C., about -40° C., about -35° C., or about -30° C. for at least 1 day, at least 2 days, at least 1 week, at least 2 weeks, at least 4 weeks, at least 1 month, at least 2 months, at least 3 months, at least 6 months, at least 8 months, or at least 1 year.

Embodiment 245. The method, empty LNP, empty-LNP solution, loaded LNP, loaded-LNP solution, or LNP formulation of any one of the preceding embodiments, wherein the storing comprises storing the empty LNP or the loaded LNP at a temperature of about -40° C., about -35° C., about -30° C., about -25° C., about -20° C., about -15° C., about -10° C., about -5° C., about 0° C., about 5° C., about 10° C., about 15° C., about 20° C., or about 25° C. for at least 1 day, at least 2 days, at least 1 week, at least 2 weeks, at least 4 weeks, at least 1 month, at least 2 months, at least 3 months, at least 6 months, at least 8 months, or at least 1 year.

Embodiment 246. The method, empty LNP, empty-LNP solution, loaded LNP, loaded-LNP solution, or LNP formulation of any one of the preceding embodiments, wherein the storing comprises storing the empty LNP or the loaded LNP at a temperature of about -40° C. to about 0° C., from about -35° C. to about -5° C., from about -30° C. to about -10° C., from about -25° C. to about -15° C., from about -22° C. to about -18° C., or from about -21° C. to about -19° C. for at least 1 day, at least 2 days, at least 1 week, at least 2 weeks, at least 4 weeks, at least 1 month, at least 2 months, at least 3 months, at least 6 months, at least 8 months, or at least 1 year.

Embodiment 247. The method, empty LNP, empty-LNP solution, loaded LNP, loaded-LNP solution, or LNP formulation of any one of the preceding embodiments, wherein the storing comprises storing the empty LNP or the loaded LNP at a temperature of about -20° C. for at least 1 day, at least 2 days, at least 1 week, at least 2 weeks, at least 4 weeks, at least 1 month, at least 2 months, at least 3 months, at least 6 months, at least 8 months, or at least 1 year.

Embodiment 248. The method, empty LNP, empty-LNP solution, loaded LNP, loaded-LNP solution, or LNP formulation of any one of the preceding embodiments, wherein the step of processing the empty-LNP solution or loaded-LNP solution further comprises packing.

Embodiment 249. The method, empty LNP, empty-LNP solution, loaded LNP, loaded-LNP solution, or LNP formulation of any one of the preceding embodiments, wherein the mixing step is performed with a T-junction, confined impinging jets, microfluidic mixer, or vortex mixer.

Embodiment 250. The method, empty LNP, empty-LNP solution, loaded LNP, loaded-LNP solution, or LNP formulation of any one of the preceding embodiments, wherein the loading step is performed with a T-junction, confined impinging jets, microfluidic mixer, or vortex mixer.

Embodiment 251. The method, empty LNP, empty-LNP solution, loaded LNP, loaded-LNP solution, or LNP formulation of any one of the preceding embodiments, wherein the mixing step is performed at a temperature of less than about 30° C., less than about 28° C., less than about 26° C., less than about 24° C., less than about 22° C., less than about 20° C., or less than about ambient temperature.

Embodiment 252. The method, empty LNP, empty-LNP solution, loaded LNP, loaded-LNP solution, or LNP formulation of any one of the preceding embodiments, wherein the loading step is performed at a temperature of less than about 30° C., less than about 28° C., less than about 26° C., less than about 24° C., less than about 22° C., less than about 20° C., or less than about ambient temperature.

Embodiment 253. The method, empty LNP, empty-LNP solution, loaded LNP, loaded-LNP solution, or LNP formulation of any one of the preceding embodiments, wherein the first adding step is performed at a temperature of less than about 30° C., less than about 28° C., less than about 26° C., less than about 24° C., less than about 22° C., less than about 20° C., or less than about ambient temperature.

Embodiment 254. The method, empty LNP, empty-LNP solution, loaded LNP, loaded-LNP solution, or LNP formulation of any one of the preceding embodiments, wherein the second adding step is performed at a temperature of less than about 30° C., less than about 28° C., less than about 26° C., less than about 24° C., less than about 22° C., less than about 20° C., or less than about ambient temperature.

Embodiment 255. The method, empty LNP, empty-LNP solution, loaded LNP, loaded-LNP solution, or LNP formulation of any one of the preceding embodiments, wherein a residence time between the mixing step and the first adding step is in a range of about 1.0 milliseconds to about 60 minutes, about 2.0 milliseconds to about 30 minutes, about 3.0 milliseconds to about 15 minutes, about 4.0 milliseconds to about 10 minutes, about 5.0 milliseconds to about 5 minutes about 10.0 milliseconds to about 2 minutes, about 100.0 milliseconds to about 1.0 minute, about 1000 milliseconds to about 1.0 minute.

Embodiment 256. The method, empty LNP, empty-LNP solution, loaded LNP, loaded-LNP solution, or LNP formulation of any one of the preceding embodiments, wherein the aqueous buffer solution has a pH in a range of about 4.5 to about 6.5, about 4.6 to about 6.0, about 4.7 to about 5.75, about 4.8 to about 5.5, or about 4.9 to about 5.25.

Embodiment 257. The method, empty LNP, empty-LNP solution, loaded LNP, loaded-LNP solution, or LNP formulation of any one of the preceding embodiments, wherein the aqueous buffer solution has a pH of about 5.0.

Embodiment 258. The method, empty LNP, empty-LNP solution, loaded LNP, loaded-LNP solution, or LNP formulation of any one of the preceding embodiments, wherein the lipid solution has a pH in a range of about 7.0 to about 8.0, about 7.1 to about 7.8, about 7.2 to about 7.6, or about 7.3 to about 7.5.

Embodiment 259. The method, empty LNP, empty-LNP solution, loaded LNP, loaded-LNP solution, or LNP formulation of any one of the preceding embodiments, wherein the empty-LNP solution has a pH in a range of about 4.5 to about 6.25, about 4.6 to about 6.0, about 4.8 to about 5.8, about 5.0 to about 5.75, about 5.0 to about 5.5.

Embodiment 260. The method, empty LNP, empty-LNP solution, loaded LNP, loaded-LNP solution, or LNP formulation of any one of the preceding embodiments, wherein the nucleic acid solution has a pH in a range of about 4.5 to about 6.5, about 4.8 to about 6.25, about 4.8 to about 6.0, about 5.0 to about 5.8, or about 5.2 to about 5.5.

Embodiment 261. The method, empty LNP, empty-LNP solution, loaded LNP, loaded-LNP solution, or LNP formulation of any one of the preceding embodiments, wherein the pH of the nucleic acid solution, the empty-LNP solution, and the LNP formulation are in a range of about 5.0 to about 6.0, about 5.1 to about 5.75, or about 5.2 to about 5.5.

Embodiment 262. The method, empty LNP, empty-LNP solution, loaded LNP, loaded-LNP solution, or LNP formulation of any one of the preceding embodiments, wherein the loaded-LNP solution has a pH in a range of about 4.5 to about 6.0, about 4.6 to about 5.8, about 4.8 to about 5.6, about 5.0 to about 5.5, or about 5.1 to about 5.4.

Embodiment 263. The method, empty LNP, empty-LNP solution, loaded LNP, loaded-LNP solution, or LNP formulation of any one of the preceding embodiments, wherein the lipid solution further comprises a first organic solvent.

Embodiment 264. The method, empty LNP, empty-LNP solution, loaded LNP, loaded-LNP solution, or LNP formulation of any one of the preceding embodiments, wherein the empty-LNP solution or loaded-LNP solution further comprises a first organic solvent.

Embodiment 265. The method, empty LNP, empty-LNP solution, loaded LNP, loaded-LNP solution, or LNP formulation of any one of the preceding embodiments, wherein the first organic solvent is an alcohol.

Embodiment 266. The method, empty LNP, empty-LNP solution, loaded LNP, loaded-LNP solution, or LNP formulation of any one of the preceding embodiments, wherein the first organic solvent is ethanol.

Embodiment 267. The method, empty LNP, empty-LNP solution, loaded LNP, loaded-LNP solution, or LNP formulation of any one of the preceding embodiments, wherein the first buffering agent comprises a first aqueous buffer.

Embodiment 268. The method, empty LNP, empty-LNP solution, loaded LNP, loaded-LNP solution, or LNP formulation of any one of the preceding embodiments, wherein the first aqueous buffer is selected from the group consisting of an acetate buffer, a citrate buffer, a phosphate buffer, and a tris buffer.

Embodiment 269. The method, empty LNP, empty-LNP solution, loaded LNP, loaded-LNP solution, or LNP formulation of any one of the preceding embodiments, wherein the first aqueous buffer comprise greater than about 10 mM citrate, acetate, phosphate or tris, greater than about 15 mM citrate, acetate, phosphate or tris, greater than about 20 mM citrate, acetate, phosphate or tris, greater than about 25 mM citrate, acetate, phosphate or tris, or greater than about 30 mM citrate, acetate, phosphate or tris.

Embodiment 270. The method, empty LNP, empty-LNP solution, loaded LNP, loaded-LNP solution, or LNP formulation of any one of the preceding embodiments, wherein the first aqueous buffer comprises greater than about 1 mM citrate, acetate, phosphate or tris, greater than about 2 mM citrate, acetate, phosphate or tris, greater than about 5 mM citrate, acetate, phosphate or tris, greater than about 10 mM citrate, acetate, phosphate or tris, greater than about 15 mM citrate, acetate, phosphate or tris, greater than about 20 mM citrate, acetate, phosphate or tris, greater than about 25 mM citrate, acetate, phosphate or tris, or greater than about 30 mM citrate, acetate, phosphate or tris.

Embodiment 271. The method, empty LNP, empty-LNP solution, loaded LNP, loaded-LNP solution, or LNP formulation of any one of the preceding embodiments, wherein the first aqueous buffer comprises about 1 mM to about 30 mM citrate, acetate, phosphate or tris, about 2 mM to about 20 mM citrate, acetate, phosphate or tris, about 3 mM to about 10 mM citrate, acetate, phosphate or tris, about 4 mM to about 8 mM citrate, acetate, phosphate or tris, or about 5 mM to about 6 mM citrate, acetate, phosphate or tris.

Embodiment 272. The method, empty LNP, empty-LNP solution, loaded LNP, loaded-LNP solution, or LNP formulation of any one of the preceding embodiments, wherein thefirst aqueous buffer comprises about 5 mM citrate, acetate, phosphate or tris.

Embodiment 273. The method, empty LNP, empty-LNP solution, loaded LNP, loaded-LNP solution, or LNP formulation of any one of the preceding embodiments, wherein thefirst aqueous buffer comprises about 5 mM acetate, wherein the aqueous buffer solution has a pH of about 5.0.

Embodiment 274. The method, empty LNP, empty-LNP solution, loaded LNP, loaded-LNP solution, or LNP formulation of any one of the preceding embodiments, wherein the empty-LNP solution or loaded-LNP solution further comprises a tonicity agent.

Embodiment 275. The method, empty LNP, empty-LNP solution, loaded LNP, loaded-LNP solution, or LNP formulation of any one of the preceding embodiments, wherein the empty-LNP solution or loaded-LNP solution is stored before the loading step with a tonicity agent.

Embodiment 276. The method, empty LNP, empty-LNP solution, loaded LNP, loaded-LNP solution, or LNP formulation of any one of the preceding embodiments, wherein the tonicity agent is a sugar.

Embodiment 277. The method, empty LNP, empty-LNP solution, loaded LNP, loaded-LNP solution, or LNP formulation of any one of the preceding embodiments, wherein the sugar is sucrose.

Embodiment 278. The method, empty LNP, empty-LNP solution, loaded LNP, loaded-LNP solution, or LNP formulation of any one of the preceding embodiments, wherein the empty-LNP solution or loaded-LNP solution further comprises about 0.01 g/mL to about 1.0g/mL, about 0.05 g/mL to about 0.5 g/mL, about 0.1 g/mL to about 0.4 g/mL, about 0.15 g/mL to about 0.3 g/mL, or about 0.2 g/mL to about 0.25 g/mL tonicity agent.

Embodiment 279. The method, empty LNP, empty-LNP solution, loaded LNP, loaded-LNP solution, or LNP formulation of any one of the preceding embodiments, wherein the empty-LNP solution or loaded-LNP solution further comprises about 0.2 g/mL to about 0.25 g/mL tonicity agent.

Embodiment 280. The method, empty LNP, empty-LNP solution, loaded LNP, loaded-LNP solution, or LNP formulation of any one of the preceding embodiments, wherein the empty-LNP solution or loaded-LNP solution further comprises about 0.2 g/mL sucrose.

Embodiment 281. The method, empty LNP, empty-LNP solution, loaded LNP, loaded-LNP solution, or LNP formulation of any one of the preceding embodiments, wherein the nucleic acid solution comprises about 0.01 to about 1.0 mg/mL of the nucleic acid, about 0.05 to about 0.5 mg/mL of the nucleic acid, or about 0.1 to about 0.25 mg/mL of the nucleic acid.

Embodiment 282. The method, empty LNP, empty-LNP solution, loaded LNP, loaded-LNP solution, or LNP formulation of any one of the preceding embodiments, wherein the nucleic acid solution comprises about 0.001 to about 1.0 mg/mL of the nucleic acid, about 0.0025 to about 0.5 mg/mL of the nucleic acid, or about 0.005 to about 0.2 mg/mL of the nucleic acid.

Embodiment 283. The method, empty LNP, empty-LNP solution, loaded LNP, loaded-LNP solution, or LNP formulation of any one of the preceding embodiments, wherein the nucleic acid solution comprises about 0.005 to about 0.2 mg/mL of the nucleic acid.

Embodiment 284. The method, empty LNP, empty-LNP solution, loaded LNP, loaded-LNP solution, or LNP formulation of any one of the preceding embodiments, wherein the nucleic acid solution comprises a buffer selected from the group consisting of an acetate buffer, a citrate buffer, a phosphate buffer, and a tris buffer.

Embodiment 285. The method, empty LNP, empty-LNP solution, loaded LNP, loaded-LNP solution, or LNP formulation of any one of the preceding embodiments, wherein the nucleic acid solution comprises an acetate buffer.

Embodiment 286. The method, empty LNP, empty-LNP solution, loaded LNP, loaded-LNP solution, or LNP formulation of any one of the preceding embodiments, wherein the nucleic acid solution comprises about 1 mM to about 200 mM acetate buffer, about 2 mM to about 180 mM acetate buffer, about 3 mM to about 160 mM acetate buffer, about 4 mM to about 150 mM acetate buffer, about 4 mM to about 140 mM acetate buffer, about 5 mM to about 130 mM acetate buffer, about 6 mM to about 120 mM acetate buffer, about 7 mM to about 110 mM acetate buffer, about 8 mM to about 100 mM acetate buffer, about 9 mM to about 90 mM acetate buffer, about 10 mM to about 80 mM acetate buffer, about 15 mM to about 70 mM acetate buffer, about 20 mM to about 60 mM acetate buffer, about 25 mM to about 50 mM acetate buffer, or about 30 mM to about 40 mM acetate buffer.

Embodiment 287. The method, empty LNP, empty-LNP solution, loaded LNP, loaded-LNP solution, or LNP formulation of any one of the preceding embodiments, wherein the nucleic acid solution comprises about 8.8 mM acetate buffer.

Embodiment 288. The method, empty LNP, empty-LNP solution, loaded LNP, loaded-LNP solution, or LNP formulation of any one of the preceding embodiments, wherein the nucleic acid solution comprises about 130 mM acetate buffer.

Embodiment 289. The method, empty LNP, empty-LNP solution, loaded LNP, loaded-LNP solution, or LNP formulation of any one of the preceding embodiments, wherein the nucleic acid solution and the empty-LNP solution are mixed at a volumetric flow ratio of about 5:1 to about 7:1, about 4:1 to about 6:1, about 3:1 to about 5:1, or about 2:1 to about 4:1 during the loading step.

Embodiment 290. The method, empty LNP, empty-LNP solution, loaded LNP, loaded-LNP solution, or LNP formulation of any one of the preceding embodiments, wherein the nucleic acid solution and the empty-LNP solution are mixed at a volumetric flow ratio of about 3:1 during the loading step.

Embodiment 291. The method, empty LNP, empty-LNP solution, loaded LNP, loaded-LNP solution, or LNP formulation of any one of the preceding embodiments, wherein the empty-LNP solution or loaded-LNP solution comprises an acetate buffer.

Embodiment 292. The method, empty LNP, empty-LNP solution, loaded LNP, loaded-LNP solution, or LNP formulation of any one of the preceding embodiments, wherein the empty-LNP solution or loaded-LNP solution comprises about 5 mM acetate buffer, wherein the acetate buffer has a pH of about 5.0.

Embodiment 293. The method, empty LNP, empty-LNP solution, loaded LNP, loaded-LNP solution, or LNP formulation of any one of the preceding embodiments, wherein the lipid solution, the empty LNP, the empty-LNP solution, the loaded LNP, the loaded-LNP solution, and/or the LNP formulation further comprises an encapsulation agent.

Embodiment 294. The method, empty LNP, empty-LNP solution, loaded LNP, loaded-LNP solution, or LNP formulation of any one of the preceding embodiments, wherein the encapsulation agent is a compound of Formula (EA-I):

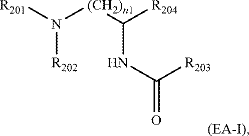

or a salt or isomer thereof, wherein
R₂₀₁ and R₂₀₂ are each independently selected from the group consisting of H, C₁-C₆ alkyl, C₂-C₆ alkenyl, and (C═NH)N(R₁₀₁)₂ wherein each R₁₀₁ is independently selected from the group consisting of H, C₁-C₆ alkyl, and C₂-C₆ alkenyl;
R₂₀₃ is selected from the group consisting of C₁-C₂₀ alkyl and C₂-C₂₀ alkenyl;
R₂₀₄ is selected from the group consisting of H, C₁-C₂₀ alkyl, C₂-C₂₀ alkenyl, C(O)(OC₁-C₂₀ alkyl), C(O)(OC₂-C₂₀ alkenyl), C(O)(NHC₁-C₂₀ alkyl), and C(O)(NHC₂-C₂₀ alkenyl); and
n1 is selected from 1, 2, 3, 4, 5, 6, 7, 8, 9, and 10.

Embodiment 295. The method, empty LNP, empty-LNP solution, loaded LNP, loaded-LNP solution, or LNP formulation of any one of the preceding embodiments, wherein the encapsulation agent is a compound of Formula (EA-II):

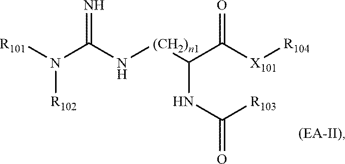

or a salt or isomer thereof, wherein
X₁₀₁ is a bond, NH, or O;
R₁₀₁ and R₁₀₂ are each independently selected from the group consisting of H, C₁-C₆ alkyl, and C₂-C₆ alkenyl;
R₁₀₃ and R₁₀₄ are each independently selected from the group consisting of C₁-C₂₀ alkyl and C₂-C₂₀ alkenyl; and
n1 is selected from 1, 2, 3, 4, 5, 6, 7, 8, 9, and 10.

Embodiment 296. The method, empty LNP, empty-LNP solution, loaded LNP, loaded-LNP solution, or LNP formulation of any one of the preceding embodiments, wherein the encapsulation agent is ethyl lauroyl arginate or a salt or isomer thereof.

Embodiment 297. The method, empty LNP, empty-LNP solution, loaded LNP, loaded-LNP solution, or LNP formulation of any one of the preceding embodiments, wherein the wt/wt ratio of the LNP formulation to the nucleic acid is in a range of from about 5:1 to about 60:1.

Embodiment 298. The method, empty LNP, empty-LNP solution, loaded LNP, loaded-LNP solution, or LNP formulation of any one of the preceding embodiments, wherein the wt/wt ratio of the LNP formulation to the nucleic acid is in a range of from about 10:1 to about 50:1.

Embodiment 299. The method, empty LNP, empty-LNP solution, loaded LNP, loaded-LNP solution, or LNP formulation of any one of the preceding embodiments, wherein the lipid solution, empty LNP, the empty-LNP solution, the loaded LNP, the loaded-LNP solution, and/or the LNP formulation further comprises a phospholipid, a PEG lipid, a structural lipid, or any combination thereof.

Embodiment 300. The method, empty LNP, empty-LNP solution, loaded LNP, loaded-LNP solution, or LNP formulation of any one of the preceding embodiments, wherein the empty LNP, the loaded LNP, and/or the LNP formulation comprises
about 30-60 mol% ionizable lipid;
about 0-30 mol% phospholipid;
about 15-50 mol% structural lipid; and
about 0.1-0.5 mol% PEG lipid.

Embodiment 301. The method, empty LNP, empty-LNP solution, loaded LNP, loaded-LNP solution, or LNP formulation of any one of the preceding embodiments, wherein the empty LNP, the loaded LNP, and/or the LNP formulation comprises
about 30-60 mol% ionizable lipid;
about 0-30 mol% phospholipid;
about 15-50 mol% structural lipid; and
about 0.01-10 mol% PEG lipid.

Embodiment 302. The method, empty LNP, empty-LNP solution, loaded LNP, loaded-LNP solution, or LNP formulation of any one of the preceding embodiments, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol.

Embodiment 303. The method, empty LNP, empty-LNP solution, loaded LNP, loaded-LNP solution, or LNP formulation of any one of the preceding embodiments, wherein the PEG lipid is a compound of Formula (PL-I):

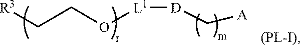

or a salt thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, -OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

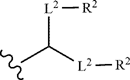

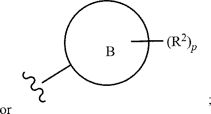

each instance of of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), -NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), -NR^{N}C(O)N(R^{N}), C(O)O, OC(O), OC(O)O, OC(O)N(R^{N}), NR^{N}C(O)O, C(O)S, SC(O), -C(=NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), -S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

Embodiment 304. The method, empty LNP, empty-LNP solution, loaded LNP, loaded-LNP solution, or LNP formulation of any one of the preceding embodiments, wherein the PEG lipid is a compound of Formula (PL-I-OH):

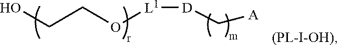

or a salt thereof.

Embodiment 305. The method, empty LNP, empty-LNP solution, loaded LNP, loaded-LNP solution, or LNP formulation of any one of the preceding embodiments, wherein the PEG lipid is a compound of Formula (PL-II):

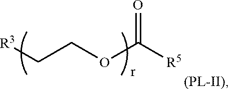

or a salt thereof, wherein:
R³ is —OR^{O};
R^{O} is hydrogen, optionally substituted alkyl or an oxygen protecting group;
r is an integer between 1 and 100;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), -C(O)N(R^{N}), NR^{N}C(O), NR^{N}C(O)N(R^{N}), C(O)O, OC(O), OC(O)O, OC(O)N(R^{N}), -NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, -S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)2N(R^{N}), or N(R^{N})S(O)₂O; and
each instance of R^{N} is independently hydrogen, optionally substituted alkyl, or a nitrogen protecting group.

Embodiment 306. The method, empty LNP, empty-LNP solution, loaded LNP, loaded-LNP solution, or LNP formulation of any one of the preceding embodiments, wherein the PEG lipid is a compound of Formula (PL-II-OH):

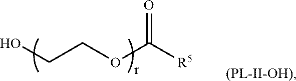

or a salt thereof, wherein:
r is an integer between 1 and 100;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), -C(O)N(R^{N}), NR^{N}C(O), NR^{N}C(O)N(R^{N}), C(O)O, OC(O), OC(O)O, OC(O)N(R^{N}), -NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, -S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)2N(R^{N}), OS(O)2N(R^{N}), or N(R^{N})S(O)₂O; and
each instance of R^{N} is independently hydrogen, optionally substituted alkyl, or a nitrogen protecting group.

Embodiment 307. The method, empty LNP, empty-LNP solution, loaded LNP, loaded-LNP solution, or LNP formulation of any one of the preceding embodiments, wherein r is an integer between 40 and 50.

Embodiment 308. The method, empty LNP, empty-LNP solution, loaded LNP, loaded-LNP solution, or LNP formulation of any one of the preceding embodiments, wherein r is 45.

Embodiment 309. The method, empty LNP, empty-LNP solution, loaded LNP, loaded-LNP solution, or LNP formulation of any one of the preceding embodiments, wherein R⁵ is C₁₇ alkyl.

Embodiment 310. The method, empty LNP, empty-LNP solution, loaded LNP, loaded-LNP solution, or LNP formulation of any one of the preceding embodiments, wherein the PEG lipid is a compound of Formula (PL-II) is:

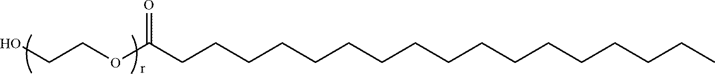

or a salt thereof.

Embodiment 311. The method, empty LNP, empty-LNP solution, loaded LNP, loaded-LNP solution, or LNP formulation of any one of the preceding embodiments, wherein the PEG lipid is a compound of Formula (PL-II) is

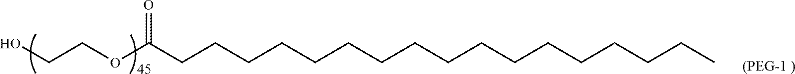

Embodiment 312. The method, empty LNP, empty-LNP solution, loaded LNP, loaded-LNP solution, or LNP formulation of any one of the preceding embodiments, wherein the PEG lipid is a compound of Formula (PL-III):

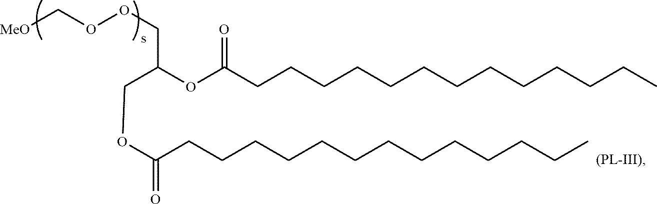

or a salt or isomer thereof, wherein s is an integer between 1 and 100.

Embodiment 313. The method, empty LNP, empty-LNP solution, loaded LNP, loaded-LNP solution, or LNP formulation of any one of the preceding embodiments, wherein the PEG lipid is a compound of following formula:

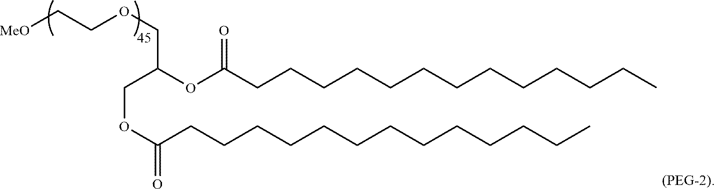

Embodiment 314. The method, empty LNP, empty-LNP solution, loaded LNP, loaded-LNP solution, or LNP formulation of any one of the preceding embodiments, wherein the structural lipid is selected from the group consisting of cholesterol, fecosterol, sitosterol, ergosterol, campesterol, stigmasterol, brassicasterol, tomatidine, ursolic acid, alpha-tocopherol, and derivatives thereof.

Embodiment 315. The method, empty LNP, empty-LNP solution, loaded LNP, loaded-LNP solution, or LNP formulation of any one of the preceding embodiments, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and derivatives thereof.

Embodiment 316. The method, empty LNP, empty-LNP solution, loaded LNP, loaded-LNP solution, or LNP formulation of any one of the preceding embodiments, wherein the phospholipid is 1,2-distearoyl-sn-glycero-3-phosphocholine (DSPC).

Embodiment 317. The method, empty LNP, empty-LNP solution, loaded LNP, loaded-LNP solution, or LNP formulation of any one of the preceding embodiments, wherein the ionizable lipid comprises an ionizable amino lipid.

Embodiment 318. The method, empty LNP, empty-LNP solution, loaded LNP, loaded-LNP solution, or LNP formulation of any one of the preceding embodiments, wherein the ionizable lipid is a compound of Formula (IL-1):

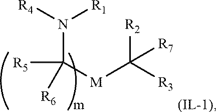

or their N-oxides, or a salt or isomer thereof, wherein:
R₁ is selected from the group consisting of C₅₋₃₀ alkyl, C₅₋₂₀ alkenyl, -R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)—M″—C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—,—S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, -OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR, and H;
each R″ is independently selected from the group consisting of C₃₋₁₅ alkyl and C₃₋₁₅ alkenyl;
each R* is independently selected from the group consisting of C₁-₁₂ alkyl and C₂₋₁₂ alkenyl;
each Y is independently a C₃-₆ carbocycle;
each X is independently selected from the group consisting of F, Cl, Br, and I; and
m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

Embodiment 319. The method, empty LNP, empty-LNP solution, loaded LNP, loaded-LNP solution, or LNP formulation of any one of the preceding embodiments, wherein the ionizable lipid is a compound of Formula (IL-IA):

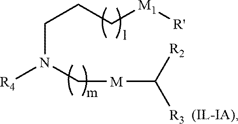

or its N-oxide, or a salt or isomer thereof, wherein 1 is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)—M″— C(O)O—, —C(O)N(R′)—, —P(O)(OR′)O—, —S—S—, an aryl group, and a heteroaryl group,; and R₂ and R₃ are independently selected from the group consisting of H, C₁₋₁₄ alkyl, and C₂₋₁₄ alkenyl. In some embodiments, m is 5, 7, or 9. In some embodiments, Q is OH, —NHC(S)N(R)₂, or —NHC(O)N(R)₂. In some embodiments, Q is —N(R)C(O)R, or —N(R)S(O)₂R.

Embodiment 320. The method, empty LNP, empty-LNP solution, loaded LNP, loaded-LNP solution, or LNP formulation of any one of the preceding embodiments, wherein the ionizable lipid is a compound of Formula (IL-IB):

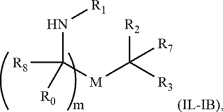

or its N-oxide, or a salt or isomer thereof, in which all variables are as defined herein. In some embodiments, m is selected from 5, 6, 7, 8, and 9; R₄ is hydrogen, unsubstituted C₁₋₃ alkyl, or —(CH₂)ₙQ, in which Q is —OH, —NHC(S)N(R)₂, —NHC(O)N(R)₂, —N(R)C(O)R, —N(R)S(O)₂R, —N(R)R8, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)—M″— C(O)O—, —C(O)N(R′)—, —P(O)(OR′)O—, —S—S—, an aryl group, and a heteroaryl group,; and R₂ and R₃ are independently selected from the group consisting of H, C₁₋₁₄ alkyl, and C₂₋₁₄ alkenyl. In some embodiments, m is 5, 7, or 9. In some embodiments, Q is OH, —NHC(S)N(R)₂, or —NHC(O)N(R)₂. In some embodiments, Q is —N(R)C(O)R, or —N(R)S(O)₂R.

Embodiment 321. The method, empty LNP, empty-LNP solution, loaded LNP, loaded-LNP solution, or LNP formulation of any one of the preceding embodiments, wherein the ionizable lipid is a compound of Formula (IL-II):

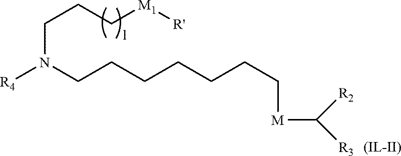

or its N-oxide, or a slat or isomer thereof, wherein 1 is selected from 1, 2, 3, 4 and 5; M1 is a bond or M′; R₄ is hydrogen, unsubstituted C₁₋₃ alkyl, or —(CH₂)ₙQ, in which n is 2, 3, or 4, and Q is —OH, — NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)—M″—C(O)O—, —C(O)N(R′)—, —P(O)(OR′)O—, —S—S—, an aryl group, and a heteroaryl group,; and R₂ and R₃ are independently selected from the group consisting of H, C₁₋₁₄ alkyl, and C₂₋₁₄ alkenyl.

Embodiment 322. The method, empty LNP, empty-LNP solution, loaded LNP, loaded-LNP solution, or LNP formulation of any one of the preceding embodiments, wherein the ionizable lipid is a compound of Formula (IL-IIa):

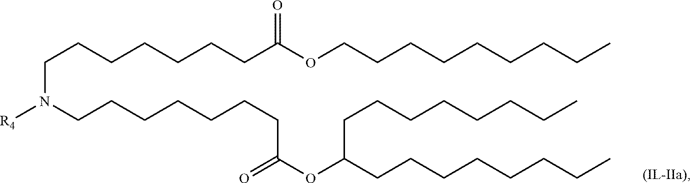

or their N-oxides, or a salt or isomer thereof, wherein R₄ is as described herein.

Embodiment 323. The method, empty LNP, empty-LNP solution, loaded LNP, loaded-LNP solution, or LNP formulation of any one of the preceding embodiments, wherein the ionizable lipid is a compound of Formula (IL-IIb):

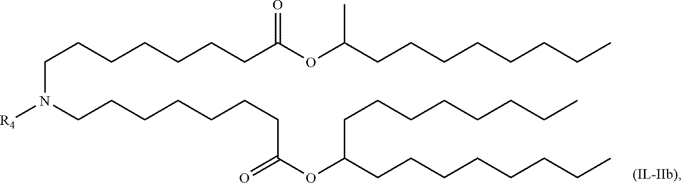

or their N-oxides, or a salt or isomer thereof, wherein R₄ is as described herein.

Embodiment 324. The method, empty LNP, empty-LNP solution, loaded LNP, loaded-LNP solution, or LNP formulation of any one of the preceding embodiments, wherein the ionizable lipid is a compound of Formula (IL-IIc) or (IL-IIe):

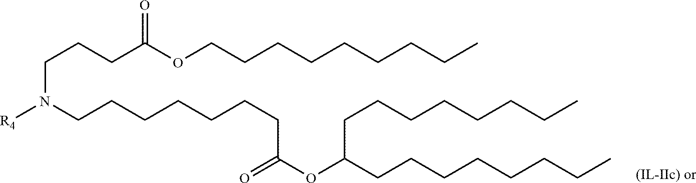

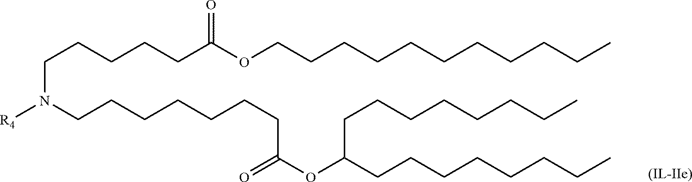

or their N-oxides, or a salt or isomer thereof, wherein R₄ is as described herein.

Embodiment 325. The method, empty LNP, empty-LNP solution, loaded LNP, loaded-LNP solution, or LNP formulation of any one of the preceding embodiments, wherein the ionizable lipid is a compound of Formula (IL-IIf):

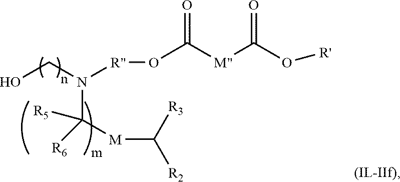

or their N-oxides, or a salt or isomer thereof, wherein M is —C(O)O— or —OC(O)—, M″ is C₁₋₆ alkyl or C₂-₆ alkenyl, R₂ and R₃ are independently selected from the group consisting of C₅₋₁₄ alkyl and C₅₋₁₄ alkenyl, and n is selected from 2, 3, and 4.

Embodiment 326. The method, empty LNP, empty-LNP solution, loaded LNP, loaded-LNP solution, or LNP formulation of any one of the preceding embodiments, wherein the ionizable lipid is a compound of Formula (IL-IId):

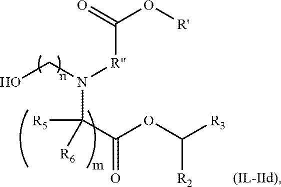

or their N-oxides, or a salt or isomer thereof, wherein n is 2, 3, or 4; and m, R′, R″, and R₂ through R₆ are as described herein. In some embodiments, each of R₂ and R₃ may be independently selected from the group consisting of C₅₋₁₄ alky and C₅₋₁₄ alkenyl.

Embodiment 327. The method, empty LNP, empty-LNP solution, loaded LNP, loaded-LNP solution, or LNP formulation of any one of the preceding embodiments, wherein the ionizable lipid is a compound of Formula (IL-IIg):

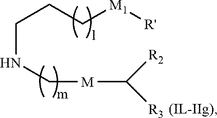

or their N-oxides, or a salt or isomer thereof, wherein 1 is selected from 1, 2, 3, 4, and 5; m is selected from 5, 6, 7, 8, and 9; M₁ is a bond or M′; M and M′ are independently selected from from —C(O)O—, —OC(O)—, —OC(O)—M″—C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. In some embodiments, M″ is C₁₋₆ alkyl (e.g., C₁₋₄ alkyl) or C₂-₆ alkenyl (e.g. C₂₋₄ alkenyl). In some embodiments, R₂ and R₃ are independently selected from the group consisting of C₅₋₁₄ alkyl and C₅₋₁₄ alkenyl.

Embodiment 328. The method, empty LNP, empty-LNP solution, loaded LNP, loaded-LNP solution, or LNP formulation of any one of the preceding embodiments, wherein the ionizable lipid is

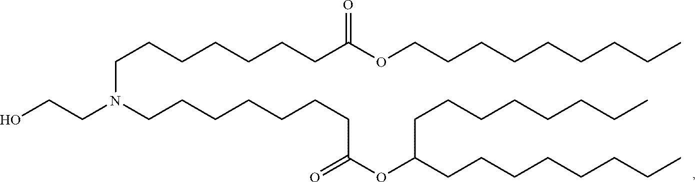

or a salt thereof.

Embodiment 329. The method, empty LNP, empty-LNP solution, loaded LNP, loaded-LNP solution, or LNP formulation of any one of the preceding embodiments, wherein the ionizable lipid is

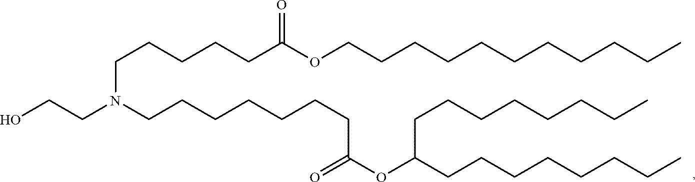

or a salt thereof.

Embodiment 330. The method, empty LNP, empty-LNP solution, loaded LNP, loaded-LNP solution, or LNP formulation of any one of the preceding embodiments, wherein the

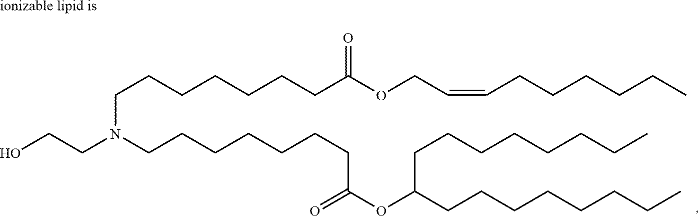

or a salt thereof.

Embodiment 331. The method, empty LNP, empty-LNP solution, loaded LNP, loaded-LNP solution, or LNP formulation of any one of the preceding embodiments, wherein the

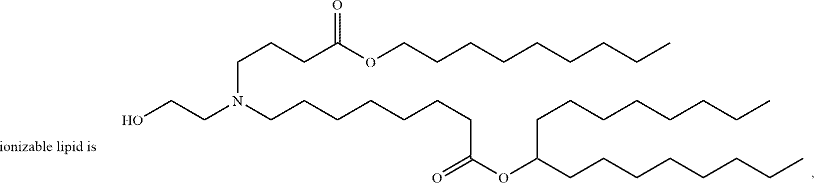

or a salt thereof.

Embodiment 332. The method, empty LNP, empty-LNP solution, loaded LNP, loaded-LNP solution, or LNP formulation of any one of the preceding embodiments, wherein the ionizable lipid is a compound of formula (IL-III):

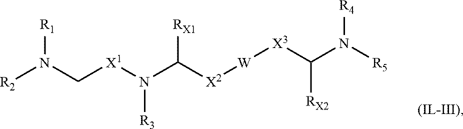

or a salt or isomer thereof, wherein,

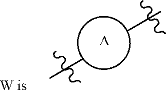

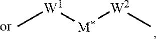

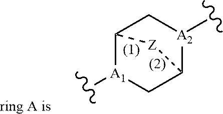

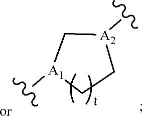

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent; R₁, R₂, R₃, R₄, and R⁵ are independently selected from the group consisting of C₅₋₂₀ alkyl, C₅₋₂₀ alkenyl, —R″MR′, —R*YR″, —YR″, and —R*OR″; R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl; X¹, X², and X³ are independently selected from the group consisting of a bond, —CH₂—, —(CH₂)₂—, —CHR—, —CHY—, —C(O)—, —C(O)O—, —OC(O)—, —(CH2)An⁻C(O)—, —C(O)—(CH2)n⁻, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

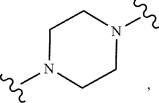

then
i) at least one of X¹, X², and X³ is not —CH₂—; and/or
ii) at least one of R₁, R₂, R₃, R₄, and R⁵ is —R″MR′.

Embodiment 333. The method, empty LNP, empty-LNP solution, loaded LNP, loaded-LNP solution, or LNP formulation of any one of the preceding embodiments, wherein the ionizable lipid is a compound of any of formulae (IL-IIIal)-(IL-IIIa8):

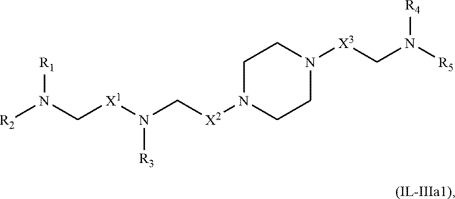

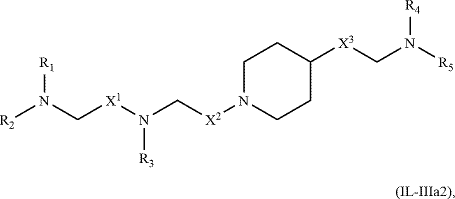

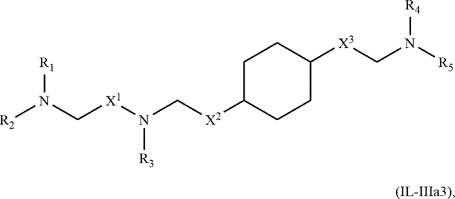

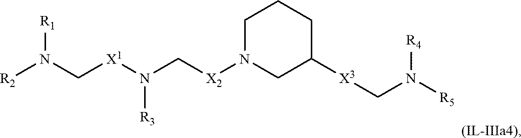

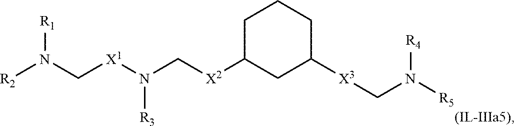

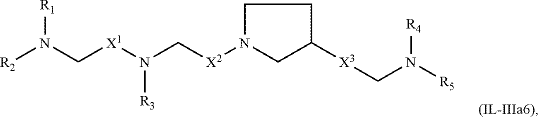

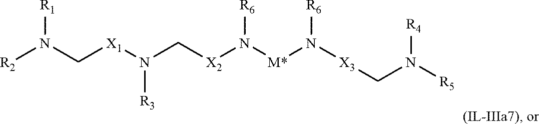

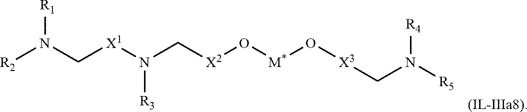

Embodiment 334. The method, empty LNP, empty-LNP solution, loaded LNP, loaded-LNP solution, or LNP formulation of any one of the preceding embodiments, wherein the ionizable lipid is

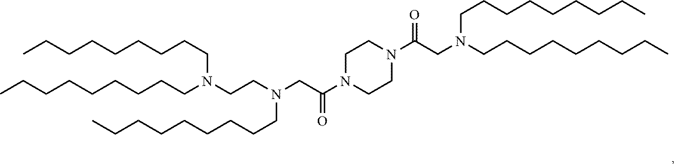

or a salt thereof.

Embodiment 335. The method, empty LNP, empty-LNP solution, loaded LNP, loaded-LNP solution, or LNP formulation of any one of the preceding embodiments, wherein the ionizable lipid is a compound of formula (IL-VIVa):

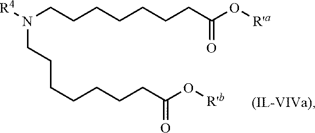

or its N-oxide, or a salt or isomer thereof,
wherein R′^{a} is R’branched or R’^{cyclic}. wherein R^{aγ}
R’branched is

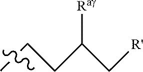

and R’^{cyclic} is:

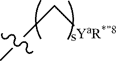

and R’^{b} is

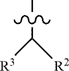

or;

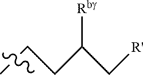

;
wherein

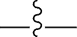

denotes a point of attachment;
wherein R^{aγ} and R^{bγ} are each independently a C₂₋₁₂ alkyl or C₂₋₁₂ alkenyl;
R₂ and R³ are each independently selected from the group consisting of C₁₋₁₄ alkyl and C₂₋₁₄ alkenyl;
R₄ is —(CH₂)₂OH;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3.

Embodiment 336. The method, empty LNP, empty-LNP solution, loaded LNP, loaded-LNP solution, or LNP formulation of any one of the preceding embodiments, wherein the ionizable lipid is a compound of formula (IL-VIVb):

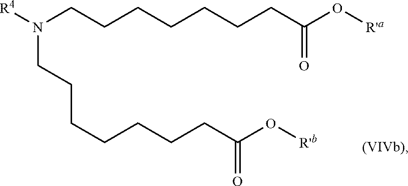

or its N-oxide, or a salt or isomer thereof,
wherein R′^{a} is R’^{branched} or R’^{cycllc.} wherein R^{aγ}
R’branched is

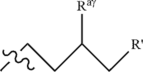

and R′ cyclic is:

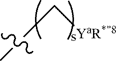

and
R′b is:

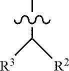

or;

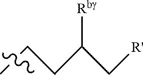

wherein

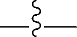

denotes a point of attachment;
wherein R^{aγ} and R^{bγ} are each independently a C₂₋₁₂ alkyl or C₂₋₁₂ alkenyl;
R₂ and R³ are each independently selected from the group consisting of C₁₋₁₄ alkyl and
C₂₋₁₄ alkenyl;
R₄ is

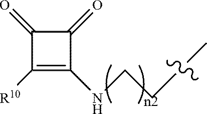

, wherein

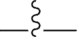

denotes a point of attachment;
R¹⁰ is N(R)₂; each R is independently selected from the group consisting of C₁-₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and s is 2 or 3.

Embodiment 337. The method, empty LNP, empty-LNP solution, loaded LNP, loaded-LNP solution, or LNP formulation of any one of the preceding embodiments, wherein the ionizable lipid is selected from:

| | |
|---|---|
| 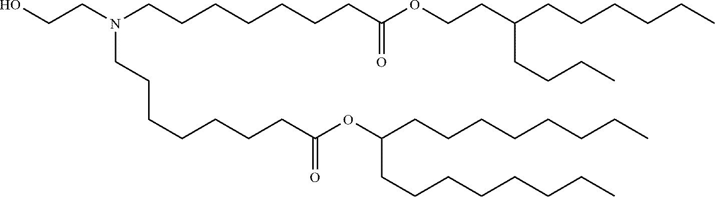 [SMILES: CCCCCCCCC(CCCCCCCC)OC(=O)CCCCCCCN(CCO)CCCCCCCC(=O)OCCC(CCCC)CCCCCC] | 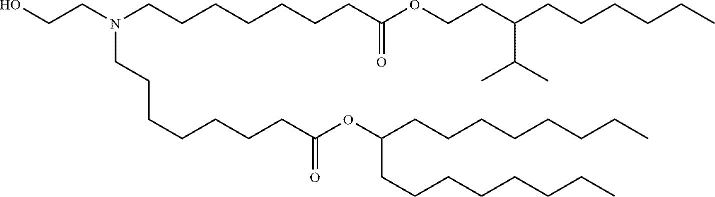 [SMILES: CCCCCCCCC(CCCCCCCC)OC(=O)CCCCCCCN(CCO)CCCCCCCC(=O)OCCC(CCCCCC)C(C)C] |
| 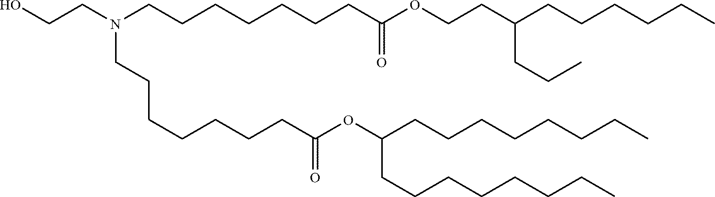 [SMILES: CCCCCCCCC(CCCCCCCC)OC(=O)CCCCCCCN(CCO)CCCCCCCC(=O)OCCC(CCC)CCCCCC] | 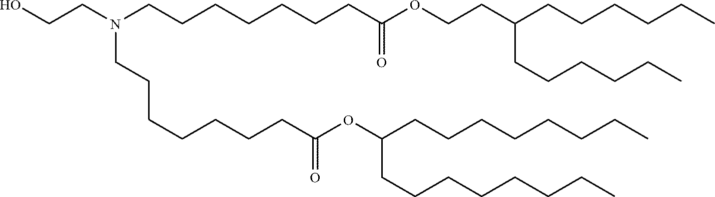 [SMILES: CCCCCCCCC(CCCCCCCC)OC(=O)CCCCCCCN(CCO)CCCCCCCC(=O)OCCC(CCCCCC)CCCCCC] |
| 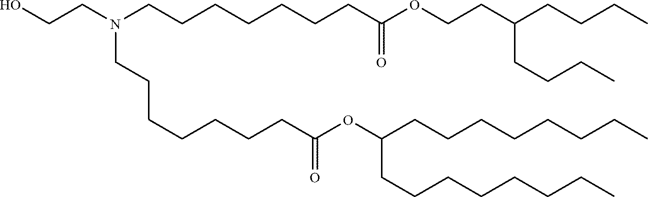 [SMILES: CCCCCCCCC(CCCCCCCC)OC(=O)CCCCCCCN(CCO)CCCCCCCC(=O)OCCC(CCCC)CCCC] | 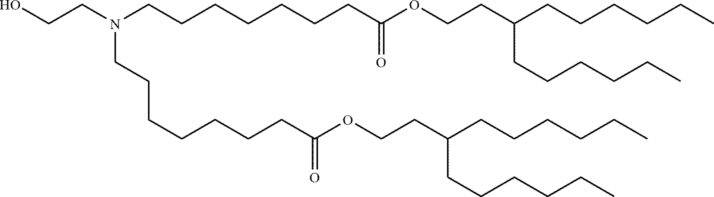 [SMILES: CCCCCCC(CCCCCC)CCOC(=O)CCCCCCCN(CCO)CCCCCCCC(=O)OCCC(CCCCCC)CCCCCC] |
| 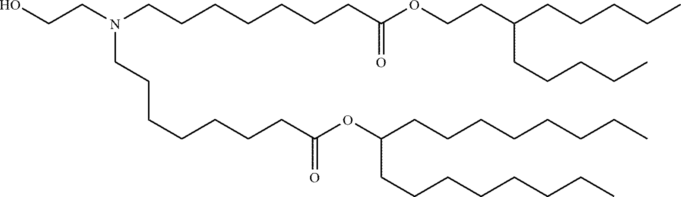 [SMILES: CCCCCCCCC(CCCCCCCC)OC(=O)CCCCCCCN(CCO)CCCCCCCC(=O)OCCC(CCCCC)CCCCC] | 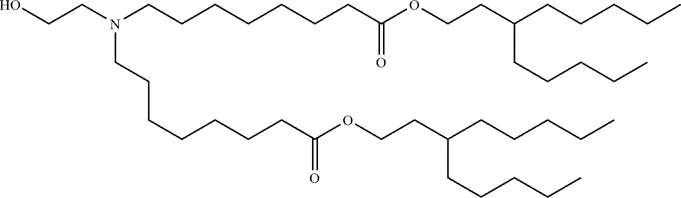 [SMILES: CCCCCC(CCCCC)CCOC(=O)CCCCCCCN(CCO)CCCCCCCC(=O)OCCC(CCCCC)CCCCC] |
| 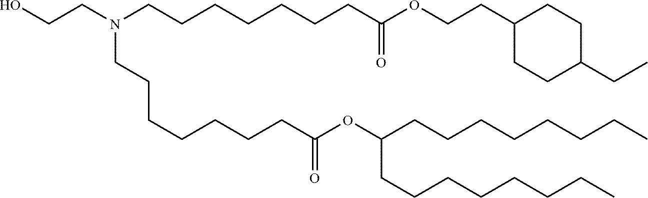 [SMILES: CCCCCCCCC(CCCCCCCC)OC(=O)CCCCCCCN(CCO)CCCCCCCC(=O)OCCC1CCC(CC)CC1] | 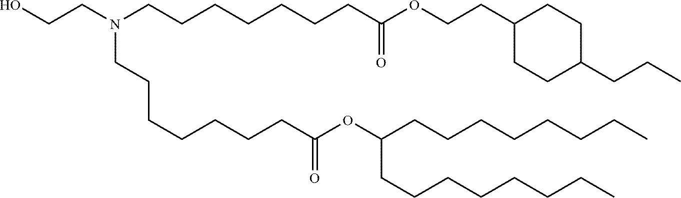 [SMILES: CCCCCCCCC(CCCCCCCC)OC(=O)CCCCCCCN(CCO)CCCCCCCC(=O)OCCC1CCC(CCC)CC1] |
| 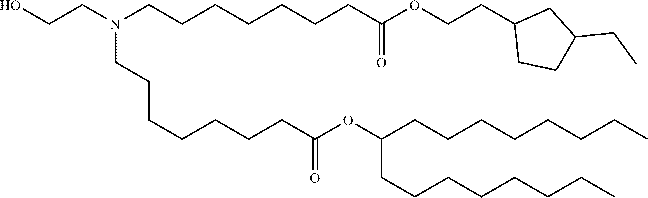 [SMILES: CCCCCCCCC(CCCCCCCC)OC(=O)CCCCCCCN(CCO)CCCCCCCC(=O)OCCC1CCC(CC)C1] | 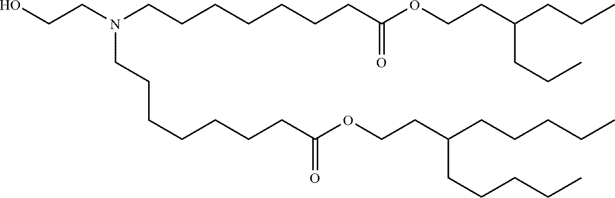 [SMILES: CCCCCC(CCCCC)CCOC(=O)CCCCCCCN(CCO)CCCCCCCC(=O)OCCC(CCC)CCC] |
| 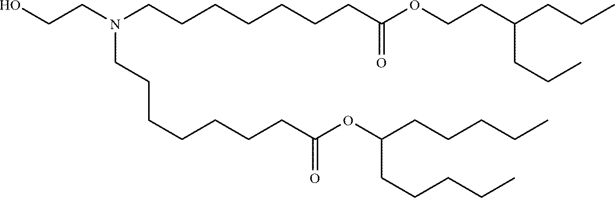 [SMILES: CCCCCC(CCCCC)OC(=O)CCCCCCCN(CCO)CCCCCCCC(=O)OCCC(CCC)CCC] | 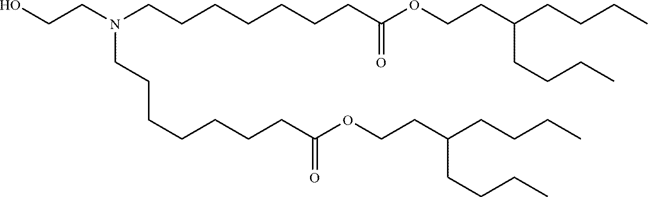 [SMILES: CCCCC(CCCC)CCOC(=O)CCCCCCCN(CCO)CCCCCCCC(=O)OCCC(CCCC)CCCC] |
| 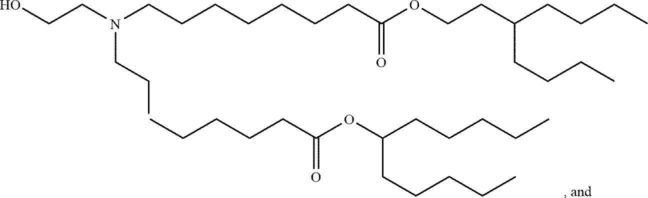 | 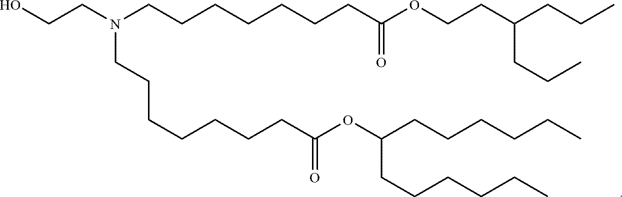 |

Embodiment 338. The method, empty LNP, empty-LNP solution, loaded LNP, loaded-LNP solution, or LNP formulation of any one of the preceding embodiments, wherein the ionizable lipid is selected from the group consisting of 3-(didodecylamino)-N1,N1,4-tridodecyl-1-piperazineethanamine (KL10), N1-[2-(didodecylamino)ethyl] -N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3P)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).

Embodiment 339. The method, empty LNP, empty-LNP solution, loaded LNP, loaded-LNP solution, or LNP formulation of any one of the preceding embodiments, wherein the nucleic acid is a ribonucleic acid.

Embodiment 340. The method, empty LNP, empty-LNP solution, loaded LNP, loaded-LNP solution, or LNP formulation of any one of the preceding embodiments, wherein the ribonucleic acid is at least one ribonucleic acid selected from the group consisting of a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and a long non-coding RNA (lncRNA).

Embodiment 341. The method, empty LNP, empty-LNP solution, loaded LNP, loaded-LNP solution, or LNP formulation of any one of the preceding embodiments, wherein the nucleic acid is a messenger RNA (mRNA).

Embodiment 342. The method, empty LNP, empty-LNP solution, loaded LNP, loaded-LNP solution, or LNP formulation of any one of the preceding embodiments, wherein the mRNA includes at least one motif selected from the group consisting of a stem loop, a chain terminating nucleoside, a poly A sequence, a polyadenylation signal, and a 5′ cap structure.

Embodiment 343. The method, empty LNP, empty-LNP solution, loaded LNP, loaded-LNP solution, or LNP formulation of any one of the preceding embodiments, wherein the mRNA is at least 30 nucleotides in length.

Embodiment 344. The method, empty LNP, empty-LNP solution, loaded LNP, loaded-LNP solution, or LNP formulation of any one of the preceding embodiments, wherein the mRNA is at least 300 nucleotides in length.

Embodiment 345. The method, empty LNP, empty-LNP solution, loaded LNP, loaded-LNP solution, or LNP formulation of any one of the preceding embodiments, wherein the LNP formulation has a N:P ratio from about 1.1:1 to about 30.1.

Embodiment 346. The method, empty LNP, empty-LNP solution, loaded LNP, loaded-LNP solution, or LNP formulation of any one of the preceding embodiments, wherein the LNP formulation has a N:P ratio from about 2:1 to about 20:1.

Embodiment 347. The method, empty LNP, empty-LNP solution, loaded LNP, loaded-LNP solution, or LNP formulation of any one of the preceding embodiments, wherein the LNP formulation has a N:P ratio from about 2:1 to about 10:1 or about 2:1 to about 5:1.

Embodiment 348. The method, empty LNP, empty-LNP solution, loaded LNP, loaded-LNP solution, or LNP formulation of any one of the preceding embodiments, wherein the LNP comprises about 0.01 to about 500 mg/mL of the nucleic acid, about 0.1 to about 100 mg/mL, about 0.25 to about 50 mg/mL, about 0.5 to about 10 mg/mL, or about 1.0 to about 10 mg/mL of the nucleic acid.

Embodiment 349. The method, empty LNP, empty-LNP solution, loaded LNP, loaded-LNP solution, or LNP formulation of any one of the preceding embodiments, wherein the empty LNP, the loaded LNP, and/or LNP formulation has a polydispersity index (PDI) from about 0.01 to about 0.25.

Embodiment 350. The method, empty LNP, empty-LNP solution, loaded LNP, loaded-LNP solution, or LNP formulation of any one of the preceding embodiments, wherein the LNP formulation has an encapsulation efficiency of at least about 50%, at least about 60%, at least about 70%, at least about 80%, or at least about 90%.

Embodiment 351. The method, empty LNP, empty-LNP solution, loaded LNP, loaded-LNP solution, or LNP formulation of any one of the preceding embodiments, wherein the LNP formulation has an encapsulation efficiency of at least about 85%, at least about 90%, or at least about 95%.

Embodiment 352. The method, empty LNP, empty-LNP solution, loaded LNP, loaded-LNP solution, or LNP formulation of any one of the preceding embodiments, wherein the LNP formulation has an encapsulation efficiency of at least about 90%, at least about 92%, at least about 94%, at least about 96%, or at least about 98%.

Embodiment 353. The method, empty LNP, empty-LNP solution, loaded LNP, loaded-LNP solution, or LNP formulation of any one of the preceding embodiments, wherein the nucleic acid expression (e.g., the mRNA expression) of the LNP formulation is about 20% or higher, about 25% or higher, about 30% or higher, about 35% or higher, about 40% or higher, about 45% or higher, about 50% or higher, about 55% or higher, about 60% or higher, about 65% or higher, about 70% or higher, about 75% or higher, about 80% or higher, about 85% or higher, about 90% or higher, about 95% or higher, about 96% or higher, about 97% or higher, about 98% or higher, or about 99% or higher.

Embodiment 354. The method, empty LNP, empty-LNP solution, loaded LNP, loaded-LNP solution, or LNP formulation of any one of the preceding embodiments, wherein the empty LNP, the loaded LNP, and/or the LNP formulation has an average lipid nanoparticle diameter of about 200 nm or less, about 175 nm or less, about 150 nm or less, about 125 nm or less, about 100 nm or less, about 90 nm or less, about 80 nm or less, about 75 nm or less, about 70 nm or less, about 65 nm or less, about 60 nm or less, about 55 nm or less, about 50 nm or less, about 45 nm or less, about 40 nm or less, about 35 nm or less, about 30 nm or less, about 25 nm or less, or about 20 nm or less.

Embodiment 355. The method, empty LNP, empty-LNP solution, loaded LNP, loaded-LNP solution, or LNP formulation of any one of the preceding embodiments, wherein the empty LNP, the loaded LNP, and/or the LNP formulation has an average lipid nanoparticle diameter of about 20 nm to about 150 nm, about 25 nm to about 125 nm, about 30 nm to about 110 nm, about 35 nm to about 100 nm, about 40 nm to about 90 nm, about 45 nm to about 80 nm, or about 50 nm to about 70 nm.

Embodiment 356. The method, empty LNP, empty-LNP solution, loaded LNP, loaded-LNP solution, or LNP formulation of any one of the preceding embodiments, wherein the empty LNP, the loaded LNP, and/or LNP formulation has an average lipid nanoparticle diameter of about 15 nm to about 55 nm, about 20 nm to about 50 nm, about 25 nm to about 45 nm, or about 30 nm to about 40 nm.

Embodiment 357. The method, empty LNP, empty-LNP solution, loaded LNP, loaded-LNP solution, or LNP formulation of any one of the preceding embodiments, wherein the empty LNP, the loaded LNP, and/or LNP formulation has an average lipid nanoparticle diameter of about 25 to about 45 nm.

Embodiment 358. The method, empty LNP, empty-LNP solution, loaded LNP, loaded-LNP solution, or LNP formulation of any one of the preceding embodiments, wherein the polydispersity index (PDI) of the empty LNP, the empty-LNP solution, the loaded LNP, the loaded-LNP solution, and/or the LNP formulation increases by less than about 0.25, less than about 0.20, less than about 0.15, less than about 0.10, less than about 0.05, less than about 0.04, less than about 0.03, less than about 0.02, or less than about 0.01 after storage of the LNP formulation at about -5-25° C., about 0-10° C., or about 2-8° C. for at least 1 day, at least 2 days, at least 1 week, at least 2 weeks, at least 4 weeks, at least 1 month, at least 2 months, at least 3 months, at least 6 months, at least 8 months, or at least 1 year.

Embodiment 359. The method, empty LNP, empty-LNP solution, loaded LNP, loaded-LNP solution, or LNP formulation of any one of the preceding embodiments, wherein the polydispersity index (PDI) of the empty LNP, the empty-LNP solution, the loaded LNP, the loaded-LNP solution, and/or the LNP formulation increases by less than about 25%, less than about 20%, less than about 15%, less than about 10%, less than about 5%, less than about 4%, less than about 3%, less than about 2%, or less than about 1% after storage of the LNP formulation at about -100° C. to about 80° C., about -80° C. to about 60° C., about -40° C. to about 40° C., about -20° C. to about 30° C., about -5° C. to about 25° C., about 0° C. to about 10° C., or about 2° C. to about 8° C. for at least 1 day, at least 2 days, at least 1 week, at least 2 weeks, at least 4 weeks, at least 1 month, at least 2 months, at least 3 months, at least 6 months, at least 8 months, or at least 1 year.

Embodiment 360. The method, empty LNP, empty-LNP solution, loaded LNP, loaded-LNP solution, or LNP formulation of any one of the preceding embodiments, wherein the empty LNP, the empty-LNP solution, the loaded LNP, the loaded-LNP solution, and/or the LNP formulation has a less than about 25% decrease in encapsulation efficiency, less than about 20% decrease, less than about 15% decrease, less than about 10% decrease, less than about 5% decrease, less than about 4% decreases, less than about 3% decrease, less than about 2% decrease, or less than about 1% decrease in encapsulation efficiency after storage of the LNP formulation at about -100° C. to about 80° C., about -80° C. to about 60° C., about -40° C. to about 40° C., about -20° C. to about 30° C., about -5° C. to about 25° C., about 0° C. to about 10° C., or about 2° C. to about 8° C. for at least 1 day, at least 2 days, at least 1 week, at least 2 weeks, at least 4 weeks, at least 1 month, at least 2 months, at least 3 months, at least 6 months, at least 8 months, or at least 1 year.

Embodiment 361. The method, empty LNP, empty-LNP solution, loaded LNP, loaded-LNP solution, or LNP formulation of any one of the preceding embodiments, wherein the average lipid nanoparticle diameter of the empty LNP, the loaded LNP, and/or the LNP formulation is about 99% or less, about 98% or less, about 97% or less, about 96% or less, about 95% or less, about 90% or less, about 85% or less, about 80% or less, about 75% or less, about 70% or less, about 65% or less, about 60% or less, about 55% or less, about 50% or less, about 40% or less, about 30% or less, about 20% or less, or about 10% or less as compared to the LNP formulation produced by a comparable method.

Embodiment 362. The method, empty LNP, empty-LNP solution, loaded LNP, loaded-LNP solution, or LNP formulation of any one of the preceding embodiments, wherein the encapsulation efficiency of the LNP formulation is higher than the encapsulation efficiency of the LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 fold or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

Embodiment 363. The method, empty LNP, empty-LNP solution, loaded LNP, loaded-LNP solution, or LNP formulation of any one of the preceding embodiments, wherein the nucleic acid expression (e.g., the mRNA expression) of the LNP formulation is higher than the nucleic acid expression (e.g., the mRNA expression) of the LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 fold or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

Embodiment 364. A method of treating or preventing a disease or disorder, the method comprising administering to a subject in need thereof the empty LNP of any one of the preceding embodiments.

Embodiment 365. A method of treating or preventing a disease or disorder, the method comprising administering to a subject in need thereof the empty-LNP solution of any one of the preceding embodiments.

Embodiment 366. A method of treating or preventing a disease or disorder, the method comprising administering to a subject in need thereof the loaded LNP of any one of the preceding embodiments.

Embodiment 367. A method of treating or preventing a disease or disorder, the method comprising administering to a subject in need thereof the loaded-LNP solution of any one of the preceding embodiments.

Embodiment 368. A method of treating or preventing a disease or disorder, the method comprising administering to a subject in need thereof the LNP formulation of any one of the preceding embodiments.

Embodiment 369. The method of any one of the preceding embodiments, wherein the administering is performed parenterally.

Embodiment 370. The method of any one of the preceding embodiments, wherein the administering is performed intramuscularly, intradermally, subcutaneously, and/or intravenously.

Embodiment 371. The empty LNP of any one of the preceding embodiments for use in treating or preventing a disease or disorder in a subject.

Embodiment 372. The empty-LNP solution of any one of the preceding embodiments for use in treating or preventing a disease or disorder in a subject.

Embodiment 373. The loaded LNP of any one of the preceding embodiments for use in treating or preventing a disease or disorder in a subject.

Embodiment 374. The loaded-LNP solution of any one of the preceding embodiments for use in treating or preventing a disease or disorder in a subject.

Embodiment 375. The LNP formulation of any one of the preceding embodiments for use in treating or preventing a disease or disorder in a subject.

Embodiment 376. Use of the empty LNP of any one of the preceding embodiments in the manufacture of a medicament for treating or preventing a disease or disorder.

Embodiment 377. Use of the empty-LNP solution of any one of the preceding embodiments in the manufacture of a medicament for treating or preventing a disease or disorder.

Embodiment 378. Use of the loaded LNP of any one of the preceding embodiments in the manufacture of a medicament for treating or preventing a disease or disorder.

Embodiment 379. Use of the loaded-LNP solution of any one of the preceding embodiments in the manufacture of a medicament for treating or preventing a disease or disorder.

Embodiment 380. A pharmaceutical kit comprising the empty LNP, empty-LNP solution, loaded LNP, loaded-LNP solution, or LNP formulation of any one of the preceding embodiments.

Embodiment 381. A pharmaceutical kit comprising the empty LNP, empty-LNP solution, loaded LNP, loaded-LNP solution, or LNP formulation of any one of the preceding embodiments.

Embodiment 382. A pharmaceutical kit comprising the empty LNP or the empty-LNP solution of any one of the preceding embodiments.

Embodiment 383. A pharmaceutical kit comprising the loaded LNP or the loaded-LNP solution, or LNP formulation of any one of the preceding embodiments.

Embodiment 384. A pharmaceutical kit comprising LNP formulation of any one of the preceding embodiments.

Embodiment 385. A pharmaceutical kit comprising a medicament comprising the loaded LNP of any one of the preceding embodiments.

Embodiment 386. A pharmaceutical kit comprising a medicament comprising a preparation comprising lipid nanoparticles (LNPs) of any one of the preceding embodiments.

Embodiment 387. A pharmaceutical kit comprising a population of empty-LNPs of any one of the preceding embodiments.

Embodiment 388. A pharmaceutical kit comprising a population of empty-LNPs of any one of the preceding embodiments and a therapeutic or prophylactic agent solution.

Embodiment 389. A pharmaceutical kit comprising
(a) a first container comprising the empty-LNP of any one of the preceding embodiments; and
(b) a second container comprising a solution comprising a therapeutic or prophylactic agent.

Embodiment 390. A pharmaceutical kit comprising
(a) a first container comprising the empty-LNP of any one of the preceding embodiments;
(b) a second container comprising a solution comprising a therapeutic or prophylactic agent; and
(c) instructions for combining the content of the first container and the second container.

Embodiment 391. The pharmaceutical kit of any one of the preceding embodiments wherein the first container is a polytetrafluoroethylene (PTFE) bag.

Embodiment 392. The pharmaceutical kit of any one of the preceding embodiments wherein the second container is a polytetrafluoroethylene (PTFE) bag.

Embodiment 393. A container comprising a medicament comprising the loaded LNP of any one of the preceding embodiments.

Embodiment 394. A container comprising a medicament comprising a preparation comprising lipid nanoparticles (LNPs) of any one of the preceding embodiments.

Embodiment 395. A container comprising the empty-LNP any one of the preceding embodiments.

Embodiment 396. A container comprising a population of empty-LNPs of any one of the preceding embodiments.

Embodiment 397. The container of any one of the preceding embodiments, wherein the container is a polytetrafluoroethylene (PTFE) bag.

Embodiment 398. The pharmaceutical kit of any one of the preceding embodiments, wherein the therapeutic or prophylactic agent is a nucleic acid.

Embodiment 399. The pharmaceutical kit of any one of the preceding embodiments, wherein the nucleic acid is a ribonucleic acid.

Embodiment 400. The pharmaceutical kit of any one of the preceding embodiments, wherein the ribonucleic acid is at least one ribonucleic acid selected from the group consisting of a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and a long non-coding RNA (lncRNA).

Embodiment 401. The pharmaceutical kit of any one of the preceding embodiments, wherein the nucleic acid is a messenger RNA (mRNA).

1. A method of preparing an empty-lipid nanoparticle solution (empty-LNP solution) comprising an empty lipid nanoparticle (empty LNP), comprising:
(i) a mixing step, comprising mixing a lipid solution comprising an ionizable lipid, a phospholipid, a PEG lipid and a structural lipid with an aqueous buffer solution comprising a first buffering agent, thereby forming the empty-LNP solution comprising the empty LNP, wherein the empty LNP comprises from about 0.1 mol% to about 0.5 mol% of the PEG lipid,
wherein the empty-LNP solution comprises an acetate buffer and has a pH in the range of about 4.6 to about 6.0.

2. The method of claim 1, further comprising processing the empty-LNP solution.

3. A method of preparing a loaded lipid nanoparticle solution (loaded-LNP solution) comprising a loaded lipid nanoparticle (loaded LNP), comprising:
(i) a mixing step, comprising mixing a lipid solution comprising an ionizable lipid, a phospholipid, a PEG lipid and a structural lipid with an aqueous buffer solution comprising a first buffering agent, thereby forming the empty-LNP solution comprising the empty LNP, wherein the empty LNP comprises from about 0.1 mol% to about 0.5 mol% of the PEG lipid, wherein the empty-LNP solution comprises an acetate buffer and has a pH in the range of about 4.6 to about 6.0; and
(ii) a loading step, comprising mixing a nucleic acid solution comprising a nucleic acid with the empty-LNP solution, thereby forming a loaded-LNP solution comprising a loaded LNP.

4. The method of any one of the preceding claims, further comprising processing the loaded-LNP solution, thereby forming a lipid nanoparticle formulation (LNP formulation).

5. The method of any one of the preceding claims, wherein the step of processing the loaded-LNP solution comprises a first adding step, comprising adding a polyethylene glycol lipid (PEG lipid) to the loaded LNP.

6. The method of any one of the preceding claims, wherein the first adding step comprises adding a polyethylene glycol solution (PEG solution) comprising the PEG lipid to the loaded-LNP solution.

7. The method of any one of the preceding claims, wherein the first adding step comprises adding from about 0.1 mol% to about 3.0 mol% PEG lipid, from about 0.2 mol% to about 2.5 mol% PEG lipid, from about 0.5 mol% to about 2.0 mol% PEG lipid, from about 0.75 mol% to about 1.5 mol% PEG lipid, or from about 1.0 mol% to about 1.25 mol% PEG lipid to the empty LNP or the loaded LNP.

8. The method of any one of the preceding claims, wherein the step of processing the empty-LNP solution further comprises pH adjusting.

9. The method of any one of the preceding claims, wherein the pH adjusting comprises adding a second buffering agent.

10. The method of any one of the preceding claims, wherein the second buffering agent comprises a second aqueous buffer solution.

11. The method of any one of the preceding claims, wherein the second aqueous buffer is selected from the group consisting of an acetate buffer, a citrate buffer, a phosphate buffer, and a tris buffer.

12. The method of any one of the preceding claims, wherein the step of processing the empty-LNP solution further comprises filtering.

13. The method of any one of the preceding claims, wherein the filtering is performed by a tangential flow filtration.

14. The method of any one of the preceding claims, wherein the step of processing the loaded-LNP solution further comprises buffer exchanging.

15. The method of any one of the preceding claims, wherein the buffer exchanging comprises addition of an aqueous buffer solution comprising a third buffering agent.

16. The method of any one of the preceding claims, wherein the third buffering agent comprises a third aqueous buffer solution.

17. The method of any one of the preceding claims, wherein the third aqueous buffer solution is selected from the group consisting of an acetate buffer, a citrate buffer, a phosphate buffer, and a tris buffer.

18. The method of any one of the preceding claims, wherein the third aqueous buffer solution has a pH in a range of about 6.5 to about 8.5, about 7.0 to about 8.0, about 7.2 to about 7.8, or about 7.4 to about 7.6.

19. The method of any one of the preceding claims, wherein the third aqueous buffer solution has a pH of about 7.5.

20. The method of any one of the preceding claims, wherein the first adding step is performed prior to the buffer exchanging.

21. The method of any one of the preceding claims, wherein the first adding step is performed after the buffer exchanging.

22. The method of any one of the preceding claims, wherein the step of processing the loaded-LNP solution comprises a second adding step, comprising adding a polyethylene glycol lipid (PEG lipid) to the loaded LNP.

23. The method of any one of the preceding claims, wherein the second adding step is performed prior to the buffer exchanging.

24. The method of any one of the preceding claims, wherein the second adding step is performed after the buffer exchanging.

25. The method of any one of the preceding claims, wherein the step of processing the empty-LNP solution further comprises diluting the empty-LNP solution.

26. The method of any one of the preceding claims, wherein the step of processing the empty-LNP solution or loaded-LNP solution further comprises freezing the empty-LNP solution or loaded-LNP solution.

27. The method of any one of the preceding claims, wherein the step of processing the empty-LNP solution or loaded-LNP solution further comprises lyophilizing the empty-LNP solution or loaded-LNP solution.

28. The method of any one of the preceding claims, wherein the step of processing the empty-LNP solution or loaded-LNP solution further comprises storing the empty-LNP solution or loaded-LNP solution.

29. The method of any one of the preceding claims, wherein the mixing step is performed with a T-junction, confined impinging jets, microfluidic mixer, or vortex mixer.

30. The method of any one of the preceding claims, wherein the loading step is performed with a T-junction, confined impinging jets, microfluidic mixer, or vortex mixer.

31. The method of any one of the preceding claims, wherein the aqueous buffer solution has a pH in a range of from about 4.5 to about 6.5, from about 4.6 to about 6.0, from about 4.7 to about 5.75, from about 4.8 to about 5.5, or from about 4.9 to about 5.25.

32. The method of any one of the preceding claims, wherein the aqueous buffer solution has a pH of about 5.0.

33. The method of any one of the preceding claims, wherein the empty-LNP solution has a pH in a range of from about 4.8 to about 5.8, from about 5.0 to about 5.75, or from about 5.0 to about 5.5.

34. The method of any one of the preceding claims, wherein the nucleic acid solution has a pH in a range of from about 4.5 to about 6.5, from about 4.8 to about 6.25, from about 4.8 to about 6.0, from about 5.0 to about 5.8, or from about 5.2 to about 5.5.

35. The method of any one of the preceding claims, wherein the pH of the nucleic acid solution, the empty-LNP solution, and the LNP formulation are in a range of from about 5.0 to about 6.0, from about 5.1 to about 5.75, or from about 5.2 to about 5.5.

36. The method of any one of the preceding claims, wherein the loaded-LNP solution has a pH in a range of from about 4.5 to about 6.0, from about 4.6 to about 5.8, from about 4.8 to about 5.6, from about 5.0 to about 5.5, or from about 5.1 to about 5.4.

37. The method of any one of the preceding claims, wherein the lipid solution further comprises a first organic solvent.

38. The method of any one of the preceding claims, wherein the empty-LNP solution or loaded-LNP solution further comprises a first organic solvent.

39. The method of any one of the preceding claims, wherein the first organic solvent is an alcohol.

40. The method of any one of the preceding claims, wherein the first organic solvent is ethanol.

41. The method of any one of the preceding claims, wherein the first buffering agent comprises a first aqueous buffer solution.

42. The method of any one of the preceding claims, wherein the first aqueous buffer is selected from the group consisting of an acetate buffer, a citrate buffer, a phosphate buffer, and a tris buffer.

43. The method of any one of the preceding claims, wherein the first aqueous buffer solution comprises greater than about 1 mM citrate, acetate, phosphate or tris, greater than about 2 mM citrate, acetate, phosphate or tris, greater than about 5 mM citrate, acetate, phosphate or tris, greater than about 10 mM citrate, acetate, phosphate or tris, greater than about 15 mM citrate, acetate, phosphate or tris, greater than about 20 mM citrate, acetate, phosphate or tris, greater than about 25 mM citrate, acetate, phosphate or tris, or greater than about 30 mM citrate, acetate, phosphate or tris.

44. The method of any one of the preceding claims, wherein the first aqueous buffer solution comprises about 1 mM to about 30 mM citrate, acetate, phosphate or tris, about 2 mM to about 20 mM citrate, acetate, phosphate or tris, about 3 mM to about 10 mM citrate, acetate, phosphate or tris, about 4 mM to about 8 mM citrate, acetate, phosphate or tris, or about 5 mM to about 6 mM citrate, acetate, phosphate or tris.

45. The method of any one of the preceding claims, wherein the first aqueous buffer solution comprises about 5 mM citrate, acetate, phosphate or tris.

46. The method of any one of the preceding claims, wherein the first aqueous buffer solution comprises about 5 mM acetate, wherein the aqueous buffer solution has a pH of about 5.0.

47. The method of any one of the preceding claims, wherein the empty-LNP solution or loaded-LNP solution further comprises a tonicity agent.

48. The method of any one of the preceding claims, wherein the tonicity agent is a sugar.

49. The method of any one of the preceding claims, wherein the sugar is sucrose.

50. The method of any one of the preceding claims, wherein the empty-LNP solution or loaded-LNP solution comprises from about 0.01 g/mL to about 1.0 g/mL, from about 0.05 g/mL to about 0.5 g/mL, from about 0.1 g/mL to about 0.4 g/mL, from about 0.15 g/mL to about 0.3 g/mL, or from about 0.2 g/mL to about 0.25 g/mL tonicity agent.

51. The method of any one of the preceding claims, wherein the empty-LNP solution or loaded-LNP solution further comprises from about 0.2 g/mL to about 0.25 g/mL tonicity agent.

52. The method of any one of the preceding claims, wherein the empty-LNP solution or loaded-LNP solution further comprises about 0.2 g/mL sucrose.

53. The method of any one of the preceding claims, wherein the nucleic acid solution comprises about 0.01 to about 1.0 mg/mL of the nucleic acid, about 0.05 to about 0.5 mg/mL of the nucleic acid, or about 0.1 to about 0.25 mg/mL of the nucleic acid.

54. The method of any one of the preceding claims, wherein the nucleic acid solution comprises a buffer selected from the group consisting of an acetate buffer, a citrate buffer, a phosphate buffer, and a tris buffer.

55. The method of any one of the preceding claims, wherein the nucleic acid solution comprises an acetate buffer.

56. The method of any one of the preceding claims, wherein the nucleic acid solution comprises from about 1 mM to about 200 mM acetate buffer, from about 2 mM to about 180 mM acetate buffer, from about 3 mM to about 160 mM acetate buffer, from about 4 mM to about 150 mM acetate buffer, from about 4 mM to about 140 mM acetate buffer, from about 5 mM to about 130 mM acetate buffer, from about 6 mM to about 120 mM acetate buffer, from about 7 mM to about 110 mM acetate buffer, from about 8 mM to about 100 mM acetate buffer, from about 9 mM to about 90 mM acetate buffer, from about 10 mM to about 80 mM acetate buffer, from about 15 mM to about 70 mM acetate buffer, from about 20 mM to about 60 mM acetate buffer, from about 25 mM to about 50 mM acetate buffer, or from about 30 mM to about 40 mM acetate buffer.

57. The method of any one of the preceding claims, wherein the nucleic acid solution and the empty-LNP solution are mixed at a volumetric flow ratio of from about 5:1 to about 7:1, from about 4:1 to about 6:1, from about 3:1 to about 5:1, or from about 2:1 to about 4:1 during the loading step.

58. The method of any one of the preceding claims, wherein the loaded-LNP solution comprises an acetate buffer.

59. The method of any one of the preceding claims, wherein the lipid solution, the empty LNP, the empty-LNP solution, the loaded LNP, the loaded-LNP solution, and/or the LNP formulation further comprises an encapsulation agent.

60. The method of any one of the preceding claims, wherein the lipid solution, the empty-LNP solution, the loaded LNP, the loaded-LNP solution, and/or the LNP formulation further comprises a phospholipid, a PEG lipid, a structural lipid, or any combination thereof.

61. The method of any one of the preceding claims, wherein the empty LNP comprises about 30-60 mol% ionizable lipid;
about 0-30 mol% phospholipid;
about 15-50 mol% structural lipid; and
about 0.1-0.5 mol% PEG lipid.

62. The method of any one of the preceding claims, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol.

63. The method of any one of the preceding claims, wherein the structural lipid is selected from the group consisting of cholesterol, fecosterol, sitosterol, ergosterol, campesterol, stigmasterol, brassicasterol, tomatidine, ursolic acid, alpha-tocopherol, and derivatives thereof.

64. The method of any one of the preceding claims, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine,1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and derivatives thereof.

65. The method of any one of the preceding claims, wherein the ionizable lipid comprises an ionizable amino lipid.

66. The method of any one of the preceding claims, wherein the nucleic acid is a ribonucleic acid.

67. The method of any one of the preceding claims, wherein the ribonucleic acid is at least one ribonucleic acid selected from the group consisting of a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and a long non-coding RNA (lncRNA).

68. The method of any one of the preceding claims, wherein the nucleic acid is a messenger RNA (mRNA).

69. The method of any one of the preceding claims, wherein the mRNA includes at least one motif selected from the group consisting of a stem loop, a chain terminating nucleoside, a polyA sequence, a polyadenylation signal, and a 5′ cap structure.

70. The method of any one of the preceding claims, wherein the mRNA is at least 30 nucleotides in length.

71. The method of any one of the preceding claims, wherein the mRNA is at least 300 nucleotides in length.

72. The method of any one of the preceding claims, wherein the LNP formulation has a N:P ratio from about 1.1:1 to about 30.1.

73. The method of any one of the preceding claims, wherein the LNP formulation has a N:P ratio from about 2:1 to about 20:1.

74. The method of any one of the preceding claims, wherein the LNP formulation has a N:P ratio from about 2:1 to about 10:1 or about 2:1 to about 5:1.

75. The method of any one of the preceding claims, wherein the LNP formulation comprises from about 0.01 to about 500 mg/mL of the nucleic acid, from about 0.1 to about 100 mg/mL, from about 0.25 to about 50 mg/mL, from about 0.5 to about 10 mg/mL, or from about 1.0 to about 10 mg/mL of the nucleic acid.

76. An empty LNP comprising from about 0.1 mol% to about 0.5 mol% of a PEG lipid.

77. An empty LNP prepared by the method of any one of the preceding claims.

78. An empty-LNP solution prepared by the method of any one of the preceding claims.

79. An empty-LNP solution comprising an empty LNP, wherein the empty LNP comprises from about 0.1 mol% to about 0.5 mol% of a PEG lipid.

80. A loaded LNP prepared by the method of any one of the preceding claims.

81. A loaded-LNP solution prepared by the method of any one of the preceding claims.

82. A LNP formulation prepared by the method of any one of the preceding claims.

83. A method of treating or preventing a disease or disorder, the method comprising administering to a subject in need thereof the loaded LNP of any one of the preceding claims.

84. A method of treating or preventing a disease or disorder, the method comprising administering to a subject in need thereof the loaded-LNP solution of any one of the preceding claims.

85. A method of treating or preventing a disease or disorder, the method comprising administering to a subject in need thereof the LNP formulation of any one of the preceding claims.

86. The method of any one of the preceding claims, wherein the administering is performed parenterally.

87. The method of any one of the preceding claims, wherein the administering is performed intramuscularly, intradermally, subcutaneously, and/or intravenously.

88. The loaded LNP of any one of the preceding claims for use in treating or preventing a disease or disorder in a subject.

89. The loaded-LNP solution any one of the preceding claims for use in treating or preventing a disease or disorder in a subject.

90. The LNP formulation of any one of the preceding claims for use in treating or preventing a disease or disorder in a subject.

91. Use of the loaded LNP of any one of the preceding claims in the manufacture of a medicament for treating or preventing a disease or disorder.

92. Use of the loaded-LNP solution of any one of the preceding claims in the manufacture of a medicament for treating or preventing a disease or disorder.

93. Use of the LNP formulation of any one of the preceding claims in the manufacture of a medicament for treating or preventing a disease or disorder.

94. A pharmaceutical kit, comprising the empty LNP, empty-LNP solution, loaded LNP, loaded-LNP solution, or LNP formulation of any one of claims 77-83.

95. An empty LNP comprising from about 0.1 mol% to about 1.25 mol% of a PEG lipid.

96. An empty LNP comprising from about 0.1 mol% to about 0.5 mol% of a PEG lipid.

97. The empty LNP of any one of the preceding claims, further comprising an ionizable lipid.

98. The empty LNP of any one of the preceding claims, further comprising a phospholipid and a structural lipid.

99. An empty LNP comprising about 30-60 mol% ionizable lipid; about 0-30 mol% phospholipid; about 15-50 mol% structural lipid; and about 0.1-10 mol% PEG lipid.

100. An empty-LNP solution comprising the empty LNP of any one of the preceding claims.

101. The empty-LNP solution of any one of the preceding claims, further comprising an acetate buffer.

102. The empty-LNP solution of any one of the preceding claims, further comprising a tonicity agent.

103. The empty-LNP solution of any one of the preceding claims, wherein the tonicity agent is sucrose.

104. An empty-LNP solution comprising:
(i) an empty LNP comprising from about 0.1 mol% to about 1.25 mol% of a PEG lipid; and
(ii) an acetate buffer.

105. An empty-LNP solution comprising:
(i) an empty LNP comprising from about 0.1 mol% to about 1.25 mol% of a PEG lipid;
(ii) an acetate buffer; and
(iii) sucrose.

106. An empty-LNP solution comprising:
(i) an empty LNP comprising from about 0.1 mol% to about 0.5 mol% of a PEG lipid; and
(ii) an acetate buffer.

107. An empty-LNP solution comprising:
(i) an empty LNP comprising from about 0.1 mol% to about 0.5 mol% of a PEG lipid;
(ii) an acetate buffer; and
(iii) sucrose.

108. The empty-LNP solution of any one of the preceding claims, having a pH value of from about 4.5 to about 6.25, from about 4.6 to about 6.0, from about 4.8 to about 5.8, from about 5.0 to about 5.75, or from about 5.0 to about 5.5.

109. The empty-LNP solution of any one of the preceding claims, comprising about 5 mM acetate buffer, wherein the acetate buffer has a pH of about 5.0.

110. The empty-LNP solution of any one of the preceding claims, comprising about 0.2 g/mL sucrose.

111. The empty-LNP solution of any one of the preceding claims, wherein the empty LNP comprises from about 30 mol% to about 60 mol% of the ionizable lipid, from about 0 mol% to about 30 mol% of the phospholipid, from about 15 mol% to about 50 mol% of the structural lipid, and from about 0.1 mol% to about 0.5 mol% of the PEG lipid.

112. The empty-LNP solution of any one of the preceding claims, wherein the empty LNP comprises from about 40 mol% to about 60 mol%% of the ionizable lipid, from about 5 mol% to about 20 mol% of the phospholipid, from about 30 mol% to about 50 mol% of the structural lipid, and from about 0.1 mol % to about 1.25 mol % of the PEG lipid.

113. The empty-LNP solution of any one of the preceding claims, wherein the PEG lipid is present at a concentration of about 0.2 mol% to about 0.7 mol%.

114. The empty-LNP solution of any one of the preceding claims, wherein the PEG lipid is present at a concentration of about 0.5 mol%.

115. The empty-LNP solution of any one of the preceding claims, wherein the empty-LNP solution comprises an acetate buffer having a concentration of from about 2 mM to about 40 mM.

116. The empty-LNP solution of any one of the preceding claims, wherein the empty-LNP solution comprises an acetate buffer having a concentration of from about 2 mM to about 30 mM.

117. The empty-LNP solution of any one of the preceding claims, wherein the empty-LNP solution comprises an acetate buffer having a concentration of from about 2 mM to about 20 mM.

118. The empty-LNP solution of any one of the preceding claims, wherein the empty-LNP solution comprises an acetate buffer having a concentration of from about 2 mM to about 10 mM.

119. The empty-LNP solution of any one of the preceding claims, wherein the empty-LNP solution comprises an acetate buffer having a concentration of about 5 mM.

120. The empty-LNP solution of any one of the preceding claims, wherein the buffer has a pH of at least 1 unit less than the pKa of the ionizable lipid.

121. The empty-LNP solution of any one of the preceding claims, wherein the buffer has a pH of less than 5.5.

122. The empty-LNP solution of any one of the preceding claims, wherein the buffer has a pH of about 5.0.

123. The empty-LNP solution of any one of the preceding claims, wherein the empty-LNP solution has a pH of at least 1 unit less than the pKa of the ionizable lipid.

124. The empty-LNP solution of any one of the preceding claims, wherein the empty-LNP solution has a pH of less than 5.5.

125. The empty-LNP solution of any one of the preceding claims, wherein the empty-LNP solution has a pH of about 5.0.

126. The empty-LNP solution of any one of the preceding claims, wherein the LNPs comprise about 45 mol% to about 50 mol% ionizable lipid.

127. The empty-LNP solution of any one of the preceding claims, wherein the ionizable lipid is, 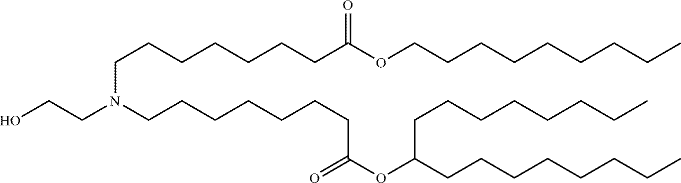 [SMILES: CCCCCCCCCOC(=O)CCCCCCCN(CCO)CCCCCCCC(=O)OC(CCCCCCCC)CCCCCCCC]
or a salt thereof.

128. The empty-LNP solution of any one of the preceding claims, wherein the ionizable lipid is, 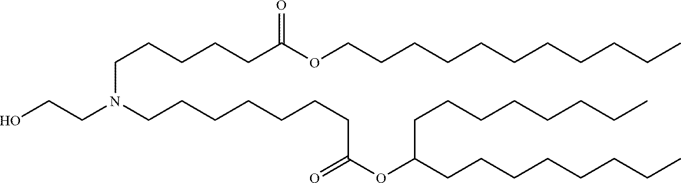 [SMILES: CCCCCCCCCCCOC(=O)CCCCCN(CCO)CCCCCCCC(=O)OC(CCCCCCCC)CCCCCCCC]
or a salt thereof.

129. The empty-LNP solution of any one of the preceding claims, wherein the PEG lipid is PEG₂ₖ-DMG.

130. The empty-LNP solution of any one of the preceding claims, wherein the structural lipid is cholesterol.

131. The empty-LNP solution of any one of the preceding claims, wherein the phospholipid is 1,2-distearoyl-sn-glycero-3-phosphocholine (DSPC).

132. A preparation comprising lipid nanoparticles (LNPs), wherein:
(a) the LNPs comprise
from about 40 mol% to about 50 mol% ionizable lipid,
from about 30 mol% to about 50 mol% structural lipid,
from about 5 mol% to about 20 mol% phospholipid, and
from about 0.1 mol% to about 1.25 mol% of a PEG lipid;
(b) the LNPs are substantially free of a therapeutic or prophylactic agent; and
(c) the preparation comprises an acetate buffer having a concentration of from about 2 mM to about 40 mM.

133. The preparation of any one of the preceding claims, wherein the PEG lipid is present at a concentration of about 0.2 mol% to about 0.7 mol%.

134. The preparation of any one of the preceding claims, wherein the PEG lipid is present at a concentration of about 0.5 mol%.

135. The preparation of any one of the preceding claims, wherein the preparation comprises an acetate buffer having a concentration of from about 2 mM to about 30 mM.

136. The preparation of any one of the preceding claims, wherein the preparation comprises an acetate buffer having a concentration of from about 2 mM to about 20 mM.

137. The preparation of any one of the preceding claims, wherein the preparation comprises an acetate buffer having a concentration of from about 2 mM to about 10 mM.

138. The preparation of any one of the preceding claims, wherein the preparation comprises an acetate buffer having a concentration of about 5 mM.

139. The preparation of any one of the preceding claims, wherein the buffer has a pH of at least 1 unit less than the pKa of the ionizable lipid.

140. The preparation of any one of the preceding claims, wherein the buffer has a pH of less than 5.5.

141. The preparation of any one of the preceding claims, wherein the buffer has a pH of about 5.0.

142. The preparation of any one of the preceding claims, wherein the LNPs comprise about 45 mol% to about 50 mol% ionizable lipid.

143. The preparation of any one of the preceding claims, wherein the ionizable lipid is 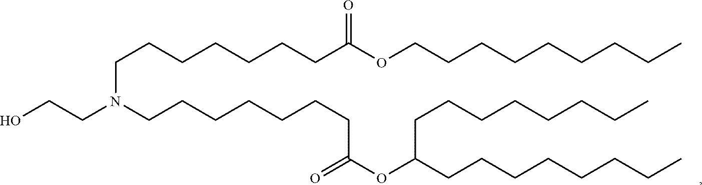
or a salt thereof.

144. The preparation of any one of the preceding claims, wherein the ionizable lipid is 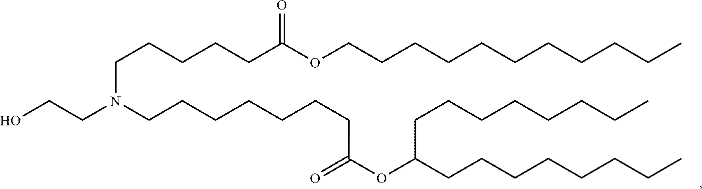
or a salt thereof.

145. The preparation of any one of the preceding claims, wherein the PEG lipid is PEG₂ₖ-DMG.

146. The preparation of any one of the preceding claims, wherein the structural lipid is cholesterol.

147. The preparation of any one of the preceding claims, wherein the phospholipid is 1,2-distearoyl-sn-glycero-3-phosphocholine (DSPC).
